SEQUENCE LISTING (375 sequences)

SEQ ID NO: 1 - protein, 107 aa, synthetic construct
EIVMTQSPATLSVSPGERATLSCRASQSVSSNLAWFQQKPGQAPRLLIYGASTRATGIPARFSGSGSGTEFTLTISSLQPEDFAVYYCLQDNNFPPTFGQGTKVDIK

SEQ ID NO: 2 - protein, 11 aa, synthetic construct
RASQSVSSNLA

SEQ ID NO: 3 - protein, 7 aa, synthetic construct
GASTRAT

SEQ ID NO: 4 - protein, 9 aa, synthetic construct
LQDNNFPPT

SEQ ID NO: 5 - protein, 121 aa, synthetic construct
EVQLVQSGAEVKKPGESLKISCKGSGYSFTSYWIGWVRQMPGKGLEWMGIIYPGDADARYSPSFQGQVTISADKSISTAYLQWSSLKASDTAMYFCARRRQGIFGDALDFWGQGTLVTVSS

SEQ ID NO: 6 - protein, 5 aa, synthetic construct
SYWIG

SEQ ID NO: 7 - protein, 17 aa, synthetic construct
IIYPGDADARYSPSFQG

SEQ ID NO: 8 - protein, 12 aa, synthetic construct
RRQGIFGDALDF

SEQ ID NO: 9 - protein, 214 aa, synthetic construct
EIVMTQSPATLSVSPGERATLSCRASQSVSSNLAWFQQKPGQAPRLLIYGASTRATGIPARFSGSGSGTEFTLTISSLQPEDFAVYYCLQDNNFPPTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 451 aa, synthetic construct
EVQLVQSGAEVKKPGESLKISCKGSGYSFTSYWIGWVRQMPGKGLEWMGIIYPGDADARYSPSFQGQVTISADKSISTAYLQWSSLKASDTAMYFCARRRQGIFGDALDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 11 aa, synthetic construct
RASQSVNNNLA

SEQ ID NO: 12 - protein, 12 aa, synthetic construct
RASQIISSNYLA

SEQ ID NO: 13 - protein, 12 aa, synthetic construct
RASQSVSSSYLA

SEQ ID NO: 14 - protein, 11 aa, synthetic construct
RASQDINSWLA

SEQ ID NO: 15 - protein, 11 aa, synthetic construct
RASQGISNWLA

SEQ ID NO: 16 - protein, 11 aa, synthetic construct
RASQGISSWLA

SEQ ID NO: 17 - protein, 11 aa, synthetic construct
RASQGISNYLA

SEQ ID NO: 18 - protein, 11 aa, synthetic construct
RASQGIRNDLG

SEQ ID NO: 19 - protein, 11 aa, synthetic construct
YKSDSDKQQGS

SEQ ID NO: 20 - protein, 7 aa, synthetic construct
QDSKRPS

SEQ ID NO: 21 - protein, 7 aa, synthetic construct
QDTKRPS

SEQ ID NO: 22 - protein, 7 aa, synthetic construct
WASTRES

SEQ ID NO: 23 - protein, 7 aa, synthetic construct
GASTRAT

SEQ ID NO: 24 - protein, 7 aa, synthetic construct
SASSRAT

SEQ ID NO: 25 - protein, 7 aa, synthetic construct
GASSRAT

SEQ ID NO: 26 - protein, 7 aa, synthetic construct
AASSLQT

SEQ ID NO: 27 - protein, 7 aa, synthetic construct
AASSLQV

SEQ ID NO: 28 - protein, 7 aa, synthetic construct
AASSLQN

SEQ ID NO: 29 - protein, 7 aa, synthetic construct
AASSLQS

SEQ ID NO: 30 - protein, 7 aa, synthetic construct
AASSLPS

SEQ ID NO: 31 - protein, 9 aa, synthetic construct
MIWYSSAVV

SEQ ID NO: 32 - protein, 9 aa, synthetic construct
QAWDSNTVV

SEQ ID NO: 33 - protein, 9 aa, synthetic construct
QAWDSSTVV

SEQ ID NO: 34 - protein, 9 aa, synthetic construct
QQYYITPIT

SEQ ID NO: 35 - protein, 9 aa, synthetic construct
QQYYSTPLT

SEQ ID NO: 36 - protein, 9 aa, synthetic construct
HQYYSTPCS

SEQ ID NO: 37 - protein, 9 aa, synthetic construct
LQDNNWPPT

SEQ ID NO: 38 - protein, 9 aa, synthetic construct
QQYNNWPRT

SEQ ID NO: 39 - protein, 9 aa, synthetic construct
QQFDSSPIT

SEQ ID NO: 40 - protein, 9 aa, synthetic construct
QQYDTSPFT

SEQ ID NO: 41 - protein, 9 aa, synthetic construct
QQSNSFPIT

SEQ ID NO: 42 - protein, 9 aa, synthetic construct
QQADSFPRN

SEQ ID NO: 43 - protein, 9 aa, synthetic construct
QQADSFPRT

SEQ ID NO: 44 - protein, 9 aa, synthetic construct
QQYSTYPFT

SEQ ID NO: 45 - protein, 9 aa, synthetic construct
LQHNSYPWT

SEQ ID NO: 46 - protein, 115 aa, synthetic construct
QAVPTQPSSLSASPGVLASLTCTLRSGINVGTYRIYWYQQKPGSPPQYLLRYKSDSDKQQGSGVPSRFSGSKDASANAGILLISGLQSEDEADYYCMIWYSSAVVFGGGTKLTVL

SEQ ID NO: 47 - protein, 106 aa, synthetic construct
SYELTQPPSVSVSPGQTASITCSGDKLGDKYVCWYQQKPGQSPVLVIYQDSKRPSGIPERFSGSNSGNTATLTISGTQAMDEADYYCQAWDSNTVVFGGGTKLTVL

SEQ ID NO: 48 - protein, 106 aa, synthetic construct
SFELTQPPSVSVSPGQTASITCSGDKLGDKYVCWYQQKPGQSPMLVIYQDTKRPSGIPERFSGSNSGNTATLTISGTQAMDEADYYCQAWDSSTVVFGGGTKLTVL

SEQ ID NO: 49 - protein, 106 aa, synthetic construct
SYELTQPPSVSVSPGQTASITCSGDKLGDKYVCWYQQKPGQSPVLVIFQDSKRPSGIPERFSGSNSGNTATLTISGTQAMDEADYYCQAWDSSTVVFGGGTKLTVL

SEQ ID NO: 50 - protein, 106 aa, synthetic construct
SYELTQPPSVSVSPGQTASITCSGDKLGDKYVCWYQQKPGQSPVLVIYQDSKRPSGIPERFSGSNSGNTATLTISGTQAMDEADYYCQAWDSSTVVFGGGTKLTVL

SEQ ID NO: 51 - protein, 113 aa, synthetic construct
GIVMTQSPDSLAVSLGERATINCKSSRSVLYSSNNKNYLAWYQQKPGQPPKVLIYWASTRESGVPDRFSGSGSGTDFTLTISSLQAEDVAVYNCQQYYITPITFGQGTRLEIK

SEQ ID NO: 52 - protein, 113 aa, synthetic construct
DIVMTQSPDSLAVSLGERATINCKSSQSVLYSSNNKHFLAWYQQKPGQPPKLLIYWASTRESGVPDRFSGSGSGTDFTLTISSLQAEDVAFYYCQQYYSTPLTFGGGTKVEIK

SEQ ID NO: 53 - protein, 113 aa, synthetic construct
DIVMTQSPDSLAVSLGERATITCKSSHNVLYSSNNKNYLAWYQQKPGQPPKLLIYWASTRESGVPDRFSGSGSGTDFTLTISSLQAEDVAVYYCHQYYSTPCSFGQGTKLEIK

SEQ ID NO: 54 - protein, 107 aa, synthetic construct
EIVMTQSPATLSVSPGERATLSCRASQSVSSNLAWFQQKPGQAPRLLIYGASTRATGIPARFSVSGSGTEFTLTISSLQSEDFAFYYCLQDNNWPPTFGPGTKVDIK

SEQ ID NO: 55 - protein, 107 aa, synthetic construct
EIVMTQSPATLSVSPGERATLSCRASQSVSSNLAWFQQKPGQAPRLLIYGASTRATGIPARFSVSGSGTEFTLTISSLQSEDFAVYYCLQDNNWPPTFGPGTKVDIK

SEQ ID NO: 56 - protein, 107 aa, synthetic construct
EKVMTQSPATLSVSPGERATLSCRASQSVNNNLAWYQQKPGQAPRLLIYGASTRATGIPARFSGSGSGTEFTLTISSLQSEDFAVYYCQQYNNWPRTFGQGTKVEIK

SEQ ID NO: 57 - protein, 108 aa, synthetic construct
EFVLTQSPGTLSLSPGERATLSCRASQIISSNYLAWYQQKPGQAPRLLIYSASSRATGIPDRFSGSGSGTDFTLTISRLEPEDFAVYYCQQFDSSPITFGRGTRLDIK

SEQ ID NO: 58 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFALYYCQQYDTSPFTFGPGTKVDIK

SEQ ID NO: 59 - protein, 107 aa, synthetic construct
DIQMTQSPSSVSASVGDRVTVTCRASQDINSWLAWYQQKPGKAPKLLIYAASSLQTGVPSRFSGSGSGTDFTLTISSLQPEDFATYSCQQSNSFPITFGQGTRLEIK

SEQ ID NO: 60 - protein, 107 aa, synthetic construct
DIQMTQSPSSVSASVGDRVTITCRASQGISNWLAWYQQKPGKAPKLLIYAASSLQVGVPLRFSGSGSGTDFTLTISSLQPEDFATYYCQQADSFPRNFGQGTKLEIK

SEQ ID NO: 61 - protein, 107 aa, synthetic construct
DIQMTQSPSSVSASVGDRVTITCRASQGISSWLAWYQQKPGKAPKLLIYAASSLQNGVPSRFSGSGSGTDFTLTISSLQPEDFATYFCQQADSFPRTFGQGTKLEIK

SEQ ID NO: 62 - protein, 107 aa, synthetic construct
DIQMTQSPSSLSASVGDRVTITCRASQGISNYLAWFQQKPGKAPKSLIYAASSLQSGVPSKFSGSGSGTDFTLTISSLQPEDFATYYCQQYSTYPFTFGPGTKVDIK

SEQ ID NO: 63 - protein, 107 aa, synthetic construct
DIQMTQSPSSLSASVGDRVTITCRASQGIRNDLGWYQQKPGNAPKRLIYAASSLPSGVPSRFSGSGSGPEFTLTISSLQPEDFATYYCLQHNSYPWTFGQGTKVEIT

SEQ ID NO: 64-76 - skipped (13 entries)

SEQ ID NO: 77 - protein, 5 aa, synthetic construct
SYAMS

SEQ ID NO: 78 - protein, 5 aa, synthetic construct
SFGMS

SEQ ID NO: 79 - protein, 5 aa, synthetic construct
NYAMS

SEQ ID NO: 80 - protein, 5 aa, synthetic construct
NYWIG

SEQ ID NO: 81 - protein, 5 aa, synthetic construct
SYWIG

SEQ ID NO: 82 - protein, 5 aa, synthetic construct
SYYWS

SEQ ID NO: 83 - protein, 5 aa, synthetic construct
NYGIS

SEQ ID NO: 84 - protein, 5 aa, synthetic construct
NYWIA

SEQ ID NO: 85 - protein, 5 aa, synthetic construct
SYWIA

SEQ ID NO: 86 - protein, 5 aa, synthetic construct
GYYIH

SEQ ID NO: 87 - protein, 17 aa, synthetic construct
AIGGGGVSTYCADSVKG

SEQ ID NO: 88 - protein, 17 aa, synthetic construct
YISSSSFTIYYADSVKG

SEQ ID NO: 89 - protein, 17 aa, synthetic construct
YISKSSFTIYYADSVKG

SEQ ID NO: 90 - protein, 17 aa, synthetic construct
AISGSGGSTYYADSVKG

SEQ ID NO: 91 - protein, 17 aa, synthetic construct
IIYPGDSDTRYSPSFQG

SEQ ID NO: 92 - protein, 16 aa, synthetic construct
EINHSGNTNYNPSLKS

SEQ ID NO: 93 - protein, 17 aa, synthetic construct
WISAYNGNTNYAQKLQG

SEQ ID NO: 94 - protein, 17 aa, synthetic construct
WINPYSGGTTSAQKFQG

SEQ ID NO: 95 - protein, 12 aa, synthetic construct
FYIAVAGSHFDY

SEQ ID NO: 96 - protein, 17 aa, synthetic construct
EGGLTMVRGVSSYGLDV

SEQ ID NO: 97 - protein, 17 aa, synthetic construct
EGGITMVRGVSSYGMDV

SEQ ID NO: 98 - protein, 10 aa, synthetic construct
AYTPMAFFDY

SEQ ID NO: 99 - protein, 7 aa, synthetic construct
GGWELFY

SEQ ID NO: 100 - protein, 12 aa, synthetic construct
RRQGIWGDALDI

SEQ ID NO: 101 - protein, 12 aa, synthetic construct
RRQGIWGDALDF

SEQ ID NO: 102 - protein, 16 aa, synthetic construct
EGYYDILTGYHDAFDI

SEQ ID NO: 103 - protein, 13 aa, synthetic construct
HDIIPAAPGAFDI

SEQ ID NO: 104 - protein, 13 aa, synthetic construct
QAIAVTGLGGFDP

SEQ ID NO: 105 - protein, 10 aa, synthetic construct
RGYSYGSFDY

SEQ ID NO: 106 - protein, 14 aa, synthetic construct
QRTFYYDSSGYFDY

SEQ ID NO: 107 - protein, 14 aa, synthetic construct
QRTFYYDSSDYFDY

SEQ ID NO: 108 - protein, 12 aa, synthetic construct
DGGYLALYGTDV

SEQ ID NO: 109 - protein, 13 aa, synthetic construct
QGIEVTGTGGLDV

SEQ ID NO: 110 - protein, 121 aa, synthetic construct
EVQLLESGGGLVQPGGSLRLSCAASGFTFSSYAMSWVRQAPGKGLEWVSAIGGGGVSTYCADSVKGRFTISRDNSKNTLYLQMNSLRAEDTAVYYCAKFYIAVAGSHFDYWGQGTLVTVSS

SEQ ID NO: 111 - protein, 126 aa, synthetic construct
EVQLVESGGALVQRGGSLRLSCAASRFTFSSFGMSWVRQAPGKGLEWVSYISSSSFTIYYADSVKGRFTISRDNAKNSFYLQMNSLRDEDTAVYYCAREGGLTMVRGVSSYGLDVWGQGTTVTVSS

SEQ ID NO: 112 - protein, 126 aa, synthetic construct
EVQLVESGGALVQPGGSLRLSCAASGFTFSSFGMSWVRQAPGKGLEWVSYISSSSFTIYYADSVKGRFTISRDNAKNSFYLQMNSLRDEDTAVYFCVREGGITMVRGVSSYGMDVWGQGTTVTVSS

SEQ ID NO: 113 - protein, 126 aa, synthetic construct
EVQLVESGGALVQPGGSLRLSCAASGFTFSSFGMSWVRQAPGKGLEWISYISSSSFTIYYADSVKGRFTISRDNAKNSFYLQMNSLRDEDTAVYFCAREGGITMVRGVSSYGMDVWGQGTTVTVSS

SEQ ID NO: 114 - protein, 126 aa, synthetic construct
EVQLVESGGALVQPGGSLRLSCAASGFTFSSFGMSWVRQAPGKGLEWVSYISKSSFTIYYADSVKGRFTISRDNAKNSFYLQMNSLRDEDTAVYYCAREGGLTMVRGVSSYGLDVWGQGTTVTVSS

SEQ ID NO: 115 - protein, 119 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKAYTPMAFFDYWGQGTLVTVSS

SEQ ID NO: 116 - protein, 116 aa, synthetic construct
EVQVLESGGGLVQPGGSLRLSCAASGFTFSNYAMSWVRQAPGKGLEWVSAISGSGGSTYYADSVKGRFTISRDNSKNTLYLQMNSLRAEDTAVYYCAKGGWELFYWGQGTLVTVSS

SEQ ID NO: 117 - protein, 121 aa, synthetic construct
EVQLVQSGAEVKKPGESLMISCKGSGYSFTNYWIGWVRQMPGKGLEWMGIIYPGDSDTRYSPSFQGQVTISADKSISTAYLQWSSLKASDTAMYFCARRRQGIWGDALDIWGQGTLVTVSS

SEQ ID NO: 118 - protein, 121 aa, synthetic construct
EVQLVQSGAEVKKPGESLMISCKGSGYSFTSYWIGWVRQMPGKGLEWMGIIYPGDSDTRYSPSFQGQVTISADKSISTAYLQWSSLKASDTAMYFCARRRQGIWGDALDFWGQGTLVTVSS

SEQ ID NO: 119 - protein, 124 aa, synthetic construct
QVQLQQWGAGLLKPSETLSLTCAVYGGSFSSYYWSWIRQPPGKGLEWIGEINHSGNTNYNPSLKSRVTISVDTSKNQFSLKLSSVTAADTAVYYCAREGYYDILTGYHDAFDIWDQGTMVTVFS

SEQ ID NO: 120 - protein, 122 aa, synthetic construct
EVQLVQSGAEVKKPGESLKISCKGSGYSFTSYWIGWVRQMPGKGLEWMGIIYPGDSDTRYSPSFQGQVTISADKSISTAYLQWSTLKASDTAIYYCARHDIIPAAPGAFDIWGQGTMVTVSS

SEQ ID NO: 121 - protein, 122 aa, synthetic construct
EVQLVQSGAEVKKPGESLKISCKGSGYTFTSYWIGWVRQMPGKGLEWMGIIYPGDSDTRYSPSFQGQVTISVDKSSSTAYLQWSSLKASDTAIYYCTRQAIAVTGLGGFDPWGQGTLVTVSS

SEQ ID NO: 122 - protein, 119 aa, synthetic construct
QVQLVQSGAEVKKPGASVKVSCKASGYTFTNYGISWVRQAPGQGLEWMGWISAYNGNTNYAQKLQGRVTMTTDTSTSTVYMELRSLRSDDTAVYYCARRGYSYGSFDYWGQGTLVTVSS

SEQ ID NO: 123 - protein, 123 aa, synthetic construct
EVQLVQSGAEVKKPGESLKISCKGSGHSFTNYWIAWVRQMPGKGLEWMGIIYPGDSDTRYSPSFQGQVTISADKSISTAYLQWSSLKASDTAVYFCARQRTFYYDSSGYFDYWGQGTLVTVSS

SEQ ID NO: 124 - protein, 123 aa, synthetic construct
EVQLVQSGAEVKKPGESLKISCKGSGYSFTSYWIAWVRQMPGKGLEWMGIIYPGDSDTRYSPSFQGQVTISADKSISTAYLQWSSLKASDTAMYFCARQRTFYYDSSDYFDYWGQGTLVTVSS

SEQ ID NO: 125 - protein, 121 aa, synthetic construct
QVQLVQSGAEVKKPGASVKVSCKASGYTFTGYYIHWVRQAPGLGLEWMGWINPYSGGTTSAQKFQGRVTMTRDTSISSAYMELSRLRSDDTAVYYCARDGGYLALYGTDVWGQGTTVTVSS

SEQ ID NO: 126 - protein, 122 aa, synthetic construct
EVQLVQSGAEVKKPGESLKISCKGSGYSFPSYWIAWVRQMPGKGLEWMGIIYPGDSDTRYSPSFQGQVTISADKSISTAFLKWSSLKASDTAMYFCARQGIEVTGTGGLDVWGQGTTVTVSS

SEQ ID NO: 127-138 - skipped (12 entries)

SEQ ID NO: 139 - protein, 7 aa, synthetic construct
  VARIANT 1: note = X is A or G
  VARIANT 5: note = X is L or R
  VARIANT 7: note = X is N, K, R, L, or T
XASSXQX

SEQ ID NO: 140 - protein, 9 aa, synthetic construct
  VARIANT 1: note = X is Q or G
  VARIANT 5: note = X is S or R
  VARIANT 6: note = X is F, L, or Y
  VARIANT 8: note = X is R or H
XQADXXPXT

SEQ ID NO: 141 - protein, 17 aa, synthetic construct
  VARIANT 1: note = X is I or T
  VARIANT 9: note = X is T or V
  VARIANT 11: note = X is Y or L
  VARIANT 12: note = X is S or A
  VARIANT 14: note = X is S, G, or E
  VARIANT 17: note = X is G or D
XIYPGDSDXRXXPXFQX

SEQ ID NO: 142 - protein, 14 aa, synthetic construct
  VARIANT 1: note = X is Q, G, S, or M
  VARIANT 12: note = X is F or S
XRTFYYDSSDYXDY

SEQ ID NO: 143 - protein, 7 aa, synthetic construct
AASSRQN

SEQ ID NO: 144 - protein, 7 aa, synthetic construct
AASSLQK

SEQ ID NO: 145 - protein, 7 aa, synthetic construct
AASSLQR

SEQ ID NO: 146 - protein, 7 aa, synthetic construct
AASSLQL

SEQ ID NO: 147 - protein, 7 aa, synthetic construct
GASSLQN

SEQ ID NO: 148 - protein, 9 aa, synthetic construct
QQADRFPRT

SEQ ID NO: 149 - protein, 9 aa, synthetic construct
QQADSFPHT

SEQ ID NO: 150 - protein, 9 aa, synthetic construct
QQADSLPRT

SEQ ID NO: 151 - protein, 9 aa, synthetic construct
QQADSYPRT

SEQ ID NO: 152 - protein, 9 aa, synthetic construct
GQADSFPRT

SEQ ID NO: 153 - protein, 107 aa, synthetic construct
DIQMTQSPSSVSASVGDRVTITCRASQGISSWLAWYQQKPGKAPKLLIYAASSRQNGVPSRFSGSGSGTDFTLTISSLQPEDFATYFCQQADRFPRTFGQGTKLEIK

SEQ ID NO: 154 - protein, 107 aa, synthetic construct
DIQMTQSPSSVSASVGDRVTITCRASQGISSWLAWYQQKPGKAPKLLIYAASSLQKGVPSRFSGSGSGTDFTLTISSLQPEDFATYFCQQADSFPHTFGQGTKLEIK

SEQ ID NO: 155 - protein, 107 aa, synthetic construct
DIQMTQSPSSVSASVGDRVTITCRASQGISSWLAWYQQKPGKAPKLLIYAASSLQRGVPSRFSGSGSGTDFTLTISSLQPEDFATYFCQQADSFPRTFGQGTKLEIK

SEQ ID NO: 156 - protein, 107 aa, synthetic construct
DIQMTQSPSSVSASVGDRVTITCRASQGISSWLAWYQQKPGKAPKLLIYAASSLQLGVPSRFSGSGSGTDFTLTISSLQPEDFATYFCQQADRFPRTFGQGTKLEIK

SEQ ID NO: 157 - protein, 107 aa, synthetic construct
DIQMTQSPSSVSASVGDRVTITCRASQGISSWLAWYQQKPGKAPKLLIYAASSLQTGVPSRFSGSGSGTDFTLTISSLQPEDFATYFCQQADSLPRTFGQGTKLEIK

SEQ ID NO: 158 - protein, 107 aa, synthetic construct
DIQMTQSPSSVSASVGDRVTITCRASQGISSWLAWYQQKPGKAPKLLIYAASSRQNGVPSRFSGSGSGTDFTLTISSLQPEDFATYFCQQADSYPRTFGQGTKLEIK

SEQ ID NO: 159 - protein, 107 aa, synthetic construct
DIQMTQSPSSVSASVGDRVTITCRASQGISSWLAWYQQKPGKAPKLLIYAASSLQRGVPSRFSGSGSGTDFTLTISSLQPEDFATYFCQQADRFPRTFGQGTKLEIK

SEQ ID NO: 160 - protein, 107 aa, synthetic construct
DIQMTQSPSSVSASVGDRVTITCRASQGISSWLAWYQQKPGKAPKLLIYAASSLQRGVPSRFSGSGSGTDFTLTISSLQPEDFATYFCGQADSFPRTFGQGTKLEIK

SEQ ID NO: 161 - protein, 107 aa, synthetic construct
DIQMTQSPSSVSASVGDRVTITCRASQGISSWLAWYQQKPGKAPKLLIYAASSLQKGVPSRFSGSGSGRDFTLTISSLQPEDFATYFCQQADSFPRTFGQGTKLEIK

SEQ ID NO: 162 - protein, 107 aa, synthetic construct
DIQMTQSPSSVSASVGDRVTITCRASQGISSWLAWYQQKPGKAPKLLIYGASSLQNGVPSRFSGSGSGTDFTLTISSLQPEDFATYFCQQADSFPRTFGQGTKLEIK

SEQ ID NO: 163 - protein, 4 aa, synthetic construct
YSFT

SEQ ID NO: 164 - protein, 4 aa, synthetic construct
YSFA

SEQ ID NO: 165 - protein, 4 aa, synthetic construct
YSFG

SEQ ID NO: 166 - protein, 4 aa, synthetic construct
YSFN

SEQ ID NO: 167 - protein, 4 aa, synthetic construct
YSFE

SEQ ID NO: 168 - protein, 4 aa, synthetic construct
YHFT

SEQ ID NO: 169 - protein, 4 aa, synthetic construct
YGFT

SEQ ID NO: 170 - protein, 17 aa, synthetic construct
IIYPGDSDTRYSPSFQD

SEQ ID NO: 171 - protein, 17 aa, synthetic construct
IIYPGDSDVRYSPSFQG

SEQ ID NO: 172 - protein, 17 aa, synthetic construct
IIYPGDSDTRYAPSFQG

SEQ ID NO: 173 - protein, 17 aa, synthetic construct
TIYPGDSDTRLSPSFQG

SEQ ID NO: 174 - protein, 17 aa, synthetic construct
IIYPGDSDTRYSPGFQG

SEQ ID NO: 175 - protein, 17 aa, synthetic construct
IIYPGDSDTRYSPEFQG

SEQ ID NO: 176 - protein, 14 aa, synthetic construct
GRTFYYDSSDYFDY

SEQ ID NO: 177 - protein, 14 aa, synthetic construct
SRTFYYDSSDYFDY

SEQ ID NO: 178 - protein, 14 aa, synthetic construct
QRTFYYDSSDYSDY

SEQ ID NO: 179 - protein, 14 aa, synthetic construct
MRTFYYDSSDYFDY

SEQ ID NO: 180 - protein, 123 aa, synthetic construct
EVQLVQSGAEVKKPGESLKISCKGSGYSFASYWIAWVRQMPGKGLEWMGIIYPGDSDTRYSPSFQDQVTISADKSISTAYLQWSSLKASDTAMYFCARGRTFYYDSSDYFDYWGQGTLVTVSS

SEQ ID NO: 181 - protein, 123 aa, synthetic construct
EVQLVQSGAEVKKPGESLKISCKGSGYSFTSYWIAWVRQMPGKGLEWMGIIYPGDSDVRYSPSFQGQVTISADKSISTAYLQWSSLKASDTAMYFCARSRTFYYDSSDYFDYWGQGTLVTVSS

SEQ ID NO: 182 - protein, 123 aa, synthetic construct
EVQLVQSGAEVKKPGESLKISCKGSGYSFTSYWIAWVRQMPGKGLEWMGIIYPGDSDTRYAPSFQGQVTISADKSISTAYLQWSSLKASDTAMYFCVRSRTFYYDSSDYFDYWGQGTLVTVSS

SEQ ID NO: 183 - protein, 123 aa, synthetic construct
EVQLVQSGAEVKKPGESLKISCKGSGYSFGSYWIAWVRQMPGKGLEWMGIIYPGDSDVRYSPSFQGQVTISADKSISTAYLQWSSLKASDTAMYFCARQRTFYYDSSDYSDYWGQGTLVTVSS

SEQ ID NO: 184 - protein, 123 aa, synthetic construct
EVQLVQSGAEVKKPGESLKISCKGSGYSFGSYWIAWVRQMPGKGLEWMGIIYPGDSDVRYSPSFQGQVTISADKSISTAYLQWSSLKASDTAMYFCARMRTFYYDSSDYFDYWGQGTLVTVSS

SEQ ID NO: 185 - protein, 123 aa, synthetic construct
EVQLVQSGAEVKKPGESLKISCKGSGYSFNSYWIAWVRQMPGKGLEWMGTIYPGDSDTRLSPSFQGQVTISADKSISTAYLQWSSLKASDTAMYFCARSRTFYYDSSDYFDYWGQGTLVTVSS

SEQ ID NO: 186 - protein, 123 aa, synthetic construct
EVQLVQSGAEVKKPGESLKISCKGSGYSFESYWIAWVRQMPGKGLEWMGIIYPGDSDTRYSPSFQGQVTISADKSISTAYLQWSSLKASDTAMYFCARGRTFYYDSSDYFDYWGQGTLVTVSS

SEQ ID NO: 187 - protein, 123 aa, synthetic construct
EVQLVQSGAEVKKPGESLKISCKGSGYHFTSYWIAWVRQMPGKGLEWMGIIYPGDSDVRYSPSFQGQVTISADKSISTAYLQWSSLKASDTAMYFCARQRTFYYDSSDYSDYWGQGTLVTVSS

SEQ ID NO: 188 - protein, 123 aa, synthetic construct
EVQLVQSGAEVKKPGESLKISCKGSGYSFGSYWIAWVRQMPGKGLEWMGIIYPGDSDTRYSPGFQGQVTISADKSISTAYLQWSSLKASDTAMYFCARGRTFYYDSSDYFDYWGQGTLVTVSS

SEQ ID NO: 189 - protein, 123 aa, synthetic construct
EVQLVQSGAEVKKPGESLKISCKGSGYSFGSYWIAWVRQMPGKGLEWMGIIYPGDSDTRYSPEFQGQVTISADKSISTAYLQWSSLKASDTAMYFCARQRTFYYDSSDYSDYWGQGTLVTVSS

SEQ ID NO: 190 - protein, 123 aa, synthetic construct
EVQLVQSGAEVKKPGESLKISCKGSGYGFTSYWIAWVRQMPGKGLEWMGIIYPGDSDTRYSPSFQGQVTISADKSISTAYLQWSSLKASDTAMYFCARQRTFYYDSSDYSDYWGQGTLVTVSS

SEQ ID NO: 191-207 - skipped (17 entries)

SEQ ID NO: 208 - DNA, 345 bp, synthetic construct
  source 1..345: mol_type = other DNA
caggctgtgccgactcagccgtcttccctctctgcatctcctggagtattagccagtctcacctgcaccttacgcagtggcatcaatgttggtacctacaggatatactggtaccagcagaagccagggagtcctccccagtatctcctgaggtacaaatcagactcagataagcagcagggctctggagtccccagccgcttctctggatccaaggatgcttcggccaatgcagggattttactcatctctgggctccagtctgaggatgaggctgactattactgtatgatttggtacagcagtgctgtggtattcggcggagggaccaaactgaccgtccta

SEQ ID NO: 209 - DNA, 318 bp, synthetic construct
  source 1..318: mol_type = other DNA
tcctatgagctgactcagccaccctcagtgtccgtgtccccaggacagacagccagcatcacctgctctggagataaattgggagataagtatgtttgctggtatcagcagaagccaggccagtcccctgtgctggtcatctatcaagatagcaagcggccctcagggatccctgagcgattctctggctccaactctgggaacacagccactctgaccatcagcgggacccaggctatggatgaggctgactattactgtcaggcgtgggacagtaacactgtggtattcggcggagggaccaagctgaccgtccta

SEQ ID NO: 210 - DNA, 318 bp, synthetic construct
  source 1..318: mol_type = other DNA
tcctttgagctgactcagccaccctcagtgtccgtgtccccaggacagacagccagcatcacctgctctggagataaattgggggataagtatgtttgctggtatcagcagaagccaggccagtcccctatgttggtcatctatcaagataccaagcggccctcagggatccctgaacgattctctggctccaactctgggaacacagccactctgaccatcagcgggacccaggctatggatgaggctgactattactgtcaggcgtgggacagcagcactgtggtcttcggcggagggaccaagctgaccgtccta

SEQ ID NO: 211 - DNA, 318 bp, synthetic construct
  source 1..318: mol_type = other DNA
tcctatgagctgactcagccaccctcagtgtccgtgtccccaggacagacagccagcatcacctgctctggagataaattgggggataagtatgtttgctggtatcagcagaagccaggccagtcccctgtgttggtcatctttcaagatagcaagcggccctcagggatccctgagcgattctctggctccaactctgggaacacagccactctgaccatcagcgggacccaggctatggatgaggctgactattactgtcaggcgtgggacagcagcactgtggtattcggcggagggaccaagctgaccgtccta

SEQ ID NO: 212 - DNA, 318 bp, synthetic construct
  source 1..318: mol_type = other DNA
tcctatgagctgactcagccaccctcagtgtccgtgtccccaggacagacagccagcatcacctgctctggagataaattgggggataagtatgtttgctggtatcagcagaagccaggccagtcccctgtgttggtcatctatcaagatagcaagcggccctcagggatccctgagcgattctctggctccaactctgggaacacagccactctgaccatcagcgggacccaggctatggatgaggctgactattactgtcaggcgtgggacagtagcactgtggtattcggcggagggaccaagctgaccgtccta

SEQ ID NO: 213 - DNA, 339 bp, synthetic construct
  source 1..339: mol_type = other DNA
ggcatcgtgatgacccagtctccagactccctggctgtgtctctgggcgagagggccaccatcaactgcaagtccagccggagtgttttgtacagctccaacaataagaactacttagcttggtaccagcagaaaccaggacagcctcctaaggtgctcatttactgggcatctacccgggaatccggggtccctgaccgattcagtggcagcgggtctgggacagatttcactctcaccatcagcagcctgcaggctgaagatgtggcagtttataactgtcagcaatattatattactccgatcaccttcggccaagggacacgactggagattaaa

SEQ ID NO: 214 - DNA, 339 bp, synthetic construct
  source 1..339: mol_type = other DNA
gacatcgtgatgacccagtctccagactccctggctgtgtctctgggcgagagggccaccatcaactgcaagtccagccagagtgttttatacagctccaacaataagcacttcttagcttggtaccagcagaaaccaggacagcctcctaagctgctcatttactgggcatctacccgggagtccggggtccctgaccgattcagtggcagcgggtctgggacagatttcactctcaccatcagcagcctgcaggctgaagatgtggcattttattactgtcagcaatattatagtactccgctcactttcggcggagggaccaaggtggagatcaaa

SEQ ID NO: 215 - DNA, 339 bp, synthetic construct
  source 1..339: mol_type = other DNA
gacatcgtgatgacccagtctccagactccctggctgtgtctctgggcgagagggccaccatcacctgcaagtccagccacaatgttttatacagctccaacaataagaactacttagcttggtatcagcagaaaccaggacagcctcctaaactgctcatttactgggcatctacccgggaatccggggtccctgaccgattcagtggcagcgggtctgggacagatttcactctcaccatcagcagcctgcaggctgaagatgtggcagtttattactgtcaccaatattatagtactccgtgcagttttggccaggggaccaagctggagatcaaa

SEQ ID NO: 216 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagcaacttagcctggttccagcagaaacctggccaggctcccaggctcctcatctatggtgcttccaccagggccactggtattccagccaggttcagtgtcagtgggtctgggacagagttcactctcaccatcagcagcctgcagtctgaagattttgcattttattactgtctgcaggataataattggcctcccactttcggccctgggaccaaagtggatatcaaa

SEQ ID NO: 217 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagcaacttagcctggttccagcagaaacctggccaggctcccaggctcctcatctatggtgcttccaccagggccactggtattccagccaggttcagtgtcagtgggtctgggacagagttcactctcaccatcagcagcctgcagtctgaagattttgcagtttattactgtctgcaggataataattggcctcccactttcggccctgggaccaaagtggatatcaaa

SEQ ID NO: 218 - DNA, 321 bp, synthetic construct
  source 1..321: mol_type = other DNA
gaaaaagtgatgacgcagtctccagccaccctgtctgtgtctccaggggaaagagccaccctctcctgcagggccagtcagagtgttaacaacaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctcggacgttcggccaagggaccaaggtggaaatcaaa

SEQ ID NO: 219 - DNA, 324 bp, synthetic construct
  source 1..324: mol_type = other DNA
gaatttgtgttgacgcagtctccaggcaccctgtctttgtctccgggggaaagagccaccctctcctgcagggccagtcagattattagcagcaactacttagcctggtaccagcagaaacctggccaggctcccaggctcctcatctatagtgcatccagcagggccactggcatcccagacaggttcagtggcagtgggtctgggacagacttcactctcaccatcagcagactggagcctgaagattttgcagtgtattactgtcagcagtttgatagctcaccgatcaccttcggccgagggacacgactggacattaaa

SEQ ID NO: 220 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcactgtattactgtcagcagtatgatacctcaccattcactttcggccctgggaccaaagtggatatcaaa

SEQ ID NO: 221 - DNA, 321 bp, synthetic construct
  source 1..321: mol_type = other DNA
gacatccagatgacccagtctccatcttccgtgtctgcatctgtaggagacagagtcaccgtcacttgtcgggcgagtcaggatattaacagctggttagcctggtatcagcagaaaccagggaaagcccctaagctcctgatctatgctgcatcctctttgcaaactggggtcccttcaaggttcagcggcagtggatctgggacagatttcactctcaccatcagcagcctgcagcctgaagattttgcaacttactcttgtcaacagtctaacagtttcccgatcaccttcggccaagggacacgactggagattaaa

SEQ ID NO: 222 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcaactggttagcctggtatcagcagaaaccagggaaagcccctaagctcctgatctatgctgcatccagtttgcaagttggggtcccattaaggttcagcggcagtggatctgggacagatttcactctcaccatcagcagcctgcagcctgaagattttgcaacttactattgtcaacaggctgacagtttccctcgcaattttggccaggggaccaagctggagatcaaa

SEQ ID NO: 223 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaaatggggtcccatcaaggttcagcggcagtggatctgggacagatttcactctcaccatcagcagcctgcagcctgaagattttgcaacttacttttgtcaacaggctgacagtttccctcgcacttttggccaggggaccaagctggagatcaaa

SEQ ID NO: 224 - DNA, 321 bp, synthetic construct
  source 1..321: mol_type = other DNA
gacatccagatgacccagtctccatcctcactgtctgcatctgtaggagacagagtcaccatcacttgtcgggcgagtcagggcattagcaattatttagcctggtttcagcagaaaccagggaaagcccctaaatccctgatctatgctgcatccagtttgcaaagtggggtcccatcaaagttcagcggcagtggatctgggacagatttcactctcaccatcagcagcctgcagcctgaagattttgcaacttattactgccaacagtatagtacttacccattcactttcggccctgggaccaaagtggatatcaaa

SEQ ID NO: 225 - DNA, 321 bp, synthetic construct
  source 1..321: mol_type = other DNA
gacatccagatgacccagtctccatcctccctgtctgcatctgtaggagacagagtcaccatcacttgccgggcaagtcagggcataagaaatgatttaggctggtatcagcagaaaccagggaatgcccctaagcgcctgatctatgctgcatccagtttgccaagtggggtcccatcaaggttcagcggcagtggatctgggccagaattcactctcacaatcagcagtctgcagcctgaagattttgcaacttattactgtctacagcataatagttacccgtggacgttcggccaagggaccaaggtggaaatcaca

SEQ ID NO: 226 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtaggcaaaatggggtcccatcaaggttcagcggcagtggatctgggacagatttcactctcaccatcagcagcctgcagcctgaagattttgcaacttacttttgtcaacaggctgacaggttccctcgcacttttggccaggggaccaagctggagatcaaa

SEQ ID NO: 227 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaaagggggtcccatcaaggttcagcggcagtggatctgggacagatttcactctcaccatcagcagcctgcagcctgaagattttgcaacttacttttgtcaacaggctgacagtttccctcatacttttggccaggggaccaagctggagatcaaa

SEQ ID NO: 228 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaacgtggggtcccatcaaggttcagcggcagtggatctgggacagatttcactctcaccatcagcagcctgcagcctgaagattttgcaacttacttttgtcaacaggctgacagtttccctcgcacttttggccaggggaccaagctggagatcaaa

SEQ ID NO: 229 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaacttggggtcccatcaaggttcagcggcagtggatctgggacagatttcactctcaccatcagcagcctgcagcctgaagattttgcaacttacttttgtcaacaggctgaccgtttccctcgcacttttggccaggggaccaagctggagatcaaa

SEQ ID NO: 230 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaacgggggtcccatcaaggttcagcggcagtggatctgggacagatttcactctcaccatcagcagcctgcagcctgaagattttgcaacttacttttgtcaacaggctgacagtttgcctcgcacttttggccaggggaccaagctggagatcaaa

SEQ ID NO: 231 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtcggcaaaatggggtcccatcaaggttcagcggcagtggatctgggacagatttcactctcaccatcagcagcctgcagcctgaagattttgcaacttacttttgtcaacaggctgacagttatcctcgcacttttggccaggggaccaagctggagatcaaa

SEQ ID NO: 232 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaaggggggtcccatcaaggttcagcggcagtggatctgggacagatttcactctcaccatcagcagcctgcagcctgaagattttgcaacttacttttgtcaacaggctgaccgtttccctcgcacttttggccaggggaccaagctggagatcaaa

SEQ ID NO: 233 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaaggggggtcccatcaaggttcagcggcagtggatctgggacagatttcactctcaccatcagcagcctgcagcctgaagattttgcaacttacttttgtgggcaggctgacagtttccctcgcacttttggccaggggaccaagctggagatcaaa

SEQ ID NO: 234 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtcgtcaaaatggggtcccatcaaggttcagcggcagtggatctgggacagatttcactctcaccatcagcagcctgcagcctgaagattttgcaacttacttttgtcaacaggctgacagttatcctcgcacttttggccaggggaccaagctggagatcaaa

SEQ ID NO: 235 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaaagggggtcccatcaaggttcagcggcagtggatctgggagagatttcactctcaccatcagcagcctgcagcctgaagattttgcaacttacttttgtcaacaggctgacagtttccctcgcacttttggccaggggaccaagctggagatcaaa

SEQ ID NO: 236 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatggtgcatccagtttgcaaaatggggtcccatcaaggttcagcggcagtggatctgggacagatttcactctcaccatcagcagcctgcagcctgaagattttgcaacttacttttgtcaacaggctgacagtttcccgcgcacttttggccaggggaccaagctggagatcaaa

SEQ ID NO: 237 - DNA, 363 bp, synthetic construct
  source 1..363: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattggtggtggtggtgttagcacatactgcgcagactccgtgaagggccggttcaccatctccagagacaattccaagaatacgctgtatctgcaaatgaacagcctgagagccgaggacacggccgtatattactgtgcgaaattttatatagcagtggctggttctcactttgactactggggccagggaaccctggtcaccgtctcctca

SEQ ID NO: 238 - DNA, 378 bp, synthetic construct
  source 1..378: mol_type = other DNA
gaggtgcaactggtggagtctgggggagccttggtacagcggggggggtccctgagactctcctgtgcagcctctagattcaccttcagtagctttggcatgagctgggtccgccaggctccagggaaggggctggagtgggtttcatacattagtagtagtagttttaccatatattacgcagactctgtgaagggccgattcaccatctccagagacaatgccaagaattcattctatctgcaaatgaacagcctgagagacgaggacacggctgtgtattactgtgcgagagaggggggtcttactatggttcggggagtctcttcctacggtttggacgtctggggccaagggaccacggtcaccgtctcctca

SEQ ID NO: 239 - DNA, 378 bp, synthetic construct
  source 1..378: mol_type = other DNA
gaggtgcaactggtggagtctgggggagccttggtacagcctggggggtccctgagactctcctgtgcagcctctggattcaccttcagtagctttggcatgagctgggtccgccaggctccagggaaggggctggagtgggtttcatacattagtagtagtagttttaccatatactacgcagactctgtgaagggccgattcaccatctccagagacaatgccaagaattcgttctatctgcaaatgaacagcctgagagacgaggacacggctgtgtatttctgtgtgagagaggggggtataactatggttcggggagtctcttcctacggtatggacgtctggggccaagggaccacggtcaccgtctcctca

SEQ ID NO: 240 - DNA, 378 bp, synthetic construct
  source 1..378: mol_type = other DNA
gaggtgcaactggtggagtctgggggagccttggtacagcctggggggtccctgagactctcctgtgcagcctctggattcaccttcagtagctttggcatgagctgggtccgccaggctccagggaaggggctggagtggatttcatacattagtagtagtagttttaccatatactacgcagactctgtgaagggccgattcaccatctccagagacaatgccaagaattcattctatctgcaaatgaacagcctgagagacgaggacacggctgtgtatttctgtgcgagagaggggggtattactatggttcggggagtctcttcctacggtatggacgtctggggccaagggaccacggtcaccgtctcctca

SEQ ID NO: 241 - DNA, 378 bp, synthetic construct
  source 1..378: mol_type = other DNA
gaggtgcaactggtggagtctgggggagccttggtacagcctggggggtccctgagactctcctgtgcagcctctggattcaccttcagtagctttggcatgagctgggtccgccaggctccagggaagggcctggagtgggtttcatacattagtaaaagtagttttaccatatactacgcagactctgtgaagggccgattcaccatctccagagacaatgccaagaattcattctatctgcaaatgaacagcctgagagacgaggacacggctgtgtattactgtgcgagagaggggggtcttactatggttcggggagtctcttcctacggtttggacgtctggggccaagggaccacggtcaccgtctcctca

SEQ ID NO: 242 - DNA, 357 bp, synthetic construct
  source 1..357: mol_type = other DNA
gaggtgcagctgttggagtctgggggaggcttggtacagcctggggggtccctgagactctcctgtgcagcctctggattcacctttagcagctatgccatgagctgggtccgccaggctccagggaagggactggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaggcgtatacacctatggcattctttgactactggggccagggaaccctggtcaccgtctcctca

SEQ ID NO: 243 - DNA, 348 bp, synthetic construct
  source 1..348: mol_type = other DNA
gaggtgcaggtgttggagtctgggggaggcttggtacagcctggggggtccctgagactctcctgtgcagcctctggattcacctttagcaactatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaggagggtgggagctattttactggggccagggaaccctggtcaccgtctcctca

SEQ ID NO: 244 - DNA, 363 bp, synthetic construct
  source 1..363: mol_type = other DNA
gaggtgcagctggtgcagtctggagcagaggtgaaaaagcccggggagtctctgatgatctcctgtaagggttctggatacagctttaccaactactggatcggctgggtgcgccagatgcccgggaaaggcctggagtggatggggatcatctatcctggagactctgataccagatacagcccgtccttccaaggccaggtcaccatctcagccgacaagtccatcagcaccgcctacctgcagtggagcagcctgaaggcctcggacaccgccatgtatttctgtgcgagacggagacaggggatctggggtgatgctcttgatatctggggccaagggacattggtcaccgtctcttca

SEQ ID NO: 245 - DNA, 363 bp, synthetic construct
  source 1..363: mol_type = other DNA
gaggtgcagctggtgcagtctggagcagaggtgaaaaagcccggggagtctctgatgatctcctgtaagggttctggatacagctttaccagctactggatcggctgggtgcgccagatgcccgggaaaggcctggagtggatggggatcatctatcctggagactctgataccagatacagcccgtccttccaaggccaggtcaccatctcagccgacaagtccatcagcaccgcctacctgcagtggagcagcctgaaggcctcggacaccgccatgtatttctgtgcgagacggagacaggggatctggggtgatgctcttgatttctggggccaagggacattggtcaccgtctcttca

SEQ ID NO: 246 - DNA, 372 bp, synthetic construct
  source 1..372: mol_type = other DNA
caggtgcagctacagcagtggggcgcaggactgttgaagccttcggagaccctgtccctcacctgcgctgtctatggtgggtccttcagtagttactactggagctggatccgccagcccccagggaaggggctggagtggattggggaaatcaatcatagtggaaacaccaactacaacccgtccctcaagagtcgagtcaccatatcagtagacacgtccaagaaccagttctccctgaagctgagctctgtgaccgccgcggacacggctgtgtattactgtgcgagagaggggtattacgatatcttgactggttatcatgatgcttttgatatttgggaccaagggacaatggtcaccgtnttttca

SEQ ID NO: 247 - DNA, 366 bp, synthetic construct
  source 1..366: mol_type = other DNA
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcaccctgaaggcctcggacaccgccatatattactgtgcgcgacatgacattataccagcagcccctggtgcttttgatatctggggccaagggacaatggtcaccgtctcttca

SEQ ID NO: 248 - DNA, 366 bp, synthetic construct
  source 1..366: mol_type = other DNA
gaggtgcagctggtgcagtctggagcagaggtgaaaaagcccggggagtctctgaagatctcctgtaagggttctggatacacctttaccagctactggatcggctgggtgcgccagatgcccgggaaaggcctggagtggatggggatcatctatcctggtgactctgataccagatacagcccgtccttccaaggccaggtcaccatctcagtcgacaagtccagcagcaccgcctacctgcagtggagcagcctgaaggcctcggacaccgccatatattactgtacgagacaggccatagcagtgactggtttggggggtttcgacccctggggccagggaaccctggtcaccgtctcctca

SEQ ID NO: 249 - DNA, 357 bp, synthetic construct
  source 1..357: mol_type = other DNA
caggttcagctggtgcagtctggagctgaggtgaagaagcctggggcctcagtgaaggtctcctgcaaggcttctggttacacctttaccaactatggtatcagctgggtgcgacaggcccctggacaagggcttgagtggatgggatggatcagcgcttacaatggtaacacaaactatgcacagaagctccagggcagagtcaccatgaccacagacacatccacgagtacagtctacatggagctgaggagcctgagatctgacgacacggccgtgtattactgtgcgagacggggatacagctatggttcctttgactactggggccagggaaccctggtcaccgtctcctca

SEQ ID NO: 250 - DNA, 369 bp, synthetic construct
  source 1..369: mol_type = other DNA
gaggtgcagctggtgcagtctggagcagaagtgaaaaagcccggggagtctctgaagatctcctgtaagggttctggacacagttttaccaactactggatcgcctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccgtgtatttctgtgcgagacaaaggacgttttactatgatagtagtggttattttgactactggggccagggaaccctggtcaccgtctcctca

SEQ ID NO: 251 - DNA, 369 bp, synthetic construct
  source 1..369: mol_type = other DNA
gaggtgcagctggtgcagtctggagcagaggtgaaaaagcccggggagtctctgaagatctcctgtaagggttctggatacagttttaccagctactggatcgcctgggtgcgccagatgcccgggaaaggcctggagtggatggggatcatctatcctggtgactctgataccagatacagcccgtccttccaaggccaggtcaccatctcagccgacaagtccatcagcaccgcctacctacagtggagcagcctgaaggcctcggacaccgccatgtatttctgtgcgagacaaaggacgttttattatgatagtagtgattattttgactactggggccagggaaccctggtcaccgtctcctca

SEQ ID NO: 252 - DNA, 363 bp, synthetic construct
  source 1..363: mol_type = other DNA
caggtgcagctggtgcagtctggggctgaggtgaagaagcctggggcctcagtgaaggtctcctgcaaggcttctggatacaccttcaccggctactatatacactgggtgcgacaggcccctggactagggcttgagtggatgggatggatcaacccttacagtggtggcacaacctctgcacagaagtttcagggcagggtcaccatgaccagggacacgtccatcagctcagcctacatggaactgagcaggctgagatctgacgacacggccgtgtattactgtgcgagagatggaggctacctggccctctacggtacggacgtctggggccaagggaccacggtcaccgtctcctca

SEQ ID NO: 253 - DNA, 366 bp, synthetic construct
  source 1..366: mol_type = other DNA
gaggtgcagctggtgcagtctggagcagaggtgaaaaagcccggggagtctctgaagatctcctgtaagggttctggatacagctttcccagctactggatcgcctgggtgcgccagatgcccgggaaaggcctggagtggatggggatcatctatcctggtgactctgataccagatacagcccgtccttccaaggccaggtcaccatctcagccgacaagtccatcagcaccgcctttttgaagtggagtagcctgaaggcctcggacaccgccatgtatttctgtgcgcgacagggtatagaagtgactggtacgggaggtttggacgtctggggccaagggaccacggtcaccgtctcctca

SEQ ID NO: 254 - DNA, 369 bp, synthetic construct
  source 1..369: mol_type = other DNA
gaggtgcagctggtgcagtctggagcagaggtgaaaaagcccggggagtctctgaagatctcctgtaagggttctggatacagttttgcgagctactggatcgcctgggtgcgccagatgcccgggaaaggcctggagtggatggggatcatctatcctggtgactctgataccagatacagcccgtccttccaagatcaggtcaccatctcagccgacaagtccatcagcaccgcctacctacagtggagcagcctgaaggcctcggacaccgccatgtatttctgtgcgagagggaggacgttttattatgatagtagtgattattttgactactggggccagggaaccctggtcaccgtgtcctca

SEQ ID NO: 255 - DNA, 369 bp, synthetic construct
  source 1..369: mol_type = other DNA
gaggtgcagctggtgcagtctggagcagaggtgaaaaagcccggggagtctctgaagatctcctgtaagggttctggatacagttttaccagctactggattgcctgggtgcgccagatgcccgggaaaggcctggagtggatggggatcatctatcctggtgactctgatgtgagatacagcccgtccttccaaggccaggtcaccatctcagccgacaagtccatcagcaccgcctacctacagtggagcagcctgaaggcctcggacaccgccatgtatttctgtgcgagatctaggacgttttattatgatagtagtgattattttgactactggggccagggaaccctggtcaccgtgtcctca

SEQ ID NO: 256 - DNA, 369 bp, synthetic construct
  source 1..369: mol_type = other DNA
gaggtgcagctggtgcagtctggagcagaggtgaaaaagcccggggagtctctgaagatctcctgtaagggttctggatacagttttaccagctactggatcgcctgggtgcgccagatgcccgggaaaggcctggagtggatggggatcatctatcctggtgactctgataccagatacgctccgtccttccaaggccaggtcaccatctcagccgacaagtccatcagcaccgcctacctacagtggagcagcctgaaggcctcggacaccgccatgtatttctgtgtgagaagtaggacgttttattatgatagtagtgattattttgactactggggccagggaaccctggtcaccgtgtcctca

SEQ ID NO: 257 - DNA, 369 bp, synthetic construct
  source 1..369: mol_type = other DNA
gaggtgcagctggtgcagtctggagcagaggtgaaaaagcccggggagtctctgaagatctcctgtaagggttctggatacagttttgggagctactggatcgcctgggtgcgccagatgcccgggaaaggcctggagtggatggggatcatctatcctggtgactctgatgttagatacagcccgtccttccaaggccaggtcaccatctcagccgacaagtccatcagcaccgcctacctacagtggagcagcctgaaggcctcggacaccgccatgtatttctgtgcgagacaaaggacgttttattatgatagtagtgattattcggactactggggccagggaaccctggtcaccgtgtcctca

SEQ ID NO: 258 - DNA, 369 bp, synthetic construct
  source 1..369: mol_type = other DNA
gaggtgcagctggtgcagtctggagcagaggtgaaaaagcccggggagtctctgaagatctcctgtaagggttctggatacagttttggtagctactggatcgcctgggtgcgccagatgcccgggaaaggcctggagtggatggggatcatctatcctggtgactctgatgtgagatacagcccgtccttccaaggccaggtcaccatctcagccgacaagtccatcagcaccgcctacctacagtggagcagcctgaaggcctcggacaccgccatgtatttctgtgcgagaatgaggacgttttattatgatagtagtgattattttgactactggggccagggaaccctggtcaccgtgtcctca

SEQ ID NO: 259 - DNA, 369 bp, synthetic construct
  source 1..369: mol_type = other DNA
gaggtgcagctggtgcagtctggagcagaggtgaaaaagcccggggagtctctgaagatctcctgtaagggttctggatacagttttaatagctactggatcgcctgggtgcgccagatgcccgggaaaggcctggagtggatggggacgatctatcctggtgactctgataccagactgagcccgtccttccaaggccaggtcaccatctcagccgacaagtccatcagcaccgcctacctacagtggagcagcctgaaggcctcggacaccgccatgtatttctgtgcgagaagtaggacgttttattatgatagtagtgattattttgactactggggccagggaaccctggtcaccgtgtcctca

SEQ ID NO: 260 - DNA, 369 bp, synthetic construct
  source 1..369: mol_type = other DNA
gaggtgcagctggtgcagtctggagcagaggtgaaaaagcccggggagtctctgaagatctcctgtaagggttctggatacagttttgagagctactggatcgcctgggtgcgccagatgcccgggaaaggcctggagtggatggggatcatctatcctggtgactctgataccagatacagcccgtccttccaaggccaggtcaccatctcagccgacaagtccatcagcaccgcctacctacagtggagcagcctgaaggcctcggacaccgccatgtatttctgtgcgagagggaggacgttttattatgatagtagtgattattttgactactggggccagggaaccctggtcaccgtgtcctca

SEQ ID NO: 261 - DNA, 369 bp, synthetic construct
  source 1..369: mol_type = other DNA
gaggtgcagctggtgcagtctggagcagaggtgaaaaagcccggggagtctctgaagatctcctgtaagggttctggataccattttaccagctactggatcgcctgggtgcgccagatgcccgggaaaggcctggagtggatggggatcatctatcctggtgactctgatgtgagatacagcccgtccttccaaggccaggtcaccatctcagccgacaagtccatcagcaccgcctacctacagtggagcagcctgaaggcctcggacaccgccatgtatttctgtgcgagacaaaggacgttttattatgatagtagtgattatagtgactactggggccagggaaccctggtcaccgtgtcctca

SEQ ID NO: 262 - DNA, 369 bp, synthetic construct
  source 1..369: mol_type = other DNA
gaggtgcagctggtgcagtctggagcagaggtgaaaaagcccggggagtctctgaagatctcctgtaagggttctggatacagttttggtagctactggatcgcctgggtgcgccagatgcccgggaaaggcctggagtggatggggatcatctatcctggtgactctgataccagatacagcccggggttccaaggccaggtcaccatctcagccgacaagtccatcagcaccgcctacctacagtggagcagcctgaaggcctcggacaccgccatgtatttctgtgcgagagggaggacgttttattatgatagtagtgattattttgactactggggccagggaaccctggtcaccgtgtcctca

SEQ ID NO: 263 - DNA, 369 bp, synthetic construct
  source 1..369: mol_type = other DNA
gaggtgcagctggtgcagtctggagcagaggtgaaaaagcccggggagtctctgaagatctcctgtaagggttctggatacagttttgggagctactggatcgcctgggtgcgccagatgcccgggaaaggcctggagtggatggggatcatctatcctggtgactctgataccagatacagcccggagttccaaggccaggtcaccatctcagccgacaagtccatcagcaccgcctacctacagtggagcagcctgaaggcctcggacaccgccatgtatttctgtgcgagacaaaggacgttttattatgatagtagtgattatagtgactactggggccagggaaccctggtcaccgtgtcctca

SEQ ID NO: 264 - DNA, 369 bp, synthetic construct
  source 1..369: mol_type = other DNA
gaggtgcagctggtgcagtctggagcagaggtgaaaaagcccggggagtctctgaagatctcctgtaagggttctggatacgggtttaccagctactggatcgcctgggtgcgccagatgcccgggaaaggcctggagtggatggggatcatctatcctggtgacagtgataccagatacagcccgtccttccaaggccaggtcaccatctcagccgacaagtccatcagcaccgcctacctacagtggagcagcctgaaggcctcggacaccgccatgtatttctgtgcgagacaaaggacgttttattatgatagtagtgattattcggactactggggccagggaaccctggtcaccgtgtcctca

SEQ ID NO: 265-270 - skipped (6 entries)

SEQ ID NO: 271 - protein, 9 aa, synthetic construct
QQAVSFPRT

SEQ ID NO: 272-283 - skipped (12 entries)

SEQ ID NO: 284 - protein, 11 aa, synthetic construct
  VARIANT 1: note = X is R or A
  VARIANT 8: note = X is S or R
XASQGISXWLA

SEQ ID NO: 285 - protein, 7 aa, synthetic construct
  VARIANT 1: note = X is A or S
  VARIANT 3: note = x is S or G
XAXSLQN

SEQ ID NO: 286 - protein, 9 aa, synthetic construct
  VARIANT 4: note = X is D or V
  VARIANT 8: note = X is R or L
QQAXSFPXT

SEQ ID NO: 287 - protein, 5 aa, synthetic construct
  VARIANT 2: note = X is Y or E
SXWIA

SEQ ID NO: 288 - protein, 17 aa, synthetic construct
  VARIANT 5: note = X is G or S
IIYPXDSDTRYSPSFQG

SEQ ID NO: 289 - protein, 14 aa, synthetic construct
  VARIANT 3: note = X is T or G
  VARIANT 5: note = X is Y or R
  VARIANT 6: note = X is Y or G
QRXFXXDSSDYFDY

SEQ ID NO: 290 - protein, 11 aa, synthetic construct
AASQGISSWLA

SEQ ID NO: 291 - protein, 11 aa, synthetic construct
RASQGISRWLA

SEQ ID NO: 292 - protein, 7 aa, synthetic construct
SASSLQN

SEQ ID NO: 293 - protein, 7 aa, synthetic construct
AAGSLQN

SEQ ID NO: 294 - protein, 9 aa, synthetic construct
QQADSFPLT

SEQ ID NO: 295 - protein, 107 aa, synthetic construct
DIQMTQSPSSVSASVGDRVTITCRASQGISSWLAWYQQKPGKAPKLLIYSASSLQNGVPSRFSGSGSGTDFTLTISSLQPEDFATYFCQQADSFPRTFGQGTKLEIK

SEQ ID NO: 296 - protein, 107 aa, synthetic construct
DIQMTQSPSSVSASVGDRVTITCRASQGISSWLAWYQQKPGKAPKLLIYAASSLQNGVPSRFSGSGSGTDFTLTISSLQPEDFATYFCQQADSFPLTFGQGTKLEIK

SEQ ID NO: 297 - protein, 107 aa, synthetic construct
DIQMTQSPSSVSASVGDRVTITCAASQGISSWLAWYQQKPGKAPKLLIYAASSLQNGVPSRFSGSGSGTDFTLTISSLQPEDFATYFCQQADSFPRTFGQGTKLEIK

SEQ ID NO: 298 - protein, 107 aa, synthetic construct
DIQMTQSPSSVSASVGDRVTITCRASQGISSWLAWYQQKPGKAPKLLIYAAGSLQNGVPSRFSGSGSGTDFTLTISSLQPEDFATYFCQQADSFPRTFGQGTKLEIK

SEQ ID NO: 299 - protein, 107 aa, synthetic construct
DIQMTQSPSSVSASVGDRVTITCRASQGISSWLAWYQQKPGKAPKLLIYAASSLQNGVPSRFSGSGSGTDFTLTISSLQPEDFATYFCQQAVSFPRTFGQGTKLEIK

SEQ ID NO: 300 - protein, 107 aa, synthetic construct
DIQMTQSPSSVSASVGDRVTITCRASQGISRWLAWYQQKPGKAPKLLIYAASSLQNGVPSRFSGSGSGTDFTLTISSLQPEDFATYFCQQADSFPRTFGQGTKLEIK

SEQ ID NO: 301 - protein, 4 aa, synthetic construct
SSFT

SEQ ID NO: 302 - protein, 5 aa, synthetic construct
SEWIA

SEQ ID NO: 303 - protein, 17 aa, synthetic construct
IIYPSDSDTRYSPSFQG

SEQ ID NO: 304 - protein, 14 aa, synthetic construct
QRGFYYDSSDYFDY

SEQ ID NO: 305 - protein, 14 aa, synthetic construct
QRTFYGDSSDYFDY

SEQ ID NO: 306 - protein, 14 aa, synthetic construct
QRTFRYDSSDYFDY

SEQ ID NO: 307 - protein, 123 aa, synthetic construct
EVQLVQSGAEVKKPGESLKISCKGSGYSFTSYWIAWVRQMPGKGLEWMGIIYPGDSDTRYSPSFQGQVTISADKSISTAYLQWSSLKASDTAMYFCARQRGFYYDSSDYFDYWGQGTLVTVSS

SEQ ID NO: 308 - protein, 123 aa, synthetic construct
EVQLVQSGAEVKKPGESLKISCKGSGSSFTSYWIAWVRQMPGKGLEWMGIIYPGDSDTRYSPSFQGQVTISADKSISTAYLQWSSLKASDTAMYFCARQRTFYYDSSDYFDYWGQGTLVTVSS

SEQ ID NO: 309 - protein, 123 aa, synthetic construct
EVQLVQSGAEVKKPGESLKISCKGSGYSFTSYWIAWVRQMPGKGLEWMGIIYPGDSDTRYSPSFQGQVTISADKSISTAYLQWSSLKASDTAMYFCARQRTFYGDSSDYFDYWGQGTLVTVSS

SEQ ID NO: 310 - protein, 123 aa, synthetic construct
EVQLVQSGAEVKKPGESLKISCKGSGYSFTSYWIAWVRQMPGKGLEWMGIIYPSDSDTRYSPSFQGQVTISADKSISTAYLQWSSLKASDTAMYFCARQRTFYYDSSDYFDYWGQGTLVTVSS

SEQ ID NO: 311 - protein, 123 aa, synthetic construct
EVQLVQSGAEVKKPGESLKISCKGSGYSFTSYWIAWVRQMPGKGLEWMGIIYPGDSDTRYSPSFQGQVTISADKSISTAYLQWSSLKASDTAMYFCARQRTFRYDSSDYFDYWGQGTLVTVSS

SEQ ID NO: 312 - protein, 123 aa, synthetic construct
EVQLVQSGAEVKKPGESLKISCKGSGYSFTSEWIAWVRQMPGKGLEWMGIIYPGDSDTRYSPSFQGQVTISADKSISTAYLQWSSLKASDTAMYFCARQRTFYYDSSDYFDYWGQGTLVTVSS

SEQ ID NO: 313 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctattctgcatccagtttgcaaaatggggtcccatcaaggttcagcggcagtggatctgggacagatttcactctcaccatcagcagcctgcagcctgaagattttgcaacttacttttgtcaacaggctgacagtttccctcgcacttttggccaggggaccaagctggagatcaaa

SEQ ID NO: 314 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaaatggggtcccatcaaggttcagcggcagtggatctgggacagatttcactctcaccatcagcagcctgcagcctgaagattttgcaacttacttttgtcaacaggctgacagtttccctcttacttttggccaggggaccaagctggagatcaaa

SEQ ID NO: 315 - DNA, 321 bp, synthetic construct
  source 1..321: mol_type = other DNA
gacatccagatgacccagtctccatcttccgtgtctgcatctgtaggagacagagtcaccatcacttgtgcggcgagtcagggtattagcagctggttagcctggtatcagcagaaaccagggaaagcccctaagctcctgatctatgctgcatccagtttgcaaaatggggtcccatcaaggttcagcggcagtggatctgggacagatttcactctcaccatcagcagcctgcagcctgaagattttgcaacttacttttgtcaacaggctgacagtttccctcgcacttttggccaggggaccaagctggagatcaaa

SEQ ID NO: 316 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcagggagtttgcaaaatggggtcccatcaaggttcagcggcagtggatctgggacagatttcactctcaccatcagcagcctgcagcctgaagattttgcaacttacttttgtcaacaggctgacagtttccctcgcacttttggccaggggaccaagctggagatcaaa

SEQ ID NO: 317 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaaatggggtcccatcaaggttcagcggcagtggatctgggacagatttcactctcaccatcagcagcctgcagcctgaagattttgcaacttacttttgtcaacaggctgtgagtttccctcgcacttttggccaggggaccaagctggagatcaaa

SEQ ID NO: 318 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagatggttagcctggtatcagcagaaaccagggaaagcccctaagctcctgatctatgctgcatccagtttgcaaaatggggtcccatcaaggttcagcggcagtggatctgggacagatttcactctcaccatcagcagcctgcagcctgaagattttgcaacttacttttgtcaacaggctgacagtttccctcgcacttttggccaggggaccaagctggagatcaaa

SEQ ID NO: 319 - DNA, 369 bp, synthetic construct
  source 1..369: mol_type = other DNA
gaggtgcagctggtgcagtctggagcagaggtgaaaaagcccggggagtctctgaagatctcctgtaagggttctggatacagttttaccagctactggatcgcctgggtgcgccagatgcccgggaaaggcctggagtggatggggatcatctatcctggtgactctgataccagatacagcccgtccttccaaggccaggtcaccatctcagccgacaagtccatcagcaccgcctacctacagtggagcagcctgaaggcctcggacaccgccatgtatttctgtgcgagacaaagggggttttattatgatagtagtgattattttgactactggggccagggaaccctggtcaccgtgtcctca

SEQ ID NO: 320 - DNA, 369 bp, synthetic construct
  source 1..369: mol_type = other DNA
gaggtgcagctggtgcagtctggagcagaggtgaaaaagcccggggagtctctgaagatctcctgtaagggttctggatacagttttaccagctactggatcgcctgggtgcgccagatgcccgggaaaggcctggagtggatggggatcatctatcctggtgactctgataccagatacagcccgtccttccaaggccaggtcaccatctcagccgacaagtccatcagcaccgcctacctacagtggagcagcctgaaggcctcggacaccgccatgtatttctgtgcgagacaaaggacgttttattatgatagtagtgattattttgactactggggccagggaaccctggtcaccgtgtcctca

SEQ ID NO: 321 - DNA, 369 bp, synthetic construct
  source 1..369: mol_type = other DNA
gaggtgcagctggtgcagtctggagcagaggtgaaaaagcccggggagtctctgaagatctcctgtaagggttctggatcgagttttaccagctactggatcgcctgggtgcgccagatgcccgggaaaggcctggagtggatggggatcatctatcctggtgactctgataccagatacagcccgtccttccaaggccaggtcaccatctcagccgacaagtccatcagcaccgcctacctacagtggagcagcctgaaggcctcggacaccgccatgtatttctgtgcgagacaaaggacgttttattatgatagtagtgattattttgactactggggccagggaaccctggtcaccgtgtcctca

SEQ ID NO: 322 - DNA, 369 bp, synthetic construct
  source 1..369: mol_type = other DNA
gaggtgcagctggtgcagtctggagcagaggtgaaaaagcccggggagtctctgaagatctcctgtaagggttctggatacagttttaccagctactggatcgcctgggtgcgccagatgcccgggaaaggcctggagtggatggggatcatctatcctggtgactctgataccagatacagcccgtccttccaaggccaggtcaccatctcagccgacaagtccatcagcaccgcctacctacagtggagcagcctgaaggcctcggacaccgccatgtatttctgtgcgagacaaaggacgttttatggggatagtagtgattattttgactactggggccagggaaccctggtcaccgtgtcctca

SEQ ID NO: 323 - DNA, 369 bp, synthetic construct
  source 1..369: mol_type = other DNA
gaggtgcagctggtgcagtctggagcagaggtgaaaaagcccggggagtctctgaagatctcctgtaagggttctggatacagttttaccagctactggatcgcctgggtgcgccagatgcccgggaaaggcctggagtggatggggatcatctatcctagtgactctgataccagatacagcccgtccttccaaggccaggtcaccatctcagccgacaagtccatcagcaccgcctacctacagtggagcagcctgaaggcctcggacaccgccatgtatttctgtgcgagacaaaggacgttttattatgatagtagtgattattttgactactggggccagggaaccctggtcaccgtgtcctca

SEQ ID NO: 324 - DNA, 369 bp, synthetic construct
  source 1..369: mol_type = other DNA
gaggtgcagctggtgcagtctggagcagaggtgaaaaagcccggggagtctctgaagatctcctgtaagggttctggatacagttttaccagctactggatcgcctgggtgcgccagatgcccgggaaaggcctggagtggatggggatcatctatcctggtgactctgataccagatacagcccgtccttccaaggccaggtcaccatctcagccgacaagtccatcagcaccgcctacctacagtggagcagcctgaaggcctcggacaccgccatgtatttctgtgcgagacaaaggacgtttaggtatgatagtagtgattattttgactactggggccagggaaccctggtcaccgtgtcctca

SEQ ID NO: 325 - DNA, 369 bp, synthetic construct
  source 1..369: mol_type = other DNA
gaggtgcagctggtgcagtctggagcagaggtgaaaaagcccggggagtctctgaagatctcctgtaagggttctggatacagttttaccagcgagtggatcgcctgggtgcgccagatgcccgggaaaggcctggagtggatggggatcatctatcctggtgactctgataccagatacagcccgtccttccaaggccaggtcaccatctcagccgacaagtccatcagcaccgcctacctacagtggagcagcctgaaggcctcggacaccgccatgtatttctgtgcgagacaaaggacgttttattatgatagtagtgattattttgactactggggccagggaaccctggtcaccgtgtcctca

SEQ ID NO: 326 - protein, 113 aa, synthetic construct
DIVMTQSPDSLAVSLGERATINCKSSQSVLYSSNNKHFLAWYQQKPGQPPKLLIYWASTRESGVPDRFSGSGSGTDFTLTISSLQAEDVAVYYCQQYYSTPLTFGGGTKVEIK

SEQ ID NO: 327 - protein, 119 aa, synthetic construct
EVQLLESGGGLVQPGGSLRLSCAASGFTFSSYAMSWVRQAPGKGLEWVSAISGSGGSTYYAESVKGRFTISRDNSKNTLYLQMNSLRAEDTAVYYCAKAYTPMAFFDYWGQGTLVTVSS

SEQ ID NO: 328 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYFCQQADAFPRTFGQGTKLEIK

SEQ ID NO: 329 - protein, 123 aa, synthetic construct
EVQLVQSGAEVKKPGESLKISCKGSGYSFTSYWIAWVRQMPGKGLEWMGIIYPGDADARYSPSFQGQVTISADKSISTAYLQWSSLKASDTAMYFCARQRTFYYDSSDYFDYWGQGTLVTVSS

SEQ ID NO: 330 - protein, 107 aa, synthetic construct; identical to SEQ ID NO: 1

SEQ ID NO: 331 - protein, 121 aa, synthetic construct; identical to SEQ ID NO: 5

SEQ ID NO: 332 - protein, 107 aa, synthetic construct
DIQMTQSPSSLSASVGDRVTITCRASQGISNYLAWYQQKPGKAPKSLIYAASSLQSGVPSRFSGSGSGTDFTLTISSLQPEDFATYYCQQYSTYPFTFGQGTKVDIK

SEQ ID NO: 333 - protein, 121 aa, synthetic construct
QVQLVQSGAEVKKPGASVKVSCKASGYTFTGYYIHWVRQAPGQGLEWMGWINPYSGGTTSAQKFQGRVTMTRDTSTSSAYMELSRLRSDDTAVYYCARDAGYLALYGTDVWGQGTLVTVSS

SEQ ID NO: 334 - protein, 242 aa, synthetic construct
MDMRVPAQLLGLLLLWLRGARCDIVMTQSPDSLAVSLGERATINCKSSQSVLYSSNNKHFLAWYQQKPGQPPKLLIYWASTRESGVPDRFSGSGSGTDFTLTISSLQAEDVAVYYCQQYYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 335 - protein, 471 aa, synthetic construct
MDMRVPAQLLGLLLLWLRGARCEVQLLESGGGLVQPGGSLRLSCAASGFTFSSYAMSWVRQAPGKGLEWVSAISGSGGSTYYAESVKGRFTISRDNSKNTLYLQMNSLRAEDTAVYYCAKAYTPMAFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 336 - protein, 468 aa, synthetic construct
MDMRVPAQLLGLLLLWLRGARCEVQLLESGGGLVQPGGSLRLSCAASGFTFSSYAMSWVRQAPGKGLEWVSAISGSGGSTYYAESVKGRFTISRDNSKNTLYLQMNSLRAEDTAVYYCAKAYTPMAFFDYWGQGTLVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 337 - protein, 236 aa, synthetic construct
MDMRVPAQLLGLLLLWLRGARCDIQMTQSPSSVSASVGDRVTITCRASQGISSWLAWYQQKPGKAPKLLIYAASSLQSGVPSRFSGSGSGTDFTLTISSLQPEDFATYFCQQADAFPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 338 - protein, 472 aa, synthetic construct
MDMRVPAQLLGLLLLWLRGARCEVQLVQSGAEVKKPGESLKISCKGSGYSFTSYWIAWVRQMPGKGLEWMGIIYPGDADARYSPSFQGQVTISADKSISTAYLQWSSLKASDTAMYFCARQRTFYYDSSDYFDYWGQGTLVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 339 - protein, 236 aa, synthetic construct
MDMRVPAQLLGLLLLWLRGARCEIVMTQSPATLSVSPGERATLSCRASQSVSSNLAWFQQKPGQAPRLLIYGASTRATGIPARFSGSGSGTEFTLTISSLQPEDFAVYYCLQDNNFPPTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 340 - protein, 473 aa, synthetic construct
MDMRVPAQLLGLLLLWLRGARCEVQLVQSGAEVKKPGESLKISCKGSGYSFTSYWIGWVRQMPGKGLEWMGIIYPGDADARYSPSFQGQVTISADKSISTAYLQWSSLKASDTAMYFCARRRQGIFGDALDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 341 - protein, 236 aa, synthetic construct
MDMRVPAQLLGLLLLWLRGARCDIQMTQSPSSLSASVGDRVTITCRASQGISNYLAWYQQKPGKAPKSLIYAASSLQSGVPSRFSGSGSGTDFTLTISSLQPEDFATYYCQQYSTYPFTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 342 - protein, 470 aa, synthetic construct
MDMRVPAQLLGLLLLWLRGARCQVQLVQSGAEVKKPGASVKVSCKASGYTFTGYYIHWVRQAPGQGLEWMGWINPYSGGTTSAQKFQGRVTMTRDTSTSSAYMELSRLRSDDTAVYYCARDAGYLALYGTDVWGQGTLVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 343 - protein, 220 aa, synthetic construct
DIVMTQSPDSLAVSLGERATINCKSSQSVLYSSNNKHFLAWYQQKPGQPPKLLIYWASTRESGVPDRFSGSGSGTDFTLTISSLQAEDVAVYYCQQYYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 344 - protein, 449 aa, synthetic construct
EVQLLESGGGLVQPGGSLRLSCAASGFTFSSYAMSWVRQAPGKGLEWVSAISGSGGSTYYAESVKGRFTISRDNSKNTLYLQMNSLRAEDTAVYYCAKAYTPMAFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 345 - protein, 445 aa, synthetic construct
EVQLLESGGGLVQPGGSLRLSCAASGFTFSSYAMSWVRQAPGKGLEWVSAISGSGGSTYYAESVKGRFTISRDNSKNTLYLQMNSLRAEDTAVYYCAKAYTPMAFFDYWGQGTLVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 346 - protein, 214 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYFCQQADAFPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 347 - protein, 449 aa, synthetic construct
EVQLVQSGAEVKKPGESLKISCKGSGYSFTSYWIAWVRQMPGKGLEWMGIIYPGDADARYSPSFQGQVTISADKSISTAYLQWSSLKASDTAMYFCARQRTFYYDSSDYFDYWGQGTLVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 348 - protein, 214 aa, synthetic construct; identical to SEQ ID NO: 9

SEQ ID NO: 349 - protein, 450 aa, synthetic construct
EVQLVQSGAEVKKPGESLKISCKGSGYSFTSYWIGWVRQMPGKGLEWMGIIYPGDADARYSPSFQGQVTISADKSISTAYLQWSSLKASDTAMYFCARRRQGIFGDALDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 350 - protein, 214 aa, synthetic construct
DIQMTQSPSSLSASVGDRVTITCRASQGISNYLAWYQQKPGKAPKSLIYAASSLQSGVPSRFSGSGSGTDFTLTISSLQPEDFATYYCQQYSTYPFTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 351 - protein, 447 aa, synthetic construct
QVQLVQSGAEVKKPGASVKVSCKASGYTFTGYYIHWVRQAPGQGLEWMGWINPYSGGTTSAQKFQGRVTMTRDTSTSSAYMELSRLRSDDTAVYYCARDAGYLALYGTDVWGQGTLVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 352 - protein, 214 aa, synthetic construct; identical to SEQ ID NO: 9

SEQ ID NO: 353 - protein, 451 aa, synthetic construct; identical to SEQ ID NO: 10

SEQ ID NO: 354 - protein, 229 aa, Homo sapiens
MEPLRLLILLFVTELSGAHNTTVFQGVAGQSLQVSCPYDSMKHWGRRKAWCRQLGEKGPCQRWSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 355 - protein, 173 aa, Homo sapiens
MEPLRLLILLFVTELSGAHNTTVFQGVAGQSLQVSCPYDSMKHWGRRKAWCRQLGEKGPCQRWSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTS

SEQ ID NO: 356 - protein, 14 aa, synthetic construct
TLRSGINVGTYRIY

SEQ ID NO: 357 - protein, 11 aa, synthetic construct
SGDKLGDKYVC

SEQ ID NO: 358 - protein, 17 aa, synthetic construct
KSSRSVLYSSNNKNYLA

SEQ ID NO: 359 - protein, 17 aa, synthetic construct
KSSQSVLYSSNNKHFLA

SEQ ID NO: 360 - protein, 17 aa, synthetic construct
KSSHNVLYSSNNKNYLA

SEQ ID NO: 361 - protein, 11 aa, synthetic construct
RASQSVSSNLA

SEQ ID NO: 362-367 - skipped (6 entries)

SEQ ID NO: 368 - protein, 17 aa, synthetic construct
AISGSGGSTYYAESVKG

SEQ ID NO: 369 - protein, 7 aa, synthetic construct
AASSLQS

SEQ ID NO: 370 - protein, 9 aa, synthetic construct
QQADAFPRT

SEQ ID NO: 371 - protein, 17 aa, synthetic construct
IIYPGDADARYSPSFQG

SEQ ID NO: 372 - protein, 9 aa, synthetic construct
LQDNNFPPT

SEQ ID NO: 373 - protein, 17 aa, synthetic construct
IIYPGDADARYSPSFQG

SEQ ID NO: 374 - protein, 12 aa, synthetic construct
RRQGIFGDALDF

SEQ ID NO: 375 - protein, 12 aa, synthetic construct
DAGYLALYGTDV

(19) United States
(12) Patent Application Publication
(10) Pub. No.: US 2025/0019437 A1
(43) Pub. Date: Jan. 16, 2025

(54) ANTI-TREM2 ANTIBODY AND USES THEREOF

(71) Applicant: Vigil Neuroscience, Inc., Watertown, MA (US)

(72) Inventors: Spyridon PAPAPETROPOULOS, Watertown, MA (US); Evan Andrew THACKABERRY, Watertown, MA (US); David K. STILES, Watertown, MA (US); Andrew J. MARSH, Watertown, MA (US); Ryan O'MARA, Watertown, MA (US)

(73) Assignee: Vigil Neuroscience, Inc., Watertown, MA (US)

(21) Appl. No.: 18/712,171

(22) PCT Filed: Nov. 22, 2022
(86) PCT No.: PCT/US2022/080342

§ 371 Date: May 21, 2024

Related U.S. Application Data
(60) Provisional application No. 63/264,428, filed on Nov. 22, 2021.
(60) Provisional application No. 63/381,897, filed on Nov. 1, 2022.

(30) Foreign Application Priority Data
Nov. 22, 2021 (GR) 20210100820

(51) Int. Cl. C07K 16/28; A61K 39/00

(52) U.S. Cl. CPC C07K 16/2803; A61K 2039/505; A61K 2039/545; C07K 2317/94

(57) ABSTRACT

The present invention provides anti-TREM2 antibodies, formulations thereof, and methods of use thereof.

### RELATED APPLICATIONS

This application claims benefit to U.S. Provisional Application No. 63/381,897, filed on Nov. 1, 2022, U.S. Provisional Application No. 63/264,428, filed on Nov. 22, 2021, and Greece Application No. 20210100820, filed on Nov. 22, 2021, the contents of which are herein incorporated by reference in their entireties.

### SEQUENCE LISTING

The content of the electronically submitted Sequence Listing XML (Name: 403433-013WO_SL; Size: 331,607 bytes; Created: Nov. 18, 2022) is herein incorporated by reference in its entirety.

### TECHNICAL FIELD OF THE INVENTION

The present invention provides an anti-TREM2 antibody, formulations thereof, and methods of use thereof for treating adult-onset leukoencephalopathy with axonal spheroids and pigmented glia (ALSP) in a human patient.

### BACKGROUND OF THE INVENTION

Mutations in triggering receptor expressed on myeloid cells 2 (TREM2), a receptor with expression restricted to microglia in the central nervous system, increase the risk of neurodegenerative diseases, such as Alzheimer's disease and frontal temporal dementia (Ulland and Colonna, Nature Reviews, 14, 2018). The TREM2 receptor influences microglial state and function through its interaction with a variety of ligands, and such ligands are critical for sensing tissue damage and minimizing neuropathogenesis (Deczkowska, Cell, 181, 2020). In addition, TREM2 receptor agonism is required for progression of microglia to a neuroprotective, disease-associated (DAM) phenotype (Keren-Shaul Cell, 169, 2017).

### SUMMARY OF THE INVENTION

Disclosed herein are compositions of anti-triggering receptor expressed on myeloid cells 2 (TREM2) antibodies, as well as methods of use thereof. The response of microglial cells to changes in the environment of the CNS is activated through TREM2 and its associated protein kinase complex, DAP12. The TREM2/DAP12 signal functions as the primary regulator that transforms microglia from a homeostatic to a neural disease-associated state and produces an anti-inflammatory response and neurotrophic factors to protect injured neurons and to enable nerve tissue regeneration. In an embodiment, the anti-TREM2 antibody is “Ab-1,” a monoclonal antibody able to bind TREM2, also described herein as VGL101. Anti-TREM2 antibody “Ab-1” can be useful for modulating the activity of such cells without suppressing or compromising the immune system. Without wishing to be bound by any specific theory, anti-TREM2 antibody “Ab-1” activates TREM2, slows disease progression, and enhances the neural tissue repair mechanisms regulated by microglia.

Accordingly, in one aspect, the present invention provides a liquid formulation of an anti-TREM2 antibody. In an embodiment, the anti-TREM2 antibody is “Ab-1”, and the liquid formulation further comprises a pharmaceutically acceptable excipient and/or carrier. In some embodiments, a liquid formulation of the invention comprises sodium acetate. In some embodiments, a liquid formulation of the invention comprises sucrose. In some embodiments, a liquid formulation of the invention comprises Polysorbate 80.

In another aspect, the present invention provides a method for treating adult-onset leukoencephalopathy with axonal spheroids and pigmented glia (ALSP) in a human patient, comprising administering to the patient in need thereof a therapeutically effective amount of an anti-TREM2 antibody. In some embodiments, an anti-TREM2 antibody is anti-TREM2 antibody “Ab-1.” In some embodiments, a method of the present invention comprises administering to a patient in need thereof a liquid formulation comprising anti-TREM2 antibody “Ab-1,” as described herein.

### BRIEF DESCRIPTION OF THE DRAWINGS

FIG. 1 is a graph showing the mean serum concentration of anti-TREM2 antibody “Ab-1” in pg/mL over time in healthy volunteers who received a single intravenous infusion of 1, 3, 10, 20, or 30 mg/kg. N=6 in each cohort except for 10 mg/kg, N=5. Data for 30 mg/kg dose was only available for 28 days post-dose.

FIG. 2 is a graph showing the mean serum concentration of anti-TREM2 antibody “Ab-1” in pg/mL over time in healthy volunteers who received a dose of 20 mg/kg, administered intravenously every 28 days, for a total of three infusions. N=12

FIG. 3A is a graph showing the effect of anti-TREM2 antibody “Ab-1” on sTREM2 in cerebrospinal fluid (CSF) of 18 healthy volunteers administered single intravenous doses at 3, 10, and 20 mg/kg. The data are plotted as mean change from baseline in pg/mL of sTREM2 in the CSF over 336 hours (14 days). N=6 per dose. FIG. 3B is a graph showing the effect of anti-TREM2 antibody “Ab-1” on CSF sTREM2 in 6 healthy volunteers administered 20 mg/kg intravenously every 28 days, for a total of three infusions. Mean CSF levels of sTREM2 were evaluated at baseline (prior to dosing), 48 hours, and 672 hours (28 days) following the third and final dose. * p<0.05.

FIG. 4A is a graph showing the effect of anti-TREM2 antibody “Ab-1” on sCSF1R in CSF of 18 healthy volunteers administered single intravenous doses at 3, 10, and 20 mg/kg. The data are plotted as mean change from baseline in pg/mL of sCSF1R in the CSF over 336 hours (14 days). N=6 per dose. FIG. 4B is a graph showing the effect of anti-TREM2 antibody “Ab-1” on CSF sCSF1R in 6 healthy volunteers administered 20 mg/kg intravenously every 28 days, for a total of three infusions. Mean CSF levels of sCSF1R were evaluated at baseline (prior to dosing), 48 hours, and 672 hours (28 days) following the third and final dose. * p<0.05.

### DETAILED DESCRIPTION OF THE INVENTION

### 1. General Description of Certain Embodiments of the Invention

Described herein are formulations of an anti-TREM2 antibody, such as “Ab-1”, suitable for administering to a subject, e.g., a human subject. It has been found that, in the in vitro cytokine release assay, at concentrations up to 10 μg/mL, anti-TREM2 antibody “Ab-1” had no effect on the release of granulocyte colony-stimulating factor (CSF), monocyte chemoattractant protein-1 (MCP-1), interferon-gamma inducible protein of 10 kDa (IP-10), interleukin (IL)-1β, IL-2, IL-4, IL-6, IL-10, IL-12p40, IL-13, and IFN-γ. A minimal but dose-related increase in tumor necrosis factor-α release was observed at the two highest (4 and 10 μg/mL) concentrations. However, this increase was in the range reported for other marketed monoclonal antibodies and no increase in TNF-α was observed in vivo in the GLP non-human primate (NHP) study at any dose tested.

Anti-TREM2 antibody “Ab-1” was well-tolerated in NHPs upon repeat dose weekly IV administration of up to 200 mg/kg for 1 month. No test article-related effects were observed on clinical signs, body weight, food consumption, ophthalmological exams, or clinical pathology parameters (serum chemistry, hematology, coagulation, and urinalysis). Macroscopic and microscopic examination revealed no adverse findings or effects on organ weights. Safety pharmacology parameters were included in the GLP toxicology study in NHPs, in keeping with the International Council for Harmonisation (ICH) Tripartite Guideline for Good Clinical Practice (GCP) S6 guidance (ICH S6). No CNS effects were observed upon detailed neurobehavioral exams, and electrocardiograms (ECGs) revealed no anti-TREM2 antibody “Ab-1” related findings.

The no-observed-adverse-effect level (NOAEL) is considered to be 200 mg/kg. The exposures observed after the 4th once every 7 days (q7d) dose of 200 mg/kg were:
Area under the serum curve from predose (time 0) to the time of the quantifiable concentration at 168 hr (AUC0-168 hr) for 200 mg/kg on Day 22: 838000 h*pg/mL
Maximum serum/cerebrospinal fluid concentration (Cmax) for 200 mg/kg on Day 22: 8320 μg/mL

Doses were administered q7d for 29 days in the cynomolgus monkey. The expected dose regimen to be administered in humans as part of the MAD portion of the study is once every 28 days. To calculate the NOAEL limits, based on the q7d dosing in the cynomolgus monkey, the AUC0-168 hr on Day 22 was multiplied by 4 (the number of doses the monkey received over a 28 day period); the Cmax NOAEL limit remains unchanged:
NOAEL limit for area under the serum or CSF concentration-time curve from predose (time 0) extrapolated to infinite time (AUClast+Clast/λz) (AUC0−∞): 838,000 h*μg/mL×4=3,350,000 h*μg/mL
NOAEL limit for Cmax: 8,320 pg/mL
   The NHP NOAEL of 200 mg/kg provides safety margins of 344× and 337× over the expected Cmax and AUC_{INF} at 1 mg/kg starting dose in healthy volunteers.

Accordingly, in one aspect, the present invention provides a liquid formulation comprising anti-TREM2 antibody “Ab-1”, and a pharmaceutically acceptable excipient and/or carrier. In some embodiments, a pharmaceutically acceptable excipient and/or carrier of the invention is selected from those as described herein.

In another aspect, the present invention provides a method for treating adult-onset leukoencephalopathy with axonal spheroids and pigmented glia (ALSP) in a human patient, comprising administering to the patient in need thereof a therapeutically effective amount of an anti-TREM2 antibody. In some embodiments, a method of the invention comprises administering to a patient in need thereof a therapeutically effective amount of a liquid formulation as described herein.

In some embodiments, an anti-TREM2 antibody comprises a light chain variable region comprising a CDRL1 having an amino acid sequence according to SEQ ID NO: 2; a CDRL2 having an amino acid sequence according to SEQ ID NO: 3; and a CDRL3 having an amino acid sequence according to SEQ ID NO: 4, and a heavy chain variable region comprising a CDRH1 having an amino acid sequence according to SEQ ID NO: 6; a CDRH2 having an amino acid sequence according to SEQ ID NO: 7; and a CDRH3 having an amino acid sequence according to SEQ ID NO: 8.

In some embodiments, an anti-TREM2 antibody comprises a light chain variable region having an amino acid sequence according to SEQ ID NO: 1, and a heavy chain variable region having an amino acid sequence according to SEQ ID NO: 5.

In some embodiments, an anti-TREM2 antibody is an IgG, optionally an IgG₁.

In some embodiments, an anti-TREM2 antibody comprises a kappa light constant region.

In some embodiments, an anti-TREM2 antibody is an IgG₁ comprising a variant constant region having one or more mutations selected from R292C, N297G, V302C, D356E, or L358M, according to EU numbering.

In some embodiments, an anti-2 antibody is anti-TREM2 antibody “Ab-1.”

### 2. Definitions

As used herein, the term “anti-TREM2 antibody Ab-F” refers to an anti-TREM2 antibody “Ab-1”, comprising a light chain having an amino acid sequence of SEQ ID NO: 9, and a heavy chain having amino acid sequence of SEQ ID NO: 10. “Ab-1” is used interchangeably with VGL101, and describes an antibody having a CAS No. 2733621-19-5 having the amino acid sequences summarized in Table 1 below.

TABLE 1
List of Amino Acid Sequences.

| SEQ ID NO. | Amino Acid Sequence |
|---|---|
| SEQ ID NO: 1 | EIVMTQSPATLSVSPGERATLSCRASQSVSSNLAWFQQKPGQAPRLLIYGA |
| | STRATGIPARFSGSGSGTEFTLTISSLQPEDFAVYYCLQDNNFPPTFGQGT |
| | KVDIK |
| SEQ ID NO: 2 | RASQSVSSNLA |
| SEQ ID NO: 3 | GASTRAT |
| SEQ ID NO: 4 | LQDNNFPPT |
| SEQ ID NO: 5 | EVQLVQSGAEVKKPGESLKISCKGSGYSFTSYWIGWVRQMPGKGLEWMGII |
| | YPGDADARYSPSFQGQVTISADKSISTAYLQWSSLKASDTAMYFCARRRQG |
| | IFGDALDFWGQGTLVTVSS |
| SEQ ID NO: 6 | SYWIG |
| SEQ ID NO: 7 | IIYPGDADARYSPSFQG |
| SEQ ID NO: 8 | RRQGIFGDALDF |
| SEQ ID NO: 9 | EIVMTQSPATLSVSPGERATLSCRASQSVSSNLAWFQQKPGQAPRLLIYGA |
| | STRATGIPARFSGSGSGTEFTLTISSLQPEDFAVYYCLQDNNFPPTFGQGT |
| | KVDIKRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSSP |
| | VTKSFNRGEC |
| SEQ ID NO: 10 | EVQLVQSGAEVKKPGESLKISCKGSGYSFTSYWIGWVRQMPGKGLEWMGII |
| | YPGDADARYSPSFQGQVTISADKSISTAYLQWSSLKASDTAMYFCARRRQG |
| | IFGDALDFWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPCEEQYGSTYRC |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGK |

In some embodiments, the anti-TREM2 antibody is an anti-TREM2 antibody recited in one or more of WO 2018/195506; U.S. Pat. No. 8,231,878; U.S. Patent Publication No. 2019/0010230; WO 2017/062672; WO 2019/028292; WO 2018/015573; WO 2019/055841; WO 2019/118513; WO 2020/055975; WO 2020/079580; KR Patent Publication No. KR20200048069; each of which is incorporated herein by reference in its entirety. In some embodiments, the anti-TREM2 antibody is AL002. In some embodiments, the anti-TREM2 antibody is DNL919. In some embodiments, the anti-TREM2 antibody is not “Ab-1” (i.e., VGL101).

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid, or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium, and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

As used herein, the terms “about” or “approximately” have the meaning of within 20% of a given value or range. In some embodiments, the term “about” refers to within 20%, 19%, 18%, 17%, 16%, 15%, 14%, 13%, 12%, 11%, 10%, 9%, 8%, 7%, 6%, 5%, 4%, 3%, 2%, or 1% of a given value.

The present invention relates to antibodies that specifically bind to TREM2, particularly human TREM2. In humans, the TREM2 gene is located within a TREM gene cluster at chromosome 6p21.1. The TREM gene cluster encodes four TREM proteins (TREM1, TREM2, TREM4, and TREM5) as well as two TREM-like proteins (TLT-1 and TLT-2). The TREM2 gene encodes a 230 amino acid protein consisting of an extracellular domain, a transmembrane region, and a short cytoplasmic tail (Paradowska-Gorycka et al., Human Immunology, Vol. 74: 730-737, 2013). The extracellular domain contains a single type V Ig-super family domain, with three potential N-glycosylation sites. The wild-type human TREM2 amino acid sequence (NCBI Reference Sequence: NP_061838.1) is provided below as SEQ ID NO: 354.

(SEQ ID NO: 354)
MEPLRLLILLFVTELSGAHNTTVFQGVAGQSLQVSCPYDSMKHWGRRKAW
CRQLGEKGPCQRWSTHNLWLLSFLRRWNGSTAITDDTLGGTLTITLRNLQ
PHDAGLYQCQSLHGSEADTLRKVLVEVLADPLDHRDAGDLWFPGESESFE
DAHVEHSISRSLLEGEIPFPPTSILLLLACIFLIKILAASALWAAAWHGQ
KPGTHPPSELDCGHDPGYQLQTLPGLRDT

Amino acids 1 to 18 of the wild-type human TREM2 protein (SEQ ID NO: 354) is a signal peptide, which is generally removed from the mature protein. The mature human TREM2 protein comprises an extracellular domain at amino acids 19-174 of SEQ ID NO: 354, a transmembrane domain at amino acids 175-195 of SEQ ID NO: 354, and a cytoplasmic domain at amino acids 196-230 of SEQ ID NO: 354. The amino acid sequence of the extracellular domain (including the signal peptide) of human TREM2 is provided below as SEQ ID NO: 355.

(SEQ ID NO: 355)
MEPLRLLILLFVTELSGAHNTTVFQGVAGQSLQVSCPYDSMKHWGRRKAW
CRQLGEKGPCQRWSTHNLWLLSFLRRWNGSTAITDDTLGGTLTITLRNLQ
PHDAGLYQCQSLHGSEADTLRKVLVEVLADPLDHRDAGDLWFPGESESFE
DAHVEHSISRSLLEGEIPFPPTS

The term ‘Human triggering receptor expressed on myeloid cells-2” or “human TREM2” can refer to a polypeptide of SEQ ID NO: 354, a polypeptide of SEQ ID NO: 355, polypeptides of SEQ ID NO: 354 or SEQ ID NO: 355 minus the signal peptide (amino acids 1-18), allelic variants of human TREM2, or splice variants of human TREM2. In some embodiments, the term “human TREM2” includes naturally occurring variants of TREM2, such as mutations R47H, Q33× (X is a stop codon), Y38C, T66M, D87N, H157Y, R98W, and S116C.

### 3. Description of Exemplary Embodiments

### Antibodies

In one aspect, the present invention provides a method of treating ALSP in a human patient, the method comprising administering to the patient an effective amount of an antigen binding protein or an antibody, or an antigen-binding fragment thereof, which increases the activity of TREM2. In some embodiments, the antibody is an agonist of TREM2. In some embodiments, the antibody is an agonist of TREM2 that specifically binds to and activates human TREM2. In some embodiments, the antibody is anti-human TREM2 antibody VGL101.

The TREM2 agonist antibodies specifically bind to human TREM2 (SEQ ID NO: 354) or an extra cellular domain (ECD) of human TREM2 (e.g., ECD set forth in SEQ ID NO: 355), for example with an equilibrium dissociation constant (K_{D}) less than 50 nM, less than 25 nM, less than 10 nM, or less than 5 nM. In some embodiments, the TREM2 agonist antibodies do not cross-react with other TREM proteins, such as human TREM1. In some embodiments, the TREM2 agonist antibodies do not bind to human TREM1.

In some embodiments, the TREM2 antibody specifically binds to human TREM2 residues 19-174 (SEQ ID NO: 354). In some embodiments, the TREM2 antibody specifically binds to IgV region of human TREM2, for example human TREM2 residues 19-140 (SEQ ID NO: 354).

In certain embodiments, anti-TREM2 antibodies of the present disclosure bind to one or more amino acids within amino acid residues 29-112 of human TREM 2 (SEQ ID NO: 354), or within amino acid residues on a TREM2 protein corresponding to amino acid residues 29-112 of SEQ ID NO: 354. In some embodiments, anti-TREM2 antibodies of the present disclosure bind to one or more amino acids within amino acid residues 29-41 of human TREM 2 (SEQ ID NO: 354), or within amino acid residues on a TREM2 protein corresponding to amino acid residues 29-41 of SEQ ID NO: 354. In some embodiments, anti-TREM2 antibodies of the present disclosure bind to one or more amino acids within amino acid residues 47-69 of human TREM 2 (SEQ ID NO: 354), or within amino acid residues on a TREM2 protein corresponding to amino acid residues 47-69 of SEQ ID NO: 354. In some embodiments, anti-TREM2 antibodies of the present disclosure bind to one or more amino acids within amino acid residues 76-86 of human TREM 2 (SEQ ID NO: 354), or within amino acid residues on a TREM2 protein corresponding to amino acid residues 76-86 of SEQ ID NO: 354. In some embodiments, anti-TREM2 antibodies of the present disclosure bind to one or more amino acids within amino acid residues 91-100 of human TREM2 (SEQ ID NO: 354), or within amino acid residues on a TREM2 protein corresponding to amino acid residues 91-100 of SEQ ID NO: 354. In some embodiments, anti-TREM2 antibodies of the present disclosure bind to one or more amino acids within amino acid residues 99-115 of human TREM 2 (SEQ ID NO: 354), or within amino acid residues on a TREM2 protein corresponding to amino acid residues 99-115 of SEQ ID NO: 354. In some embodiments, anti-TREM2 antibodies of the present disclosure bind to one or more amino acids within amino acid residues 104-112 of human TREM 2 (SEQ ID NO: 354), or within amino acid residues on a TREM2 protein corresponding to amino acid residues 104-112 of SEQ ID NO: 354. In some embodiments, anti-TREM2 antibodies of the present disclosure bind to one or more amino acids within amino acid residues 114-118 of human TREM 2 (SEQ ID NO: 354), or within amino acid residues on a TREM2 protein corresponding to amino acid residues 114-118 of SEQ ID NO: 354. In some embodiments, anti-TREM2 antibodies of the present disclosure bind to one or more amino acids within amino acid residues 130-171 of human TREM 2 (SEQ ID NO: 354), or within amino acid residues on a TREM2 protein corresponding to amino acid residues 130-171 of SEQ ID NO: 354. In some embodiments, anti-TREM2 antibodies of the present disclosure bind to one or more amino acids within amino acid residues 139-153 of human TREM 2 (SEQ ID NO: 354), or within amino acid residues on a TREM2 protein corresponding to amino acid residues 139-153 of SEQ ID NO: 354. In some embodiments, anti-TREM2 antibodies of the present disclosure bind to one or more amino acids within amino acid residues 139-146 of human TREM 2 (SEQ ID NO: 354), or within amino acid residues on a TREM2 protein corresponding to amino acid residues 139-146 of SEQ ID NO: 354. In some embodiments, anti-TREM2 antibodies of the present disclosure bind to one or more amino acids within amino acid residues 130-144 of human TREM 2 (SEQ ID NO: 354), or within amino acid residues on a TREM2 protein corresponding to amino acid residues 130-144 of SEQ ID NO: 354. In some embodiments, anti-TREM2 antibodies of the present disclosure bind to one or more amino acids within amino acid residues 158-171 of human TREM 2 (SEQ ID NO: 354), or within amino acid residues on a TREM2 protein corresponding to amino acid residues 158-171 of SEQ ID NO: 354.

In some embodiments, anti-TREM2 antibodies of the present disclosure bind to one or more amino acids within amino acid residues 43-50 of human TREM 2 (SEQ ID NO: 354), or within amino acid residues on a TREM2 protein corresponding to amino acid residues 43-50 of SEQ ID NO: 354. In some embodiments, anti-TREM2 antibodies of the present disclosure bind to one or more amino acids within amino acid residues 49-57 of human TREM 2 (SEQ ID NO: 354), or within amino acid residues on a TREM2 protein corresponding to amino acid residues 49-57 of SEQ ID NO: 354. In some embodiments, anti-TREM2 antibodies of the present disclosure bind to one or more amino acids within amino acid residues 139-146 of human TREM 2 (SEQ ID NO: 354), or within amino acid residues on a TREM2 protein corresponding to amino acid residues 139-146 of SEQ ID NO: 354. In some embodiments, anti-TREM2 antibodies of the present disclosure bind to one or more amino acids within amino acid residues 140-153 of human TREM 2 (SEQ ID NO: 354), or within amino acid residues on a TREM2 protein corresponding to amino acid residues 140-153 of SEQ ID NO: 354. In some embodiments, the TREM2 antibody specifically binds to the stalk region of human TREM2, for example amino acid residues 145-174 of human TREM2.

In some embodiments, the antibody, or an antigen-binding fragment thereof, specifically binds TREM2 and prevents the degradation or cleavage of TREM2.

In some embodiments, the antibody is a polyclonal antibody. In some embodiments, the antibody is a monoclonal antibody. In some embodiments, the antibody is a chimeric antibody. In some embodiments, the antibody is a humanized antibody. In some embodiments, the antibody is a human antibody, particularly a fully human antibody. In some embodiments, the antibody is a bispecific or other multivalent antibody. In some embodiments, the antibody is a single chain antibody.

In some embodiments, a TREM2 activating antibody comprise a light chain variable region comprising complementarity determining regions CDRL1, CDRL2, and CDRL3 and a heavy chain variable region comprising complementarity determining regions CDRH1, CDRH2, and CDRH3 described herein.

In certain embodiments, the TREM2 agonist antigen binding proteins of the invention comprise at least one light chain variable region comprising a CDRL1, CDRL2, and CDRL3, and at least one heavy chain variable region comprising a CDRH1, CDRH2, and CDRH3 from an anti-TREM2 agonist antibody described herein.

In some embodiments, a TREM2 activating antibody comprises a light chain variable region and a heavy chain variable region described herein. The light chain and heavy chain variable regions or CDRs may be from any of the anti-TREM2 antibodies or a variant thereof described herein.

A. PCT Patent Application Publication No. WO20181195506A1

In some embodiments, the TREM2 agonist is an antigen binding protein or an antibody, or an antigen-binding fragment thereof, as described in PCT Patent Application Publication No. WO2018/195506A1, which is incorporated by reference herein, in its entirety.

In some embodiments, the TREM2 agonist antigen binding protein comprises a CDRL1 or a variant thereof having one, two, three or four amino acid substitutions; a CDRL2, or a variant thereof having one, two, three or four amino acid substitutions; a CDRL3, or a variant thereof having one, two, three or four amino acid substitutions; a CDRH1, or a variant thereof having one, two, three or four amino acid substitutions; a CDRH2, or a variant thereof having one, two, three or four amino acid substitutions; and a CDRH3, or a variant thereof having one, two, three or four amino acid substitutions, where the amino acid sequences of the CDRL1, CDRL2, CDRL3, CDRH1, CDRH2, and CDRH3 are provided in Tables 1A and 1B below, along with exemplary light chain and variable regions.

TABLE 1A
Exemplary Anti-Human TREM2 Antibody Light Chain
Variable Region Amino Acid Sequences

| Ab | VI | VL Amino Acid | | | |
|---|---|---|---|---|---|
| ID. | Group | Sequence | CDRL1 | CDRL2 | CDRL3 |
| 12G10 | LV-01 | QAVPTQPSSLSASPGV | TLRSGINVGTY | YKSDSDK | MIWYSSAVV |
| | | LASLTCTLRSGINVGT | RIY | QQGS | (SEQ ID |
| | | YRIYWYQQKPGSPPQY | (SEQ ID NO: | (SEQ ID: | NO: 31) |
| | | LLRYKSDSDKQQGSGV | 356) | NO: 19) | |
| | | PSRESGSKDASANAGI | | | |
| | | LLISGLQSEDEADYYC | | | |
| | | MIWYSSAVVFGGGTKL | | | |
| | | TVL (SEQ ID NO: | | | |
| | | 46) | | | |
| 26A10 | LV-02 | SYELTQPPSVSVSPGQ | SGDKLGDKYVC | QDSKRPS | QAWDSNTVV |
| | | TASITCSGDKLGDKYV | (SEQ ID NO: | (SEQ ID: | (SEQ ID |
| | | CWYQQKPGQSPVLVIY | 357) | NO: 20) | NO: 32) |
| | | QDSKRPSGIPERFSGS | | | |
| | | NSGNTATLTISGTQAM | | | |
| | | DEADYYCQAWDSNTVV | | | |
| | | FGGGTKLTVL (SEQ | | | |
| | | ID NO: 47) | | | |
| 26C10 | LV-03 | SFELTQPPSVSVSPGQ | SGDKLGDKYVC | QDTKRPS | QAWDSSTVV |
| | | TASITCSGDKLGDKYV | (SEQ ID NO: | (SEQ ID: | (SEQ ID |
| | | CWYQQKPGQSPMLVIY | 357) | NO: 21) | NO: 33) |
| | | QDTKRPSGIPERFSGS | | | |
| | | NSGNTATLTISGTQAM | | | |
| | | DEADYYCQAWDSSTVV | | | |
| | | FGGGTKLTVL (SEQ | | | |
| | | ID NO: 48) | | | |
| 26F2 | LV-04 | SYELTQPPSVSVSPGQ | SGDKLGDKYVC | QDSKRPS | QAWDSSTVV |
| | | TASITCSGDKLGDKYV | (SEQ ID NO: | (SEQ ID: | (SEQ ID |
| | | CWYQQKPGQSPVLVIF | 357) | NO: 20) | NO: 33) |
| | | QDSKRPSGIPERFSGS | | | |
| | | NSGNTATLTISGTQAM | | | |
| | | DEADYYCQAWDSSTVV | | | |
| | | FGGGTKLTVL (SEQ | | | |
| | | ID NO: 49) | | | |
| 33B12 | LV-05 | SYELTQPPSVSVSPGQ | SGDKLGDKYVC | QDSKRPS | QAWDSSTVV |
| | | TASITCSGDKLGDKYV | (SEQ ID NO: | (SEQ ID: | (SEQ ID |
| | | CWYQQKPGQSPVLVIY | 357) | NO: 20) | NO: 33) |
| | | QDSKRPSGIPERFSGS | | | |
| | | NSGNTATLTISGTQAM | | | |
| | | DEADYYCQAWDSSTVV | | | |
| | | FGGGTKLTVL (SEQ | | | |
| | | ID NO: 50) | | | |
| 24C12 | LV-06 | GIVMTQSPDSLAVSLG | KSSRSVLYSSN | WASTRES | QQYYITPIT |
| | | ERATINCKSSRSVLYS | NKNYLA | (SEQ ID: | (SEQ ID |
| | | SNNKNYLAWYQQKPGQ | (SEQ ID NO: | NO: 22) | NO: 34) |
| | | PPKVLIYWASTRESGV | 358) | | |
| | | PDRFSGSGSGTDFTLT | | | |
| | | ISSLQAEDVAVYNCQQ | | | |
| | | YYITPITFGQGTRLEI | | | |
| | | K (SEQ ID NO: | | | |
| | | 51) | | | |
| 24G6 | LV-07 | DIVMTQSPDSLAVSLG | KSSQSVLYSSN | WASTRES | QQYYSTPLT |
| | | ERATINCKSSQSVLYS | NKHELA | (SEQ ID: | (SEQ ID |
| | | SNNKHFLAWYQQKPGQ | (SEQ ID NO: | NO: 22) | NO: 35) |
| | | PPKLLIYWASTRESGV | 359) | | |
| | | PDRFSGSGSGTDFTLT | | | |
| | | ISSLQAEDVAFYYCQQ | | | |
| | | YYSTPLTFGGGTKVEI | | | |
| | | K (SEQ ID NO: | | | |
| | | 52) | | | |
| 24A10 | LV-08 | DIVMTQSPDSLAVSLG | KSSHNVLYSSN | WASTRES | HQYYSTPCS |
| | | ERATITCKSSHNVLYS | NKNYLA | (SEQ ID: | (SEQ ID |
| | | SNNKNYLAWYQQKPGQ | (SEQ ID NO: | NO: 22) | NO: 36) |
| | | PPKLLIYWASTRESGV | 360) | | |
| | | PDRFSGSGSGTDETLT | | | |
| | | ISSLQAEDVAVYYCHQ | | | |
| | | YYSTPCSFGQGTKLEI | | | |
| | | K (SEQ ID NO: | | | |
| | | 53) | | | |
| 10E3 | LV-09 | EIVMTQSPATLSVSPG | RASQSVSSNLA | GASTRAT | LQDNNWPPT |
| | | ERATLSCRASQSVSSN | (SEQ ID NO: | (SEQ ID: | (SEQ ID |
| | | LAWFQQKPGQAPRLLI | 361) | NO: 23) | NO: 37) |
| | | YGASTRATGIPARFSV | | | |
| | | SGSGTEFTLTISSLQS | | | |
| | | EDFAFYYCLQDNNWPP | | | |
| | | TFGPGTKVDIK (SEQ | | | |
| | | ID NO: 54) | | | |
| 13E7 | LV-10 | EIVMTQSPATLSVSPG | RASQSVSSNLA | GASTRAT | LQDNNWPPT |
| 14C12 | | ERATLSCRASQSVSSN | (SEQ ID NO: | (SEQ ID: | (SEQ ID |
| | | LAWFQQKPGQAPRLLI | 361) | NO: 23) | NO: 37) |
| | | YGASTRATGIPARFSV | | | |
| | | SGSGTEFTLTISSLOS | | | |
| | | EDFAVYYCLQDNNWPP | | | |
| | | TFGPGTKVDIK (SEQ | | | |
| | | ID NO: 55) | | | |
| 25F12 | LV-11 | EKVMTQSPATLSVSPG | RASQSVNNNLA | GASTRAT | QQYNNWPRT |
| | | ERATLSCRASQSVNNN | (SEQ ID NO: | (SEQ ID: | (SEQ ID |
| | | LAWYQQKPGQAPRLLI | 11) | NO: 23) | NO: 38) |
| | | YGASTRATGIPARESG | | | |
| | | SGSGTEFTLTISSLOS | | | |
| | | EDFAVYYCQQYNNWPR | | | |
| | | TFGQGTKVEIK (SEQ | | | |
| | | ID NO: 56) | | | |
| 32E3 | LV-12 | EFVLTQSPGTLSLSPG | RASQIISSNYL | SASSRAT | QQFDSSPIT |
| | | ERATLSCRASQIISSN | A | (SEQ ID: | (SEQ ID |
| | | YLAWYQQKPGQAPRLL | (SEQ ID NO: | NO: 24) | NO: 39) |
| | | IYSASSRATGIPDRES | 12) | | |
| | | GSGSGTDETLTISRLE | | | |
| | | PEDFAVYYCQQFDSSP | | | |
| | | ITFGRGTRLDIK | | | |
| | | (SEQ ID NO: 57) | | | |
| 24F4 | LV-13 | EIVLTQSPGTLSLSPG | RASQSVSSSYL | GASSRAT | QQYDTSPFT |
| | | ERATLSCRASQSVSSS | A | (SEQ ID: | (SEQ ID |
| | | YLAWYQQKPGQAPRLL | (SEQ ID NO: | NO: 25) | NO: 40) |
| | | IYGASSRATGIPDRES | 13) | | |
| | | GSGSGTDFTLTISRLE | | | |
| | | PEDFALYYCQQYDTSP | | | |
| | | FTFGPGTKVDIK | | | |
| | | (SEQ ID NO: 58) | | | |
| 16B8 | LV-14 | DIQMTQSPSSVSASVG | RASQDINSWLA | AASSLQT | QQSNSFPIT |
| | | DRVTVTCRASQDINSW | (SEQ ID NO: | (SEQ ID: | (SEQ ID |
| | | LAWYQQKPGKAPKLLI | 14) | NO: 26) | NO: 41) |
| | | YAASSLQTGVPSRESG | | | |
| | | SGSGTDFTLTISSLOP | | | |
| | | EDFATYSCQQSNSFPI | | | |
| | | TFGQGTRLEIK (SEQ | | | |
| | | ID NO: 59) | | | |
| 4C5 | LV-15 | DIQMTQSPSSVSASVG | RASQGISNWLA | AASSLQV | QQADSFPRN |
| | | DRVTITCRASQGISNW | (SEQ ID NO: | (SEQ ID: | (SEQ ID |
| | | LAWYQQKPGKAPKLLI | 15) | NO: 27) | NO: 42) |
| | | YAASSLQVGVPLRESG | | | |
| | | SGSGTDFTLTISSLQP | | | |
| | | EDFATYYCQQADSFPR | | | |
| | | NFGQGTKLEIK (SEQ | | | |
| | | ID NO: 60) | | | |
| 6E7 | LV-16 | DIQMTQSPSSVSASVG | RASQGISSWLA | AASSLQN | QQADSFPRT |
| | | DRVTITCRASQGISSW | (SEQ ID NO: | (SEQ ID: | (SEQ ID |
| | | LAWYQQKPGKAPKLLI | 16) | NO: 28) | NO: 43) |
| | | YAASSLQNGVPSRESG | | | |
| | | SGSGTDFTLTISSLQP | | | |
| | | EDFATYFCQQADSFPR | | | |
| | | TFGQGTKLEIK (SEQ | | | |
| | | ID NO: 61) | | | |
| 5E3 | LV-17 | DIQMTQSPSSLSASVG | RASQGISNYLA | AASSLQS | QQYSTYPFT |
| | | DRVTITCRASQGISNY | (SEQ ID NO: | (SEQ ID: | (SEQ ID |
| | | LAWFQQKPGKAPKSLI | 17) | NO: 29) | NO: 44) |
| | | YAASSLQSGVPSKESG | | | |
| | | SGSGTDFTLTISSLQP | | | |
| | | EDFATYYCQQYSTYPF | | | |
| | | TFGPGTKVDIK (SEQ | | | |
| | | ID NO: 62) | | | |
| 4G10 | LV-18 | DIQMTQSPSSLSASVG | RASQGIRNDLG | AASSLPS | LOHNSYPWT |
| | | DRVTITCRASQGIRND | (SEQ ID NO: | (SEQ ID: | (SEQ ID |
| | | LGWYQQKPGNAPKRLI | 18) | NO: 30) | NO: 45) |
| | | YAASSLPSGVPSRESG | | | |
| | | SGSGPEFTLTISSLQP | | | |
| | | EDFATYYCLQHNSYPW | | | |
| | | TFGQGTKVEIT (SEQ | | | |
| | | ID NO: 63) | | | |

TABLE 1B
Exemplary Anti-Human TREM2 Antibody Heavy Chain Variable
Region Amino Acid Sequences

| Ab | VH | VH Amino Acid | | | |
|---|---|---|---|---|---|
| ID. | Group | Sequence | CDRH1 | CDRH2 | CDRH3 |
| 12G10 | HV-01 | EVQLLESGGGLVQPGG | SYAMS | AIGGGGVSTYCADSV | FYIAVAGSHEDY |
| 24C12 | | SLRLSCAASGFTESSY | (SEQ ID | KG (SEQ ID NO: | (SEQ ID NO: 95) |
| | | AMSWVRQAPGKGLEWV | NO: 77) | 87) | |
| | | SAIGGGGVSTYCADSV | | | |
| | | KGRFTISRDNSKNTLY | | | |
| | | LQMNSLRAEDTAVYYC | | | |
| | | AKFYIAVAGSHEDYWG | | | |
| | | QGTLVTVSS | | | |
| | | (SEQ ID NO: 110) | | | |
| 26A10 | HV-02 | EVQLVESGGALVQRGG | SFGMS | YISSSSFTIYYADSV | EGGLTMVRGVSSYGL |
| | | SLRLSCAASRFTESSE | (SEQ ID | KG (SEQ ID NO: | DV (SEQ ID NO: |
| | | GMSWVRQAPGKGLEWV | NO: 78) | 88) | 96) |
| | | SYISSSSFTIYYADSV | | | |
| | | KGRFTISRDNAKNSFY | | | |
| | | LQMNSLRDEDTAVYYC | | | |
| | | AREGGLTMVRGVSSYG | | | |
| | | LDVWGQGTTVTVSS | | | |
| | | (SEQ ID NO: 111) | | | |
| 26C10 | HV-03 | EVQLVESGGALVQPGG | SFGMS | YISSSSFTIYYADSV | EGGITMVRGVSSYGM |
| | | SLRLSCAASGFTESSF | (SEQ ID | KG (SEQ ID NO: | DV (SEQ ID NO: |
| | | GMSWVRQAPGKGLEWV | NO: 78) | 88) | 97) |
| | | SYISSSSFTIYYADSV | | | |
| | | KGRFTISRDNAKNSFY | | | |
| | | LQMNSLRDEDTAVYFC | | | |
| | | VREGGITMVRGVSSYG | | | |
| | | MDVWGQGTTVTVSS | | | |
| | | (SEQ ID NO: 112) | | | |
| 26F2 | HV-04 | EVQLVESGGALVQPGG | SFGMS | YISSSSFTIYYADSV | EGGITMVRGVSSYGM |
| | | SLRLSCAASGFTESSF | (SEQ ID | KG (SEQ ID NO: | DV (SEQ ID NO: |
| | | GMSWVRQAPGKGLEWI | NO: 78) | 88) | 97) |
| | | SYISSSSFTIYYADSV | | | |
| | | KGRFTISRDNAKNSFY | | | |
| | | LQMNSLRDEDTAVYFC | | | |
| | | AREGGITMVRGVSSYG | | | |
| | | MDVWGQGTTVTVSS | | | |
| | | (SEQ ID NO: 113) | | | |
| 33B12 | HV-05 | EVQLVESGGALVQPGG | SFGMS | YISKSSFTIYYADSV | EGGLTMVRGVSSYGL |
| | | SLRLSCAASGFTESSE | (SEQ ID | KG (SEQ ID NO: | DV (SEQ ID NO: |
| | | GMSWVRQAPGKGLEWV | NO: 78) | 89) | 96) |
| | | SYISKSSFTIYYADSV | | | |
| | | KGRFTISRDNAKNSFY | | | |
| | | LQMNSLRDEDTAVYYC | | | |
| | | AREGGLTMVRGVSSYG | | | |
| | | LDVWGQGTTVTVSS | | | |
| | | (SEQ ID NO: 114) | | | |
| 24G6 | HV-06 | EVQLLESGGGLVQPGG | SYAMS | AISGSGGSTYYADSV | AYTPMAFFDY |
| | | SLRLSCAASGFTESSY | (SEQ ID | KG (SEQ ID NO: | (SEQ ID NO: 98) |
| | | AMSWVRQAPGKGLEWV | NO: 77) | 90) | |
| | | SAISGSGGSTYYADSV | | | |
| | | KGRFTISRDNSKNTLY | | | |
| | | LQMNSLRAEDTAVYYC | | | |
| | | AKAYTPMAFFDYWGQG | | | |
| | | TLVTVSS | | | |
| | | (SEQ ID NO: 115) | | | |
| 24A10 | HV-07 | EVQVLESGGGLVQPGG | NYAMS | AISGSGGSTYYADSV | GGWELFY |
| | | SLRLSCAASGFTESNY | (SEQ ID | KG (SEQ ID NO: | (SEQ ID NO: 99) |
| | | AMSWVRQAPGKGLEWV | NO: 79) | 90) | |
| | | SAISGSGGSTYYADSV | | | |
| | | KGRFTISRDNSKNTLY | | | |
| | | LOMNSLRAEDTAVYYC | | | |
| | | AKGGWELFYWGQGTLV | | | |
| | | TVSS | | | |
| | | (SEQ ID NO: 116) | | | |
| 10E3 | HV-08 | EVQLVQSGAEVKKPGE | NYWIG | IIYPGDSDTRYSPSE | RRQGIWGDALDI |
| | | SLMISCKGSGYSFTNY | (SEQ ID | QG (SEQ ID NO: | (SEQ ID NO: |
| | | WIGWVRQMPGKGLEWM | NO: 80) | 91) | 100) |
| | | GIIYPGDSDTRYSPSF | | | |
| | | QGQVTISADKSISTAY | | | |
| | | LOWSSLKASDTAMYFC | | | |
| | | ARRRQGIWGDALDIWG | | | |
| | | QGTLVTVSS | | | |
| | | (SEQ ID NO: 117) | | | |
| 13E7 | HV-09 | EVQLVQSGAEVKKPGE | SYWIG | IIYPGDSDTRYSPSF | RRQGIWGDALDE |
| 14C12 | | SLMISCKGSGYSFTSY | (SEQ ID | QG (SEQ ID NO: | (SEQ ID NO: |
| | | WIGWVRQMPGKGLEWM | NO: 81) | 91) | 101) |
| | | GIIYPGDSDTRYSPSF | | | |
| | | QGQVTISADKSISTAY | | | |
| | | LOWSSLKASDTAMYFC | | | |
| | | ARRRQGIWGDALDEWG | | | |
| | | QGTLVTVSS | | | |
| | | (SEQ ID NO: 118) | | | |
| 25F12 | HV-10 | QVQLQQWGAGLLKPSE | SYYWS | EINHSGNTNYNPSLK | EGYYDILTGYHDAFD |
| | | TLSLTCAVYGGSESSY | (SEQ ID | S (SEQ ID NO: | I (SEQ ID NO: |
| | | YWSWIRQPPGKGLEWI | NO: 82) | 92) | 102) |
| | | GEINHSGNTNYNPSLK | | | |
| | | SRVTISVDTSKNQFSL | | | |
| | | KLSSVTAADTAVYYCA | | | |
| | | REGYYDILTGYHDAFD | | | |
| | | IWDQGTMVTVES | | | |
| | | (SEQ ID NO: 119) | | | |
| 32E3 | HV-11 | EVQLVQSGAEVKKPGE | SYWIG | IIYPGDSDTRYSPSF | HDIIPAAPGAFDI |
| | | SLKISCKGSGYSFTSY | (SEQ ID | QG (SEQ ID NO: | (SEQ ID NO: |
| | | WIGWVRQMPGKGLEWM | NO: 81) | 91) | 103) |
| | | GIIYPGDSDTRYSPSF | | | |
| | | QGQVTISADKSISTAY | | | |
| | | LOWSTLKASDTAIYYC | | | |
| | | ARHDIIPAAPGAFDIW | | | |
| | | GQGTMVTVSS | | | |
| | | (SEQ ID NO: 120) | | | |
| 24F4 | HV-12 | EVQLVQSGAEVKKPGE | SYWIG | IIYPGDSDTRYSPSF | QAIAVTGLGGFDP |
| | | SLKISCKGSGYTFTSY | (SEQ ID | QG (SEQ ID NO: | (SEQ ID NO: |
| | | WIGWVRQMPGKGLEWM | NO: 81) | 91) | 104) |
| | | GIIYPGDSDTRYSPSF | | | |
| | | QGQVTISVDKSSSTAY | | | |
| | | LOWSSLKASDTAIYYC | | | |
| | | TRQAIAVTGLGGFDPW | | | |
| | | GQGTLVTVSS | | | |
| | | (SEQ ID NO: 121) | | | |
| 16B8 | HV-13 | QVQLVQSGAEVKKPGA | NYGIS | WISAYNGNTNYAQKL | RGYSYGSFDY |
| | | SVKVSCKASGYTFTNY | (SEQ ID | QG (SEQ ID NO: | (SEQ ID NO: |
| | | GISWVRQAPGQGLEWM | NO: 83) | 93) | 105) |
| | | GWISAYNGNTNYAQKL | | | |
| | | QGRVTMTTDTSTSTVY | | | |
| | | MELRSLRSDDTAVYYC | | | |
| | | ARRGYSYGSFDYWGQG | | | |
| | | TLVTVSS | | | |
| | | (SEQ ID NO: 122) | | | |
| 4C5 | HV-14 | EVQLVQSGAEVKKPGE | NYWIA | IIYPGDSDTRYSPSF | QRTFYYDSSGYFDY |
| | | SLKISCKGSGHSFTNY | (SEQ ID | QG (SEQ ID NO: | (SEQ ID NO: |
| | | WIAWVRQMPGKGLEWM | NO: 84) | 91) | 106) |
| | | GIIYPGDSDTRYSPSE | | | |
| | | QGQVTISADKSISTAY | | | |
| | | LOWSSLKASDTAVYFC | | | |
| | | ARQRTFYYDSSGYEDY | | | |
| | | WGQGTLVTVSS | | | |
| | | (SEQ ID NO: 123) | | | |
| 6E7 | HV-15 | EVQLVQSGAEVKKPGE | SYWIA | IIYPGDSDTRYSPSF | QRTFYYDSSDYFDY |
| | | SLKISCKGSGYSFTSY | (SEQ ID | QG (SEQ ID NO: | (SEQ ID NO: |
| | | WIAWVRQMPGKGLEWM | NO: 85) | 91) | 107) |
| | | GIIYPGDSDTRYSPSF | | | |
| | | QGQVTISADKSISTAY | | | |
| | | LQWSSLKASDTAMYFC | | | |
| | | ARQRTFYYDSSDYFDY | | | |
| | | WGQGTLVTVSS | | | |
| | | (SEQ ID NO: 124) | | | |
| 5E3 | HV-16 | QVQLVQSGAEVKKPGA | GYYIH | WINPYSGGTTSAQKF | DGGYLALYGTDV |
| | | SVKVSCKASGYTFTGY | (SEQ ID | QG (SEQ ID NO: | (SEQ ID NO: |
| | | YIHWVRQAPGLGLEWM | NO: 86) | 94) | 108) |
| | | GWINPYSGGTTSAQKE | | | |
| | | QGRVTMTRDTSISSAY | | | |
| | | MELSRLRSDDTAVYYC | | | |
| | | ARDGGYLALYGTDVWG | | | |
| | | QGTTVTVSS | | | |
| | | (SEQ ID NO: 125) | | | |
| 4G10 | HV-17 | EVQLVQSGAEVKKPGE | SYWIA | IIYPGDSDTRYSPSF | QGIEVTGTGGLDV |
| | | SLKISCKGSGYSFPSY | (SEQ ID | QG (SEQ ID NO: | (SEQ ID NO: |
| | | WIAWVRQMPGKGLEWM | NO: 85) | 91) | 109) |
| | | GIIYPGDSDTRYSPSF | | | |
| | | QGQVTISADKSISTAF | | | |
| | | LKWSSLKASDTAMYFC | | | |
| | | ARQGIEVTGTGGLDVW | | | |
| | | GQGTTVTVSS | | | |
| | | (SEQ ID NO: 126) | | | |

As noted above, a TREM2 agonist antigen binding protein may comprise one or more of the CDRs presented in Table 1 (VGL101), Table 1A (light chain CDRs; i.e., CDRLs), or Table 1B (heavy chain CDRs, i.e., CDRHs).

In some embodiments, the TREM2 agonist antigen binding protein comprises one or more light chain CDRs selected from (i) a CDRL1 selected from SEQ ID NOs: 11 to 18, or 356 to 361 (ii) a CDRL2 selected from SEQ ID NOs: 19 to 30, and (iii) a CDRL3 selected from SEQ ID NOs: 31 to 45, and (iv) a CDRL of (i), (ii) and (iii) that contains one or more, e.g., one, two, three, four or more amino acid substitutions (e.g., conservative amino acid substitutions), deletions or insertions of no more than five, four, three, two, or one amino acids. In these and other embodiments, the TREM2 agonist antigen binding proteins comprise one or more heavy chain CDRs selected from (i) a CDRH1 selected from SEQ ID NOs: 77 to 86, (ii) a CDRH2 selected from SEQ ID NOs: 87 to 94, and (iii) a CDRH3 selected from SEQ ID NOs: 95 to 109, and (iv) a CDRH of (i), (ii) and (iii) that contains one or more, e.g., one, two, three, four or more amino acid substitutions (e.g., conservative amino acid substitutions), deletions or insertions of no more than five, four, three, two, or one amino acids amino acids.

In some embodiments, the TREM2 agonist antigen binding protein may comprise 1, 2, 3, 4, 5, or 6 variant forms of the CDRs listed in Tables 1A and 1B, each having at least 80%, 85%, 90% or 95% sequence identity to a CDR sequence listed in Tables 1A and 1B. In some embodiments, the TREM2 agonist antigen binding protein includes 1, 2, 3, 4, 5, or 6 of the CDRs listed in Tables 1A and 1B, each differing by no more than 1, 2, 3, 4 or 5 amino acids from the CDRs listed in these tables.

In some embodiments, the TREM2 agonist antigen binding protein comprises a CDRL1 comprising a sequence selected from SEQ ID NOs: 11-18 or 356-361 or a variant thereof having one, two, three or four amino acid substitutions; a CDRL2 comprising a sequence selected from SEQ ID NOs: 19-30 or a variant thereof having one, two, three or four amino acid substitutions; a CDRL3 comprising a sequence selected from SEQ ID NOs: 31-45 or a variant thereof having one, two, three or four amino acid substitutions; a CDRH1 comprising a sequence selected from SEQ ID NOs: 77-86 or a variant thereof having one, two, three or four amino acid substitutions; a CDRH2 comprising a sequence selected from SEQ ID NOs: 87-94 or a variant thereof having one, two, three or four amino acid substitutions; and a CDRH3 comprising a sequence selected from SEQ ID NOs: 95-109 or a variant thereof having one, two, three or four amino acid substitutions.

In some embodiments, the TREM2 agonist antigen binding proteins of the invention comprise a CDRL1 comprising a sequence selected from SEQ ID NOs: 11-18 or 356-361; a CDRL2 comprising a sequence selected from SEQ ID NOs: 19-30; a CDRL3 comprising a sequence selected from SEQ ID NOs: 31-45; a CDRH1 comprising a sequence selected from SEQ ID NOs: 77-86; a CDRH2 comprising a sequence selected from SEQ ID NOs: 87-94; and a CDRH3 comprising a sequence selected from SEQ ID NOs: 95-109.

In some embodiments, the TREM2 agonist antigen binding protein comprise a light chain variable region comprising a CDRL1, a CDRL2, and a CDRL3, wherein:
(a) CDRL1, CDRL2, and CDRL3 have the sequence of SEQ ID NOs: 356, 19, and 31, respectively;
(b) CDRL1, CDRL2, and CDRL3 have the sequence of SEQ ID NOs: 357, 20, and 32, respectively;
(c) CDRL1, CDRL2, and CDRL3 have the sequence of SEQ ID NOs: 357, 21, and 33, respectively;
(d) CDRL1, CDRL2, and CDRL3 have the sequence of SEQ ID NOs: 357, 20, and 33, respectively;
(e) CDRL1, CDRL2, and CDRL3 have the sequence of SEQ ID NOs: 358, 22, and 34, respectively;
(f) CDRL1, CDRL2, and CDRL3 have the sequence of SEQ ID NOs: 359, 22, and 35, respectively;
(g) CDRL1, CDRL2, and CDRL3 have the sequence of SEQ ID NOs: 360, 22, and 36, respectively;
(h) CDRL1, CDRL2, and CDRL3 have the sequence of SEQ ID NOs: 361, 23, and 37, respectively;
(i) CDRL1, CDRL2, and CDRL3 have the sequence of SEQ ID NOs: 11, 23, and 38, respectively;
(j) CDRL1, CDRL2, and CDRL3 have the sequence of SEQ ID NOs: 12, 24, and 39, respectively;
(k) CDRL1, CDRL2, and CDRL3 have the sequence of SEQ ID NOs: 13, 25, and 40, respectively;
(l) CDRL1, CDRL2, and CDRL3 have the sequence of SEQ ID NOs: 14, 26, and 41, respectively;
(m) CDRL1, CDRL2, and CDRL3 have the sequence of SEQ ID NOs: 15, 27, and 42, respectively;
(n) CDRL1, CDRL2, and CDRL3 have the sequence of SEQ ID NOs: 16, 28, and 43, respectively;
(o) CDRL1, CDRL2, and CDRL3 have the sequence of SEQ ID NOs: 17, 29, and 44, respectively, or
(p) CDRL1, CDRL2, and CDRL3 have the sequence of SEQ ID NOs: 18, 30, and 45, respectively.

In some embodiments, the TREM2 agonist antigen binding protein comprises a heavy chain variable region comprising a CDRH1, a CDRH2, and a CDRH3, wherein:
(a) CDRH1, CDRH2, and CDRH3 have the sequence of SEQ ID NOs: 77, 87, and 95, respectively;
(b) CDRH1, CDRH2, and CDRH3 have the sequence of SEQ ID NOs: 78, 88, and 96, respectively;
(c) CDRH1, CDRH2, and CDRH3 have the sequence of SEQ ID NOs: 78, 88, and 97, respectively;
(d) CDRH1, CDRH2, and CDRH3 have the sequence of SEQ ID NOs: 78, 89, and 96, respectively;
(e) CDRH1, CDRH2, and CDRH3 have the sequence of SEQ ID NOs: 77, 90, and 98, respectively;
(f) CDRH1, CDRH2, and CDRH3 have the sequence of SEQ ID NOs: 79, 90, and 99, respectively;
(g) CDRH1, CDRH2, and CDRH3 have the sequence of SEQ ID NOs: 80, 91, and 100, respectively;
(h) CDRH1, CDRH2, and CDRH3 have the sequence of SEQ ID NOs: 81, 91, and 101, respectively;
(i) CDRH1, CDRH2, and CDRH3 have the sequence of SEQ ID NOs: 82, 92, and 102, respectively;
(j) CDRH1, CDRH2, and CDRH3 have the sequence of SEQ ID NOs: 81, 91, and 103, respectively;
(k) CDRH1, CDRH2, and CDRH3 have the sequence of SEQ ID NOs: 81, 91, and 104, respectively;
(l) CDRH1, CDRH2, and CDRH3 have the sequence of SEQ ID NOs: 83, 93, and 105, respectively;
(m) CDRH1, CDRH2, and CDRH3 have the sequence of SEQ ID NOs: 84, 91, and 106, respectively;
(n) CDRH1, CDRH2, and CDRH3 have the sequence of SEQ ID NOs: 85, 91, and 107, respectively;
(o) CDRH1, CDRH2, and CDRH3 have the sequence of SEQ ID NOs: 86, 94, and 108, respectively; or
(p) CDRH1, CDRH2, and CDRH3 have the sequence of SEQ ID NOs: 85, 91, and 109, respectively.

In some embodiments, the TREM2 agonist antigen binding protein comprises a light chain variable region comprising a CDRL1, a CDRL2, and a CDRL3 and a heavy chain variable region comprising a CDRH1, a CDRH2, and a CDRH3, wherein:
(a) CDRL1, CDRL2, and CDRL3 have the sequence of SEQ ID NOs: 356, 19, and 31, respectively, and CDRH1, CDRH2, and CDRH3 have the sequence of SEQ ID NOs: 77, 87, and 95, respectively;
(b) CDRL1, CDRL2, and CDRL3 have the sequence of SEQ ID NOs: 357, 20, and 32, respectively, and CDRH1, CDRH2, and CDRH3 have the sequence of SEQ ID NOs: 78, 88, and 96, respectively;
(c) CDRL1, CDRL2, and CDRL3 have the sequence of SEQ ID NOs: 357, 21, and 33, respectively, and CDRH1, CDRH2, and CDRH3 have the sequence of SEQ ID NOs: 78, 88, and 97, respectively;
(d) CDRL1, CDRL2, and CDRL3 have the sequence of SEQ ID NOs: 357, 20, and 33, respectively, and CDRH1, CDRH2, and CDRH3 have the sequence of SEQ ID NOs: 78, 88, and 97, respectively;
(e) CDRL1, CDRL2, and CDRL3 have the sequence of SEQ ID NOs: 357, 20, and 33, respectively, and CDRH1, CDRH2, and CDRH3 have the sequence of SEQ ID NOs: 78, 89, and 96, respectively;
(f) CDRL1, CDRL2, and CDRL3 have the sequence of SEQ ID NOs: 358, 22, and 34, respectively, and CDRH1, CDRH2, and CDRH3 have the sequence of SEQ ID NOs: 77, 87, and 95, respectively;
(g) CDRL1, CDRL2, and CDRL3 have the sequence of SEQ ID NOs: 359, 22, and 35, respectively, and CDRH1, CDRH2, and CDRH3 have the sequence of SEQ ID NOs: 77, 90, and 98, respectively;
(h) CDRL1, CDRL2, and CDRL3 have the sequence of SEQ ID NOs: 360, 22, and 36, respectively, and CDRH1, CDRH2, and CDRH3 have the sequence of SEQ ID NOs: 79, 90, and 99, respectively;
(i) CDRL1, CDRL2, and CDRL3 have the sequence of SEQ ID NOs: 361, 23, and 37, respectively, and CDRH1, CDRH2, and CDRH3 have the sequence of SEQ ID NOs: 80, 91, and 100, respectively;
(j) CDRL1, CDRL2, and CDRL3 have the sequence of SEQ ID NOs: 361, 23, and 37, respectively, and CDRH1, CDRH2, and CDRH3 have the sequence of SEQ ID NOs: 81, 91, and 101, respectively;
(k) CDRL1, CDRL2, and CDRL3 have the sequence of SEQ ID NOs: 11, 23, and 38, respectively, and CDRH1, CDRH2, and CDRH3 have the sequence of SEQ ID NOs: 82, 92, and 102, respectively;
(l) CDRL1, CDRL2, and CDRL3 have the sequence of SEQ ID NOs: 12, 24, and 39, respectively, and CDRH1, CDRH2, and CDRH3 have the sequence of SEQ ID NOs: 81, 91, and 103, respectively;
(m) CDRL1, CDRL2, and CDRL3 have the sequence of SEQ ID NOs: 13, 25, and 40, respectively, and CDRH1, CDRH2, and CDRH3 have the sequence of SEQ ID NOs: 81, 91, and 104, respectively;
(n) CDRL1, CDRL2, and CDRL3 have the sequence of SEQ ID NOs: 14, 26, and 41, respectively, and CDRH1, CDRH2, and CDRH3 have the sequence of SEQ ID NOs: 83, 93, and 105, respectively;
(o) CDRL1, CDRL2, and CDRL3 have the sequence of SEQ ID NOs: 15, 27, and 42, respectively, and CDRH1, CDRH2, and CDRH3 have the sequence of SEQ ID NOs: 84, 91, and 106, respectively;
(p) CDRL1, CDRL2, and CDRL3 have the sequence of SEQ ID NOs: 16, 28, and 43, respectively, and CDRH1, CDRH2, and CDRH3 have the sequence of SEQ ID NOs: 85, 91, and 107, respectively;
(q) CDRL1, CDRL2, and CDRL3 have the sequence of SEQ ID NOs: 17, 29, and 44, respectively, and CDRH1, CDRH2, and CDRH3 have the sequence of SEQ ID NOs: 86, 94, and 108, respectively; or
(r) CDRL1, CDRL2, and CDRL3 have the sequence of SEQ ID NOs: 18, 30, and 45, respectively, and CDRH1, CDRH2, and CDRH3 have the sequence of SEQ ID NOs: 85, 91, and 109, respectively.

In some embodiments, the TREM2 agonist antigen binding protein comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOs: 361, 23, and 37, respectively, and CDRH1, CDRH2, and CDRH3 have the sequence of SEQ ID NOs: 80, 91, and 100, respectively. In some embodiments, the TREM2 agonist antigen binding protein comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOs: 361, 23, and 37, respectively, and CDRH1, CDRH2, and CDRH3 have the sequence of SEQ ID NOs: 81, 91, and 101, respectively. In some embodiments, the TREM2 agonist antigen binding protein comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOs: 15, 27, and 42, respectively, and CDRH1, CDRH2, and CDRH3 have the sequence of SEQ ID NOs: 84, 91, and 106, respectively. In some embodiments, the TREM2 agonist antigen binding protein comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOs: 16, 28, and 43, respectively, and CDRH1, CDRH2, and CDRH3 have the sequence of SEQ ID NOs: 85, 91, and 107, respectively. In some embodiments, the TREM2 agonist antigen binding protein comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOs: 17, 29, and 44, respectively, and CDRH1, CDRH2, and CDRH3 have the sequence of SEQ ID NOs: 86, 94, and 108, respectively. In some embodiments, the TREM2 agonist antigen binding protein comprises a light chain variable region comprising a CDRL1, a CDRL2, and a CDRL3 and a heavy chain variable region comprising a CDRH1, a CDRH2, and a CDRH3, wherein CDRL1, CDRL2, and CDRL3 have the sequence of SEQ ID NOs: 359, 22, and 35, respectively, and CDRH1, CDRH2, and CDRH3 have the sequence of SEQ ID NOs: 77, 90, and 98, respectively.

In some embodiments, the TREM2 agonist antigen binding proteins comprise a light chain variable region comprising a sequence selected from SEQ ID NOs: 46-63 and a heavy chain variable region comprising a sequence selected from SEQ ID NOs: 110-126.In some embodiments, the TREM2 agonist antigen binding protein comprises a light chain variable region comprising the sequence of SEQ ID NO: 46 and a heavy chain variable region comprising the sequence of SEQ ID NO: 110. In some embodiments, the TREM2 agonist antigen binding protein comprises a light chain variable region comprising the sequence of SEQ ID NO: 47 and a heavy chain variable region comprising the sequence of SEQ ID NO: 111. In some embodiments, the TREM2 agonist antigen binding protein comprises a light chain variable region comprising the sequence of SEQ ID NO: 48 and a heavy chain variable region comprising the sequence of SEQ ID NO: 112. In some embodiments, the TREM2 agonist antigen binding protein comprises a light chain variable region comprising the sequence of SEQ ID NO: 49 and a heavy chain variable region comprising the sequence of SEQ ID NO: 113. In some embodiments, the TREM2 agonist antigen binding protein comprises a light chain variable region comprising the sequence of SEQ ID NO: 50 and a heavy chain variable region comprising the sequence of SEQ ID NO: 114. In some embodiments, the TREM2 agonist antigen binding protein comprises a light chain variable region comprising the sequence of SEQ ID NO: 51 and a heavy chain variable region comprising the sequence of SEQ ID NO: 110. In some embodiments, the TREM2 agonist antigen binding protein comprises a light chain variable region comprising the sequence of SEQ ID NO: 53 and a heavy chain variable region comprising the sequence of SEQ ID NO: 116. In some embodiments, the TREM2 agonist antigen binding protein comprises a light chain variable region comprising the sequence of SEQ ID NO: 54 and a heavy chain variable region comprising the sequence of SEQ ID NO: 117. In some embodiments, the TREM2 agonist antigen binding protein comprises a light chain variable region comprising the sequence of SEQ ID NO: 55 and a heavy chain variable region comprising the sequence of SEQ ID NO: 118. In some embodiments, the TREM2 agonist antigen binding protein comprises a light chain variable region comprising the sequence of SEQ ID NO: 56 and a heavy chain variable region comprising the sequence of SEQ ID NO: 119. In some embodiments, the TREM2 agonist antigen binding protein comprises a light chain variable region comprising the sequence of SEQ ID NO: 57 and a heavy chain variable region comprising the sequence of SEQ ID NO: 120. In some embodiments, the TREM2 agonist antigen binding protein comprises a light chain variable region comprising the sequence of SEQ ID NO: 58 and a heavy chain variable region comprising the sequence of SEQ ID NO: 121. In some embodiments, the TREM2 agonist antigen binding protein comprises a light chain variable region comprising the sequence of SEQ ID NO: 59 and a heavy chain variable region comprising the sequence of SEQ ID NO: 122. In some embodiments, the TREM2 agonist antigen binding protein comprises a light chain variable region comprising the sequence of SEQ ID NO: 60 and a heavy chain variable region comprising the sequence of SEQ ID NO: 123. In some embodiments, the TREM2 agonist antigen binding protein comprises a light chain variable region comprising the sequence of SEQ ID NO: 61 and a heavy chain variable region comprising the sequence of SEQ ID NO: 124. In some embodiments, the TREM2 agonist antigen binding protein comprises a light chain variable region comprising the sequence of SEQ ID NO: 62 and a heavy chain variable region comprising the sequence of SEQ ID NO: 125. In some embodiments, the TREM2 agonist antigen binding protein comprises a light chain variable region comprising the sequence of SEQ ID NO: 63 and a heavy chain variable region comprising the sequence of SEQ ID NO: 126. In yet another embodiment, the TREM2 agonist antigen binding protein comprises a light chain variable region comprising the sequence of SEQ ID NO: 52 and a heavy chain variable region comprising the sequence of SEQ ID NO: 115.

In some embodiments, the TREM2 agonist antigen binding protein comprises a light chain variable region selected from LV-01, LV-02, LV-03, LV-04, LV-05, LV-06, LV-07, LV-08, LV-09, LV-10, LV-11, LV-12, LV-13, LV-14, LV-15, LV-16, LV-17, and LV-18, as shown in Table 1A, and/or a heavy chain variable region selected from HV-01, HV-02, HV-03, HV-04, HV-05, HV-06, HV-07, HV-08, HV-09, HV-10, HV-11, HV-12, HV-13, HV-14, HV-15, HV-16, and HV-17, as shown in Table 1B, and functional fragments, derivatives, muteins and variants of these light chain and heavy chain variable regions.

In some embodiments, each of the light chain variable regions listed in Table 1A may be combined with any of the heavy chain variable regions listed in Table 1B to form an anti-TREM2 binding domain of the antigen binding proteins of the invention. Examples of such combinations include, but are not limited to: LV-01 (SEQ ID NO: 46) and HV-0 (SEQ ID NO: 110); LV-02 (SEQ ID NO: 47) and HV-02 (SEQ ID NO: 111); LV-03 (SEQ ID NO: 48) and HV-03 (SEQ ID NO: 112); LV-04 (SEQ ID NO: 49) and HV-04 (SEQ ID NO: 113); LV-05 (SEQ ID NO: 50) and HV-05 (SEQ ID NO: 114); LV-06 (SEQ ID NO: 51) and HV-01 (SEQ ID NO: 110); LV-07 (SEQ ID NO: 52) and HV-06 (SEQ ID NO: 115); LV-08 (SEQ ID NO: 53) and HV-07 (SEQ ID NO: 116); LV-09 (SEQ ID NO: 54) and HV-08 (SEQ ID NO: 117); LV-10 (SEQ ID NO: 55) and HV-09 (SEQ ID NO: 118); LV-11 (SEQ ID NO: 56) and HV-10 (SEQ ID NO: 119); LV-12 (SEQ ID NO: 57) and HV-11 (SEQ ID NO: 120); LV-13 (SEQ ID NO: 58) and HV-12 (SEQ ID NO: 121); LV-14 (SEQ ID NO: 59) and HV-13 (SEQ ID NO: 122); LV-15 (SEQ ID NO: 60) and HV-14 (SEQ ID NO: 123); LV-16 (SEQ ID NO: 61) and HV-15 (SEQ ID NO: 124); LV-17 (SEQ ID NO: 62) and HV-16 (SEQ ID NO: 125); and LV-18 (SEQ ID NO: 63) and HV-17 (SEQ ID NO: 126).

In certain embodiments, the TREM2 agonist antigen binding proteins of the invention comprise a light chain variable region comprising the sequence of LV-09 (SEQ ID NO: 54) and a heavy chain variable region comprising the sequence of HV-08 (SEQ ID NO: 117). In some embodiments, the TREM2 agonist antigen binding proteins of the invention comprise a light chain variable region comprising the sequence of LV-10 (SEQ ID NO: 55) and a heavy chain variable region comprising the sequence of HV-09 (SEQ ID NO: 118). In other embodiments, the TREM2 agonist antigen binding proteins of the invention comprise a light chain variable region comprising the sequence of LV-15 (SEQ ID NO: 60) and a heavy chain variable region comprising the sequence of HV-14 (SEQ ID NO: 123). In still other embodiments, the TREM2 agonist antigen binding proteins of the invention comprise a light chain variable region comprising the sequence of LV-16 (SEQ ID NO: 61) and a heavy chain variable region comprising the sequence of HV-15 (SEQ ID NO: 124). In some embodiments, the TREM2 agonist antigen binding proteins of the invention comprise a light chain variable region comprising the sequence of LV-17 (SEQ ID NO: 62) and a heavy chain variable region comprising the sequence of HV-16 (SEQ ID NO: 125). In certain embodiments, the TREM2 agonist antigen binding proteins of the invention comprise a light chain variable region comprising the sequence of LV-07 (SEQ ID NO: 52) and a heavy chain variable region comprising the sequence of HV-06 (SEQ ID NO: 115).

In some embodiments, the TREM2 agonist antigen binding proteins comprise a light chain variable region comprising a sequence of contiguous amino acids that differs from the sequence of a light chain variable region in Table 1A, i.e., a VL selected from LV-01, LV-02, LV-03, LV-04, LV-05, LV-06, LV-07, LV-08, LV-09, LV-10, LV-11, LV-12, LV-13, LV-14, LV-15, LV-16, LV-17, or LV-18, at only 1, 2, 3, 4, 5, 6, 7, 8, 9, 10, 11, 12, 13, 14 or 15 amino acid residues, wherein each such sequence difference is independently either a deletion, insertion or substitution of one amino acid, with the deletions, insertions and/or substitutions resulting in no more than 15 amino acid changes relative to the foregoing variable domain sequences. The light chain variable region in some TREM2 agonist antigen binding proteins comprises a sequence of amino acids that has at least 70%, at least 75%, at least 80%, at least 85%, at least 90%, at least 95%, at least 97% or at least 99% sequence identity to the amino acid sequences of SEQ ID NOs: 46-63 (i.e. the light chain variable regions in Table 1A). In one embodiment, the TREM2 agonist antigen binding protein comprises a light chain variable region comprising a sequence that is at least 90% identical to a sequence selected from SEQ ID NOs: 46-63. In another embodiment, the TREM2 agonist antigen binding protein comprises a light chain variable region comprising a sequence that is at least 95% identical to a sequence selected from SEQ ID NOs: 46-63. In yet another embodiment, the TREM2 agonist antigen binding protein comprises a light chain variable region comprising a sequence selected from SEQ ID NOs: 46-63. In some embodiments, the TREM2 agonist antigen binding protein comprises a light chain variable region comprising a sequence of SEQ ID NO: 54. In other embodiments, the TREM2 agonist antigen binding protein comprises a light chain variable region comprising a sequence of SEQ ID NO: 55. In yet other embodiments, the TREM2 agonist antigen binding protein comprises a light chain variable region comprising a sequence of SEQ ID NO: 60. In still other embodiments, the TREM2 agonist antigen binding protein comprises a light chain variable region comprising a sequence of SEQ ID NO: 61. In certain embodiments, the TREM2 agonist antigen binding protein comprises a light chain variable region comprising a sequence of SEQ ID NO: 62. In other embodiments, the TREM2 agonist antigen binding protein comprises a light chain variable region comprising a sequence of SEQ ID NO: 52.

In these and other embodiments, the TREM2 agonist antigen binding proteins comprise a heavy chain variable region comprising a sequence of contiguous amino acids that differs from the sequence of a heavy chain variable region in Table 1B, i.e., a VH selected from HV-01, HV-02, HV-03, HV-04, HV-05, HV-06, HV-07, HV-08, HV-09, HV-10, HV-11, HV-12, HV-13, HV-14, HV-15, HV-16, or HV-17, at only 1, 2, 3, 4, 5, 6, 7, 8, 9, 10, 11, 12, 13, 14 or 15 amino acid residues, wherein each such sequence difference is independently either a deletion, insertion or substitution of one amino acid, with the deletions, insertions and/or substitutions resulting in no more than 15 amino acid changes relative to the foregoing variable domain sequences. The heavy chain variable region in some TREM2 agonist antigen binding proteins comprises a sequence of amino acids that has at least 70%, at least 75%, at least 80%, at least 85%, at least 90%, at least 95%, at least 97% or at least 99% sequence identity to the amino acid sequences of SEQ ID NOs: 110-126 (i.e. the heavy chain variable regions in Table 1B). In one embodiment, the TREM2 agonist antigen binding protein comprises a heavy chain variable region comprising a sequence that is at least 90% identical to a sequence selected from SEQ ID NOs: 110-126. In another embodiment, the TREM2 agonist antigen binding protein comprises a heavy chain variable region comprising a sequence that is at least 95% identical to a sequence selected from SEQ ID NOs: 110-126. In yet another embodiment, the TREM2 agonist antigen binding protein comprises a heavy chain variable region comprising a sequence selected from SEQ ID NOs: 110-126. In some embodiments, the TREM2 agonist antigen binding protein comprises a heavy chain variable region comprising a sequence of SEQ ID NO: 117. In other embodiments, the TREM2 agonist antigen binding protein comprises a heavy chain variable region comprising a sequence of SEQ ID NO: 118. In yet other embodiments, the TREM2 agonist antigen binding protein comprises a heavy chain variable region comprising a sequence of SEQ ID NO: 123. In still other embodiments, the TREM2 agonist antigen binding protein comprises a heavy chain variable region comprising a sequence of SEQ ID NO: 124. In certain embodiments, the TREM2 agonist antigen binding protein comprises a heavy chain variable region comprising a sequence of SEQ ID NO: 125. In other embodiments, the TREM2 agonist antigen binding protein comprises a heavy chain variable region comprising a sequence of SEQ ID NO: 115.

In some embodiments, variants of the anti-TREM2 antibodies can be generated by substituting one or more amino acids in the light chain or heavy chain variable regions to address chemical liabilities (e.g., aspartate isomerization, asparagine deamidation, tryptophan and methionine oxidation) or correct covanance violations (see e.g., WO 2012/125495, which is hereby incorporated by reference in its entirety). Such variants can have improved biophysical, expression, and/or stability properties as compared with the parental antibody. In some embodiments, the TREM2 agonist antigen binding proteins of the invention comprise a light chain variable region and/or heavy chain variable region having one or more of the amino acid substitutions set forth in any of Tables 2A-2F below.

In some embodiments, additional variants of the anti-TREM2 antibodies described herein can be generated by affinity modulating any of the anti-TREM2 antibodies described herein. An “affinity-modulated antibody” is an antibody that comprises one or more amino acid substitutions in its light chain variable region sequence and/or heavy chain variable region sequence that increases or decreases the affinity of the antibody for the target antigen as compared to the parental antibody that does not contain the amino acid substitutions. Antibody affinity modulation methods are known to those of skill in the art and can include CDR walking mutagenesis (Yang et al., J. Mol. Biol., 254, 392-403, 1995), chain shuffling (Marks et al., Bio/Technology, 10, 779-783, 1992), use of mutation strains of E. coli (Low et al., J. Mol. Biol., 250, 350-368, 1996), DNA shuffling (Patten et al., Curr. Opin. Biotechnol., 1997, 8:724-733), phage display (Thompson et al., J. Mol. Biol., 1996, 256:7-88), PCR techniques (Crameri, et al., Nature, 1998, 391:288-291), and other mutagenesis strategies (Barbas et al., Proc Nat. Acad. Sci. USA 91:3809-3813, 1994; Schier et al., Gene 169:147-155, 1995; Yelton et al., J. Immunol. 155:1994-2004, 1995; Jackson et al., J. Immunol. 154(7):3310-9, 1995; and Hawkins et al., J. Mol. Biol., 1992, 226:889-896). Methods of affinity modulation are discussed in Hoogenboom, Trends in Biotechnology, 1995, 15:62-70, and Vaughan et al., Nature Biotechnology, 1998, 16535-539. One specific method for generating affinity-modulated variants of the anti-TREM2 antibodies described herein is the use of a yeast-display Fab mutagenesis library.

In some embodiments, the TREM2 agonist antigen binding proteins comprise a light chain variable region that is a variant of a light chain variable region of any of the anti-TREM2 antibodies described herein. Thus, in some embodiments, the light chain variable region of the TREM2 agonist antigen binding proteins comprises a sequence that is at least 90% identical, at least 91% identical, at least 92% identical, at least 93% identical, at least 94% identical, or at least 95% identical to a sequence selected from SEQ ID NOs: 46-63. In some embodiments, the TREM2 agonist antigen binding proteins can comprise a light chain variable region from any of the engineered anti-TREM2 antibody variants set forth in Tables 2A-2F below.

In some embodiments, the TREM2 agonist antigen binding protein comprises a light chain variable region comprising the sequence of SEQ ID NO: 54 with a mutation at one or more amino acid positions 64, 79, 80, 85, 94, and/or 100. In some such embodiments, the mutation is V64G, V64A, Q79E, Q79D, S80P, S80A, F85V, F85L, F85A, F85D, F85I, F85L, F85M, F85T, W94F, W94Y, W94S, W94T, W94A, W94H, W94I, W94Q, P100R, P100Q, P100G, or combinations thereof. In another embodiment, the TREM2 agonist antigen binding protein comprises a light chain variable region comprising the sequence of SEQ ID NO: 55 with a mutation at one or more amino acid positions 64, 79, 80, 94, and/or 100. Such mutations can include V64G, V64A, Q79E, Q79D, S80P, S80A, W94F, W94Y, W94S, W94T, W94A, W94H, W94I, W94Q, P100R, P100Q, P100G, or combinations thereof. In certain embodiments, the mutation is V64G, V64A, Q79E, S80P, S80A, W94Y, W94S, P100R, P100Q, or combinations thereof. In another embodiment, the TREM2 agonist antigen binding protein comprises a light chain variable region comprising the sequence of SEQ ID NO: 60 with a mutation at one or more amino acid positions 60, 92, and/or 93. The mutation in such embodiments can be selected from L60S, L60P, L60D, L60A, D92E, D92Q, D92T, D92N, S93A, S93N, S93Q, S93V, or combinations thereof. In yet another embodiment, the TREM2 agonist antigen binding protein comprises a light chain variable region comprising the sequence of SEQ ID NO: 61 with a mutation at one or more amino acid positions 56, 57, 92, and/or 93. In such embodiments, the mutation can be N56S, N56T, N56Q, N56E, G57A, G57V, D92E, D92Q, D92T, D92N, S93A, S93N, S93Q, S93V, or combinations thereof. In certain embodiments, the mutation is N56S, N56Q, G57A, D92E, D92Q, S93A, or combinations thereof. In still another embodiment, the TREM2 agonist antigen binding protein comprises a light chain variable region comprising the sequence of SEQ ID NO: 62 with a mutation at amino acid position 36, 46, 61 and/or 100. Such mutations can include F36Y, S46L, S46R, S46V, S46F, K61R, P100Q, P100G, P100R or combinations thereof. In particular embodiments, the mutation is F36Y, K61R, P100Q, or combinations thereof. In another embodiment, the TREM2 agonist antigen binding protein comprises a light chain variable region comprising the sequence of SEQ ID NO: 52 with a mutation at amino acid position 91, which can be selected from F91V, F911, F91T, F91L, or F91D. In one embodiment, the mutation is F91V.

In some embodiments, the TREM2 agonist antigen binding proteins comprise a heavy chain variable region that is a variant of a heavy chain variable region from any of the anti-TREM2 antibodies described herein. Thus, in some embodiments, the heavy chain variable region of the TREM2 agonist antigen binding proteins comprises a sequence that is at least 90% identical, at least 91% identical, at least 92% identical, at least 93% identical, at least 94% identical, or at least 95% identical to a sequence selected from SEQ ID NOs: 110-126. For instance, the TREM2 agonist antigen binding proteins can comprise a heavy chain variable region from any of the engineered anti-TREM2 antibody variants set forth in Tables 2A-2F below. In one embodiment, the TREM2 agonist antigen binding protein comprises a heavy chain variable region comprising the sequence of SEQ ID NO: 117 with a mutation at one or more amino acid positions 19, 55, 56, 57, 58, and/or 104. In some such embodiments, the mutation is M19K, M19R, M19T, M19E, M19N, M19Q, D55E, D55Q, D55N, D55T, S56A, S56Q, S56V, D57S, D57E, D57Q, T58A, T58V, W104F, W104Y, W104T, W104S, W104A, W104H, W1041, W104Q, or combinations thereof. In another embodiment, the TREM2 agonist antigen binding protein comprises a heavy chain variable region comprising the sequence of SEQ ID NO: 118 with a mutation at one or more amino acid positions 19, 55, 56, 57, 58, and/or 104. Such mutations can include M19K, M19R, M19T, M19E, M19N, M19Q, D55E, D55Q, D55N, D55T, S56A, S56Q, S56V, D57S, D57E, D57Q, T58A, T58V, W104F, W104Y, W104T, W104S, W104A, W104H, W1041, W104Q, or combinations thereof. In certain embodiments, the mutation is M19K, D55E, S56A, D57E, T58A, W104Y, W104T, or combinations thereof. In another embodiment, the TREM2 agonist antigen binding protein comprises a heavy chain variable region comprising the sequence of SEQ ID NO: 123 with a mutation at one or more amino acid positions 27, 55, 56, 57, 58, 105, and/or 106. In some embodiments, the mutation is selected from H27Y, H27D, H27F, H27N, D55E, D55Q, D55N, D55T, S56A, S56Q, S56V, D57S, D57E, D57Q, T58A, T58V, D105E, D105Q, D105T, D105N, D105G, S106A, S106Q, S106V, S106T, or combinations thereof. In yet another embodiment, the TREM2 agonist antigen binding protein comprises a heavy chain variable region comprising the sequence of SEQ ID NO: 124 with a mutation at one or more amino acid positions 55, 56, 57, 58, 105, and/or 106. The mutation in such embodiments can be selected from D55E, D55Q, D55N, D55T, S56A, S56Q, S56V, D57S, D57E, D57Q, T58A, T58V, D105E, D105Q, D105T, D105N, D105G, S106A, S106Q, S106V, S106T, or combinations thereof. In certain embodiments, the mutation is D55E, D55Q, S56A, D57E, T58A, D105E, D105N, S106A, or combinations thereof. In still another embodiment, the TREM2 agonist antigen binding protein comprises a heavy chain variable region comprising the sequence of SEQ ID NO: 125 with a mutation at one or more amino acid positions 43, 76, 85, 99, 100, and/or 116. Such mutations can include L43Q, L43K, L43H, I76T, R85S, R85G, R85N, R85D, D99E, D99Q, D99S, D99T, G100A, G100Y, G100V, T116L, T116M, T116P, T116R, or combinations thereof. In certain embodiments, the mutation is L43Q, R85S, D99E, G100A, G100Y, T116L, or combinations thereof. In another embodiment, the TREM2 agonist antigen binding protein comprises a heavy chain variable region comprising the sequence of SEQ ID NO: 115 with a mutation at amino acid position 62 and/or 63. In such embodiments, the mutation can be selected from D62E, D62Q, D62T, D62N, S63A, S63Q, S63V, or combinations thereof. In some embodiments, the mutation is D62E, D62Q, S63A, or combinations thereof. In some embodiments, the TREM2 agonist antigen binding proteins comprise a light chain variable region and/or heavy chain variable region from any of the anti-TREM2 variant antibodies set forth in Tables 2A, 2B, 3A, 3B, and 19. Accordingly, in some embodiments, the light chain variable region of the TREM2 agonist antigen binding proteins comprises a sequence that is at least 90% identical, at least 91% identical, at least 92% identical, at least 93% identical, at least 94% identical, or at least 95% identical to a sequence selected from SEQ ID NOs: 61, 153-162, and 295-300. In these and other embodiments, the heavy chain variable region of the TREM2 agonist antigen binding proteins comprises a sequence that is at least 90% identical, at least 91% identical, at least 92% identical, at least 93% identical, at least 94% identical, or at least 95% identical to a sequence selected from SEQ ID NOs: 124, 180-190, and 307-312.

In some embodiments, the TREM2 agonist antigen binding protein comprises a light chain variable region comprising the sequence of SEQ ID NO: 54 with a mutation at one or more amino acid positions 64, 79, 80, 85, 94, and/or 100. Such mutations can include V64G, V64A, Q79E, Q79D, S80P, S80A, F85V, F85L, F85A, F85D, F85I, F85L, F85M, F85T, W94F, W94Y, W94S, W94T, W94A, W94H, W94I, W94Q, P100R, P100Q, P100G, or combinations thereof. In these and other embodiments, the TREM2 agonist antigen binding protein comprises a heavy chain variable region comprising the sequence of SEQ ID NO: 117 with a mutation at one or more amino acid positions 19, 55, 56, 57, 58, and/or 104. In certain embodiments, the mutation is selected from M19K, M19R, M19T, M19E, M19N, M19Q, D55E, D55Q, D55N, D55T, S56A, S56Q, S56V, D57S, D57E, D57Q, T58A, T58V, W104F, W104Y, W104T, W104S, W104A, W104H, W1041, W104Q, or combinations thereof.

In other embodiments, the TREM2 agonist antigen binding protein comprises a light chain variable region comprising the sequence of SEQ ID NO: 55 with a mutation at one or more amino acid positions 64, 79, 80, 94, and/or 100. In some embodiments, the mutation is selected from V64G, V64A, Q79E, Q79D, S80P, S80A, W94F, W94Y, W94S, W94T, W94A, W94H, W94I, W94Q, P100R, P100Q, P100G, or combinations thereof. In certain embodiments, the mutation is selected from V64G, V64A, Q79E, S80P, S80A, W94Y, W94S, P100R, P100Q, or combinations thereof. For instance, in some embodiments, the TREM2 agonist antigen binding protein comprises a light chain variable region comprising the sequence of SEQ ID NO: 55 with one or more mutations selected from V64G, Q79E, S80P, W94Y, and P100Q. In these and other embodiments, the TREM2 agonist antigen binding protein comprises a heavy chain variable region comprising the sequence of SEQ ID NO: 118 with a mutation at one or more amino acid positions 19, 55, 56, 57, 58, and/or 104. Such mutations can include M19K, M19R, M19T, M19E, M19N, M19Q, D55E, D55Q, D55N, D55T, S56A, S56Q, S56V, D57S, D57E, D57Q, T58A, T58V, W104F, W104Y, W104T, W104S, W104A, W104H, W1041, W104Q, or combinations thereof. In certain embodiments, the mutation is selected from M19K, D55E, S56A, D57E, T58A, W104Y, W104T, or combinations thereof.

In certain other embodiments, the TREM2 agonist antigen binding protein comprises a light chain variable region comprising the sequence of SEQ ID NO: 60 with a mutation at one or more amino acid positions 60, 92, and/or 93. The mutation can be selected from L60S, L60P, L60D, L60A, D92E, D92Q, D92T, D92N, S93A, S93N, S93Q, S93V, or combinations thereof. In these and other embodiments, the TREM2 agonist antigen binding protein comprises a heavy chain variable region comprising the sequence of SEQ ID NO: 123 with a mutation at one or more amino acid positions 27, 55, 56, 57, 58, 105, and/or 106. In some embodiments, the mutation is selected from H27Y, H27D, H27F, H27N, D55E, D55Q, D55N, D55T, S56A, S56Q, S56V, D57S, D57E, D57Q, T58A, T58V, D105E, D105Q, D105T, D105N, D105G, S106A, S106Q, S106V, S106T, or combinations thereof.

In some embodiments, the TREM2 agonist antigen binding protein comprises a light chain variable region comprising the sequence of SEQ ID NO: 61 with a mutation at one or more amino acid positions 56, 57, 92, and/or 93. In certain embodiments, the mutation is selected from N56S, N56T, N56Q, N56E, G57A, G57V, D92E, D92Q, D92T, D92N, S93A, S93N, S93Q, S93V, or combinations thereof In some embodiments, the mutation is selected from N56S, N56Q, G57A, D92E, D92Q, S93A, or combinations thereof. In particular embodiments, the TREM2 agonist antigen binding protein comprises a light chain variable region comprising the sequence of SEQ ID NO: 61 with one or more mutations selected from N56S, D92E, and S93A. In these and other embodiments, the TREM2 agonist antigen binding protein comprises a heavy chain variable region comprising the sequence of SEQ ID NO: 124 with a mutation at one or more amino acid positions 55, 56, 57, 58, 105, and/or 106. The mutation can be selected from D55E, D55Q, D55N, D55T, S56A, S56Q, S56V, D57S, D57E, D57Q, T58A, T58V, D105E, D105Q, D105T, D105N, D105G, S106A, S106Q, S106V, S106T, or combinations thereof. In certain embodiments, the mutation is D55E, D55Q, S56A, D57E, T58A, D105E, D105N, S106A, or combinations thereof. In some embodiments, the TREM2 agonist antigen binding protein comprises a heavy chain variable region comprising the sequence of SEQ ID NO: 124 with one or more mutations selected from D55E, S56A, D57E, D105E, and S106A.

In other embodiments, the TREM2 agonist antigen binding protein comprises a light chain variable region comprising the sequence of SEQ ID NO: 62 with a mutation at amino acid position 36, 46, 61 and/or 100. In particular embodiments, the mutation is selected from F36Y, S46L, S46R, S46V, S46F, K61R, P100Q, P100G, P100R or combinations thereof. In some embodiments, the mutation is F36Y, K61R, P100Q, or combinations thereof. In some embodiments, the mutation is S46L, P100Q, or combinations thereof. In these and other embodiments, the TREM2 agonist antigen binding protein comprises a heavy chain variable region comprising the sequence of SEQ ID NO: 125 with a mutation at one or more amino acid positions 43, 76, 85, 99, 100, and/or 116. The mutation can be selected from L43Q, L43K, L43H, I76T, R85S, R85G, R85N, R85D, D99E, D99Q, D99S, D99T, G100A, G100Y, G100V, T116L, T116M, T116P, T116R, or combinations thereof. In certain embodiments, the mutation is L43Q, I76T, R85S, D99E, G100A, G100Y, T116L, or combinations thereof.

In still other embodiments, the TREM2 agonist antigen binding protein comprises a light chain variable region comprising the sequence of SEQ ID NO: 52 with a mutation at amino acid position 91. The mutation can be selected from F91V, F91I, F91T, F91L, or F91D. In one embodiment, the mutation is F91V. In these and other embodiments, the TREM2 agonist antigen binding protein comprises a heavy chain variable region comprising the sequence of SEQ ID NO: 115 with a mutation at amino acid position 62 and/or 63. In particular embodiments, the mutation is selected from D62E, D62Q, D62T, D62N, S63A, S63Q, S63V, or combinations thereof. In some embodiments, the mutation is selected from D62E, D62Q, S63A, or combinations thereof.

TABLE 2A
Engineered Variants of 10E3 Antibody

| | | | | |
|---|---|---|---|---|
| Position in 10E3 | | | | |
| VL Sequence or | | | Parent Amino | Amino Acid |
| VH sequence | Region | Hot Spot | Acid | Substitutions |

Light chain variable sequence (SEQ ID NO: 54)

| | | | | |
|---|---|---|---|---|
| 64 | FR3 | Covariance violator | V | G, A |
| 79 | FR3 | Covariance violator | Q | E, D |
| 80 | FR3 | Covariance violator | S | P, A |
| 85 | FR3 | Covariance violator | F | V, L, A, D, I, L, M, T |
| 94 | CDR3 | Potential Tryptophan | W | F, Y, S, T, A, H, I, Q |
| | | Oxidation Site | | |
| 100 | FR4 | Covariance violator | P | R, Q, G |

Heavy chain variable sequence (SEQ ID NO: 117)

| | | | | |
|---|---|---|---|---|
| 19 | FR1 | Covariance violator | M | K, R, T, E, N, Q |
| 55-56 | CDR2 | Potential Isomerization Site | DS | ES, QS, DA, NS, DQ, |
| | | | | TS, DV |
| 57-58 | CDR2 | Potential Isomerization Site | DT | ST, ET, DA, DV, QT |
| 104 | CDR3 | Potential Tryptophan | W | F, Y, T, S, A, H, I, Q |
| | | Oxidation Site | | |

TABLE 2B
Engineered Variants of 13E7 Antibody

| | | | | |
|---|---|---|---|---|
| Position in 13E7 | | | | |
| VL Sequence or | | | Parent Amino | Amino Acid |
| VH sequence | Region | Hot Spot | Acid | Substitutions |

Light chain variable sequence (SEQ ID NO: 55)

| | | | | |
|---|---|---|---|---|
| 64 | FR3 | Covariance violator | V | G, A |
| 79 | FR3 | Covariance violator | Q | E, D |
| 80 | FR3 | Covariance violator | S | P, A |
| 94 | CDR3 | Potential Tryptophan | W | F, Y, S, T, A, H, I, Q |
| | | Oxidation Site | | |
| 100 | FR4 | Covariance violator | P | R, Q, G |

Heavy chain variable sequence (SEQ ID NO: 118)

| | | | | |
|---|---|---|---|---|
| 19 | FR1 | Covariance violator | M | K, R, T, E, N, Q |
| 55-56 | CDR2 | Potential Isomerization Site | DS | ES, QS, DA, DQ, NS, |
| | | | | TS, DV |
| 57-58 | CDR2 | Potential Isomerization Site | DT | ST, ET, DA, DV, QT |
| 104 | CDR3 | Potential Tryptophan | W | F, Y, T, S, A, H, I, Q |
| | | Oxidation Site | | |

TABLE 2C
Engineered Variants of 4C5 Antibody

| | | | | |
|---|---|---|---|---|
| Position in 4C5 | | | | |
| VL Sequence or | | | Parent Amino | Amino Acid |
| VH sequence | Region | Hot Spot | Acid | Substitutions |

Light chain variable sequence (SEQ ID NO: 60)

| | | | | |
|---|---|---|---|---|
| 60 | FR3 | Covariance violator | L | S, P, D, A |
| 92-93 | CDR3 | Potential Isomerization Site | DS | ES, QS, DA, DN, DQ, |
| | | | | TS, NS, DV |

Heavy chain variable sequence (SEQ ID NO: 123)

| | | | | |
|---|---|---|---|---|
| 27 | FR1 | Covariance violator | H | Y, D, F, N |
| 55-56 | CDR2 | Potential Isomerization Site | DS | ES, QS, DA, DQ, DV, |
| | | | | TS, NS |
| 57-58 | CDR2 | Potential Isomerization Site | DT | ST, ET, DA, DV, QT |
| 105-106 | CDR3 | Potential Isomerization Site | DS | ES, QS, DA, DQ, DV, |
| | | | | TS, NS, GT |

TABLE 2D
Engineered Variants of 6E7 Antibody

| | | | | |
|---|---|---|---|---|
| Position in 6E7 | | | | |
| VL Sequence or | | | Parent | Amino Acid |
| VH sequence | Region | Hot Spot | Amino Acid | Substitutions |

Light chain variable sequence (SEQ ID NO: 61)

| | | | | |
|---|---|---|---|---|
| 56-57 | CDR2/FR3 | Potential Deamidation Site | NG | SG, TG, QG, NA, EG, |
| | boundary | | | NV |
| 92-93 | CDR3 | Potential Isomerization Site | DS | ES, QS, DA, DN, DQ, |
| | | | | DV, TS, NS |

Heavy chain variable sequence (SEQ ID NO: 124)

| | | | | |
|---|---|---|---|---|
| 55-56 | CDR2 | Potential Isomerization Site | DS | ES, QS, DA, DQ, DV, |
| | | | | TS, NS |
| 57-58 | CDR2 | Potential Isomerization Site | DT | ST, ET, DA, DV, QT |
| 105-106 | CDR3 | Potential Isomerization Site | DS | ES, QS, DA, DQ, DV, |
| | | | | TS, NS, GT |

TABLE 2E
Engineered Variants of 5E3 Antibody

| | | | | |
|---|---|---|---|---|
| Position in 5E3 | | | | |
| VL Sequence or | | | Parent | Amino Acid |
| VH sequence | Region | Hot Spot | Amino Acid | Substitutions |

Light chain variable sequence (SEQ ID NO: 62)

| | | | | |
|---|---|---|---|---|
| 36 | FR2 | Consensus violator | F | Y |
| 46 | FR2 | Covariance violator | S | L, R, V, F |
| 61 | FR3 | Consensus violator | K | R |
| 100 | FR4 | Covariance violator | P | Q, G, R |

Heavy chain variable sequence (SEQ ID NO: 125)

| | | | | |
|---|---|---|---|---|
| 43 | FR2 | Covariance violator | L | Q, K, H |
| 76 | FR3 | Covariance violator | I | T |
| 85 | FR3 | Covariance violator | R | S, G, N, D |
| 99-100 | CDR3 | Potential Isomerization Site | DG | EG, DA, DY, DV, |
| | | | | QG, SG, TG |
| 116 | FR4 | Covariance violator | T | L, M, P, R |

TABLE 2F
Engineered Variants of 24G6 Antibody

| | | | | |
|---|---|---|---|---|
| Position in 24G6 | | | | |
| VL Sequence or | | | Parent | Amino Acid |
| VH sequence | Region | Hot Spot | Amino Acid | Substitutions |

Light chain variable sequence (SEQ ID NO: 52)

| | | | | |
|---|---|---|---|---|
| 91 | FR3 | Covariance violator | F | V, I, T, L, D |

Heavy chain variable sequence (SEQ ID NO: 115)

| | | | | |
|---|---|---|---|---|
| 62-63 | CDR2 | Potential Isomerization Site | DS | ES, QS, DA, DQ, TS, |
| | | | | DV, NS |

In some embodiments, the TREM2 agonist antigen binding proteins comprise one or more CDRs of a variant of the anti-TREM2 antibodies described herein. In some embodiments, the TREM2 agonist antigen binding proteins may comprise one or more CDRs of the anti-TREM2 antibody variants set forth in Tables 3A, 3B3, 3C, 3D), and 3E, below.

In certain embodiments, the TREM2 agonist antigen binding proteins of the invention comprise a light chain variable region and/or heavy chain variable region from an affinity-modulated variant of the 6E7 antibody. For instance, in some embodiments, the TREM2 agonist antigen binding proteins comprise a light chain variable region and/or a heavy chain variable region having one or more of the amino acid substitutions set forth in Table 2G.

TABLE 2G
6E7 Antibody Affinity Modulation Variants

| | | | | | | | Binding Signal (fold over | | | |
|---|---|---|---|---|---|---|---|---|---|---|
| | Substitutions with respect | | | Substitutions with respect | | | 6E7 parental antibody) | | | |
| | to 6E7 VH sequence | | | to 6E7 VL sequence | | | 1^{st} | | | |
| | (SEQ ID NO: 124) | | | (SEQ ID NO: 61) | | | screen | 2^{nd} | 2^{nd} | 2^{nd} |
| Variant | HC | HC | HC | LC | LC | LC | 110 nM | screen | screen | screen |
| Ab ID | FR1-CDR1 | CDR2 | CDR3 | CDR1 | CDR2 | CDR3 | or 10 nM^{a} | 2 nM | 10 nM | 100 nM |
| V1 | Y32S | | Q99S | | Q55T | F94Y | 1.68 | 1.29 | 1.92 | |
| V2 | Y27S | S56G | Q99S | | L54R | S93R | 2.55 | 2.23 | 2.90 | |
| V3 | T30A | G66D | Q99G | | L54R | S93R | 1.97 | 1.95 | 2.24 | |
| V4 | T30G | Y60V | Q99S | | S53R | F94Y | 6.00 | 5.88 | 5.51 | |
| V5 | | I50T | | | | F94H | 2.73 | 1.25 | 2.84 | |
| V6 | Y32M | | | | | | 0.20* | | | 0.56 |
| V7 | Y32E | | | | | | 0.11* | | | 0.32 |
| V8 | | R59K | | | | | 0.28* | | | 0.77 |
| V9 | | | T101G | | | | 0.67* | | | 0.54 |
| V10 | | | | | A50S | | 0.76* | | | 0.70 |
| V11 | | | | | | D92A | 0.79* | | | 0.42 |
| V12 | S28E | T58V | Q99G | | N56R | | 2.29 | 1.04 | 2.58 | |
| V13 | T30G | P62A | Q99G | | N56G | F94M | 1.31 | 1.15 | 1.35 | |
| V14 | T30G | S56Q | Q99G | | S53R | | 4.71 | 2.57 | 4.64 | |
| V15 | T30A | I50T | Q99S | | S53W | F94Y | 5.23 | 4.72 | 4.78 | |
| V16 | F29M | S56G | Q99S | | S53N | | 4.01 | 3.57 | 4.04 | |
| V17 | T30G | | Q99S | | L54R | F94S | 5.37 | 4.22 | 5.51 | |
| V18 | W33H | | | | | | 0.17* | | | 0.42 |
| V19 | Y32S | | | | | | 0.59* | | | 0.48 |
| V20 | | I50R | | | | | 0.18* | | | 0.52 |
| V21 | | | Y109F | | | | 0.76* | | | 0.68 |
| V22 | | | | | A50R | | 0.30* | | | 0.71 |
| V23 | | | | | | R96L | 0.40* | | | 0.40 |
| V24 | | T58V | Q99S | | N56K | R96H | 2.64 | 1.42 | 2.90 | |
| V25 | T30G | I50L | Q99S | | Q55A | F94M | 4.23 | 3.15 | 4.70 | |
| V26 | A35G | I50T | F102M, | | N56R | F94Y | 3.57 | 2.83 | 3.47 | |
| | | | Y112A | | | | | | | |
| V27 | | S61A | Q99S | | N56R | | 5.50 | 5.67 | 5.69 | |
| V28 | T30Q | I50T | Y103F | | N56S | F94L | 3.08 | 2.63 | 3.61 | |
| V29 | T30K | | | | | | 1.53 | 0.84 | 1.67 | |
| V30 | Y27S | | | | | | 0.79* | | | 0.72 |
| V31 | | D57E | | | | | 0.61* | | | 0.73 |
| V32 | | P62N | | | | | 0.82* | | | 0.89 |
| V33 | | | Y104G | | | | 0.23* | | | 0.34 |
| V34 | | | | | N56D | | 0.34* | | | 1.02 |
| V35 | | | | | | D92Y | 0.21* | | | 0.29 |
| V36 | I34L | | Q99S | | L54R | F94Y | 3.38 | 4.00 | 3.44 | |
| V37 | F29H | Q65A | Q99S | | N56W | F94Y | 3.46 | 3.69 | 3.49 | |
| V38 | T30G | T58V | | | L54R | F94H | 4.34 | 3.44 | 4.36 | |
| V39 | T30G | S61N | Q99G | | Q55V | F94S | 6.15 | 5.11 | 5.81 | |
| V40 | T30G | T58V | F110S | | N56L | S93R | 4.48 | 3.41 | 4.16 | |
| V41 | | I50T | | | | | 1.74 | 0.58 | 1.72 | |
| V42 | Y32A | | | | | | 0.45* | | | 0.41 |
| V43 | | D57G | | | | | 0.20* | | | 0.33 |
| V44 | | G54S | | | | | 0.65* | | | 0.52 |
| V45 | | | | W32F | | | 0.43* | | | 0.53 |
| V46 | | | | | S53T | | 0.83* | | | 0.96 |
| V47 | | | | | | R96M | 0.42* | | | 0.47 |
| V48 | T30G | T58V | Q99M | | N56T | F94L | 2.42 | 2.30 | 2.54 | |
| V49 | T30N | I50T, | Q99S | | L54R | F94Y | 6.51 | 5.02 | 6.58 | |
| | | Y60L | | | | | | | | |
| V50 | T30G | I50V | F110L | | L54R | F94L | 4.10 | 3.39 | 4.16 | |
| V51 | | T58V | Q99G, | | L54R | | 2.81 | 1.83 | 3.18 | |
| | | | Y112N | | | | | | | |
| V52 | T30E | | Q99G | | N56R | S93R | 3.00 | 1.78 | 3.09 | |
| V53 | | S63H | | | | | 1.25 | 0.66 | 1.17 | |
| V54 | Y32Q | | | | | | 0.55* | | | 0.54 |
| V55 | | R59I, | | | | | 0.24* | | | 0.66 |
| | | F64H | | | | | | | | |
| V56 | | S61Q | | | | | 0.23* | | | 0.59 |
| V57 | | | | R24A | | | 0.84* | | | 0.85 |
| V58 | | | | | A50K | | 0.28* | | | 0.68 |
| V59 | | | | | | Q89M | 0.19* | | | 0.60 |
| V60 | S28H | T58V | F110S | | N56R | Q89G | 3.26 | 3.35 | 3.63 | |
| V61 | T30S | S61N | Q99G | | Q55V | F94L | 5.08 | 3.63 | 5.22 | |
| V62 | T30G | S61A | D108G | | N56R | Q89G | 2.49 | 1.87 | 2.89 | |
| V63 | T30R | | Q99S | | N56R | S93R | 3.76 | 4.91 | 3.71 | |
| V64 | T30Q | | Q99G | | Q55A | F94Y | 5.41 | 4.88 | 5.48 | |
| V65 | | | Q99S | | | | 2.05 | 1.29 | 2.75 | |
| V66 | Y27T | | | | | | 0.25* | | | 0.74 |
| V67 | | I50M | | | | | 0.80* | | | 0.84 |
| V68 | | | Y103R | | | | 0.44* | | | 0.43 |
| V69 | | | | W32Y | | | 0.41* | | | 0.40 |
| V70 | | | | | S52G | | 0.79* | | | 0.84 |
| V71 | | | | | | F94E | 0.37* | | | 0.48 |
| V72 | A35G | | Q99G | | Q55V | F94Y | 3.64 | 2.50 | 4.01 | |
| V73 | T30G | S63G | Q99G | | L54R | F94Y | 5.12 | 4.17 | 5.44 | |
| V74 | T30A | T58V | Q99G | | N56L | | 3.94 | 2.54 | 4.01 | |
| V75 | | | Q99G | | N56A | F94Y | 4.64 | 3.74 | 4.52 | |
| V76 | T30G | S63E | F110S | | N56K | | 4.57 | 4.34 | 4.93 | |
| V77 | | | | | L54R | | 1.43 | 0.83 | 1.38 | |
| V78 | S28R | | | | | | 0.86* | | | 1.11 |
| V79 | | R59N | | | | | 0.70* | | | 0.52 |
| V80 | | | T101N | | | | 0.59* | | | 0.50 |
| V81 | | | | W32L | | | 0.17* | | | 0.23 |
| V82 | | | | | A51G | | 0.30* | | | 0.79 |
| V83 | | | | | | D92V | 0.20* | | | 0.29 |
| V84 | S28G | | F110S | | A50G | | 1.44 | 1.45 | 1.62 | |
| V85 | T30R | I50T | Q99S | | L54R | | 5.41 | 5.41 | 5.37 | |
| V86 | T30G, | Q65E | Q99S | | L54R | | 4.80 | 5.17 | 5.02 | |
| | I34L | | | | | | | | | |
| V87 | T30R | T58V, | Q99S | | N56W | | 3.84 | 4.86 | 3.93 | |
| | | S63D | | | | | | | | |
| V88 | T30G | | | | S53R, | F94S | 4.92 | 5.57 | 5.30 | |
| | | | | | N56R | | | | | |
| V89 | | | | | | F94H | 1.33 | 0.94 | 1.46 | |
| V90 | Y32E | | | S31R | | | 0.33* | | | 0.36 |
| V91 | | G54D | | | | | 0.25* | | | 0.61 |
| V92 | | | Y103H | | | | 0.22* | | | 0.65 |
| V93 | | | | S31G | | | 0.35* | | | 1.05 |
| V94 | | | | | S52A | | 0.31* | | | 0.87 |

Binding signal values marked with an * were obtained with the 110 nM Ab concentration, whereas the remaining values in the column were obtained with the 10 nM Ab concentration.

In some embodiments, the TREM2 agonist antigen binding protein comprises a light chain variable region comprising the sequence of SEQ ID NO: 61 with a mutation at one or more amino acid positions 24, 31, 50, 52, 54, 56, 89, 92, 93, 94 and/or 96. In certain embodiments, the mutation is selected from R24A, S31R, A50S, A50G, S52G, L54R, N56K, N56R, N56L, N56T, Q89G, D92V, S93R, F94Y, F94L, R96H, R96L, or combinations thereof. In these and other embodiments, the TREM2 agonist antigen binding protein comprises a heavy chain variable region comprising the sequence of SEQ ID NO: 124 with a mutation at one or more amino acid positions 27, 28, 30, 32, 50, 54, 58, 60, 61, 63, 66, 99, 101, 103, 104, and/or 110. In some embodiments, the mutation is selected from Y27S, S28G, S28H, T30N, T30G, T30E, T30A, Y32E, I50T, G54S, T58V, Y60L, S61A, S63G, S63E, G66D, Q99G, Q99S, Q99M, T101G, Y103R, Y104G, F110S, or combinations thereof. Amino acid sequences for light chain and heavy chain variable regions and associated CDRs of exemplary variants of the 6E7 antibody with improved affinity are set forth below in Tables 3A and 3B, respectively. Amino acid sequences for light chain and heavy chain variable regions and associated CDRs of exemplary variants of the 6E7 antibody with reduced affinity are set forth below in Tables 3C and 3D, respectively. The corresponding sequences for the 6E7 antibody are listed for comparison.

TABLE 3A
Light Chain Variable Region Amino Acid Sequences for
Improved Affinity TREM2 Antibodies

| Variant | VL | VL Amino Acid | | | |
|---|---|---|---|---|---|
| Ab ID. | Group | Sequence | CDRL1 | CDRL2 | CDRL3 |
| 6E7 | LV-16 | DIQMTQSPSSVSASVGDR | RASQGISSWL | AASSLQN | QQADSFPRT |
| | | VTITCRASQGISSWLAWY | A (SEQ ID | (SEQ ID | (SEQ ID |
| | | QQKPGKAPKLLIYAASSL | NO: 16) | NO: 28) | NO: 43 |
| | | QNGVPSRFSGSGSGTDFT | | | |
| | | LTISSLQPEDFATYFCQQ | | | |
| | | ADSFPRTFGQGTKLEIK | | | |
| | | (SEQ ID NO: 61) | | | |
| V3 | LV- | DIQMTQSPSSVSASVGDR | RASQGISSWL | AASSRQN | QQADREPRT |
| | 101 | VTITCRASQGISSWLAWY | A | (SEQ ID | (SEQ ID |
| | | QQKPGKAPKLLIYAASSR | (SEQ ID | NO: 143) | NO: 148) |
| | | QNGVPSRFSGSGSGTDET | NO: 16) | | |
| | | LTISSLQPEDFATYFCQQ | | | |
| | | ADRFPRTFGQGTKLEIK | | | |
| | | (SEQ ID NO: 153) | | | |
| V24 | LV- | DIQMTQSPSSVSASVGDR | RASQGISSWL | AASSLQK | QQADSFPHT |
| | 102 | VTITCRASQGISSWLAWY | A | (SEQ ID | (SEQ ID |
| | | QQKPGKAPKLLIYAASSL | (SEQ ID | NO: 144) | NO: 149) |
| | | QKGVPSRFSGSGSGTDFT | NO: 16) | | |
| | | LTISSLQPEDFATYFCQQ | | | |
| | | ADSFPHTFGQGTKLEIK | | | |
| | | (SEQ ID NO: 154) | | | |
| V27 | LV- | DIQMTQSPSSVSASVGDR | RASQGISSWL | AASSLQR | QQADSEPRT |
| | 103 | VTITCRASQGISSWLAWY | A | (SEQ ID | (SEQ ID |
| | | QQKPGKAPKLLIYAASSL | (SEQ ID | NO: 145) | NO: 43) |
| | | QRGVPSRFSGSGSGTDET | NO: 16) | | |
| | | LTISSLQPEDFATYFCQQ | | | |
| | | ADS FPRTFGQGTKLEIK | | | |
| | | (SEQ ID NO: 155) | | | |
| V40 | LV- | DIQMTQSPSSVSASVGDR | RASQGISSWL | AASSLQL | QQADREPRT |
| | 104 | VTITCRASQGISSWLAWY | A | (SEQ ID | (SEQ ID |
| | | QQKPGKAPKLLIYAASSL | (SEQ ID | NO: 146) | NO: 148) |
| | | QLGVPSRESGSGSGTDET | NO: 16) | | |
| | | LTISSLQPEDFATYFCQQ | | | |
| | | ADRFPRTFGQGTKLEIK | | | |
| | | (SEQ ID NO: 156) | | | |
| V48 | LV- | DIQMTQSPSSVSASVGDR | RASQGISSWL | AASSLQT | QQADSLPRT |
| | 105 | VTITCRASQGISSWLAWY | A | (SEQ ID | (SEQ ID |
| | | QQKPGKAPKLLIYAASSL | (SEQ ID | NO: 26) | NO: 150) |
| | | QTGVPSRFSGSGSGTDET | NO: 16) | | |
| | | LTISSLQPEDFATYFCQQ | | | |
| | | ADSLPRTFGQGTKLEIK | | | |
| | | (SEQ ID NO: 157) | | | |
| V49 | LV- | DIQMTQSPSSVSASVGDR | RASQGISSWL | AASSRQN | QQADSYPRT |
| V73 | 106 | VTITCRASQGISSWLAWY | A | (SEQ ID | (SEQ ID |
| | | QQKPGKAPKLLIYAASSR | (SEQ ID | NO: 143) | NO: 151) |
| | | QNGVPSRFSGSGSGTDET | NO: 16) | | |
| | | LTISSLQPEDFATYFCQQ | | | |
| | | ADSYPRTFGQGTKLEIK | | | |
| | | (SEQ ID NO: 158) | | | |
| V52 | LV- | DIQMTQSPSSVSASVGDR | RASQGISSWL | AASSLQR | QQADRFPRT |
| | 107 | VTITCRASQGISSWLAWY | A | (SEQ ID | (SEQ ID |
| | | QQKPGKAPKLLIYAASSL | (SEQ ID | NO: 145) | NO: 148) |
| | | QRGVPSRESGSGSGTDET | NO: 16) | | |
| | | LTISSLQPEDFATYFCQQ | | | |
| | | ADRFPRTFGQGTKLEIK | | | |
| | | (SEQ ID NO: 159) | | | |
| V60 | LV- | DIQMTQSPSSVSASVGDR | RASQGISSWL | AASSLQR | GQADSFPRT |
| | 108 | VTITCRASQGISSWLAWY | A | (SEQ ID | (SEQ ID |
| | | QQKPGKAPKLLIYAASSL | (SEQ ID | NO: 145) | NO: 152) |
| | | QRGVPSRESGSGSGTDET | NO: 16) | | |
| | | LTISSLQPEDFATYFCGQ | | | |
| | | ADSFPRTFGQGTKLEIK | | | |
| | | (SEQ ID NO: 160) | | | |
| V76 | LV- | DIQMTQSPSSVSASVGDR | RASQGISSWL | AASSLQK | QQADSFPRT |
| | 109 | VTITCRASQGISSWLAWY | A | (SEQ ID | (SEQ ID |
| | | QQKPGKAPKLLIYAASSL | (SEQ ID | NO: 144) | NO: 43) |
| | | QKGVPSRFSGSGSGRDFT | NO: 16) | | |
| | | LTISSLQPEDFATYFCQQ | | | |
| | | ADSFPRTFGQGTKLEIK | | | |
| | | (SEQ ID NO: 161) | | | |
| V84 | LV- | DIQMTQSPSSVSASVGDR | RASQGISSWL | GASSLQN | QQADSFPRT |
| | 110 | VTITCRASQGISSWLAWY | A | (SEQ ID | (SEQ ID |
| | | QQKPGKAPKLLIYGASSL | (SEQ ID | NO: 147) | NO: 43) |
| | | QNGVPSRFSGSGSGTDET | NO: 16) | | |
| | | LTISSLQPEDFATYFCQQ | | | |
| | | ADSFPRTFGQGTKLEIK | | | |
| | | (SEQ ID NO: 162) | | | |

TABLE 3B
Heavy Chain Variable Region Amino Acid Sequences for
Improved Affinity TREM2 Antibodies

| | | VH Amino | FR1/ | | | |
|---|---|---|---|---|---|---|
| Variant | VH | Acid | CDRH1 | | | |
| Ab ID. | Group | Sequence | Border | CDRH1 | CDRH2 | CDRH3 |
| 6E7 | HV- | EVQLVQSGAEV | YSFT | SYWIA | IIYPGDSDTRY | QRTFYYDSSDYEDY |
| | 15 | KKPGESLKISC | (SEQ ID | (SEQ ID | SPSFQG (SEQ | (SEQ ID NO: |
| | | KGSGYSFTSYW | NO: | NO: 85) | ID NO: 91) | 107) |
| | | IAWVRQMPGKG | 163) | | | |
| | | LEWMGIIYPGD | | | | |
| | | SDTRYSPSFQG | | | | |
| | | QVTISADKSIS | | | | |
| | | TAYLOWSSLKA | | | | |
| | | SDTAMYFCARQ | | | | |
| | | RTFYYDSSDYF | | | | |
| | | DYWGQGTLVTV | | | | |
| | | SS | | | | |
| | | (SEQ ID NO: | | | | |
| | | 124) | | | | |
| V3 | HV- | EVQLVQSGAEV | YSFA | SYWIA | IIYPGDSDTRY | GRTFYYDSSDYFDY |
| | 101 | KKPGESLKISC | (SEQ ID | (SEQ ID | SPSFQD (SEQ | (SEQ ID NO: |
| | | KGSGYSFASYW | NO: | NO: 85) | ID NO: 170) | 176) |
| | | IAWVRQMPGKG | 164) | | | |
| | | LEWMGIIYPGD | | | | |
| | | SDTRYSPSFQD | | | | |
| | | QVTISADKSIS | | | | |
| | | TAYLOWSSLKA | | | | |
| | | SDTAMYFCARG | | | | |
| | | RTFYYDSSDYF | | | | |
| | | DYWGQGTLVTV | | | | |
| | | SS | | | | |
| | | (SEQ ID NO: | | | | |
| | | 180) | | | | |
| V24 | HV- | EVQLVQSGAEV | YSFT | SYWIA | IIYPGDSDVRY | SRTFYYDSSDYFDY |
| | 102 | KKPGESLKISC | (SEQ ID | (SEQ ID | SPSFQG (SEQ | (SEQ ID NO: |
| | | KGSGYSFTSYW | NO: | NO: 85) | ID NO: 171) | 177) |
| | | IAWVRQMPGKG | 163) | | | |
| | | LEWMGIIYPGD | | | | |
| | | SDVRYSPSFQG | | | | |
| | | QVTISADKSIS | | | | |
| | | TAYLQWSSLKA | | | | |
| | | SDTAMYFCARS | | | | |
| | | RTFYYDSSDYF | | | | |
| | | DYWGQGTLVTV | | | | |
| | | SS | | | | |
| | | (SEQ ID NO: | | | | |
| | | 181) | | | | |
| V27 | HV- | EVQLVQSGAEV | YSFT | SYWIA | IIYPGDSDTRY | SRTFYYDSSDYFDY |
| | 103 | KKPGESLKISC | (SEQ ID | (SEQ ID | APSFQG (SEQ | (SEQ ID NO: |
| | | KGSGYSFTSYW | NO: | NO: 85) | ID NO: 172) | 177) |
| | | IAWVRQMPGKG | 163) | | | |
| | | LEWMGIIYPGD | | | | |
| | | SDTRYAPSFQG | | | | |
| | | QVTISADKSIS | | | | |
| | | TAYLQWSSLKA | | | | |
| | | SDTAMYFCVRS | | | | |
| | | RTFYYDSSDYF | | | | |
| | | DYWGQGTLVTV | | | | |
| | | SS | | | | |
| | | (SEQ ID NO: | | | | |
| | | 182) | | | | |
| V40 | HV- | EVQLVQSGAEV | YSFG | SYWIA | IIYPGDSDVRY | QRTFYYDSSDYSDY |
| | 104 | KKPGESLKISC | (SEQ ID | (SEQ ID | SPSFQG | (SEQ ID NO: |
| | | KGSGYSFGSYW | NO: | NO: 85) | (SEQ ID NO: | 178) |
| | | IAWVRQMPGKG | 165 ) | | 171) | |
| | | LEWMGIIYPGD | | | | |
| | | SDVRYSPSFQG | | | | |
| | | QVTISADKSIS | | | | |
| | | TAYLQWSSLKA | | | | |
| | | SDTAMYFCARQ | | | | |
| | | RTFYYDSSDYS | | | | |
| | | DYWGQGTLVTV | | | | |
| | | SS | | | | |
| | | (SEQ ID NO: | | | | |
| | | 183) | | | | |
| V48 | HV- | EVQLVQSGAEV | YSFG | SYWIA | IIYPGDSDVRY | MRTFYYDSSDYFDY |
| | 105 | KKPGESLKISC | (SEQ ID | (SEQ ID | SPSFQG | (SEQ ID NO: |
| | | KGSGYSFGSYW | NO: | NO: 85) | (SEQ ID NO: | 179) |
| | | IAWVRQMPGKG | 165) | | 171) | |
| | | LEWMGIIYPGD | | | | |
| | | SDVRYSPSFQG | | | | |
| | | QVTISADKSIS | | | | |
| | | TAYLOWSSLKA | | | | |
| | | SDTAMYFCARM | | | | |
| | | RTFYYDSSDYF | | | | |
| | | DYWGQGTLVTV | | | | |
| | | SS | | | | |
| | | (SEQ ID NO: | | | | |
| | | 184) | | | | |
| V49 | HV- | EVQLVQSGAEV | YSEN | SYWIA | TIYPGDSDTRL | SRTFYYDSSDYFDY |
| | 106 | KKPGESLKISC | (SEQ ID | (SEQ ID | SPSFQG (SEQ | (SEQ ID NO: |
| | | KGSGYSFNSYW | NO: | NO: 85) | ID NO: 173) | 177) |
| | | IAWVRQMPGKG | 166) | | | |
| | | LEWMGTIYPGD | | | | |
| | | SDTRLSPSFQG | | | | |
| | | QVTISADKSIS | | | | |
| | | TAYLQWSSLKA | | | | |
| | | SDTAMYFCARS | | | | |
| | | RTFYYDSSDYF | | | | |
| | | DYWGQGTLVTV | | | | |
| | | SS | | | | |
| | | (SEQ ID NO: | | | | |
| | | 185) | | | | |
| V52 | HV- | EVQLVQSGAEV | YSFE | SYWIA | IIYPGDSDTRY | GRTFYYDSSDYFDY |
| | 107 | KKPGESLKISC | (SEQ ID | (SEQ ID | SPSFQG | (SEQ ID NO: |
| | | KGSGYSFESYW | NO: | NO: 85) | (SEQ ID NO: | 176) |
| | | IAWVRQMPGKG | 167) | | 91) | |
| | | LEWMGIIYPGD | | | | |
| | | SDTRYSPSFQG | | | | |
| | | QVTISADKSIS | | | | |
| | | TAYLOWSSLKA | | | | |
| | | SDTAMYFCARG | | | | |
| | | RTFYYDSSDYF | | | | |
| | | DYWGQGTLVTV | | | | |
| | | SS | | | | |
| | | (SEQ ID NO: | | | | |
| | | 186) | | | | |
| V60 | HV- | EVQLVQSGAEV | YHFT | SYWIA | IIYPGDSDVRY | QRTFYYDSSDYSDY |
| | 108 | KKPGESLKISC | (SEQ ID | (SEQ ID | SPSFQG | (SEQ ID NO: |
| | | KGSGYHFTSYW | NO: | NO: 85) | (SEQ ID NO: | 178) |
| | | IAWVRQMPGKG | 168) | | 171) | |
| | | LEWMGIIYPGD | | | | |
| | | SDVRYSPSFQG | | | | |
| | | QVTISADKSIS | | | | |
| | | TAYLQWSSLKA | | | | |
| | | SDTAMYFCARQ | | | | |
| | | RTFYYDSSDYS | | | | |
| | | DYWGQGTLVTV | | | | |
| | | SS | | | | |
| | | (SEQ ID NO: | | | | |
| | | 187) | | | | |
| V73 | HV- | EVQLVQSGAEV | YSFG | SYWIA | IIYPGDSDTRY | GRTFYYDSSDYFDY |
| | 109 | KKPGESLKISC | (SEQ ID | (SEQ ID | SPGFQG (SEQ | (SEQ ID NO: |
| | | KGSGYSFGSYW | NO: | NO: 85) | ID NO: 174) | 176) |
| | | IAWVRQMPGKG | 165) | | | |
| | | LEWMGIIYPGD | | | | |
| | | SDTRYSPGFQG | | | | |
| | | QVTISADKSIS | | | | |
| | | TAYLQWSSLKA | | | | |
| | | SDTAMYFCARG | | | | |
| | | RTFYYDSSDYF | | | | |
| | | DYWGQGTLVTV | | | | |
| | | SS | | | | |
| | | (SEQ ID NO: | | | | |
| | | 188) | | | | |
| V76 | HV- | EVQLVQSGAEV | YSFG | SYWIA | IIYPGDSDTRY | QRTFYYDSSDYSDY |
| | 110 | KKPGESLKISC | (SEQ ID | (SEQ ID | SPEFQG (SEQ | (SEQ ID NO: |
| | | KGSGYSFGSYW | NO: | NO: 85) | ID NO: 175) | 178) |
| | | IAWVRQMPGKG | 165) | | | |
| | | LEWMGIIYPGD | | | | |
| | | SDTRYSPEFQG | | | | |
| | | QVTISADKSIS | | | | |
| | | TAYLOWSSLKA | | | | |
| | | SDTAMYFCARQ | | | | |
| | | RTFYYDSSDYS | | | | |
| | | DYWGQGTLVTV | | | | |
| | | SS | | | | |
| | | (SEQ ID NO: | | | | |
| | | 189) | | | | |
| V84 | HV- | EVQLVQSGAEV | YGFT | SYWIA | IIYPGDSDTRY | QRTFYYDSSDYSDY |
| | 111 | KKPGESLKISC | (SEQ ID | (SEQ ID | SPSFQG | (SEQ ID NO: |
| | | KGSGYGFTSYW | NO: | NO: 85) | (SEQ ID NO: | 178) |
| | | IAWVRQMPGKG | 169) | | 91) | |
| | | LEWMGIIYPGD | | | | |
| | | SDTRYSPSFQG | | | | |
| | | QVTISADKSIS | | | | |
| | | TAYLOWSSLKA | | | | |
| | | SDTAMYFCARQ | | | | |
| | | RTFYYDSSDYS | | | | |
| | | DYWGQGTLVTV | | | | |
| | | SS | | | | |
| | | (SEQ ID NO: | | | | |
| | | 190) | | | | |

In some embodiments, the TREM2 agonist antigen binding proteins of the invention may comprise one or more of the CDRs from the improved affinity variants presented in Table 3A (light chain CDRs; i.e., CDRLs) and Table 3B3 (heavy chain CDRs, i.e., CDRHs). In some embodiments, the TREM2 agonist antigen binding proteins comprise a consensus CDR sequence derived from the improved affinity variants. For instance, in some embodiments, the TREM2 agonist antigen binding proteins comprise a CDRL2 consensus sequence of X₁ASSX₂QX₃ (SEQ ID NO: 139), where X₁ is A or G; X₂ is L or R; and X₃ is N, K, R, L, or T. In another embodiment, the TREM2 agonist antigen binding proteins comprise a CDRL3 consensus sequence of X₁QADX₂X₃PX₄T (SEQ ID NO: 140), where X₁ is Q or G; X₂ is S or R; X₃ is F, L, or Y; and X₄ is R or H. In yet another embodiment, the TREM2 agonist antigen binding proteins comprise a CDRH2 consensus sequence of X₁IYPGDSDX₂RX₃X₄PX₅FQX₆ (SEQ ID NO: 141), where X₁ is I or T; X₂ is T or V; X₃ is Y or L; X₄ is S or A; X₅ is 5, G, or E; and X₆ is G or D. In some embodiments, the TREM2 agonist antigen binding proteins comprise a CDRH3 consensus sequence of X₁RTFYYDSSDYX₂DY (SEQ ID NO: 142), where X₁ is Q, G, 5, or M; and X₂ is F or S.

In some embodiments, the TREM2 agonist antigen binding proteins comprise a light chain variable region comprising complementarity determining regions CDRL1, CDRL2, and CDRL3 and a heavy chain variable region comprising complementarity determining regions CDRH1, CDRH2, and CDRH3, wherein CDRL1 comprises the sequence of SEQ ID NO: 16, CDRL2 comprises the consensus sequence of SEQ ID NO: 139, CDRL3 comprises the consensus sequence of SEQ ID NO: 140, CDRH1 comprises the sequence of SEQ ID NO: 85, CDRH2 comprises the consensus sequence of SEQ ID NO: 141, and CDRH3 comprises the consensus sequence of SEQ ID NO: 142.

In some embodiments, the TREM2 agonist antigen binding protein comprises a CDRL1 comprising the sequence of SEQ ID NO: 16; a CDRL2 comprising a sequence selected from SEQ ID NOs: 26 and 143-147; a CDRL3 comprising a sequence selected from SEQ ID NOs: 43 and 148-152; a CDRH1 comprising the sequence of SEQ ID NO: 85; a CDRH2 comprising a sequence selected from SEQ ID NOs: 91 and 170-175; and a CDRH3 comprising a sequence selected from SEQ ID NOs: 176-179.

In particular embodiments, the TREM2 agonist antigen binding proteins of the invention comprise a light chain variable region comprising a CDRL1, a CDRL2, and a CDRL3, wherein:
(a) CDRL1, CDRL2, and CDRL3 have the sequence of SEQ ID NOs: 16, 143, and 148, respectively;
(b) CDRL1, CDRL2, and CDRL3 have the sequence of SEQ ID NOs: 16, 144, and 149, respectively;
(c) CDRL1, CDRL2, and CDRL3 have the sequence of SEQ ID NOs: 16, 145, and 43, respectively;
(d) CDRL1, CDRL2, and CDRL3 have the sequence of SEQ ID NOs: 16, 146, and 148, respectively;
(e) CDRL1, CDRL2, and CDRL3 have the sequence of SEQ ID NOs: 16, 26, and 150, respectively;
(f) CDRL1, CDRL2, and CDRL3 have the sequence of SEQ ID NOs: 16, 143, and 151, respectively;
(g) CDRL1, CDRL2, and CDRL3 have the sequence of SEQ ID NOs: 16, 145, and 148, respectively;
(h) CDRL1, CDRL2, and CDRL3 have the sequence of SEQ ID NOs: 16, 145, and 152, respectively;
(i) CDRL1, CDRL2, and CDRL3 have the sequence of SEQ ID NOs: 16, 144, and 43, respectively; or
(j) CDRL1, CDRL2, and CDRL3 have the sequence of SEQ ID NOs: 16, 147, and 43, respectively.

In related embodiments, the TREM2 agonist antigen binding proteins of the invention comprise a heavy chain variable region comprising a CDRH1, a CDRH2, and a CDRH3, wherein:
(a) CDRH1, CDRH2, and CDRH3 have the sequence of SEQ ID NOs: 85, 170, and 176, respectively;
(b) CDRH1, CDRH2, and CDRH3 have the sequence of SEQ ID NOs: 85, 171, and 177, respectively;
(c) CDRH1, CDRH2, and CDRH3 have the sequence of SEQ ID NOs: 85, 172, and 177, respectively;
(d) CDRH1, CDRH2, and CDRH3 have the sequence of SEQ ID NOs: 85, 171, and 178, respectively;
(e) CDRH1, CDRH2, and CDRH3 have the sequence of SEQ ID NOs: 85, 171, and 179, respectively;
(f) CDRH1, CDRH2, and CDRH3 have the sequence of SEQ ID NOs: 85, 173, and 177, respectively;
(g) CDRH1, CDRH2, and CDRH3 have the sequence of SEQ ID NOs: 85, 91, and 176, respectively;
(h) CDRH1, CDRH2, and CDRH3 have the sequence of SEQ ID NOs: 85, 174, and 176, respectively;
(i) CDRH1, CDRH2, and CDRH3 have the sequence of SEQ ID NOs: 85, 175, and 178, respectively; or
(j) CDRH1, CDRH2, and CDRH3 have the sequence of SEQ ID NOs: 85, 91, and 178, respectively.

In some embodiments, the TREM2 agonist antigen binding proteins of the invention comprise a light chain variable region comprising a CDRL1, a CDRL2, and a CDRL3 and a heavy chain variable region comprising a CDRH1, a CDRH2, and a CDRH3, wherein:
(a) CDRL1, CDRL2, and CDRL3 have the sequence of SEQ ID NOs: 16, 143, and 148, respectively, and CDRH1, CDRH2, and CDRH3 have the sequence of SEQ ID NOs: 85, 170, and 176, respectively;
(b) CDRL1, CDRL2, and CDRL3 have the sequence of SEQ ID NOs: 16, 144, and 149, respectively, and CDRH1, CDRH2, and CDRH3 have the sequence of SEQ ID NOs: 85, 171, and 177, respectively;
(c) CDRL1, CDRL2, and CDRL3 have the sequence of SEQ ID NOs: 16, 145, and 43, respectively, and CDRH1, CDRH2, and CDRH3 have the sequence of SEQ ID NOs: 85, 172, and 177, respectively;
(d) CDRL1, CDRL2, and CDRL3 have the sequence of SEQ ID NOs: 16, 146, and 148, respectively, and CDRH1, CDRH2, and CDRH3 have the sequence of SEQ ID NOs: 85, 171, and 178, respectively;
(e) CDRL1, CDRL2, and CDRL3 have the sequence of SEQ ID NOs: 16, 26, and 150, respectively, and CDRH1, CDRH2, and CDRH3 have the sequence of SEQ ID NOs: 85, 171, and 179, respectively;
(f) CDRL1, CDRL2, and CDRL3 have the sequence of SEQ ID NOs: 16, 143, and 151, respectively, and CDRH1, CDRH2, and CDRH3 have the sequence of SEQ ID NOs: 85, 173, and 177, respectively;
(g) CDRL1, CDRL2, and CDRL3 have the sequence of SEQ ID NOs: 16, 145, and 148, respectively, and CDRH1, CDRH2, and CDRH3 have the sequence of SEQ ID NOs: 85, 91, and 176, respectively;
(h) CDRL1, CDRL2, and CDRL3 have the sequence of SEQ ID NOs: 16, 145, and 152, respectively, and CDRH1, CDRH2, and CDRH3 have the sequence of SEQ ID NOs: 85, 171, and 178, respectively;
(i) CDRL1, CDRL2, and CDRL3 have the sequence of SEQ ID NOs: 16, 143, and 151, respectively, and CDRH1, CDRH2, and CDRH3 have the sequence of SEQ ID NOs: 85, 174, and 176, respectively;
(j) CDRL1, CDRL2, and CDRL3 have the sequence of SEQ ID NOs: 16, 144, and 43, respectively, and CDRH1, CDRH2, and CDRH3 have the sequence of SEQ ID NOs: 85, 175, and 178, respectively; or
(k) CDRL1, CDRL2, and CDRL3 have the sequence of SEQ ID NOs: 16, 147, and 43, respectively, and CDRH1, CDRH2, and CDRH3 have the sequence of SEQ ID NOs: 85, 91, and 178, respectively.

In some embodiments, the TREM2 agonist antigen binding proteins of the invention may comprise a light chain variable region selected from LV-101, LV-102, LV-103, LV-104, LV-105, LV-106, LV-107, LV-108, LV-109, and LV-110, as shown in Table 3A, and/or a heavy chain variable region selected from HV-101, HV-102, HV-103, HV-104, HV-105, HV-106, HV-107, HV-108, HV-109, HV-110, and HV-111, as shown in Table 3B, or sequences that are at least 80% identical, at least 85% identical, at least 90% identical, or at least 95% identical to any of the sequences in Tables 3A and 3B. For instance, in some embodiments, the TREM2 agonist antigen binding proteins comprise a light chain variable region comprising (i) a sequence that is at least 90% identical to a sequence selected from SEQ ID NOs: 153-162, (ii) a sequence that is at least 95% identical to a sequence selected from SEQ ID NOs: 153-162, or (iii) a sequence selected from SEQ ID NOs: 153-162. In related embodiments, the TREM2 agonist antigen binding proteins comprise a heavy chain variable region comprising (i) a sequence that is at least 90% identical to a sequence selected from SEQ ID NOs: 180-190, (ii) a sequence that is at least 95% identical to a sequence selected from SEQ ID NOs: 180-190, or (iii) a sequence selected from SEQ ID NOs: 180-190.

Each of the light chain variable regions listed in Table 3A may be combined with any of the heavy chain variable regions listed in Table 3B to form an anti-TREM2 binding domain of the antigen binding proteins of the invention. Examples of such combinations include, but are not limited to: LV-101 (SEQ ID NO: 153) and HV-101 (SEQ ID NO: 180); LV-102 (SEQ ID NO: 154) and HV-102 (SEQ ID NO: 181); LV-103 (SEQ ID NO: 155) and HV-103 (SEQ ID NO: 182); LV-104 (SEQ ID NO: 156) and HV-104 (SEQ ID NO: 183); LV-105 (SEQ ID NO: 157) and HV-105 (SEQ ID NO: 184); LV-106 (SEQ ID NO: 158) and HV-106 (SEQ ID NO: 185); LV-107 (SEQ ID NO: 159) and HV-107 (SEQ ID NO: 186); LV-108 (SEQ ID NO: 160) and HV-108 (SEQ ID NO: 187); LV-106 (SEQ ID NO: 158) and HV-109 (SEQ ID NO: 188); LV-109 (SEQ ID NO: 161) and HV-110 (SEQ ID NO: 189); and LV-110 (SEQ ID NO: 162) and HV-111 (SEQ ID NO: 190).

TABLE 3C
Light Chain Variable Region Amino Acid Sequences for
Reduced Affinity TREM2 Antibodies

| Variant | VL | VL Amino Acid | | | |
|---|---|---|---|---|---|
| Ab ID. | Group | Sequence | CDRL1 | CDRL2 | CDRL3 |
| 6E7 | LV-16 | DIQMTQSPSSVSASVGDRVT | RASQGISSWLA | AASSLQN | QQADSFPRT |
| | | ITCRASQGISSWLAWYQQKP | (SEQ ID NO: | (SEQ ID | (SEQ ID |
| | | GKAPKLLIYAASSLQNGVPS | 16) | NO: 28) | NO: 43) |
| | | RFSGSGSGTDFTLTISSLQP | | | |
| | | EDFATYFCQQADSFPRTFGQ | | | |
| | | GTKLEIK (SEQ ID NO: | | | |
| | | 61) | | | |
| V9 | LV-16 | DIQMTQSPSSVSASVGDRVT | RASQGISSWLA | AASSLQN | QQADSFPRT |
| V30 | | ITCRASQGISSWLAWYQQKP | (SEQ ID NO: | (SEQ ID | (SEQ ID |
| V33 | | GKAPKLLIYAASSLQNGVPS | 16) | NO: 28) | NO: 43) |
| V44 | | RFSGSGSGTDFTLTISSLQP | | | |
| V68 | | EDFATYFCQQADSFPRTFGQ | | | |
| | | GTKLEIK (SEQ ID NO: | | | |
| | | 61) | | | |
| V10 | LV- | DIQMTQSPSSVSASVGDRVT | RASQGISSWLA | SASSLQN | QQADSFPRT |
| | 201 | ITCRASQGISSWLAWYQQKP | (SEQ ID NO: | (SEQ ID | (SEQ ID |
| | | GKAPKLLIYSASSLQNGVPS | 16) | NO: 292) | NO: 43) |
| | | RFSGSGSGTDFTLTISSLQP | | | |
| | | EDFATYFCQQADSFPRTFGQ | | | |
| | | GTKLEIK (SEQ ID NO: | | | |
| | | 295) | | | |
| V23 | LV- | DIQMTQSPSSVSASVGDRVT | RASQGISSWLA | AASSLQN | QQADSFPLT |
| | 202 | ITCRASQGISSWLAWYQQKP | (SEQ ID NO: | (SEQ ID | (SEQ ID |
| | | GKAPKLLIYAASSLQNGVPS | 16 | NO: 28) | NO: 294) |
| | | RFSGSGSGTDFTLTISSLQP | | | |
| | | EDFATYFCQQADSFPLTFGQ | | | |
| | | GTKLEIK (SEQ ID NO: | | | |
| | | 296) | | | |
| V57 | LV- | DIQMTQSPSSVSASVGDRVT | AASQGISSWLA | AASSLQN | QQADSFPRT |
| | 203 | ITCAASQGISSWLAWYQQKP | (SEQ ID NO: | (SEQ ID | (SEQ ID |
| | | GKAPKLLIYAASSLQNGVPS | 290) | NO: 28) | NO: 43) |
| | | RFSGSGSGTDFTLTISSLQP | | | |
| | | EDFATYFCQQADSFPRTFGQ | | | |
| | | GTKLEIK (SEQ ID NO: | | | |
| | | 297) | | | |
| V70 | LV- | DIQMTQSPSSVSASVGDRVT | RASQGISSWLA | AAGSLQN | QQADSFPRT |
| | 204 | ITCRASQGISSWLAWYQQKP | (SEQ ID NO: | (SEQ ID | (SEQ ID |
| | | GKAPKLLIYAAGSLQNGVPS | 16 | NO: 293) | NO: 43) |
| | | RFSGSGSGTDFTLTISSLQP | | | |
| | | EDFATYFCQQADSFPRTFGQ | | | |
| | | GTKLEIK (SEQ ID NO: | | | |
| | | 298) | | | |
| V83 | LV- | DIQMTQSPSSVSASVGDRVT | RASQGISSWLA | AASSLQN | QQAVSFPRT |
| | 205 | ITCRASQGISSWLAWYQQKP | (SEQ ID NO: | (SEQ ID | (SEQ ID |
| | | GKAPKLLIYAASSLQNGVPS | 16) | NO: 28) | NO: 271) |
| | | RFSGSGSGTDFTLTISSLQP | | | |
| | | EDFATYFCQQAVSFPRTFGQ | | | |
| | | GTKLEIK (SEQ ID NO: | | | |
| | | 299) | | | |
| V90 | LV- | DIQMTQSPSSVSASVGDRVT | RASQGISRWLA | AASSLQN | QQADSFPRT |
| | 206 | ITCRASQGISRWLAWYQQKP | (SEQ ID NO: | (SEQ ID | (SEQ ID |
| | | GKAPKLLIYAASSLQNGVPS | 291) | NO: 28) | NO: 43) |
| | | RFSGSGSGTDETLTISSLQP | | | |
| | | EDFATYFCQQADSFPRTFGQ | | | |
| | | GTKLEIK (SEQ ID NO: | | | |
| | | 300) | | | |

TABLE 3D
Heavy Chain Variable Region Amino Acid Sequences for
Reduced Affinity TREM2 Antibodies

| | | VH Amino | FR1/ | | | |
|---|---|---|---|---|---|---|
| Variant | VH | Acid | CDRH1 | | | |
| Ab ID. | Group | Sequence | border | CDRH1 | CDRH2 | CDRH3 |
| 6E7 | HV- | EVQLVQSGAEV | YSFT | SYWIA | IIYPGDSDTRYSPSFQ | QRTFYYDSSDYFDY |
| | 15 | KKPGESLKISC | (SEQ | (SEQ | G (SEQ ID NO: | (SEQ ID NO: |
| | | KGSGYSFTSYW | ID | ID | 91) | 107) |
| | | IAWVRQMPGKG | NO: | NO: | | |
| | | LEWMGIIYPGD | 163) | 85) | | |
| | | SDTRYSPSFQG | | | | |
| | | QVTISADKSIS | | | | |
| | | TAYLQWSSLKA | | | | |
| | | SDTAMYFCARQ | | | | |
| | | RTFYYDSSDYF | | | | |
| | | DYWGQGTLVTV | | | | |
| | | SS (SEQ ID | | | | |
| | | NO: 124) | | | | |
| V9 | HV- | EVQLVQSGAEV | YSFT | SYWIA | IIYPGDSDTRYSPSFQ | QRGFYYDSSDYFDY |
| | 201 | KKPGESLKISC | (SEQ | (SEQ | G (SEQ ID NO: | (SEQ ID NO: |
| | | KGSGYSFTSYW | ID | ID | 91) | 304) |
| | | IAWVRQMPGKG | NO: | NO: | | |
| | | LEWMGIIYPGD | 163) | 85) | | |
| | | SDTRYSPSFQG | | | | |
| | | QVTISADKSIS | | | | |
| | | TAYLQWSSLKA | | | | |
| | | SDTAMYFCARQ | | | | |
| | | RGFYYDSSDYF | | | | |
| | | DYWGQGTLVTV | | | | |
| | | SS (SEQ ID | | | | |
| | | NO: 307) | | | | |
| V10 | HV- | EVQLVQSGAEV | YSFT | SYWIA | IIYPGDSDTRYSPSFQ | QRTFYYDSSDYFDY |
| V23 | 15 | KKPGESLKISC | (SEQ | (SEQ | G (SEQ ID NO: | (SEQ ID NO: |
| V57 | | KGSGYSFTSYW | ID | ID | 91) | 107) |
| V70 | | IAWVRQMPGKG | NO: | NO: | | |
| V83 | | LEWMGIIYPGD | 163) | 85) | | |
| | | SDTRYSPSFQG | | | | |
| | | QVTISADKSIS | | | | |
| | | TAYLQWSSLKA | | | | |
| | | SDTAMYFCARQ | | | | |
| | | RTFYYDSSDYE | | | | |
| | | DYWGQGTLVTV | | | | |
| | | SS (SEQ ID | | | | |
| | | NO: 124) | | | | |
| V30 | HV- | EVQLVQSGAEV | SSFT | SYWIA | IIYPGDSDTRYSPSFQ | QRTFYYDSSDYFDY |
| | 202 | KKPGESLKISC | (SEQ | (SEQ | G (SEQ ID NO: | (SEQ ID NO: |
| | | KGSGSSFTSYW | ID | ID | 91) | 107) |
| | | IAWVRQMPGKG | NO: | NO: | | |
| | | LEWMGIIYPGD | 301) | 85) | | |
| | | SDTRYSPSFQG | | | | |
| | | QVTISADKSIS | | | | |
| | | TAYLOWSSLKA | | | | |
| | | SDTAMYFCARQ | | | | |
| | | RTFYYDSSDYF | | | | |
| | | DYWGQGTLVTV | | | | |
| | | SS (SEQ ID | | | | |
| | | NO: 308) | | | | |
| V33 | HV- | EVQLVQSGAEV | YSFT | SYWIA | IIYPGDSDTRYSPSFQ | QRTFYGDSSDYFDY |
| | 203 | KKPGESLKISC | (SEQ | (SEQ | G (SEQ ID NO: | (SEQ ID NO: |
| | | KGSGYSFTSYW | ID | ID | 91) | 305) |
| | | IAWVRQMPGKG | NO: | NO: | | |
| | | LEWMGIIYPGD | 163) | 85) | | |
| | | SDTRYSPSFQG | | | | |
| | | QVTISADKSIS | | | | |
| | | TAYLOWSSLKA | | | | |
| | | SDTAMYFCARQ | | | | |
| | | RTFYGDSSDYF | | | | |
| | | DYWGQGTLVTV | | | | |
| | | SS (SEQ ID | | | | |
| | | NO: 309) | | | | |
| V44 | HV- | EVQLVQSGAEV | YSFT | SYWIA | IIYPSDSDTRYSPSFQ | QRTFYYDSSDYEDY |
| | 204 | KKPGESLKISC | (SEQ | (SEQ | G (SEQ ID NO: | (SEQ ID NO: |
| | | KGSGYSFTSYW | ID | ID | 303) | 107) |
| | | IAWVRQMPGKG | NO: | NO: | | |
| | | LEWMGIIYPSD | 163) | 85) | | |
| | | SDTRYSPSFQG | | | | |
| | | QVTISADKSIS | | | | |
| | | TAYLQWSSLKA | | | | |
| | | SDTAMYFCARQ | | | | |
| | | RTFYYDSSDYF | | | | |
| | | DYWGQGTLVTV | | | | |
| | | SS (SEQ ID | | | | |
| | | NO: 310) | | | | |
| V68 | HV- | EVQLVQSGAEV | YSFT | SYWIA | IIYPGDSDTRYSPSFQ | QRTFRYDSSDYFDY |
| | 205 | KKPGESLKISC | (SEQ | (SEQ | G (SEQ ID NO: | (SEQ ID NO: |
| | | KGSGYSFTSYW | ID | ID | 91) | 306) |
| | | IAWVRQMPGKG | NO: | NO: | | |
| | | LEWMGIIYPGD | 163) | 85) | | |
| | | SDTRYSPSFQG | | | | |
| | | QVTISADKSIS | | | | |
| | | TAYLQWSSLKA | | | | |
| | | SDTAMYFCARQ | | | | |
| | | RTFRYDSSDYF | | | | |
| | | DYWGQGTLVTV | | | | |
| | | SS (SEQ ID | | | | |
| | | NO: 311) | | | | |
| V90 | HV- | EVQLVQSGAEV | YSFT | SEWIA | IIYPGDSDTRYSPSFQ | QRTFYYDSSDYEDY |
| | 206 | KKPGESLKISC | (SEQ | (SEQ | G (SEQ ID NO: | (SEQ ID NO: |
| | | KGSGYSFTSEW | ID | ID | 91) | 107) |
| | | IAWVRQMPGKG | NO: | NO: | | |
| | | LEWMGIIYPGD | 163) | 302) | | |
| | | SDTRYSPSFQG | | | | |
| | | QVTISADKSIS | | | | |
| | | TAYLQWSSLKA | | | | |
| | | SDTAMYFCARQ | | | | |
| | | RTFYYDSSDYF | | | | |
| | | DYWGQGTLVTV | | | | |
| | | SS (SEQ ID | | | | |
| | | NO: 312) | | | | |

In some embodiments, the TREM2 agonist antigen binding proteins of the invention may comprise one or more of the CDRs from the reduced affinity variants presented in Table 3C (light chain CDRs; i.e., CDRLs) and Table 3D (heavy chain CDRs, i.e., CDRHs). In some embodiments, the TREM2 agonist antigen binding proteins comprise a consensus CDR sequence derived from the reduced affinity variants. For instance, in one embodiment, the TREM2 agonist antigen binding proteins comprise a CDRL1 consensus sequence of X₁ASQGISX₂WLA (SEQ ID NO: 284), where X₁ is R or A; and X₂ is S or R. In another embodiment, the TREM2 agonist antigen binding proteins comprise a CDRL2 consensus sequence of X₁AX₂SLQN (SEQ ID NO: 285), where X₁ is A or S; and X₂ is S or G. In another embodiment, the TREM2 agonist antigen binding proteins comprise a CDRL3 consensus sequence of QQAX₁SFPX₂T (SEQ ID NO: 286), where X₁ is D or V; and X₂ is R or L. In another embodiment, the TREM2 agonist antigen binding proteins comprise a CDRH1 consensus sequence of SX₁WIA (SEQ ID NO: 287), where X₁ is Y or E. In yet another embodiment, the TREM2 agonist antigen binding proteins comprise a CDRH2 consensus sequence of IIYPX₁DSDTRYSPSFQG (SEQ ID NO: 288), where X₁ is G or S. In still another embodiment, the TREM2 agonist antigen binding proteins comprise a CDRH3 consensus sequence of QRX₁FX₂X₃DSSDYFDY (SEQ ID NO: 289), where X₁ is T or G; X₂ is Y or R; and X₃ is Y or G. In some embodiments, the TREM2 agonist antigen binding proteins comprise a light chain variable region comprising complementarity determining regions CDRL1, CDRL2, and CDRL3 and a heavy chain variable region comprising complementarity determining regions CDRH1, CDRH2, and CDRH3, wherein CDRL1 comprises the sequence of SEQ ID NO: 284, CDRL2 comprises the consensus sequence of SEQ ID NO: 285, CDRL3 comprises the consensus sequence of SEQ ID NO: 286, CDRH1 comprises the sequence of SEQ ID NO: 287, CDRH2 comprises the consensus sequence of SEQ ID NO: 288, and CDRH3 comprises the consensus sequence of SEQ ID NO: 289.

In some embodiments, the TREM2 agonist antigen binding proteins of the invention comprise a CDRL1 comprising a sequence selected from SEQ ID NOs: 16, 290, and 291; a CDRL2 comprising a sequence selected from SEQ ID NOs: 28, 292, and 293; a CDRL3 comprising a sequence selected from SEQ ID NOs: 43, 294, and 271; a CDRH1 comprising the sequence of SEQ ID NO: 85 or SEQ ID NO: 302; a CDRH2 comprising the sequence of SEQ ID NO: 91 or SEQ ID NO: 303; and a CDRH3 comprising a sequence selected from SEQ ID NOs: 107 and 304-306.

In some embodiments, the TREM2 agonist antigen binding proteins of the invention comprise a light chain variable region comprising a CDRL1, a CDRL2, and a CDRL3, wherein:
(a) CDRL1, CDRL2, and CDRL3 have the sequence of SEQ ID NOs: 16, 28, and 43, respectively;
(b) CDRL1, CDRL2, and CDRL3 have the sequence of SEQ ID NOs: 16, 292, and 43, respectively;
(c) CDRL1, CDRL2, and CDRL3 have the sequence of SEQ ID NOs: 16, 28, and 294, respectively;
(d) CDRL1, CDRL2, and CDRL3 have the sequence of SEQ ID NOs: 290, 28, and 43, respectively;
(e) CDRL1, CDRL2, and CDRL3 have the sequence of SEQ ID NOs: 16, 293, and 43, respectively;
(f) CDRL1, CDRL2, and CDRL3 have the sequence of SEQ ID NOs: 16, 28, and 271, respectively; or
(g) CDRL1, CDRL2, and CDRL3 have the sequence of SEQ ID NOs: 291, 28, and 43, respectively.

In related embodiments, the TREM2 agonist antigen binding proteins of the invention comprise a heavy chain variable region comprising a CDRH1, a CDRH2, and a CDRH3, wherein:
(a) CDRH1, CDRH2, and CDRH3 have the sequence of SEQ ID NOs: 85, 91, and 304, respectively;
(b) CDRH1, CDRH2, and CDRH3 have the sequence of SEQ ID NOs: 85, 91, and 107, respectively;
(c) CDRH1, CDRH2, and CDRH3 have the sequence of SEQ ID NOs: 85, 91, and 305, respectively;
(d) CDRH1, CDRH2, and CDRH3 have the sequence of SEQ ID NOs: 85, 303, and 107, respectively;
(e) CDRH1, CDRH2, and CDRH3 have the sequence of SEQ ID NOs: 85, 91, and 306, respectively; or
(f) CDRH1, CDRH2, and CDRH3 have the sequence of SEQ ID NOs: 302, 91, and 107, respectively.

In some embodiments, the TREM2 agonist antigen binding proteins of the invention comprise a light chain variable region comprising a CDRL1, a CDRL2, and a CDRL3 and a heavy chain variable region comprising a CDRH1, a CDRH2, and a CDRH3, wherein:
(a) CDRL1, CDRL2, and CDRL3 have the sequence of SEQ ID NOs: 16, 28, and 43, respectively, and CDRH1, CDRH2, and CDRH3 have the sequence of SEQ ID NOs: 85, 91, and 304, respectively;
(b) CDRL1, CDRL2, and CDRL3 have the sequence of SEQ ID NOs: 16, 292, and 43, respectively, and CDRH1, CDRH2, and CDRH3 have the sequence of SEQ ID NOs: 85, 91, and 107, respectively;
(c) CDRL1, CDRL2, and CDRL3 have the sequence of SEQ ID NOs: 16, 28, and 294, respectively, and CDRH1, CDRH2, and CDRH3 have the sequence of SEQ ID NOs: 85, 91, and 107, respectively;
(d) CDRL1, CDRL2, and CDRL3 have the sequence of SEQ ID NOs: 16, 28, and 43, respectively, and CDRH1, CDRH2, and CDRH3 have the sequence of SEQ ID NOs: 85, 91, and 107, respectively;
(e) CDRL1, CDRL2, and CDRL3 have the sequence of SEQ ID NOs: 16, 28, and 43, respectively, and CDRH1, CDRH2, and CDRH3 have the sequence of SEQ ID NOs: 85, 91, and 305, respectively;
(f) CDRL1, CDRL2, and CDRL3 have the sequence of SEQ ID NOs: 16, 28, and 43, respectively, and CDRH1, CDRH2, and CDRH3 have the sequence of SEQ ID NOs: 85, 303, and 107, respectively;
(g) CDRL1, CDRL2, and CDRL3 have the sequence of SEQ ID NOs: 290, 28, and 43, respectively, and CDRH1, CDRH2, and CDRH3 have the sequence of SEQ ID NOs: 85, 91, and 107, respectively;
(h) CDRL1, CDRL2, and CDRL3 have the sequence of SEQ ID NOs: 16, 28, and 43, respectively, and CDRH1, CDRH2, and CDRH3 have the sequence of SEQ ID NOs: 85, 91, and 306, respectively;
(i) CDRL1, CDRL2, and CDRL3 have the sequence of SEQ ID NOs: 16, 293, and 43, respectively, and CDRH1, CDRH2, and CDRH3 have the sequence of SEQ ID NOs: 85, 91, and 107, respectively;
(j) CDRL1, CDRL2, and CDRL3 have the sequence of SEQ ID NOs: 16, 28, and 271, respectively, and CDRH1, CDRH2, and CDRH3 have the sequence of SEQ ID NOs: 85, 91, and 107, respectively; or
(k) CDRL1, CDRL2, and CDRL3 have the sequence of SEQ ID NOs: 291, 28, and 43, respectively, and CDRH1, CDRH2, and CDRH3 have the sequence of SEQ ID NOs: 302, 91, and 107, respectively.

In some embodiments, the TREM2 agonist antigen binding proteins of the invention may comprise a light chain variable region selected from LV-16, LV-201, LV-202, LV-203, LV-204, LV-205, and LV-206, as shown in Table 3C, and/or a heavy chain variable region selected from HV-15, HV-201, HV-202, HV-203, HV-204, HV-205, and HV-206, as shown in Table 3D, or sequences that are at least 80% identical, at least 85% identical, at least 90% identical, or at least 95% identical to any of the sequences in Tables 3C and 3D. For instance, in certain embodiments, the TREM2 agonist antigen binding proteins comprise a light chain variable region comprising (i) a sequence that is at least 90% identical to a sequence selected from SEQ ID NOs: 61 and 295-300, (ii) a sequence that is at least 95% identical to a sequence selected from SEQ ID NOs: 61 and 295-300, or (iii) a sequence selected from SEQ ID NOs: 61 and 295-300. In related embodiments, the TREM2 agonist antigen binding proteins comprise a heavy chain variable region comprising (i) a sequence that is at least 90% identical to a sequence selected from SEQ ID NOs: 124 and 307-312, (ii) a sequence that is at least 95% identical to a sequence selected from SEQ ID NOs: 124 and 307-312, or (iii) a sequence selected from SEQ ID NOs: 124 and 307-312.

In some embodiments, each of the light chain variable regions listed in Table 3C may be combined with any of the heavy chain variable regions listed in Table 3D to form an anti-TREM2 binding domain of the antigen binding proteins of the invention. Examples of such combinations include, but are not limited to: LV-16 (SEQ ID NO: 61) and HV-201 (SEQ ID NO: 307); LV-201 (SEQ ID NO: 295) and HV-15 (SEQ ID NO: 124); LV-202 (SEQ ID NO: 296) and HV-15 (SEQ ID NO: 124); LV-16 (SEQ ID NO: 61) and HV-202 (SEQ ID NO: 308); LV-16 (SEQ ID NO: 61) and HV-203 (SEQ ID NO: 309); LV-16 (SEQ ID NO: 61) and HV-204 (SEQ ID NO: 310); LV-203 (SEQ ID NO: 297) and HV-15 (SEQ ID NO: 124); LV-16 (SEQ ID NO: 61) and HV-205 (SEQ ID NO: 311); LV-204 (SEQ ID NO: 298) and HV-15 (SEQ ID NO: 124); LV-205 (SEQ ID NO: 299) and HV-15 (SEQ ID NO: 124); and LV-206 (SEQ ID NO: 300) and HV-206 (SEQ ID NO: 312).

In some embodiments, the TREM2 agonist antigen binding proteins comprise one or more CDRs of the anti-TREM2 antibody variants set forth in Table 3E. In some embodiments, the TREM2 agonist antigen binding proteins comprise the light chain variable region and heavy chain variable region of the anti-TREM2 antibody variants set forth in Table 3E.

TABLE 3E
Exemplary Variable Region Amino Acid Sequences of
Engineered Antibodies

| Ab ID. | LC variable region | CDRL1 | CDRL2 | CDRL3 |
|---|---|---|---|---|
| 24G6 | DIVMTQSPDSLAVSLGERATIN | KSSQSVL | WASTRES | QQYYSTPLT |
| (SST28347 | CKSSQSVLYSSNNKHFLAWYQQ | YSSNNKH | (SEQ ID | (SEQ ID |
| and | KPGQPPKLLIYWASTRESGVPD | ELA | NO: 22) | NO: 35) |
| SST204812) | RFSGSGSGTDFTLTISSLQAED | (SEQ ID | | |
| | VAVYYCQQYYSTPLTFGGGTKV | NO: 359) | | |
| | EIK (SEQ ID NO: 326) | | | |
| 6E7 | DIQMTQSPSSVSASVGDRVTIT | RASQGIS | AASSLQS | QQADAFPRT |
| (SST29857) | CRASQGISSWLAWYQQKPGKAP | SWLA | (SEQ ID | (SEQ ID |
| | KLLIYAASSLQSGVPSRESGSG | (SEQ ID | NO: 369) | NO: 370) |
| | SGTDFTLTISSLQPEDFATYFC | NO: 16) | | |
| | QQADAFPRTFGQGTKLEIK | | | |
| | (SEQ ID NO: 328) | | | |
| 13E7 | EIVMTQSPATLSVSPGERATLS | RASQSVS | GASTRAT | LQDNNFPPT |
| (SST202443) | CRASQSVSSNLAWFQQKPGQAP | SNLA | (SEQ ID | (SEQ ID |
| | RLLIYGASTRATGIPARFSGSG | (SEQ ID | NO: 23) | NO: 372) |
| | SGTEFTLTISSLQPEDFAVYYC | NO: 361) | | |
| | LQDNNFPPTFGQGTKVDIK | | | |
| | (SEQ ID NO: 330) | | | |
| 5E3 | DIQMTQSPSSLSASVGDRVTIT | RASQGIS | AASSLQS | QQYSTYPFT |
| (SST29825) | CRASQGISNYLAWYQQKPGKAP | NYLA | (SEQ ID | (SEQ ID |
| | KSLIYAASSLQSGVPSRESGSG | (SEQ ID | NO: 29) | NO: 44) |
| | SGTDFTLTISSLQPEDFATYYC | NO: 17) | | |
| | QQYSTYPFTFGQGTKVDIK | | | |
| | (SEQ ID NO: 332) | | | |
| Ab ID. | HC variable region | CDRH1 | CDRH2 | CDRH3 |
| 24G6 | EVQLLESGGGLVQPGGSLRLSC | SYAMS | AISGSGG | AYTPMAFFD |
| (SST28347 | AASGFTESSYAMSWVRQAPGKG | (SEQ ID | STYYAES | Y (SEQ ID |
| and | LEWVSAISGSGGSTYYAESVKG | NO: 77) | VKG | NO: 98) |
| SST204812) | RFTISRDNSKNTLYLQMNSLRA | | (SEQ ID | |
| | EDTAVYYCAKAYTPMAFFDYWG | | NO: 368) | |
| | QGTLVTVSS (SEQ ID NO: | | | |
| | 327) | | | |
| 6E7 | EVQLVQSGAEVKKPGESLKISC | SYWIA | IIYPGDA | QRTFYYDSS |
| (SST29857) | KGSGYSFTSYWIAWVRQMPGKG | (SEQ ID | DARYSPS | DYFDY |
| | LEWMGIIYPGDADARYSPSFQG | NO: 85) | FQG | (SEQ ID |
| | QVTISADKSISTAYLQWSSLKA | | (SEQ ID | NO: 107) |
| | SDTAMYFCARQRTFYYDSSDYE | | NO: 371) | |
| | DYWGQGTLVTVSS (SEQ ID | | | |
| | NO: 329) | | | |
| 13E7 | EVQLVQSGAEVKKPGESLKISC | SYWIG | IIYPGDA | RRQGIFGDA |
| (SST202443) | KGSGYSFTSYWIGWVRQMPGKG | (SEQ ID | DARYSPS | LDF |
| | LEWMGIIYPGDADARYSPSFQG | NO: 81) | FQG | (SEQ ID |
| | QVTISADKSISTAYLQWSSLKA | | (SEQ ID | NO: 374) |
| | SDTAMYFCARRRQGIFGDALDE | | NO: 373) | |
| | WGQGTLVTVSS (SEQ ID | | | |
| | NO: 331) | | | |
| | QVQLVQSGAEVKKPGASVKVSC | GYYIH | WINPYSG | DAGYLALYG |
| | KASGYTFTGYYIHWVRQAPGQG | (SEQ ID | GTTSAQK | TDV |
| | LEWMGWINPYSGGTTSAQKFQG | NO: 86) | FQG | (SEQ ID |
| | RVTMTRDTSTSSAYMELSRLRS | | (SEQ ID | NO: 375) |
| | DDTAVYYCARDAGYLALYGTDV | | NO: 94) | |
| | WGQGTLVTVSS (SEQ ID | | | |
| | NO: 333) | | | |

In some embodiments, the TREM2 agonist antigen binding protein comprises a light chain variable region comprising a CDRL1, a CDRL2, and a CDRL3, wherein:
(a) CDRL1, CDRL2, and CDRL3 have the sequence of SEQ ID NOs: 16, 369, and 370, respectively;
(b) CDRL1, CDRL2, and CDRL3 have the sequence of SEQ ID NOs: 361, 23, and 372, respectively; or
(c) CDRL1, CDRL2, and CDRL3 have the sequence of SEQ ID NOs: 357, 21, and 33, respectively; (d) CDRL1, CDRL2, and CDRL3 have the sequence of SEQ ID NOs: 357, 20, and 33, respectively.

In some embodiments, the TREM2 agonist antigen binding protein comprises a heavy chain variable region comprising a CDRH1, a CDRH2, and a CDRH3, wherein:
(a) CDRH1, CDRH2, and CDRH3 have the sequence of SEQ ID NOs: 77, 368, and 98, respectively;
(b) CDRH1, CDRH2, and CDRH3 have the sequence of SEQ ID NOs: 85, 371, and 107, respectively;
(c) CDRH1, CDRH2, and CDRH3 have the sequence of SEQ ID NOs: 81, 373, and 374, respectively; or
(d) CDRH1, CDRH2, and CDRH3 have the sequence of SEQ ID NOs: 86, 94, and 375, respectively.

In some embodiments, the TREM2 agonist antigen binding protein comprises a light chain variable region comprising a CDRL1, a CDRL2, and a CDRL3 and a heavy chain variable region comprising a CDRH1, a CDRH2, and a CDRH3, wherein:
(a) CDRL1, CDRL2, and CDRL3 have the sequence of SEQ ID NOs: 359, 22, and 35, respectively, and CDRH1, CDRH2, and CDRH3 have the sequence of SEQ ID NOs: 77, 368, and 98, respectively;
(b) CDRL1, CDRL2, and CDRL3 have the sequence of SEQ ID NOs: 16, 369, and 370, respectively, and CDRH1, CDRH2, and CDRH3 have the sequence of SEQ ID NOs: 85, 371, and 107, respectively;
(c) CDRL1, CDRL2, and CDRL3 have the sequence of SEQ ID NOs: 361, 23, and 372, respectively, and CDRH1, CDRH2, and CDRH3 have the sequence of SEQ ID NOs: 81, 373, and 374, respectively; or
(d) CDRL1, CDRL2, and CDRL3 have the sequence of SEQ ID NOs: 17, 29, and 44, respectively, and CDRH1, CDRH2, and CDRH3 have the sequence of SEQ ID NOs: 86, 94, and 375, respectively.

Accordingly, in some embodiments, the TREM2 agonist antigen binding protein comprises a light chain variable region comprising a CDRL1, a CDRL2, and a CDRL3, and a heavy chain variable region comprising a CDRH1, a CDRH2, and a CDRH3, wherein the CDRL1, CDRL2, and CDRL3 have the sequence of SEQ ID NOs: 361, 23, and 372, respectively, and the CDRH1, CDRH2, and CDRH3 have the sequence of SEQ ID NOs: 81, 373, and 374, respectively.

In some embodiments therefore, the present invention provides a method of treating ALSP in a human patient, the method comprising administering to the patient an effective amount of a TREM2 agonist antigen binding protein comprising a CDRL1, CDRL2, and CDRL3 having the sequence of SEQ ID NOs: 361, 23, and 372, respectively, and a CDRH1, CDRH2, and CDRH3 having the sequence of SEQ ID NOs: 81, 373, and 374, respectively. In certain embodiments, the antibody is human. In some embodiments, the TREM2 agonist antigen binding protein comprises
(a) a light chain variable region comprising the amino acid sequence of SEQ ID NO: 326 and a heavy chain variable region comprising the amino acid sequence of SEQ ID NO: 327;
(b) a light chain variable region comprising the amino acid sequence of SEQ ID NO: 328 and a heavy chain variable region comprising the amino acid sequence of SEQ ID NO: 329;
(c) a light chain variable region comprising the amino acid sequence of SEQ ID NO: 330 and a heavy chain variable region comprising the amino acid sequence of SEQ ID NO: 331; or
(d) a light chain variable region comprising the amino acid sequence of SEQ ID NO: 332 and a heavy chain variable region comprising the amino acid sequence of SEQ ID NO: 333.

In some embodiments, the TREM2 agonist antigen binding protein comprises a light chain variable region comprising the amino acid sequence of SEQ ID NO: 330 and a heavy chain variable region comprising the amino acid sequence of SEQ ID NO: 331.

In some embodiments therefore, the present invention provides a method of treating ALSP in a human patient, the method comprising administering to the patient an effective amount of a TREM2 agonist antigen binding protein comprising a light chain variable region comprising the amino acid sequence of SEQ ID NO: 330 and a heavy chain variable region comprising the amino acid sequence of SEQ ID NO: 331. In certain embodiments, the antibody is human.

In some embodiments, the TREM2 agonist antigen binding proteins of the invention comprise a light chain variable region consisting of or consisting essentially of the amino acid sequence of SEQ ID NO: 326, 328, 330 or 332. In some embodiments, the TREM2 agonist antigen binding proteins of the invention comprise a heavy chain variable region consisting of or consisting essentially of the amino acid sequence of SEQ ID NO: 327, 329, 331 or 333. In a specific embodiment, the TREM2 agonist antigen binding proteins of the invention comprise a light chain variable region and a heavy chain variable region, wherein the light chain variable region consisting of or consisting essentially of the amino acid sequence of SEQ ID NO: 326 and the heavy chain variable region consisting of or consisting essentially of the amino acid sequence of SEQ ID NO: 327. In a specific embodiment, the TREM2 agonist antigen binding proteins of the invention comprise a light chain variable region and a heavy chain variable region, wherein the light chain variable region consisting of or consisting essentially of the amino acid sequence of SEQ ID NO: 328 and the heavy chain variable region consisting of or consisting essentially of the amino acid sequence of SEQ ID NO: 329. In a specific embodiment, the TREM2 agonist antigen binding proteins of the invention comprise a light chain variable region and a heavy chain variable region, wherein the light chain variable region consisting of or consisting essentially of the amino acid sequence of SEQ ID NO: 330 and the heavy chain variable region consisting of or consisting essentially of the amino acid sequence of SEQ ID NO: 331. In a specific embodiment, the TREM2 agonist antigen binding proteins of the invention comprise a light chain variable region and a heavy chain variable region, wherein the light chain variable region consisting of or consisting essentially of the amino acid sequence of SEQ ID NO: 332 and the heavy chain variable region consisting of or consisting essentially of the amino acid sequence of SEQ ID NO: 333.

In some embodiments, each of the light chain variable regions disclosed in Tables IA, 3A, 3C, and 3E and each of the heavy chain variable regions disclosed in Tables 11B, 3B3, 3D), and 3E may be attached to the light chain constant regions (Table 4) and heavy chain constant regions (Table 5) to form complete antibody light and heavy chains, respectively, as further discussed below. Further, each of the generated heavy and light chain sequences may be combined to form a complete antibody structure. It should be understood that the heavy chain and light chain variable regions provided herein can also be attached to other constant domains having different sequences than the exemplary sequences listed herein.

In some embodiments, exemplary TREM2 agonist antibody having a light chain variable region with a light chain constant domain and a heavy chain variable region with a heavy chain constant region are disclosed in Table 3F.

TABLE 3F
Light Chain and Heavy Chain Amino Acid Sequences of
Exemplary Antibodies

| Ab ID. | | Sequence |
|---|---|---|
| 24G6 | LC | MDMRVPAQLLGLLLLWLRGARCDIVMTQSPDSLAVSLGERATINCKSS |
| (SST28347) | | QSVLYSSNNKHFLAWYQQKPGQPPKLLIYWASTRESGVPDRESGSGSG |
| | | TDFTLTISSLQAEDVAVYYCQQYYSTPLTFGGGTKVEIKRTVAAPSVE |
| | | IFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSENRG |
| | | EC (SEQ ID NO: 334) |
| | HC | MDMRVPAQLLGLLLLWLRGARCEVQLLESGGGLVQPGGSLRLSCAASG |
| | | FTFSSYAMSWVRQAPGKGLEWVSAISGSGGSTYYAESVKGRFTISRDN |
| | | SKNTLYLQMNSLRAEDTAVYYCAKAYTPMAFFDYWGQGTLVTVSSAST |
| | | KGPSVFPLAPSSKSTSGGTAALGCLVKDYFPEPVTVSWNSGALTSGVH |
| | | TFPAVLQSSGLYSLSSVVTVPSSSLGTQTYICNVNHKPSNTKVDKKVE |
| | | PKSCDKTHTCPPCPAPELLGGPSVFLFPPKPKDTLMISRTPEVTCVVV |
| | | DVSHEDPEVKENWYVDGVEVHNAKTKPCEEQYGSTYRCVSVLTVLHQD |
| | | WLNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLYS |
| | | KLTVDKSRWQQGNVFSCSVMHEALHNHYTQKSLSLSPGK (SEQ ID |
| | | NO: 335) |
| 24G6 | LC | MDMRVPAQLLGLLLLWLRGARCDIVMTQSPDSLAVSLGERATINCKSS |
| (SST204812) | | QSVLYSSNNKHFLAWYQQKPGQPPKLLIYWASTRESGVPDRESGSGSG |
| | | TDFTLTISSLQAEDVAVYYCQQYYSTPLTFGGGTKVEIKRTVAAPSVE |
| | | IFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSENRG |
| | | EC (SEQ ID NO: 334) |
| | HC | MDMRVPAQLLGLLLLWLRGARCEVOLLESGGGLVQPGGSLRLSCAASG |
| | | FTFSSYAMSWVRQAPGKGLEWVSAISGSGGSTYYAESVKGRFTISRDN |
| | | SKNTLYLQMNSLRAEDTAVYYCAKAYTPMAFFDYWGQGTLVTVSSAST |
| | | KGPSVFPLAPSSRSTSESTAALGCLVKDYFPEPVTVSWNSGALTSGVH |
| | | TFPAVLQSSGLYSLSSVVTVPSSNFGTQTYTCNVDHKPSNTKVDKTVE |
| | | RKCCVECPPCPAPELLGGPSVFLFPPKPKDTLMISRTPEVTCVVVDVS |
| | | HEDPEVKFNWYVDGVEVHNAKTKPCEEQYGSTYRCVSVLTVLHQDWLN |
| | | GKEYKCKVSNKALPAPIEKTISKAKGQPREPQVYTLPPSREEMTKNQV |
| | | SLTCLVKGFYPSDIAVEWESNGQPENNYKTTPPVLDSDGSFFLYSKLT |
| | | VDKSRWQQGNVFSCSVMHEALHNHYTQKSLSLSPGK (SEQ ID NO: |
| | | 336) |
| 6E7 | LC | MDMRVPAQLLGLLLLWLRGARCDIQMTQSPSSVSASVGDRVTITCRAS |
| (SST29857) | | QGISSWLAWYQQKPGKAPKLLIYAASSLQSGVPSRESGSGSGTDFTLT |
| | | ISSLQPEDFATYFCQQADAFPRTFGQGTKLEIKRTVAAPSVFIFPPSD |
| | | EQLKSGTASVVCLLNNFYPREAKVQWKVDNALQSGNSQESVTEQDSKD |
| | | STYSLSSTLTLSKADYEKHKVYACEVTHQGLSSPVTKSENRGEC |
| | | (SEQ ID NO: 337) |
| | HC | MDMRVPAQLLGLLLLWLRGARCEVQLVQSGAEVKKPGESLKISCKGSG |
| | | YSFTSYWIAWVRQMPGKGLEWMGIIYPGDADARYSPSFQGQVTISADK |
| | | SISTAYLQWSSLKASDTAMYFCARQRTFYYDSSDYFDYWGQGTLVTVS |
| | | SASTKGPSVFPLAPSSRSTSESTAALGCLVKDYFPEPVTVSWNSGALT |
| | | SGVHTFPAVLQSSGLYSLSSVVTVPSSNFGTQTYTCNVDHKPSNTKVD |
| | | KTVERKCCVECPPCPAPELLGGPSVFLFPPKPKDTLMISRTPEVTCVV |
| | | VDVSHEDPEVKENWYVDGVEVHNAKTKPCEEQYGSTYRCVSVLTVLHQ |
| | | DWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLY |
| | | SKLTVDKSRWQQGNVFSCSVMHEALHNHYTQKSLSLSPGK (SEQ ID |
| | | NO: 338) |
| 13E7 | LC | MDMRVPAQLLGLLLLWLRGARCEIVMTQSPATLSVSPGERATLSCRAS |
| (SST202443) | | QSVSSNLAWFQQKPGQAPRLLIYGASTRATGIPARFSGSGSGTEFTLT |
| | | ISSLQPEDFAVYYCLQDNNFPPTFGQGTKVDIKRTVAAPSVEIFPPSD |
| | | EQLKSGTASVVCLLNNFYPREAKVQWKVDNALQSGNSQESVTEQDSKD |
| | | STYSLSSTLTLSKADYEKHKVYACEVTHQGLSSPVTKSENRGEC |
| | | (SEQ ID NO: 339) |
| | HC | MDMRVPAQLLGLLLLWLRGARCEVQLVOSGAEVKKPGESLKISCKGSG |
| | | YSFTSYWIGWVRQMPGKGLEWMGIIYPGDADARYSPSFQGQVTISADK |
| | | SISTAYLQWSSLKASDTAMYFCARRRQGIFGDALDFWGQGTLVTVSSA |
| | | STKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDKK |
| | | VEPKSCDKTHTCPPCPAPELLGGPSVELFPPKPKDTLMISRTPEVTCV |
| | | VVDVSHEDPEVKENWYVDGVEVHNAKTKPCEEQYGSTYRCVSVLTVLH |
| | | QDWLNGKEYKCKVSNKALPAPIEKTISKAKGQPREPQVYTLPPSREEM |
| | | TKNQVSLTCLVKGFYPSDIAVEWESNGQPENNYKTTPPVLDSDGSFEL |
| | | YSKLTVDKSRWQQGNVFSCSVMHEALHNHYTQKSLSLSPGK (SEQ |
| | | ID NO: 340) |
| 5E3 | LC | MDMRVPAQLLGLLLLWLRGARCDIQMTQSPSSLSASVGDRVTITCRAS |
| (SST29825) | | QGISNYLAWYQQKPGKAPKSLIYAASSLQSGVPSRESGSGSGTDFTLT |
| | | ISSLQPEDFATYYCQQYSTYPFTFGQGTKVDIKRTVAAPSVFIFPPSD |
| | | EQLKSGTASVVCLLNNFYPREAKVQWKVDNALQSGNSQESVTEQDSKD |
| | | STYSLSSTLTLSKADYEKHKVYACEVTHQGLSSPVTKSENRGEC |
| | | (SEQ ID NO: 341) |
| | HC | MDMRVPAQLLGLLLLWLRGARCQVQLVQSGAEVKKPGASVKVSCKASG |
| | | YTFTGYYIHWVRQAPGQGLEWMGWINPYSGGTTSAQKFQGRVTMTRDT |
| | | STSSAYMELSRLRSDDTAVYYCARDAGYLALYGTDVWGQGTLVTVSSA |
| | | STKGPSVFPLAPSSRSTSESTAALGCLVKDYFPEPVTVSWNSGALTSG |
| | | VHTFPAVLQSSGLYSLSSVVTVPSSNEGTQTYTCNVDHKPSNTKVDKT |
| | | VERKCCVECPPCPAPELLGGPSVELFPPKPKDTLMISRTPEVTCVVVD |
| | | VSHEDPEVKENWYVDGVEVHNAKTKPCEEQYGSTYRCVSVLTVLHQDW |
| | | LNGKEYKCKVSNKALPAPIEKTISKAKGQPREPQVYTLPPSREEMTKN |
| | | QVSLTCLVKGFYPSDIAVEWESNGQPENNYKTTPPVLDSDGSFFLYSK |
| | | LTVDKSRWQQGNVFSCSVMHEALHNHYTQKSLSLSPGK (SEQ ID |
| | | NO: 342) |
| 24G6-1 | LC | DIVMTQSPDSLAVSLGERATINCKSSQSVLYSSNNKHFLAWYQQKPGQ |
| (SST28347- | | PPKLLIYWASTRESGVPDRESGSGSGTDETLTISSLQAEDVAVYYCQQ |
| 1) | | YYSTPLTFGGGTKVEIKRTVAAPSVFIFPPSDEQLKSGTASVVCLLNN |
| | | FYPREAKVQWKVDNALQSGNSQESVTEQDSKDSTYSLSSTLTLSKADY |
| | | EKHKVYACEVTHQGLSSPVTKSENRGEC (SEQ ID NO: 343) |
| | HC | EVQLLESGGGLVQPGGSLRLSCAASGFTESSYAMSWVRQAPGKGLEWV |
| | | SAISGSGGSTYYAESVKGRFTISRDNSKNTLYLQMNSLRAEDTAVYYC |
| | | AKAYTPMAFFDYWGQGTLVTVSSASTKGPSVFPLAPSSKSTSGGTAAL |
| | | GCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLGGP |
| | | SVFLFPPKPKDTLMISRTPEVTCVVVDVSHEDPEVKENWYVDGVEVHN |
| | | AKTKPCEEQYGSTYRCVSVLTVLHQDWLNGKEYKCKVSNKALPAPIEK |
| | | TISKAKGQPREPQVYTLPPSREEMTKNQVSLTCLVKGFYPSDIAVEWE |
| | | SNGQPENNYKTTPPVLDSDGSFFLYSKLTVDKSRWQQGNVFSCSVMHE |
| | | ALHNHYTQKSLSLSPGK (SEQ ID NO: 344) |
| 24G6-1 | LC | DIVMTQSPDSLAVSLGERATINCKSSQSVLYSSNNKHELAWYQQKPGQ |
| (SST28347- | | PPKLLIYWASTRESGVPDRESGSGSGTDETLTISSLQAEDVAVYYCQQ |
| 1) | | YYSTPLTFGGGTKVEIKRTVAAPSVFIFPPSDEQLKSGTASVVCLLNN |
| | | FYPREAKVQWKVDNALQSGNSQESVTEQDSKDSTYSLSSTLTLSKADY |
| | | EKHKVYACEVTHQGLSSPVTKSENRGEC (SEQ ID NO: 343) |
| | HC | EVQLLESGGGLVQPGGSLRLSCAASGFTESSYAMSWVRQAPGKGLEWV |
| | | SAISGSGGSTYYAESVKGRFTISRDNSKNTLYLOMNSLRAEDTAVYYC |
| | | AKAYTPMAFFDYWGQGTLVTVSSASTKGPSVFPLAPSSRSTSESTAAL |
| | | GCLVKDYFPEPVTVSWNSGALTSGVHTFPAVLQSSGLYSLSSVVTVPS |
| | | SNFGTQTYTCNVDHKPSNTKVDKTVERKCCVECPPCPAPELLGGPSVE |
| | | LFPPKPKDTLMISRTPEVTCVVVDVSHEDPEVKENWYVDGVEVHNAKT |
| | | KPCEEQYGSTYRCVSVLTVLHQDWLNGKEYKCKVSNKALPAPIEKTIS |
| | | KAKGQPREPQVYTLPPSREEMTKNQVSLTCLVKGFYPSDIAVEWESNG |
| | | QPENNYKTTPPVLDSDGSFFLYSKLTVDKSRWQQGNVFSCSVMHEALH |
| | | NHYTQKSLSLSPG (SEQ ID NO: 345) |
| 6E7-1 | LC | DIQMTQSPSSVSASVGDRVTITCRASQGISSWLAWYQQKPGKAPKLLI |
| (SST29857- | | YAASSLQSGVPSRESGSGSGTDETLTISSLQPEDFATYFCQQADAFPR |
| 1) | | TFGQGTKLEIKRTVAAPSVFIFPPSDEQLKSGTASVVCLLNNFYPREA |
| | | KVQWKVDNALQSGNSQESVTEQDSKDSTYSLSSTLTLSKADYEKHKVY |
| | | ACEVTHQGLSSPVTKSENRGEC (SEQ ID NO: 346) |
| | HC | EVQLVQSGAEVKKPGESLKISCKGSGYSFTSYWIAWVRQMPGKGLEWM |
| | | GIIYPGDADARYSPSFQGQVTISADKSISTAYLOWSSLKASDTAMYFC |
| | | ARQRTFYYDSSDYFDYWGQGTLVTVSSASTKGPSVFPLAPSSRSTSES |
| | | TAALGCLVKDYFPEPVTVSWNSGALTSGVHTFPAVLQSSGLYSLSSVV |
| | | TVPSSNFGTQTYTCNVDHKPSNTKVDKTVERKCCVECPPCPAPELLGG |
| | | PSVFLFPPKPKDTLMISRTPEVTCVVVDVSHEDPEVKENWYVDGVEVH |
| | | NAKTKPCEEQYGSTYRCVSVLTVLHQDWLNGKEYKCKVSNKALPAPIE |
| | | KTISKAKGQPREPQVYTLPPSREEMTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPG (SEQ ID NO: 347) |
| 13E7-1 | LC | EIVMTQSPATLSVSPGERATLSCRASQSVSSNLAWFQQKPGQAPRLLI |
| (SST202443- | | YGASTRATGIPARFSGSGSGTEFTLTISSLQPEDFAVYYCLQDNNFPP |
| 1) | | TFGQGTKVDIKRTVAAPSVFIFPPSDEQLKSGTASVVCLLNNFYPREA |
| | | KVQWKVDNALQSGNSQESVTEQDSKDSTYSLSSTLTLSKADYEKHKVY |
| | | ACEVTHQGLSSPVTKSENRGEC (SEQ ID NO: 348) |
| | HC | EVQLVQSGAEVKKPGESLKISCKGSGYSFTSYWIGWVRQMPGKGLEWM |
| | | GIIYPGDADARYSPSFQGQVTISADKSISTAYLOWSSLKASDTAMYFC |
| | | ARRRQGIFGDALDFWGQGTLVTVSSASTKGPSVFPLAPSSKSTSGGTA |
| | | ALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPELLG |
| | | GPSVFLFPPKPKDTLMISRTPEVTCVVVDVSHEDPEVKENWYVDGVEV |
| | | HNAKTKPCEEQYGSTYRCVSVLTVLHQDWLNGKEYKCKVSNKALPAPI |
| | | EKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVM |
| | | HEALHNHYTQKSLSLSPG (SEQ ID NO: 349) |
| 5E3-1 | LC | DIQMTQSPSSLSASVGDRVTITCRASQGISNYLAWYQQKPGKAPKSLI |
| (SST29825- | | YAASSLQSGVPSRESGSGSGTDFTLTISSLQPEDFATYYCQQYSTYPF |
| 1) | | TFGQGTKVDIKRTVAAPSVFIFPPSDEQLKSGTASVVCLLNNFYPREA |
| | | KVQWKVDNALQSGNSQESVTEQDSKDSTYSLSSTLTLSKADYEKHKVY |
| | | ACEVTHQGLSSPVTKSENRGEC (SEQ ID NO: 350) |
| | HC | QVQLVQSGAEVKKPGASVKVSCKASGYTFTGYYIHWVRQAPGQGLEWM |
| | | GWINPYSGGTTSAQKFQGRVTMTRDTSTSSAYMELSRLRSDDTAVYYC |
| | | ARDAGYLALYGTDVWGQGTLVTVSSASTKGPSVFPLAPSSRSTSESTA |
| | | ALGCLVKDYFPEPVTVSWNSGALTSGVHTFPAVLQSSGLYSLSSVVTV |
| | | PSSNFGTQTYTCNVDHKPSNTKVDKTVERKCCVECPPCPAPELLGGPS |
| | | VFLFPPKPKDTLMISRTPEVTCVVVDVSHEDPEVKENWYVDGVEVHNA |
| | | KTKPCEEQYGSTYRCVSVLTVLHQDWLNGKEYKCKVSNKALPAPIEKT |
| | | ISKAKGQPREPQVYTLPPSREEMTKNQVSLTCLVKGFYPSDIAVEWES |
| | | NGQPENNYKTTPPVLDSDGSFFLYSKLTVDKSRWQQGNVFSCSVMHEA |
| | | LHNHYTQKSLSLSPG (SEQ ID NO: 351) |
| 13E7 | LC | EIVMTQSPATLSVSPGERATLSCRASQSVSSNLAWFQQKPGQAPRLLI |
| Variant | | YGASTRATGIPARFSGSGSGTEFTLTISSLQPEDFAVYYCLQDNNFPP |
| | | TFGQGTKVDIKRTVAAPSVFIFPPSDEQLKSGTASVVCLLNNFYPREA |
| | | KVQWKVDNALQSGNSQESVTEQDSKDSTYSLSSTLTLSKADYEKHKVY |
| | | ACEVTHQGLSSPVTKSENRGEC (SEQ ID NO: 352) |
| | HC | EVQLVQSGAEVKKPGESLKISCKGSGYSFTSYWIGWVRQMPGKGLEWM |
| | | GIIYPGDADARYSPSFQGQVTISADKSISTAYLOWSSLKASDTAMYFC |
| | | ARRRQGIFGDALDFWGQGTLVTVSSASTKGPSVFPLAPSSKSTSGGTA |
| | | ALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPELLG |
| | | GPSVFLFPPKPKDTLMISRTPEVTCVVVDVSHEDPEVKENWYVDGVEV |
| | | HNAKTKPCEEQYGSTYRCVSVLTVLHQDWLNGKEYKCKVSNKALPAPI |
| | | EKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVM |
| | | HEALHNHYTQKSLSLSPGK (SEQ ID NO: 353) |

In some embodiments, the TREM2 agonist antigen binding proteins of the invention comprise a light chain comprising the sequence of SEQ ID NO: 334 and a heavy chain comprising the sequence of SEQ ID NO: 335. In some embodiments, the TREM2 agonist antigen binding proteins of the invention comprise a light chain comprising the sequence of SEQ ID NO: 334 and a heavy chain comprising the sequence of SEQ ID NO: 336. In some embodiments, the TREM2 agonist antigen binding proteins of the invention comprise a light chain comprising the sequence of SEQ ID NO: 337 and a heavy chain comprising the sequence of SEQ ID NO: 338. In some embodiments, the TREM2 agonist antigen binding proteins of the invention comprise a light chain comprising the sequence of SEQ ID NO: 339 and a heavy chain comprising the sequence of SEQ ID NO: 340. In some embodiments, the TREM2 agonist antigen binding proteins of the invention comprise a light chain comprising the sequence of SEQ ID NO: 341 and a heavy chain comprising the sequence of SEQ ID NO: 342. In some embodiments, the TREM2 agonist antigen binding proteins of the invention comprise a light chain comprising the sequence of SEQ ID NO: 343 and a heavy chain comprising the sequence of SEQ ID NO: 344. In some embodiments, the TREM2 agonist antigen binding proteins of the invention comprise a light chain comprising the sequence of SEQ ID NO: 343 and a heavy chain comprising the sequence of SEQ ID NO: 345. In some embodiments, the TREM2 agonist antigen binding proteins of the invention comprise a light chain comprising the sequence of SEQ ID NO: 346 and a heavy chain comprising the sequence of SEQ ID NO: 347. In some embodiments, the TREM2 agonist antigen binding proteins of the invention comprise a light chain comprising the sequence of SEQ ID NO: 348 and a heavy chain comprising the sequence of SEQ ID NO: 349. In some embodiments, the TREM2 agonist antigen binding proteins of the invention comprise a light chain comprising the sequence of SEQ ID NO: 350 and a heavy chain comprising the sequence of SEQ ID NO: 351.

In some embodiments, the present invention provides a method of treating ALSP in a human patient, the method comprising administering to the patient an effective amount of a TREM2 agonist antigen binding protein comprising a light chain comprising the sequence of SEQ ID NO: 334 and a heavy chain comprising the sequence of SEQ ID NO: 335. In some embodiments, the present invention provides a method of treating ALSP in a human patient, the method comprising administering to the patient an effective amount of a TREM2 agonist antigen binding protein comprising a light chain comprising the sequence of SEQ ID NO: 334 and a heavy chain comprising the sequence of SEQ ID NO: 336. In some embodiments, the present invention provides a method of treating ALSP in a human patient, the method comprising administering to the patient an effective amount of a TREM2 agonist antigen binding protein comprising a light chain comprising the sequence of SEQ ID NO: 337 and a heavy chain comprising the sequence of SEQ ID NO: 338. In some embodiments, the present invention provides a method of treating ALSP in a human patient, the method comprising administering to the patient an effective amount of a TREM2 agonist antigen binding protein comprising a light chain comprising the sequence of SEQ ID NO: 339 and a heavy chain comprising the sequence of SEQ ID NO: 340. In some embodiments, the present invention provides a method of treating ALSP in a human patient, the method comprising administering to the patient an effective amount of a TREM2 agonist antigen binding protein comprising a light chain comprising the sequence of SEQ ID NO: 341 and a heavy chain comprising the sequence of SEQ ID NO: 342. In some embodiments, the present invention provides a method of treating ALSP in a human patient, the method comprising administering to the patient an effective amount of a TREM2 agonist antigen binding protein comprising a light chain comprising the sequence of SEQ ID NO: 343 and a heavy chain comprising the sequence of SEQ ID NO: 344. In some embodiments, the present invention provides a method of treating ALSP in a human patient, the method comprising administering to the patient an effective amount of a TREM2 agonist antigen binding protein comprising a light chain comprising the sequence of SEQ ID NO: 343 and a heavy chain comprising the sequence of SEQ ID NO: 345. In some embodiments, the present invention provides a method of treating ALSP in a human patient, the method comprising administering to the patient an effective amount of a TREM2 agonist antigen binding protein comprising a light chain comprising the sequence of SEQ ID NO: 346 and a heavy chain comprising the sequence of SEQ ID NO: 347. In some embodiments therefore, the present invention provides a method of treating ALSP in a human patient, the method comprising administering to the patient an effective amount of a TREM2 agonist antigen binding protein comprising a light chain comprising the sequence of SEQ ID NO: 348 and a heavy chain comprising the sequence of SEQ ID NO: 349. In some embodiments, the present invention provides a method of treating ALSP in a human patient, the method comprising administering to the patient an effective amount of a TREM2 agonist antigen binding protein comprising a light chain comprising the sequence of SEQ ID NO: 350 and a heavy chain comprising the sequence of SEQ ID NO: 351. In some embodiments, the present invention provides a method of treating ALSP in a human patient, the method comprising administering to the patient an effective amount of a TREM2 agonist antigen binding protein comprising a light chain comprising the sequence of SEQ ID NO: 352 and a heavy chain comprising the sequence of SEQ ID NO: 353.

In some embodiments, the TREM2 agonist antigen binding proteins of the invention comprise a light chain consisting of or consisting essentially of the amino acid sequence of SEQ ID NO: 334, 337, 339 or 341. In some embodiments, the TREM2 agonist antigen binding proteins of the invention comprise a light chain consisting of or consisting essentially of the amino acid sequence of SEQ ID NO: 343, 346, 348, or 350. In some embodiments, the TREM2 agonist antigen binding proteins of the invention comprise a heavy chain consisting of or consisting essentially of the amino acid sequence of SEQ ID NO: 335, 336, 338, 340, or 342. In some embodiments, the TREM2 agonist antigen binding proteins of the invention comprise a heavy chain consisting of or consisting essentially of the amino acid sequence of SEQ ID NO: 344, 345, 347, 349, or 351. In a specific embodiment, the TREM2 agonist antigen binding proteins of the invention comprise a light chain and a heavy chain, wherein:
(a) the light chain consisting of or consisting essentially of the amino acid sequence of SEQ ID NO: 334 and the heavy chain consisting of or consisting essentially of the amino acid sequence of SEQ ID NO: 335;
(b) the light chain consisting of or consisting essentially of the amino acid sequence of SEQ ID NO: 334 and the heavy chain consisting of or consisting essentially of the amino acid sequence of SEQ ID NO: 336;
(c) the light chain consisting of or consisting essentially of the amino acid sequence of SEQ ID NO: 337 and the heavy chain consisting of or consisting essentially of the amino acid sequence of SEQ ID NO: 338;
(d) the light chain consisting of or consisting of essentially of the amino acid sequence of SEQ ID NO: 339 and the heavy chain consisting of or consisting essentially of the amino acid sequence of SEQ ID NO: 340; or
(e) the light chain consisting of or consisting essentially of the amino acid sequence of SEQ ID NO: 341 and the heavy chain consisting of or consisting essentially of the amino acid sequence of SEQ ID NO: 342.

In a specific embodiment, the TREM2 agonist antigen binding proteins of the invention comprise a light chain and a heavy chain, wherein:
(a) the light chain consisting of or consisting essentially of the amino acid sequence of SEQ ID NO: 343 and the heavy chain consisting of or consisting essentially of the amino acid sequence of SEQ ID NO: 344;
(b) the light chain consisting of or consisting essentially of the amino acid sequence of SEQ ID NO: 343 and the heavy chain consisting of or consisting essentially of the amino acid sequence of SEQ ID NO: 345;
(c) the light chain consisting of or consisting essentially of the amino acid sequence of SEQ ID NO: 346 and the heavy chain consisting of or consisting essentially of the amino acid sequence of SEQ ID NO: 347;
(d) the light chain consisting of or consisting of essentially of the amino acid sequence of SEQ ID NO: 348 and the heavy chain consisting of or consisting essentially of the amino acid sequence of SEQ ID NO: 349;
(e) the light chain consisting of or consisting essentially of the amino acid sequence of SEQ ID NO: 350 and the heavy chain consisting of or consisting essentially of the amino acid sequence of SEQ ID NO: 351; or
(f) the light chain consisting of or consisting essentially of the amino acid sequence of SEQ ID NO: 352 and the heavy chain consisting of or consisting essentially of the amino acid sequence of SEQ ID NO: 353.

Unless indicated otherwise by reference to a specific sequence in Tables 1A, 1B, 3A, 3B, 3C, 3D, 3E and in related discussions, the numbering of the amino acid residues in an immunoglobulin heavy chain or light chain is according to Kabat-EU numbering as described in Kabat et al., Sequences of Proteins of Immunological Interest, 5th Ed., US Department of Health and Human Services, NIH publication No. 91-3242, pp 662,680,689 (1991) and Edelman et al., Proc. Natl. Acad. USA, Vol. 63: 78-85 (1969). The Kabat numbering scheme is typically used when referring to the position of an amino acid within the variable regions, whereas the EU numbering scheme is generally used when referring to the position of an amino acid with an immunoglobulin constant region.

In some embodiments, the TREM2 antigen binding protein comprise an antibody that competes with an antibody comprising CDRL1, CDRL2, CDRL3 or light chain variable region disclosed in Tables 1A, 3A, 3C and 3E, and a heavy chain variable region disclosed in Tables 1B, 3B, 3D and 3E. In some embodiments, a suitable assay for detecting competitive binding employs kinetic sensors used with Octet® systems (Pall ForteBio), which measures binding interactions using bio-layer interferometry methodology. One group of antibodies, antibodies 10E3, 13E7, 24F4, 4C5, 4G10, 32E3, and 6E7, competed with each other for binding to human TREM2, indicating that they share the same or similar epitope on human TREM2. Antibodies 16B8, 26A10, 26C10, 26F2, 33B12, and 5E3 compete with each other for TREM2 binding, but does not compete with antibodies in the first group or antibodies 24A10, 24G6, or 25F12, indicating that this second group of antibodies bind to a distinct epitope on human TREM2. Antibodies 24A10 and 24G6 share a similar epitope on human TREM2 as these two antibodies compete with each other for human TREM2 binding, but did not compete with any other antibody. Antibody 25F12 did not compete with any of the other tested antibodies for human TREM2 binding, indicating that this antibody binds to yet another epitope.

In some embodiments, a TREM2 agonist antigen binding protein competes with a reference antibody for binding to human TREM2, wherein the reference antibody comprises a light chain variable region comprising a sequence selected from SEQ ID NOs: 46-63 and a heavy chain variable region comprising a sequence selected from SEQ ID NOs: 110-126. In other embodiments, a TREM2 agonist antigen binding protein of the invention competes with a reference antibody for binding to human TREM2, wherein the reference antibody comprises a light chain variable region comprising a sequence selected from SEQ ID NOs: 153-162 and a heavy chain variable region comprising a sequence selected from SEQ ID NOs: 180-190. In still other embodiments, a TREM2 agonist antigen binding protein of the invention competes with a reference antibody for binding to human TREM2, wherein the reference antibody comprises a light chain variable region comprising a sequence selected from SEQ ID NOs: 61 and 295-300 and a heavy chain variable region comprising a sequence selected from SEQ ID NOs: 124 and 307-312. In certain embodiments, a TREM2 agonist antigen binding protein of the invention competes for binding to human TREM2 with one or more of the anti-TREM2 antibodies described herein, including 12G10, 26A10, 26C10, 26F2, 33B12, 24C12, 24G6, 24A10, 10E3, 13E7, 14C12, 25F12, 32E3, 24F4, 16B8, 4C5, 6E7, 5E3, 4G10, V3, V9, V10, V23, V24, V27, V30, V33, V40, V44, V48, V49, V52, V57, V60, V68, V70, V73, V76, V83, V84, and V90.

In some embodiments, the TREM2 agonist antigen binding protein competes with a reference antibody for binding to human TREM2, wherein the reference antibody comprises a light chain variable region comprising the sequence of SEQ ID NO: 61 and a heavy chain variable region comprising the sequence of SEQ ID NO: 124. In such embodiments, antigen binding proteins that compete with this reference antibody for binding to human TREM2 would bind the same or similar epitope as antibody 6E7 or any of the other antibodies 10E3, 13E7, 24F4, 4C5, 4G10, and 32E3.

In some embodiments, the TREM2 agonist antigen binding protein competes with a reference antibody for binding to human TREM2, wherein the reference antibody comprises a light chain variable region comprising the sequence of SEQ ID NO: 62 and a heavy chain variable region comprising the sequence of SEQ ID NO: 125. In such embodiments, antigen binding proteins that compete with this reference antibody for binding to human TREM2 would bind the same or similar epitope as antibody 5E3 or any of the other antibodies 16B8, 26A10, 26C10, 26F2, and 33B12.

In some embodiments, the TREM2 agonist antigen binding protein competes with a reference antibody for binding to human TREM2, wherein the reference antibody comprises a light chain variable region comprising the sequence of SEQ ID NO: 52 and a heavy chain variable region comprising the sequence of SEQ ID NO: 115. In such embodiments, antigen binding proteins that compete with this reference antibody for binding to human TREM2 would bind the same or similar epitope as antibody 24G6 or antibody 24A10.

In some embodiments, the TREM2 agonist antigen binding protein competes with a reference antibody for binding to human TREM2, wherein the reference antibody comprises a light chain variable region comprising the sequence of SEQ ID NO: 56 and a heavy chain variable region comprising the sequence of SEQ ID NO: 119. In such embodiments, antigen binding proteins that compete with this reference antibody for binding to human TREM2 would bind the same or similar epitope as antibody 25F12.

In some embodiments, isolated nucleic acids encoding the anti-TREM2 binding domain of the antigen binding proteins of the invention can be used to synthesize the antigen binding protein or used to generate variants. In some embodiments, the polynucleotide may comprise a nucleotide sequence that is at least 80% identical, at least 90% identical, at least 95% identical, or at least 98% identical to any of the nucleotide sequences listed in Table 3G.

TABLE 3G
Exemplary Anti-TREM2 Antibody Variable Region
Nucleic Acid Sequences

| | | | |
|---|---|---|---|
| | VL or | | |
| | VH | | |
| | Group | | SEQ |
| Ab | Desig- | | ID |
| ID . | nation | Nucleic Acid Sequence | NO: |

Light chain variable regions

| | | | |
|---|---|---|---|
| 12G10 | LV-01 | CAGGCTGTGCCGACTCAGCCGTCTTCCCTCTCTGCATCTCCTGGAGTATT | 208 |
| | | AGCCAGTCTCACCTGCACCTTACGCAGTGGCATCAATGTTGGTACCTACA | |
| | | GGATATACTGGTACCAGCAGAAGCCAGGGAGTCCTCCCCAGTATCTCCTG | |
| | | AGGTACAAATCAGACTCAGATAAGCAGCAGGGCTCTGGAGTCCCCAGCCG | |
| | | CTTCTCTGGATCCAAGGATGCTTCGGCCAATGCAGGGATTTTACTCATCT | |
| | | CTGGGCTCCAGTCTGAGGATGAGGCTGACTATTACTGTATGATTTGGTAC | |
| | | AGCAGTGCTGTGGTATTCGGCGGAGGGACCAAACTGACCGTCCTA | |
| 26A10 | LV-02 | TCCTATGAGCTGACTCAGCCACCCTCAGTGTCCGTGTCCCCAGGACAGAC | 209 |
| | | AGCCAGCATCACCTGCTCTGGAGATAAATTGGGAGATAAGTATGTTTGCT | |
| | | GGTATCAGCAGAAGCCAGGCCAGTCCCCTGTGCTGGTCATCTATCAAGAT | |
| | | AGCAAGCGGCCCTCAGGGATCCCTGAGCGATTCTCTGGCTCCAACTCTGG | |
| | | GAACACAGCCACTCTGACCATCAGCGGGACCCAGGCTATGGATGAGGCTG | |
| | | ACTATTACTGTCAGGCGTGGGACAGTAACACTGTGGTATTCGGCGGAGGG | |
| | | ACCAAGCTGACCGTCCTA | |
| 26C10 | LV-03 | TCCTTTGAGCTGACTCAGCCACCCTCAGTGTCCGTGTCCCCAGGACAGAC | 210 |
| | | AGCCAGCATCACCTGCTCTGGAGATAAATTGGGGGATAAGTATGTTTGCT | |
| | | GGTATCAGCAGAAGCCAGGCCAGTCCCCTATGTTGGTCATCTATCAAGAT | |
| | | ACCAAGCGGCCCTCAGGGATCCCTGAACGATTCTCTGGCTCCAACTCTGG | |
| | | GAACACAGCCACTCTGACCATCAGCGGGACCCAGGCTATGGATGAGGCTG | |
| | | ACTATTACTGTCAGGCGTGGGACAGCAGCACTGTGGTCTTCGGCGGAGGG | |
| | | ACCAAGCTGACCGTCCTA | |
| 26F2 | LV-04 | TCCTATGAGCTGACTCAGCCACCCTCAGTGTCCGTGTCCCCAGGACAGAC | 211 |
| | | AGCCAGCATCACCTGCTCTGGAGATAAATTGGGGGATAAGTATGTTTGCT | |
| | | GGTATCAGCAGAAGCCAGGCCAGTCCCCTGTGTTGGTCATCTTTCAAGAT | |
| | | AGCAAGCGGCCCTCAGGGATCCCTGAGCGATTCTCTGGCTCCAACTCTGG | |
| | | GAACACAGCCACTCTGACCATCAGCGGGACCCAGGCTATGGATGAGGCTG | |
| | | ACTATTACTGTCAGGCGTGGGACAGCAGCACTGTGGTATTCGGCGGAGGG | |
| | | ACCAAGCTGACCGTCCTA | |
| 33B12 | LV-05 | TCCTATGAGCTGACTCAGCCACCCTCAGTGTCCGTGTCCCCAGGACAGAC | 212 |
| | | AGCCAGCATCACCTGCTCTGGAGATAAATTGGGGGATAAGTATGTTTGCT | |
| | | GGTATCAGCAGAAGCCAGGCCAGTCCCCTGTGTTGGTCATCTATCAAGAT | |
| | | AGCAAGCGGCCCTCAGGGATCCCTGAGCGATTCTCTGGCTCCAACTCTGG | |
| | | GAACACAGCCACTCTGACCATCAGCGGGACCCAGGCTATGGATGAGGCTG | |
| | | ACTATTACTGTCAGGCGTGGGACAGTAGCACTGTGGTATTCGGCGGAGGG | |
| | | ACCAAGCTGACCGTCCTA | |
| 24C12 | LV-06 | GGCATCGTGATGACCCAGTCTCCAGACTCCCTGGCTGTGTCTCTGGGCGA | 213 |
| | | GAGGGCCACCATCAACTGCAAGTCCAGCCGGAGTGTTTTGTACAGCTCCA | |
| | | ACAATAAGAACTACTTAGCTTGGTACCAGCAGAAACCAGGACAGCCTCCT | |
| | | AAGGTGCTCATTTACTGGGCATCTACCCGGGAATCCGGGGTCCCTGACCG | |
| | | ATTCAGTGGCAGCGGGTCTGGGACAGATTTCACTCTCACCATCAGCAGCC | |
| | | TGCAGGCTGAAGATGTGGCAGTTTATAACTGTCAGCAATATTATATTACT | |
| | | CCGATCACCTTCGGCCAAGGGACACGACTGGAGATTAAA | |
| 24G6 | LV-07 | GACATCGTGATGACCCAGTCTCCAGACTCCCTGGCTGTGTCTCTGGGCGA | 214 |
| | | GAGGGCCACCATCAACTGCAAGTCCAGCCAGAGTGTTTTATACAGCTCCA | |
| | | ACAATAAGCACTTCTTAGCTTGGTACCAGCAGAAACCAGGACAGCCTCCT | |
| | | AAGCTGCTCATTTACTGGGCATCTACCCGGGAGTCCGGGGTCCCTGACCG | |
| | | ATTCAGTGGCAGCGGGTCTGGGACAGATTTCACTCTCACCATCAGCAGCC | |
| | | TGCAGGCTGAAGATGTGGCATTTTATTACTGTCAGCAATATTATAGTACT | |
| | | CCGCTCACTTTCGGCGGAGGGACCAAGGTGGAGATCAAA | |
| 24A10 | LV-08 | GACATCGTGATGACCCAGTCTCCAGACTCCCTGGCTGTGTCTCTGGGCGA | 215 |
| | | GAGGGCCACCATCACCTGCAAGTCCAGCCACAATGTTTTATACAGCTCCA | |
| | | ACAATAAGAACTACTTAGCTTGGTATCAGCAGAAACCAGGACAGCCTCCT | |
| | | AAACTGCTCATTTACTGGGCATCTACCCGGGAATCCGGGGTCCCTGACCG | |
| | | ATTCAGTGGCAGCGGGTCTGGGACAGATTTCACTCTCACCATCAGCAGCC | |
| | | TGCAGGCTGAAGATGTGGCAGTTTATTACTGTCACCAATATTATAGTACT | |
| | | CCGTGCAGTTTTGGCCAGGGGACCAAGCTGGAGATCAAA | |
| 10E3 | LV-09 | GAAATAGTGATGACGCAGTCTCCAGCCACCCTGTCTGTGTCTCCAGGGGA | 216 |
| | | AAGAGCCACCCTCTCCTGCAGGGCCAGTCAGAGTGTTAGCAGCAACTTAG | |
| | | CCTGGTTCCAGCAGAAACCTGGCCAGGCTCCCAGGCTCCTCATCTATGGT | |
| | | GCTTCCACCAGGGCCACTGGTATTCCAGCCAGGTTCAGTGTCAGTGGGTC | |
| | | TGGGACAGAGTTCACTCTCACCATCAGCAGCCTGCAGTCTGAAGATTTTG | |
| | | CATTTTATTACTGTCTGCAGGATAATAATTGGCCTCCCACTTTCGGCCCT | |
| | | GGGACCAAAGTGGATATCAAA | |
| 13E7 | LV-10 | GAAATAGTGATGACGCAGTCTCCAGCCACCCTGTCTGTGTCTCCAGGGGA | 217 |
| | | AAGAGCCACCCTCTCCTGCAGGGCCAGTCAGAGTGTTAGCAGCAACTTAG | |
| | | CCTGGTTCCAGCAGAAACCTGGCCAGGCTCCCAGGCTCCTCATCTATGGT | |
| | | GCTTCCACCAGGGCCACTGGTATTCCAGCCAGGTTCAGTGTCAGTGGGTC | |
| | | TGGGACAGAGTTCACTCTCACCATCAGCAGCCTGCAGTCTGAAGATTTTG | |
| | | CAGTTTATTACTGTCTGCAGGATAATAATTGGCCTCCCACTTTCGGCCCT | |
| | | GGGACCAAAGTGGATATCAAA | |
| 25F12 | LV-11 | GAAAAAGTGATGACGCAGTCTCCAGCCACCCTGTCTGTGTCTCCAGGGGA | 218 |
| | | AAGAGCCACCCTCTCCTGCAGGGCCAGTCAGAGTGTTAACAACAACTTAG | |
| | | CCTGGTACCAGCAGAAACCTGGCCAGGCTCCCAGGCTCCTCATCTATGGT | |
| | | GCATCCACCAGGGCCACTGGTATCCCAGCCAGGTTCAGTGGCAGTGGGTC | |
| | | TGGGACAGAGTTCACTCTCACCATCAGCAGCCTGCAGTCTGAAGATTTTG | |
| | | CAGTTTATTACTGTCAGCAGTATAATAACTGGCCTCGGACGTTCGGCCAA | |
| | | GGGACCAAGGTGGAAATCAAA | |
| 32E3 | LV-12 | GAATTTGTGTTGACGCAGTCTCCAGGCACCCTGTCTTTGTCTCCGGGGGA | 219 |
| | | AAGAGCCACCCTCTCCTGCAGGGCCAGTCAGATTATTAGCAGCAACTACT | |
| | | TAGCCTGGTACCAGCAGAAACCTGGCCAGGCTCCCAGGCTCCTCATCTAT | |
| | | AGTGCATCCAGCAGGGCCACTGGCATCCCAGACAGGTTCAGTGGCAGTGG | |
| | | GTCTGGGACAGACTTCACTCTCACCATCAGCAGACTGGAGCCTGAAGATT | |
| | | TTGCAGTGTATTACTGTCAGCAGTTTGATAGCTCACCGATCACCTTCGGC | |
| | | CGAGGGACACGACTGGACATTAAA | |
| 24F4 | LV-13 | GAAATTGTGTTGACGCAGTCTCCAGGCACCCTGTCTTTGTCTCCAGGGGA | 220 |
| | | AAGAGCCACCCTCTCCTGCAGGGCCAGTCAGAGTGTTAGCAGCAGCTACT | |
| | | TAGCCTGGTACCAGCAGAAACCTGGCCAGGCTCCCAGGCTCCTCATCTAT | |
| | | GGTGCATCCAGCAGGGCCACTGGCATCCCAGACAGGTTCAGTGGCAGTGG | |
| | | GTCTGGGACAGACTTCACTCTCACCATCAGCAGACTGGAGCCTGAAGATT | |
| | | TTGCACTGTATTACTGTCAGCAGTATGATACCTCACCATTCACTTTCGGC | |
| | | CCTGGGACCAAAGTGGATATCAAA | |
| 16B8 | LV-14 | GACATCCAGATGACCCAGTCTCCATCTTCCGTGTCTGCATCTGTAGGAGA | 221 |
| | | CAGAGTCACCGTCACTTGTCGGGCGAGTCAGGATATTAACAGCTGGTTAG | |
| | | CCTGGTATCAGCAGAAACCAGGGAAAGCCCCTAAGCTCCTGATCTATGCT | |
| | | GCATCCTCTTTGCAAACTGGGGTCCCTTCAAGGTTCAGCGGCAGTGGATC | |
| | | TGGGACAGATTTCACTCTCACCATCAGCAGCCTGCAGCCTGAAGATTTTG | |
| | | CAACTTACTCTTGTCAACAGTCTAACAGTTTCCCGATCACCTTCGGCCAA | |
| | | GGGACACGACTGGAGATTAAA | |
| 4C5 | LV-15 | GACATCCAGATGACCCAGTCTCCATCTTCCGTGTCTGCATCTGTAGGAGA | 222 |
| | | CAGAGTCACCATCACTTGTCGGGCGAGTCAGGGTATTAGCAACTGGTTAG | |
| | | CCTGGTATCAGCAGAAACCAGGGAAAGCCCCTAAGCTCCTGATCTATGCT | |
| | | GCATCCAGTTTGCAAGTTGGGGTCCCATTAAGGTTCAGCGGCAGTGGATC | |
| | | TGGGACAGATTTCACTCTCACCATCAGCAGCCTGCAGCCTGAAGATTTTG | |
| | | CAACTTACTATTGTCAACAGGCTGACAGTTTCCCTCGCAATTTTGGCCAG | |
| | | GGGACCAAGCTGGAGATCAAA | |
| 6E7 | LV-16 | GACATCCAGATGACCCAGTCTCCATCTTCCGTGTCTGCATCTGTAGGAGA | 223 |
| V9 | | CAGAGTCACCATCACTTGTCGGGCGAGTCAGGGTATTAGCAGCTGGTTAG | |
| V30 | | CCTGGTATCAGCAGAAACCAGGGAAAGCCCCTAAGCTCCTGATCTATGCT | |
| V33 | | GCATCCAGTTTGCAAAATGGGGTCCCATCAAGGTTCAGCGGCAGTGGATC | |
| V44 | | TGGGACAGATTTCACTCTCACCATCAGCAGCCTGCAGCCTGAAGATTTTG | |
| V68 | | CAACTTACTTTTGTCAACAGGCTGACAGTTTCCCTCGCACTTTTGGCCAG | |
| | | GGGACCAAGCTGGAGATCAAA | |
| 5E3 | LV-17 | GACATCCAGATGACCCAGTCTCCATCCTCACTGTCTGCATCTGTAGGAGA | 224 |
| | | CAGAGTCACCATCACTTGTCGGGCGAGTCAGGGCATTAGCAATTATTTAG | |
| | | CCTGGTTTCAGCAGAAACCAGGGAAAGCCCCTAAATCCCTGATCTATGCT | |
| | | GCATCCAGTTTGCAAAGTGGGGTCCCATCAAAGTTCAGCGGCAGTGGATC | |
| | | TGGGACAGATTTCACTCTCACCATCAGCAGCCTGCAGCCTGAAGATTTTG | |
| | | CAACTTATTACTGCCAACAGTATAGTACTTACCCATTCACTTTCGGCCCT | |
| | | GGGACCAAAGTGGATATCAAA | |
| 4G10 | LV-18 | GACATCCAGATGACCCAGTCTCCATCCTCCCTGTCTGCATCTGTAGGAGA | 225 |
| | | CAGAGTCACCATCACTTGCCGGGCAAGTCAGGGCATAAGAAATGATTTAG | |
| | | GCTGGTATCAGCAGAAACCAGGGAATGCCCCTAAGCGCCTGATCTATGCT | |
| | | GCATCCAGTTTGCCAAGTGGGGTCCCATCAAGGTTCAGCGGCAGTGGATC | |
| | | TGGGCCAGAATTCACTCTCACAATCAGCAGTCTGCAGCCTGAAGATTTTG | |
| | | CAACTTATTACTGTCTACAGCATAATAGTTACCCGTGGACGTTCGGCCAA | |
| | | GGGACCAAGGTGGAAATCACA | |
| V3 | LV-101 | GACATCCAGATGACCCAGTCTCCATCTTCCGTGTCTGCATCTGTAGGAGA | 226 |
| | | CAGAGTCACCATCACTTGTCGGGCGAGTCAGGGTATTAGCAGCTGGTTAG | |
| | | CCTGGTATCAGCAGAAACCAGGGAAAGCCCCTAAGCTCCTGATCTATGCT | |
| | | GCATCCAGTAGGCAAAATGGGGTCCCATCAAGGTTCAGCGGCAGTGGATC | |
| | | TGGGACAGATTTCACTCTCACCATCAGCAGCCTGCAGCCTGAAGATTTTG | |
| | | CAACTTACTTTTGTCAACAGGCTGACAGGTTCCCTCGCACTTTTGGCCAG | |
| | | GGGACCAAGCTGGAGATCAAA | |
| V24 | LV-102 | GACATCCAGATGACCCAGTCTCCATCTTCCGTGTCTGCATCTGTAGGAGA | 227 |
| | | CAGAGTCACCATCACTTGTCGGGCGAGTCAGGGTATTAGCAGCTGGTTAG | |
| | | CCTGGTATCAGCAGAAACCAGGGAAAGCCCCTAAGCTCCTGATCTATGCT | |
| | | GCATCCAGTTTGCAAAAGGGGGTCCCATCAAGGTTCAGCGGCAGTGGATC | |
| | | TGGGACAGATTTCACTCTCACCATCAGCAGCCTGCAGCCTGAAGATTTTG | |
| | | CAACTTACTTTTGTCAACAGGCTGACAGTTTCCCTCATACTTTTGGCCAG | |
| | | GGGACCAAGCTGGAGATCAAA | |
| V27 | LV-103 | GACATCCAGATGACCCAGTCTCCATCTTCCGTGTCTGCATCTGTAGGAGA | 228 |
| | | CAGAGTCACCATCACTTGTCGGGCGAGTCAGGGTATTAGCAGCTGGTTAG | |
| | | CCTGGTATCAGCAGAAACCAGGGAAAGCCCCTAAGCTCCTGATCTATGCT | |
| | | GCATCCAGTTTGCAACGTGGGGTCCCATCAAGGTTCAGCGGCAGTGGATC | |
| | | TGGGACAGATTTCACTCTCACCATCAGCAGCCTGCAGCCTGAAGATTTTG | |
| | | CAACTTACTTTTGTCAACAGGCTGACAGTTTCCCTCGCACTTTTGGCCAG | |
| | | GGGACCAAGCTGGAGATCAAA | |
| V40 | LV-104 | GACATCCAGATGACCCAGTCTCCATCTTCCGTGTCTGCATCTGTAGGAGA | 229 |
| | | CAGAGTCACCATCACTTGTCGGGCGAGTCAGGGTATTAGCAGCTGGTTAG | |
| | | CCTGGTATCAGCAGAAACCAGGGAAAGCCCCTAAGCTCCTGATCTATGCT | |
| | | GCATCCAGTTTGCAACTTGGGGTCCCATCAAGGTTCAGCGGCAGTGGATC | |
| | | TGGGACAGATTTCACTCTCACCATCAGCAGCCTGCAGCCTGAAGATTTTG | |
| | | CAACTTACTTTTGTCAACAGGCTGACCGTTTCCCTCGCACTTTTGGCCAG | |
| | | GGGACCAAGCTGGAGATCAAA | |
| V48 | LV-105 | GACATCCAGATGACCCAGTCTCCATCTTCCGTGTCTGCATCTGTAGGAGA | 230 |
| | | CAGAGTCACCATCACTTGTCGGGCGAGTCAGGGTATTAGCAGCTGGTTAG | |
| | | CCTGGTATCAGCAGAAACCAGGGAAAGCCCCTAAGCTCCTGATCTATGCT | |
| | | GCATCCAGTTTGCAAACGGGGGTCCCATCAAGGTTCAGCGGCAGTGGATC | |
| | | TGGGACAGATTTCACTCTCACCATCAGCAGCCTGCAGCCTGAAGATTTTG | |
| | | CAACTTACTTTTGTCAACAGGCTGACAGTTTGCCTCGCACTTTTGGCCAG | |
| | | GGGACCAAGCTGGAGATCAAA | |
| V49 | LV-106 | GACATCCAGATGACCCAGTCTCCATCTTCCGTGTCTGCATCTGTAGGAGA | 231 |
| | | CAGAGTCACCATCACTTGTCGGGCGAGTCAGGGTATTAGCAGCTGGTTAG | |
| | | CCTGGTATCAGCAGAAACCAGGGAAAGCCCCTAAGCTCCTGATCTATGCT | |
| | | GCATCCAGTCGGCAAAATGGGGTCCCATCAAGGTTCAGCGGCAGTGGATC | |
| | | TGGGACAGATTTCACTCTCACCATCAGCAGCCTGCAGCCTGAAGATTTTG | |
| | | CAACTTACTTTTGTCAACAGGCTGACAGTTATCCTCGCACTTTTGGCCAG | |
| | | GGGACCAAGCTGGAGATCAAA | |
| V52 | LV-107 | GACATCCAGATGACCCAGTCTCCATCTTCCGTGTCTGCATCTGTAGGAGA | 232 |
| | | CAGAGTCACCATCACTTGTCGGGCGAGTCAGGGTATTAGCAGCTGGTTAG | |
| | | CCTGGTATCAGCAGAAACCAGGGAAAGCCCCTAAGCTCCTGATCTATGCT | |
| | | GCATCCAGTTTGCAAAGGGGGGTCCCATCAAGGTTCAGCGGCAGTGGATC | |
| | | TGGGACAGATTTCACTCTCACCATCAGCAGCCTGCAGCCTGAAGATTTTG | |
| | | CAACTTACTTTTGTCAACAGGCTGACCGTTTCCCTCGCACTTTTGGCCAG | |
| | | GGGACCAAGCTGGAGATCAAA | |
| V60 | LV-108 | GACATCCAGATGACCCAGTCTCCATCTTCCGTGTCTGCATCTGTAGGAGA | 233 |
| | | CAGAGTCACCATCACTTGTCGGGCGAGTCAGGGTATTAGCAGCTGGTTAG | |
| | | CCTGGTATCAGCAGAAACCAGGGAAAGCCCCTAAGCTCCTGATCTATGCT | |
| | | GCATCCAGTTTGCAAAGGGGGGTCCCATCAAGGTTCAGCGGCAGTGGATC | |
| | | TGGGACAGATTTCACTCTCACCATCAGCAGCCTGCAGCCTGAAGATTTTG | |
| | | CAACTTACTTTTGTGGGCAGGCTGACAGTTTCCCTCGCACTTTTGGCCAG | |
| | | GGGACCAAGCTGGAGATCAAA | |
| V73 | LV-106 | GACATCCAGATGACCCAGTCTCCATCTTCCGTGTCTGCATCTGTAGGAGA | 234 |
| | | CAGAGTCACCATCACTTGTCGGGCGAGTCAGGGTATTAGCAGCTGGTTAG | |
| | | CCTGGTATCAGCAGAAACCAGGGAAAGCCCCTAAGCTCCTGATCTATGCT | |
| | | GCATCCAGTCGTCAAAATGGGGTCCCATCAAGGTTCAGCGGCAGTGGATC | |
| | | TGGGACAGATTTCACTCTCACCATCAGCAGCCTGCAGCCTGAAGATTTTG | |
| | | CAACTTACTTTTGTCAACAGGCTGACAGTTATCCTCGCACTTTTGGCCAG | |
| | | GGGACCAAGCTGGAGATCAAA | |
| V76 | LV-109 | GACATCCAGATGACCCAGTCTCCATCTTCCGTGTCTGCATCTGTAGGAGA | 235 |
| | | CAGAGTCACCATCACTTGTCGGGCGAGTCAGGGTATTAGCAGCTGGTTAG | |
| | | CCTGGTATCAGCAGAAACCAGGGAAAGCCCCTAAGCTCCTGATCTATGCT | |
| | | GCATCCAGTTTGCAAAAGGGGGTCCCATCAAGGTTCAGCGGCAGTGGATC | |
| | | TGGGAGAGATTTCACTCTCACCATCAGCAGCCTGCAGCCTGAAGATTTTG | |
| | | CAACTTACTTTTGTCAACAGGCTGACAGTTTCCCTCGCACTTTTGGCCAG | |
| | | GGGACCAAGCTGGAGATCAAA | |
| V84 | LV-110 | GACATCCAGATGACCCAGTCTCCATCTTCCGTGTCTGCATCTGTAGGAGA | 236 |
| | | CAGAGTCACCATCACTTGTCGGGCGAGTCAGGGTATTAGCAGCTGGTTAG | |
| | | CCTGGTATCAGCAGAAACCAGGGAAAGCCCCTAAGCTCCTGATCTATGGT | |
| | | GCATCCAGTTTGCAAAATGGGGTCCCATCAAGGTTCAGCGGCAGTGGATC | |
| | | TGGGACAGATTTCACTCTCACCATCAGCAGCCTGCAGCCTGAAGATTTTG | |
| | | CAACTTACTTTTGTCAACAGGCTGACAGTTTCCCGCGCACTTTTGGCCAG | |
| | | GGGACCAAGCTGGAGATCAAA | |
| V10 | LV-201 | GACATCCAGATGACCCAGTCTCCATCTTCCGTGTCTGCATCTGTAGGAGA | 313 |
| | | CAGAGTCACCATCACTTGTCGGGCGAGTCAGGGTATTAGCAGCTGGTTAG | |
| | | CCTGGTATCAGCAGAAACCAGGGAAAGCCCCTAAGCTCCTGATCTATTCT | |
| | | GCATCCAGTTTGCAAAATGGGGTCCCATCAAGGTTCAGCGGCAGTGGATC | |
| | | TGGGACAGATTTCACTCTCACCATCAGCAGCCTGCAGCCTGAAGATTTTG | |
| | | CAACTTACTTTTGTCAACAGGCTGACAGTTTCCCTCGCACTTTTGGCCAG | |
| | | GGGACCAAGCTGGAGATCAAA | |
| V23 | LV-202 | GACATCCAGATGACCCAGTCTCCATCTTCCGTGTCTGCATCTGTAGGAGA | 314 |
| | | CAGAGTCACCATCACTTGTCGGGCGAGTCAGGGTATTAGCAGCTGGTTAG | |
| | | CCTGGTATCAGCAGAAACCAGGGAAAGCCCCTAAGCTCCTGATCTATGCT | |
| | | GCATCCAGTTTGCAAAATGGGGTCCCATCAAGGTTCAGCGGCAGTGGATC | |
| | | TGGGACAGATTTCACTCTCACCATCAGCAGCCTGCAGCCTGAAGATTTTG | |
| | | CAACTTACTTTTGTCAACAGGCTGACAGTTTCCCTCTTACTTTTGGCCAG | |
| | | GGGACCAAGCTGGAGATCAAA | |
| V57 | LV-203 | GACATCCAGATGACCCAGTCTCCATCTTCCGTGTCTGCATCTGTAGGAGA | 315 |
| | | CAGAGTCACCATCACTTGTGCGGCGAGTCAGGGTATTAGCAGCTGGTTAG | |
| | | CCTGGTATCAGCAGAAACCAGGGAAAGCCCCTAAGCTCCTGATCTATGCT | |
| | | GCATCCAGTTTGCAAAATGGGGTCCCATCAAGGTTCAGCGGCAGTGGATC | |
| | | TGGGACAGATTTCACTCTCACCATCAGCAGCCTGCAGCCTGAAGATTTTG | |
| | | CAACTTACTTTTGTCAACAGGCTGACAGTTTCCCTCGCACTTTTGGCCAG | |
| | | GGGACCAAGCTGGAGATCAAA | |
| V70 | LV-204 | GACATCCAGATGACCCAGTCTCCATCTTCCGTGTCTGCATCTGTAGGAGA | 316 |
| | | CAGAGTCACCATCACTTGTCGGGCGAGTCAGGGTATTAGCAGCTGGTTAG | |
| | | CCTGGTATCAGCAGAAACCAGGGAAAGCCCCTAAGCTCCTGATCTATGCT | |
| | | GCAGGGAGTTTGCAAAATGGGGTCCCATCAAGGTTCAGCGGCAGTGGATC | |
| | | TGGGACAGATTTCACTCTCACCATCAGCAGCCTGCAGCCTGAAGATTTTG | |
| | | CAACTTACTTTTGTCAACAGGCTGACAGTTTCCCTCGCACTTTTGGCCAG | |
| | | GGGACCAAGCTGGAGATCAAA | |
| V83 | LV-205 | GACATCCAGATGACCCAGTCTCCATCTTCCGTGTCTGCATCTGTAGGAGA | 317 |
| | | CAGAGTCACCATCACTTGTCGGGCGAGTCAGGGTATTAGCAGCTGGTTAG | |
| | | CCTGGTATCAGCAGAAACCAGGGAAAGCCCCTAAGCTCCTGATCTATGCT | |
| | | GCATCCAGTTTGCAAAATGGGGTCCCATCAAGGTTCAGCGGCAGTGGATC | |
| | | TGGGACAGATTTCACTCTCACCATCAGCAGCCTGCAGCCTGAAGATTTTG | |
| | | CAACTTACTTTTGTCAACAGGCTGTGAGTTTCCCTCGCACTTTTGGCCAG | |
| | | GGGACCAAGCTGGAGATCAAA | |
| V90 | LV-206 | GACATCCAGATGACCCAGTCTCCATCTTCCGTGTCTGCATCTGTAGGAGA | 318 |
| | | CAGAGTCACCATCACTTGTCGGGCGAGTCAGGGTATTAGCAGATGGTTAG | |
| | | CCTGGTATCAGCAGAAACCAGGGAAAGCCCCTAAGCTCCTGATCTATGCT | |
| | | GCATCCAGTTTGCAAAATGGGGTCCCATCAAGGTTCAGCGGCAGTGGATC | |
| | | TGGGACAGATTTCACTCTCACCATCAGCAGCCTGCAGCCTGAAGATTTTG | |
| | | CAACTTACTTTTGTCAACAGGCTGACAGTTTCCCTCGCACTTTTGGCCAG | |
| | | GGGACCAAGCTGGAGATCAAA | |

Heavy chain variable regions

| | | | |
|---|---|---|---|
| 12G10 | HV-01 | GAGGTGCAGCTGTTGGAGTCTGGGGGAGGCTTGGTACAGCCTGGGGGGTC | 237 |
| 24C12 | | CCTGAGACTCTCCTGTGCAGCCTCTGGATTCACCTTTAGCAGCTATGCCA | |
| | | TGAGCTGGGTCCGCCAGGCTCCAGGGAAGGGGCTGGAGTGGGTCTCAGCT | |
| | | ATTGGTGGTGGTGGTGTTAGCACATACTGCGCAGACTCCGTGAAGGGCCG | |
| | | GTTCACCATCTCCAGAGACAATTCCAAGAATACGCTGTATCTGCAAATGA | |
| | | ACAGCCTGAGAGCCGAGGACACGGCCGTATATTACTGTGCGAAATTTTAT | |
| | | ATAGCAGTGGCTGGTTCTCACTTTGACTACTGGGGCCAGGGAACCCTGGT | |
| | | CACCGTCTCCTCA | |
| 26A10 | HV-02 | GAGGTGCAACTGGTGGAGTCTGGGGGAGCCTTGGTACAGCGGGGGGGGTC | 238 |
| | | CCTGAGACTCTCCTGTGCAGCCTCTAGATTCACCTTCAGTAGCTTTGGCA | |
| | | TGAGCTGGGTCCGCCAGGCTCCAGGGAAGGGGCTGGAGTGGGTTTCATAC | |
| | | ATTAGTAGTAGTAGTTTTACCATATATTACGCAGACTCTGTGAAGGGCCG | |
| | | ATTCACCATCTCCAGAGACAATGCCAAGAATTCATTCTATCTGCAAATGA | |
| | | ACAGCCTGAGAGACGAGGACACGGCTGTGTATTACTGTGCGAGAGAGGGG | |
| | | GGTCTTACTATGGTTCGGGGAGTCTCTTCCTACGGTTTGGACGTCTGGGG | |
| | | CCAAGGGACCACGGTCACCGTCTCCTCA | |
| 26C10 | HV-03 | GAGGTGCAACTGGTGGAGTCTGGGGGAGCCTTGGTACAGCCTGGGGGGTC | 239 |
| | | CCTGAGACTCTCCTGTGCAGCCTCTGGATTCACCTTCAGTAGCTTTGGCA | |
| | | TGAGCTGGGTCCGCCAGGCTCCAGGGAAGGGGCTGGAGTGGGTTTCATAC | |
| | | ATTAGTAGTAGTAGTTTTACCATATACTACGCAGACTCTGTGAAGGGCCG | |
| | | ATTCACCATCTCCAGAGACAATGCCAAGAATTCGTTCTATCTGCAAATGA | |
| | | ACAGCCTGAGAGACGAGGACACGGCTGTGTATTTCTGTGTGAGAGAGGGG | |
| | | GGTATAACTATGGTTCGGGGAGTCTCTTCCTACGGTATGGACGTCTGGGG | |
| | | CCAAGGGACCACGGTCACCGTCTCCTCA | |
| 26F2 | HV-04 | GAGGTGCAACTGGTGGAGTCTGGGGGAGCCTTGGTACAGCCTGGGGGGTC | 240 |
| | | CCTGAGACTCTCCTGTGCAGCCTCTGGATTCACCTTCAGTAGCTTTGGCA | |
| | | TGAGCTGGGTCCGCCAGGCTCCAGGGAAGGGGCTGGAGTGGATTTCATAC | |
| | | ATTAGTAGTAGTAGTTTTACCATATACTACGCAGACTCTGTGAAGGGCCG | |
| | | ATTCACCATCTCCAGAGACAATGCCAAGAATTCATTCTATCTGCAAATGA | |
| | | ACAGCCTGAGAGACGAGGACACGGCTGTGTATTTCTGTGCGAGAGAGGGG | |
| | | GGTATTACTATGGTTCGGGGAGTCTCTTCCTACGGTATGGACGTCTGGGG | |
| | | CCAAGGGACCACGGTCACCGTCTCCTCA | |
| 33B12 | HV-05 | GAGGTGCAACTGGTGGAGTCTGGGGGAGCCTTGGTACAGCCTGGGGGGTC | 241 |
| | | CCTGAGACTCTCCTGTGCAGCCTCTGGATTCACCTTCAGTAGCTTTGGCA | |
| | | TGAGCTGGGTCCGCCAGGCTCCAGGGAAGGGCCTGGAGTGGGTTTCATAC | |
| | | ATTAGTAAAAGTAGTTTTACCATATACTACGCAGACTCTGTGAAGGGCCG | |
| | | ATTCACCATCTCCAGAGACAATGCCAAGAATTCATTCTATCTGCAAATGA | |
| | | ACAGCCTGAGAGACGAGGACACGGCTGTGTATTACTGTGCGAGAGAGGGG | |
| | | GGTCTTACTATGGTTCGGGGAGTCTCTTCCTACGGTTTGGACGTCTGGGG | |
| | | CCAAGGGACCACGGTCACCGTCTCCTCA | |
| 24G6 | HV-06 | GAGGTGCAGCTGTTGGAGTCTGGGGGAGGCTTGGTACAGCCTGGGGGGTC | 242 |
| | | CCTGAGACTCTCCTGTGCAGCCTCTGGATTCACCTTTAGCAGCTATGCCA | |
| | | TGAGCTGGGTCCGCCAGGCTCCAGGGAAGGGACTGGAGTGGGTCTCAGCT | |
| | | ATTAGTGGTAGTGGTGGTAGCACATACTACGCAGACTCCGTGAAGGGCCG | |
| | | GTTCACCATCTCCAGAGACAATTCCAAGAACACGCTGTATCTGCAAATGA | |
| | | ACAGCCTGAGAGCCGAGGACACGGCCGTATATTACTGTGCGAAGGCGTAT | |
| | | ACACCTATGGCATTCTTTGACTACTGGGGCCAGGGAACCCTGGTCACCGT | |
| | | CTCCTCA | |
| 24A10 | HV-07 | GAGGTGCAGGTGTTGGAGTCTGGGGGAGGCTTGGTACAGCCTGGGGGGTC | 243 |
| | | CCTGAGACTCTCCTGTGCAGCCTCTGGATTCACCTTTAGCAACTATGCCA | |
| | | TGAGCTGGGTCCGCCAGGCTCCAGGGAAGGGGCTGGAGTGGGTCTCAGCT | |
| | | ATTAGTGGTAGTGGTGGTAGCACATACTACGCAGACTCCGTGAAGGGCCG | |
| | | GTTCACCATCTCCAGAGACAATTCCAAGAACACGCTGTATCTGCAAATGA | |
| | | ACAGCCTGAGAGCCGAGGACACGGCCGTATATTACTGTGCGAAAGGAGGG | |
| | | TGGGAGCTATTTTACTGGGGCCAGGGAACCCTGGTCACCGTCTCCTCA | |
| 10E3 | HV-08 | GAGGTGCAGCTGGTGCAGTCTGGAGCAGAGGTGAAAAAGCCCGGGGAGTC | 244 |
| | | TCTGATGATCTCCTGTAAGGGTTCTGGATACAGCTTTACCAACTACTGGA | |
| | | TCGGCTGGGTGCGCCAGATGCCCGGGAAAGGCCTGGAGTGGATGGGGATC | |
| | | ATCTATCCTGGAGACTCTGATACCAGATACAGCCCGTCCTTCCAAGGCCA | |
| | | GGTCACCATCTCAGCCGACAAGTCCATCAGCACCGCCTACCTGCAGTGGA | |
| | | GCAGCCTGAAGGCCTCGGACACCGCCATGTATTTCTGTGCGAGACGGAGA | |
| | | CAGGGGATCTGGGGTGATGCTCTTGATATCTGGGGCCAAGGGACATTGGT | |
| | | CACCGTCTCTTCA | |
| 13E7 | HV-09 | GAGGTGCAGCTGGTGCAGTCTGGAGCAGAGGTGAAAAAGCCCGGGGAGTC | 245 |
| | | TCTGATGATCTCCTGTAAGGGTTCTGGATACAGCTTTACCAGCTACTGGA | |
| | | TCGGCTGGGTGCGCCAGATGCCCGGGAAAGGCCTGGAGTGGATGGGGATC | |
| | | ATCTATCCTGGAGACTCTGATACCAGATACAGCCCGTCCTTCCAAGGCCA | |
| | | GGTCACCATCTCAGCCGACAAGTCCATCAGCACCGCCTACCTGCAGTGGA | |
| | | GCAGCCTGAAGGCCTCGGACACCGCCATGTATTTCTGTGCGAGACGGAGA | |
| | | CAGGGGATCTGGGGTGATGCTCTTGATTTCTGGGGCCAAGGGACATTGGT | |
| | | CACCGTCTCTTCA | |
| 25F12 | HV-10 | CAGGTGCAGCTACAGCAGTGGGGCGCAGGACTGTTGAAGCCTTCGGAGAC | 246 |
| | | CCTGTCCCTCACCTGCGCTGTCTATGGTGGGTCCTTCAGTAGTTACTACT | |
| | | GGAGCTGGATCCGCCAGCCCCCAGGGAAGGGGCTGGAGTGGATTGGGGAA | |
| | | ATCAATCATAGTGGAAACACCAACTACAACCCGTCCCTCAAGAGTCGAGT | |
| | | CACCATATCAGTAGACACGTCCAAGAACCAGTTCTCCCTGAAGCTGAGCT | |
| | | CTGTGACCGCCGCGGACACGGCTGTGTATTACTGTGCGAGAGAGGGGTAT | |
| | | TACGATATCTTGACTGGTTATCATGATGCTTTTGATATTTGGGACCAAGG | |
| | | GACAATGGTCACCGTNTTTTCA | |
| 32E3 | HV-11 | GAGGTGCAGCTGGTGCAGTCTGGAGCAGAGGTGAAAAAGCCCGGGGAGTC | 247 |
| | | TCTGAAGATCTCCTGTAAGGGTTCTGGATACAGCTTTACCAGCTACTGGA | |
| | | TCGGCTGGGTGCGCCAGATGCCCGGGAAAGGCCTGGAGTGGATGGGGATC | |
| | | ATCTATCCTGGTGACTCTGATACCAGATACAGCCCGTCCTTCCAAGGCCA | |
| | | GGTCACCATCTCAGCCGACAAGTCCATCAGCACCGCCTACCTGCAGTGGA | |
| | | GCACCCTGAAGGCCTCGGACACCGCCATATATTACTGTGCGCGACATGAC | |
| | | ATTATACCAGCAGCCCCTGGTGCTTTTGATATCTGGGGCCAAGGGACAAT | |
| | | GGTCACCGTCTCTTCA | |
| 24F4 | HV-12 | GAGGTGCAGCTGGTGCAGTCTGGAGCAGAGGTGAAAAAGCCCGGGGAGTC | 248 |
| | | TCTGAAGATCTCCTGTAAGGGTTCTGGATACACCTTTACCAGCTACTGGA | |
| | | TCGGCTGGGTGCGCCAGATGCCCGGGAAAGGCCTGGAGTGGATGGGGATC | |
| | | ATCTATCCTGGTGACTCTGATACCAGATACAGCCCGTCCTTCCAAGGCCA | |
| | | GGTCACCATCTCAGTCGACAAGTCCAGCAGCACCGCCTACCTGCAGTGGA | |
| | | GCAGCCTGAAGGCCTCGGACACCGCCATATATTACTGTACGAGACAGGCC | |
| | | ATAGCAGTGACTGGTTTGGGGGGTTTCGACCCCTGGGGCCAGGGAACCCT | |
| | | GGTCACCGTCTCCTCA | |
| 16B8 | HV-13 | CAGGTTCAGCTGGTGCAGTCTGGAGCTGAGGTGAAGAAGCCTGGGGCCTC | 249 |
| | | AGTGAAGGTCTCCTGCAAGGCTTCTGGTTACACCTTTACCAACTATGGTA | |
| | | TCAGCTGGGTGCGACAGGCCCCTGGACAAGGGCTTGAGTGGATGGGATGG | |
| | | ATCAGCGCTTACAATGGTAACACAAACTATGCACAGAAGCTCCAGGGCAG | |
| | | AGTCACCATGACCACAGACACATCCACGAGTACAGTCTACATGGAGCTGA | |
| | | GGAGCCTGAGATCTGACGACACGGCCGTGTATTACTGTGCGAGACGGGGA | |
| | | TACAGCTATGGTTCCTTTGACTACTGGGGCCAGGGAACCCTGGTCACCGT | |
| | | CTCCTCA | |
| 4C5 | HV-14 | GAGGTGCAGCTGGTGCAGTCTGGAGCAGAAGTGAAAAAGCCCGGGGAGTC | 250 |
| | | TCTGAAGATCTCCTGTAAGGGTTCTGGACACAGTTTTACCAACTACTGGA | |
| | | TCGCCTGGGTGCGCCAGATGCCCGGGAAAGGCCTGGAGTGGATGGGGATC | |
| | | ATCTATCCTGGTGACTCTGATACCAGATACAGCCCGTCCTTCCAAGGCCA | |
| | | GGTCACCATCTCAGCCGACAAGTCCATCAGCACCGCCTACCTGCAGTGGA | |
| | | GCAGCCTGAAGGCCTCGGACACCGCCGTGTATTTCTGTGCGAGACAAAGG | |
| | | ACGTTTTACTATGATAGTAGTGGTTATTTTGACTACTGGGGCCAGGGAAC | |
| | | CCTGGTCACCGTCTCCTCA | |
| 6E7 | HV-15 | GAGGTGCAGCTGGTGCAGTCTGGAGCAGAGGTGAAAAAGCCCGGGGAGTC | 251 |
| | | TCTGAAGATCTCCTGTAAGGGTTCTGGATACAGTTTTACCAGCTACTGGA | |
| | | TCGCCTGGGTGCGCCAGATGCCCGGGAAAGGCCTGGAGTGGATGGGGATC | |
| | | ATCTATCCTGGTGACTCTGATACCAGATACAGCCCGTCCTTCCAAGGCCA | |
| | | GGTCACCATCTCAGCCGACAAGTCCATCAGCACCGCCTACCTACAGTGGA | |
| | | GCAGCCTGAAGGCCTCGGACACCGCCATGTATTTCTGTGCGAGACAAAGG | |
| | | ACGTTTTATTATGATAGTAGTGATTATTTTGACTACTGGGGCCAGGGAAC | |
| | | CCTGGTCACCGTCTCCTCA | |
| 5E3 | HV-16 | CAGGTGCAGCTGGTGCAGTCTGGGGCTGAGGTGAAGAAGCCTGGGGCCTC | 252 |
| | | AGTGAAGGTCTCCTGCAAGGCTTCTGGATACACCTTCACCGGCTACTATA | |
| | | TACACTGGGTGCGACAGGCCCCTGGACTAGGGCTTGAGTGGATGGGATGG | |
| | | ATCAACCCTTACAGTGGTGGCACAACCTCTGCACAGAAGTTTCAGGGCAG | |
| | | GGTCACCATGACCAGGGACACGTCCATCAGCTCAGCCTACATGGAACTGA | |
| | | GCAGGCTGAGATCTGACGACACGGCCGTGTATTACTGTGCGAGAGATGGA | |
| | | GGCTACCTGGCCCTCTACGGTACGGACGTCTGGGGCCAAGGGACCACGGT | |
| | | CACCGTCTCCTCA | |
| 4G10 | HV-17 | GAGGTGCAGCTGGTGCAGTCTGGAGCAGAGGTGAAAAAGCCCGGGGAGTC | 253 |
| | | TCTGAAGATCTCCTGTAAGGGTTCTGGATACAGCTTTCCCAGCTACTGGA | |
| | | TCGCCTGGGTGCGCCAGATGCCCGGGAAAGGCCTGGAGTGGATGGGGATC | |
| | | ATCTATCCTGGTGACTCTGATACCAGATACAGCCCGTCCTTCCAAGGCCA | |
| | | GGTCACCATCTCAGCCGACAAGTCCATCAGCACCGCCTTTTTGAAGTGGA | |
| | | GTAGCCTGAAGGCCTCGGACACCGCCATGTATTTCTGTGCGCGACAGGGT | |
| | | ATAGAAGTGACTGGTACGGGAGGTTTGGACGTCTGGGGCCAAGGGACCAC | |
| | | GGTCACCGTCTCCTCA | |
| V3 | HV-101 | GAGGTGCAGCTGGTGCAGTCTGGAGCAGAGGTGAAAAAGCCCGGGGAGTC | 254 |
| | | TCTGAAGATCTCCTGTAAGGGTTCTGGATACAGTTTTGCGAGCTACTGGA | |
| | | TCGCCTGGGTGCGCCAGATGCCCGGGAAAGGCCTGGAGTGGATGGGGATC | |
| | | ATCTATCCTGGTGACTCTGATACCAGATACAGCCCGTCCTTCCAAGATCA | |
| | | GGTCACCATCTCAGCCGACAAGTCCATCAGCACCGCCTACCTACAGTGGA | |
| | | GCAGCCTGAAGGCCTCGGACACCGCCATGTATTTCTGTGCGAGAGGGAGG | |
| | | ACGTTTTATTATGATAGTAGTGATTATTTTGACTACTGGGGCCAGGGAAC | |
| | | CCTGGTCACCGTGTCCTCA | |
| V24 | HV-102 | GAGGTGCAGCTGGTGCAGTCTGGAGCAGAGGTGAAAAAGCCCGGGGAGTC | 255 |
| | | TCTGAAGATCTCCTGTAAGGGTTCTGGATACAGTTTTACCAGCTACTGGA | |
| | | TTGCCTGGGTGCGCCAGATGCCCGGGAAAGGCCTGGAGTGGATGGGGATC | |
| | | ATCTATCCTGGTGACTCTGATGTGAGATACAGCCCGTCCTTCCAAGGCCA | |
| | | GGTCACCATCTCAGCCGACAAGTCCATCAGCACCGCCTACCTACAGTGGA | |
| | | GCAGCCTGAAGGCCTCGGACACCGCCATGTATTTCTGTGCGAGATCTAGG | |
| | | ACGTTTTATTATGATAGTAGTGATTATTTTGACTACTGGGGCCAGGGAAC | |
| | | CCTGGTCACCGTGTCCTCA | |
| V27 | HV-103 | GAGGTGCAGCTGGTGCAGTCTGGAGCAGAGGTGAAAAAGCCCGGGGAGTC | 256 |
| | | TCTGAAGATCTCCTGTAAGGGTTCTGGATACAGTTTTACCAGCTACTGGA | |
| | | TCGCCTGGGTGCGCCAGATGCCCGGGAAAGGCCTGGAGTGGATGGGGATC | |
| | | ATCTATCCTGGTGACTCTGATACCAGATACGCTCCGTCCTTCCAAGGCCA | |
| | | GGTCACCATCTCAGCCGACAAGTCCATCAGCACCGCCTACCTACAGTGGA | |
| | | GCAGCCTGAAGGCCTCGGACACCGCCATGTATTTCTGTGTGAGAAGTAGG | |
| | | ACGTTTTATTATGATAGTAGTGATTATTTTGACTACTGGGGCCAGGGAAC | |
| | | CCTGGTCACCGTGTCCTCA | |
| V40 | HV-104 | GAGGTGCAGCTGGTGCAGTCTGGAGCAGAGGTGAAAAAGCCCGGGGAGTC | 257 |
| | | TCTGAAGATCTCCTGTAAGGGTTCTGGATACAGTTTTGGGAGCTACTGGA | |
| | | TCGCCTGGGTGCGCCAGATGCCCGGGAAAGGCCTGGAGTGGATGGGGATC | |
| | | ATCTATCCTGGTGACTCTGATGTTAGATACAGCCCGTCCTTCCAAGGCCA | |
| | | GGTCACCATCTCAGCCGACAAGTCCATCAGCACCGCCTACCTACAGTGGA | |
| | | GCAGCCTGAAGGCCTCGGACACCGCCATGTATTTCTGTGCGAGACAAAGG | |
| | | ACGTTTTATTATGATAGTAGTGATTATTCGGACTACTGGGGCCAGGGAAC | |
| | | CCTGGTCACCGTGTCCTCA | |
| V48 | HV-105 | GAGGTGCAGCTGGTGCAGTCTGGAGCAGAGGTGAAAAAGCCCGGGGAGTC | 258 |
| | | TCTGAAGATCTCCTGTAAGGGTTCTGGATACAGTTTTGGTAGCTACTGGA | |
| | | TCGCCTGGGTGCGCCAGATGCCCGGGAAAGGCCTGGAGTGGATGGGGATC | |
| | | ATCTATCCTGGTGACTCTGATGTGAGATACAGCCCGTCCTTCCAAGGCCA | |
| | | GGTCACCATCTCAGCCGACAAGTCCATCAGCACCGCCTACCTACAGTGGA | |
| | | GCAGCCTGAAGGCCTCGGACACCGCCATGTATTTCTGTGCGAGAATGAGG | |
| | | ACGTTTTATTATGATAGTAGTGATTATTTTGACTACTGGGGCCAGGGAAC | |
| | | CCTGGTCACCGTGTCCTCA | |
| V49 | HV-106 | GAGGTGCAGCTGGTGCAGTCTGGAGCAGAGGTGAAAAAGCCCGGGGAGTC | 259 |
| | | TCTGAAGATCTCCTGTAAGGGTTCTGGATACAGTTTTAATAGCTACTGGA | |
| | | TCGCCTGGGTGCGCCAGATGCCCGGGAAAGGCCTGGAGTGGATGGGGACG | |
| | | ATCTATCCTGGTGACTCTGATACCAGACTGAGCCCGTCCTTCCAAGGCCA | |
| | | GGTCACCATCTCAGCCGACAAGTCCATCAGCACCGCCTACCTACAGTGGA | |
| | | GCAGCCTGAAGGCCTCGGACACCGCCATGTATTTCTGTGCGAGAAGTAGG | |
| | | ACGTTTTATTATGATAGTAGTGATTATTTTGACTACTGGGGCCAGGGAAC | |
| | | CCTGGTCACCGTGTCCTCA | |
| V52 | HV-107 | GAGGTGCAGCTGGTGCAGTCTGGAGCAGAGGTGAAAAAGCCCGGGGAGTC | 260 |
| | | TCTGAAGATCTCCTGTAAGGGTTCTGGATACAGTTTTGAGAGCTACTGGA | |
| | | TCGCCTGGGTGCGCCAGATGCCCGGGAAAGGCCTGGAGTGGATGGGGATC | |
| | | ATCTATCCTGGTGACTCTGATACCAGATACAGCCCGTCCTTCCAAGGCCA | |
| | | GGTCACCATCTCAGCCGACAAGTCCATCAGCACCGCCTACCTACAGTGGA | |
| | | GCAGCCTGAAGGCCTCGGACACCGCCATGTATTTCTGTGCGAGAGGGAGG | |
| | | ACGTTTTATTATGATAGTAGTGATTATTTTGACTACTGGGGCCAGGGAAC | |
| | | CCTGGTCACCGTGTCCTCA | |
| V60 | HV-108 | GAGGTGCAGCTGGTGCAGTCTGGAGCAGAGGTGAAAAAGCCCGGGGAGTC | 261 |
| | | TCTGAAGATCTCCTGTAAGGGTTCTGGATACCATTTTACCAGCTACTGGA | |
| | | TCGCCTGGGTGCGCCAGATGCCCGGGAAAGGCCTGGAGTGGATGGGGATC | |
| | | ATCTATCCTGGTGACTCTGATGTGAGATACAGCCCGTCCTTCCAAGGCCA | |
| | | GGTCACCATCTCAGCCGACAAGTCCATCAGCACCGCCTACCTACAGTGGA | |
| | | GCAGCCTGAAGGCCTCGGACACCGCCATGTATTTCTGTGCGAGACAAAGG | |
| | | ACGTTTTATTATGATAGTAGTGATTATAGTGACTACTGGGGCCAGGGAAC | |
| | | CCTGGTCACCGTGTCCTCA | |
| V73 | HV-109 | GAGGTGCAGCTGGTGCAGTCTGGAGCAGAGGTGAAAAAGCCCGGGGAGTC | 262 |
| | | TCTGAAGATCTCCTGTAAGGGTTCTGGATACAGTTTTGGTAGCTACTGGA | |
| | | TCGCCTGGGTGCGCCAGATGCCCGGGAAAGGCCTGGAGTGGATGGGGATC | |
| | | ATCTATCCTGGTGACTCTGATACCAGATACAGCCCGGGGTTCCAAGGCCA | |
| | | GGTCACCATCTCAGCCGACAAGTCCATCAGCACCGCCTACCTACAGTGGA | |
| | | GCAGCCTGAAGGCCTCGGACACCGCCATGTATTTCTGTGCGAGAGGGAGG | |
| | | ACGTTTTATTATGATAGTAGTGATTATTTTGACTACTGGGGCCAGGGAAC | |
| | | CCTGGTCACCGTGTCCTCA | |
| V76 | HV-110 | GAGGTGCAGCTGGTGCAGTCTGGAGCAGAGGTGAAAAAGCCCGGGGAGTC | 263 |
| | | TCTGAAGATCTCCTGTAAGGGTTCTGGATACAGTTTTGGGAGCTACTGGA | |
| | | TCGCCTGGGTGCGCCAGATGCCCGGGAAAGGCCTGGAGTGGATGGGGATC | |
| | | ATCTATCCTGGTGACTCTGATACCAGATACAGCCCGGAGTTCCAAGGCCA | |
| | | GGTCACCATCTCAGCCGACAAGTCCATCAGCACCGCCTACCTACAGTGGA | |
| | | GCAGCCTGAAGGCCTCGGACACCGCCATGTATTTCTGTGCGAGACAAAGG | |
| | | ACGTTTTATTATGATAGTAGTGATTATAGTGACTACTGGGGCCAGGGAAC | |
| | | CCTGGTCACCGTGTCCTCA | |
| V84 | HV-111 | GAGGTGCAGCTGGTGCAGTCTGGAGCAGAGGTGAAAAAGCCCGGGGAGTC | 264 |
| | | TCTGAAGATCTCCTGTAAGGGTTCTGGATACGGGTTTACCAGCTACTGGA | |
| | | TCGCCTGGGTGCGCCAGATGCCCGGGAAAGGCCTGGAGTGGATGGGGATC | |
| | | ATCTATCCTGGTGACAGTGATACCAGATACAGCCCGTCCTTCCAAGGCCA | |
| | | GGTCACCATCTCAGCCGACAAGTCCATCAGCACCGCCTACCTACAGTGGA | |
| | | GCAGCCTGAAGGCCTCGGACACCGCCATGTATTTCTGTGCGAGACAAAGG | |
| | | ACGTTTTATTATGATAGTAGTGATTATTCGGACTACTGGGGCCAGGGAAC | |
| | | CCTGGTCACCGTGTCCTCA | |
| V9 | HV-201 | GAGGTGCAGCTGGTGCAGTCTGGAGCAGAGGTGAAAAAGCCCGGGGAGTC | 319 |
| | | TCTGAAGATCTCCTGTAAGGGTTCTGGATACAGTTTTACCAGCTACTGGA | |
| | | TCGCCTGGGTGCGCCAGATGCCCGGGAAAGGCCTGGAGTGGATGGGGATC | |
| | | ATCTATCCTGGTGACTCTGATACCAGATACAGCCCGTCCTTCCAAGGCCA | |
| | | GGTCACCATCTCAGCCGACAAGTCCATCAGCACCGCCTACCTACAGTGGA | |
| | | GCAGCCTGAAGGCCTCGGACACCGCCATGTATTTCTGTGCGAGACAAAGG | |
| | | GGGTTTTATTATGATAGTAGTGATTATTTTGACTACTGGGGCCAGGGAAC | |
| | | CCTGGTCACCGTGTCCTCA | |
| V10 | HV-15 | GAGGTGCAGCTGGTGCAGTCTGGAGCAGAGGTGAAAAAGCCCGGGGAGTC | 320 |
| V23 | | TCTGAAGATCTCCTGTAAGGGTTCTGGATACAGTTTTACCAGCTACTGGA | |
| V57 | | TCGCCTGGGTGCGCCAGATGCCCGGGAAAGGCCTGGAGTGGATGGGGATC | |
| V70 | | ATCTATCCTGGTGACTCTGATACCAGATACAGCCCGTCCTTCCAAGGCCA | |
| V83 | | GGTCACCATCTCAGCCGACAAGTCCATCAGCACCGCCTACCTACAGTGGA | |
| | | GCAGCCTGAAGGCCTCGGACACCGCCATGTATTTCTGTGCGAGACAAAGG | |
| | | ACGTTTTATTATGATAGTAGTGATTATTTTGACTACTGGGGCCAGGGAAC | |
| | | CCTGGTCACCGTGTCCTCA | |
| V30 | HV-202 | GAGGTGCAGCTGGTGCAGTCTGGAGCAGAGGTGAAAAAGCCCGGGGAGTC | 321 |
| | | TCTGAAGATCTCCTGTAAGGGTTCTGGATCGAGTTTTACCAGCTACTGGA | |
| | | TCGCCTGGGTGCGCCAGATGCCCGGGAAAGGCCTGGAGTGGATGGGGATC | |
| | | ATCTATCCTGGTGACTCTGATACCAGATACAGCCCGTCCTTCCAAGGCCA | |
| | | GGTCACCATCTCAGCCGACAAGTCCATCAGCACCGCCTACCTACAGTGGA | |
| | | GCAGCCTGAAGGCCTCGGACACCGCCATGTATTTCTGTGCGAGACAAAGG | |
| | | ACGTTTTATTATGATAGTAGTGATTATTTTGACTACTGGGGCCAGGGAAC | |
| | | CCTGGTCACCGTGTCCTCA | |
| V33 | HV-203 | GAGGTGCAGCTGGTGCAGTCTGGAGCAGAGGTGAAAAAGCCCGGGGAGTC | 322 |
| | | TCTGAAGATCTCCTGTAAGGGTTCTGGATACAGTTTTACCAGCTACTGGA | |
| | | TCGCCTGGGTGCGCCAGATGCCCGGGAAAGGCCTGGAGTGGATGGGGATC | |
| | | ATCTATCCTGGTGACTCTGATACCAGATACAGCCCGTCCTTCCAAGGCCA | |
| | | GGTCACCATCTCAGCCGACAAGTCCATCAGCACCGCCTACCTACAGTGGA | |
| | | GCAGCCTGAAGGCCTCGGACACCGCCATGTATTTCTGTGCGAGACAAAGG | |
| | | ACGTTTTATGGGGATAGTAGTGATTATTTTGACTACTGGGGCCAGGGAAC | |
| | | CCTGGTCACCGTGTCCTCA | |
| V44 | HV-204 | GAGGTGCAGCTGGTGCAGTCTGGAGCAGAGGTGAAAAAGCCCGGGGAGTC | 323 |
| | | TCTGAAGATCTCCTGTAAGGGTTCTGGATACAGTTTTACCAGCTACTGGA | |
| | | TCGCCTGGGTGCGCCAGATGCCCGGGAAAGGCCTGGAGTGGATGGGGATC | |
| | | ATCTATCCTAGTGACTCTGATACCAGATACAGCCCGTCCTTCCAAGGCCA | |
| | | GGTCACCATCTCAGCCGACAAGTCCATCAGCACCGCCTACCTACAGTGGA | |
| | | GCAGCCTGAAGGCCTCGGACACCGCCATGTATTTCTGTGCGAGACAAAGG | |
| | | ACGTTTTATTATGATAGTAGTGATTATTTTGACTACTGGGGCCAGGGAAC | |
| | | CCTGGTCACCGTGTCCTCA | |
| V68 | HV-205 | GAGGTGCAGCTGGTGCAGTCTGGAGCAGAGGTGAAAAAGCCCGGGGAGTC | 324 |
| | | TCTGAAGATCTCCTGTAAGGGTTCTGGATACAGTTTTACCAGCTACTGGA | |
| | | TCGCCTGGGTGCGCCAGATGCCCGGGAAAGGCCTGGAGTGGATGGGGATC | |
| | | ATCTATCCTGGTGACTCTGATACCAGATACAGCCCGTCCTTCCAAGGCCA | |
| | | GGTCACCATCTCAGCCGACAAGTCCATCAGCACCGCCTACCTACAGTGGA | |
| | | GCAGCCTGAAGGCCTCGGACACCGCCATGTATTTCTGTGCGAGACAAAGG | |
| | | ACGTTTAGGTATGATAGTAGTGATTATTTTGACTACTGGGGCCAGGGAAC | |
| | | CCTGGTCACCGTGTCCTCA | |
| V90 | HV-206 | GAGGTGCAGCTGGTGCAGTCTGGAGCAGAGGTGAAAAAGCCCGGGGAGTC | 325 |
| | | TCTGAAGATCTCCTGTAAGGGTTCTGGATACAGTTTTACCAGCGAGTGGA | |
| | | TCGCCTGGGTGCGCCAGATGCCCGGGAAAGGCCTGGAGTGGATGGGGATC | |
| | | ATCTATCCTGGTGACTCTGATACCAGATACAGCCCGTCCTTCCAAGGCCA | |
| | | GGTCACCATCTCAGCCGACAAGTCCATCAGCACCGCCTACCTACAGTGGA | |
| | | GCAGCCTGAAGGCCTCGGACACCGCCATGTATTTCTGTGCGAGACAAAGG | |
| | | ACGTTTTATTATGATAGTAGTGATTATTTTGACTACTGGGGCCAGGGAAC | |
| | | CCTGGTCACCGTGTCCTCA | |

In some embodiments, an isolated nucleic acid encoding an anti-TREM2 antibody light chain variable region comprises a sequence that is at least 80% identical, at least 90% identical, at least 95% identical, or at least 98% o identical to a sequence selected from SEQ ID NOs: 208-236 and 313-318. In certain embodiments, an isolated nucleic acid encoding an anti-TREM2 antibody light chain variable region comprises a sequence selected from SEQ ID NOs: 208-236 and 313-318. In related embodiments, an isolated nucleic acid encoding an anti-TREM2 antibody heavy chain variable region comprises a sequence that is at least 80% o identical, at least 90% o identical, at least 95% o identical, or at least 98% identical to a sequence selected from SEQ ID NOs: 237-264 and 319-325. In other related embodiments, an isolated nucleic acid encoding an anti-TREM2 antibody heavy chain variable region comprises a sequence selected from SEQ ID NOs: 237-264 and 319-325.

In some embodiments, the polynucleotide encodes the full-length light chain and full-length heavy chain. Exemplary polynucleotide sequences are provided in Table 3F.

In some embodiments, the present invention provides a method for treating adult-onset leukoencephalopathy with axonal spheroids and pigmented glia (ALSP) in a human patient, comprising administering to the patient an anti-TREM2 antibody, for example, anti-TREM2 antibody “Ab-1.” In some embodiments, a method of the invention comprises administering to a human patient a liquid formulation as described herein.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, a patient or subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment or therapy.

A “therapeutically effective amount” or “therapeutically effective dose” of a drug or therapeutic agent, such as anti-TREM2 antibody “Ab-1”, is any amount of the drug that, when used alone or in combination with another therapeutic agent, protects a patient or subject against the onset of a disease, such as ALSP, or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

In preferred embodiments, a therapeutically effective amount of the drug, such as anti-TREM2 antibody “Ab-1”, alone or in combination with another agent, results in a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to decrease severity of at least one disease symptom, to increase frequency and duration of disease symptom-free periods, or to prevent impairment or disability due to the disease affliction in the patient. Physiological safety refers to the level of toxicity, or other adverse physiological effects at the cellular, organ and/or organism level (adverse effects) resulting from administration of the drug.

As used herein, the terms “therapeutic benefit” or “benefit from therapy” refers to a decrease in severity of disease symptoms, an increase in frequency and duration of disease symptom-free periods, or a prevention of impairment or disability due to the disease affliction.

In some embodiments, the anti-TREM2 antibody, e.g., “Ab-1,” is administered to a human patient via an IV infusion. In some embodiments, an IV infusion of anti-TREM2 antibody “Ab-1” is up to about 5 hours, up to about 4 hours, up to about 3 hours, up to about 2 hours, or up to about 60 minutes. In some embodiments, an IV infusion of anti-TREM2 antibody “Ab-1” is from about 5 minutes to about 5 hours, from about 5 minutes to about 4 hours, from about 5 minutes to about 3 hours, from about 5 minutes to about 2 hours, or from about 5 minutes to about 60 minutes. In some embodiments, an IV infusion of anti-TREM2 antibody “Ab-1” is about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 70 minutes, about 80 minutes, or about 90 minutes.

In some embodiments, the anti-TREM2 antibody is administered to a human patient at a dose of up to about 200 mg/kg. In some embodiments, anti-TREM2 antibody “Ab-1” is administered to a human patient at a dose of up to about 200 mg/kg. In some embodiments, anti-TREM2 antibody “Ab-1” is administered to a human patient at a dose of up to about 150 mg/kg. In some embodiments, anti-TREM2 antibody “Ab-1” is administered to a human patient at a dose of up to about 100 mg/kg. In some embodiments, anti-TREM2 antibody “Ab-1” is administered to a human patient at a dose of from about 1 mg/kg to about 100 mg/kg, from about 1 mg/kg to about 90 mg/kg, from about 1 mg/kg to about 80 mg/kg, from about 1 mg/kg to about 70 mg/kg, or from about 1 mg/kg to about 60 mg/kg. In some embodiments, the anti-TREM2 antibody, e.g., “Ab-1,” is administered to a human patient at a dose of from about 10 mg/kg to about 100 mg/kg, from about 10 mg/kg to about 90 mg/kg, from about 10 mg/kg to about 80 mg/kg, from about 10 mg/kg to about 70 mg/kg, from about 10 mg/kg to about 60 mg/kg, from about 10 mg/kg to about 50 mg/kg, from about 10 mg/kg to about 40 mg/kg, from about 10 mg/kg to about 30 mg/kg, or from about 10 mg/kg to about 20 mg/kg. In some embodiments, the anti-TREM2 antibody, e.g., “Ab-1,” is administered to a human patient at a dose of from about 20 mg/kg to about 100 mg/kg, from about 20 mg/kg to about 90 mg/kg, from about 20 mg/kg to about 80 mg/kg, from about 20 mg/kg to about 70 mg/kg, from about 20 mg/kg to about 60 mg/kg, from about 20 mg/kg to about 50 mg/kg, from about 20 mg/kg to about 40 mg/kg, or from about 20 mg/kg to about 30 mg/kg. In some embodiments, the anti-TREM2 antibody, e.g., “Ab-1,” is administered to a human patient at a dose of from about 30 mg/kg to about 100 mg/kg, from about 30 mg/kg to about 90 mg/kg, from about 30 mg/kg to about 80 mg/kg, from about 30 mg/kg to about 70 mg/kg, from about 30 mg/kg to about 60 mg/kg, from about 30 mg/kg to about 50 mg/kg, or from about 30 mg/kg to about 40 mg/kg. In some embodiments, the anti-TREM2 antibody, e.g., “Ab-1,” is administered to a human patient at a dose of from about 40 mg/kg to about 100 mg/kg, from about 40 mg/kg to about 90 mg/kg, from about 40 mg/kg to about 80 mg/kg, from about 40 mg/kg to about 70 mg/kg, from about 40 mg/kg to about 60 mg/kg, or from about 40 mg/kg to about 50 mg/kg. In some embodiments, the anti-TREM2 antibody, e.g., “Ab-1,” is administered to a human patient at a dose of from about 50 mg/kg to about 100 mg/kg, from about 50 mg/kg to about 90 mg/kg, from about 50 mg/kg to about 80 mg/kg, from about 50 mg/kg to about 70 mg/kg, or from about 50 mg/kg to about 60 mg/kg. In some embodiments, anti-TREM2 antibody “Ab-1” is administered to a human patient at a dose of about 1 mg/kg, about 2 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, or about 60 mg/kg.

In some embodiments, an anti-TREM2 antibody is administered to a human patient once daily, 1, 2, 3 or 4 times weekly, or 1, 2, 3 or 4 times monthly. In some embodiments, anti-TREM2 antibody “Ab-1” is administered to a human patient once daily. In some embodiments, anti-TREM2 antibody “Ab-1” is administered to a human patient 1, 2, 3 or 4 times weekly. In some embodiments, anti-TREM2 antibody “Ab-1” is administered to a human patient 1, 2, 3 or 4 times monthly. In some embodiments, anti-TREM2 antibody “Ab-1” is administered to a human patient once every 1, 2, 3, or 4 weeks. In some embodiments, anti-TREM2 antibody “Ab-1” is administered to a human patient once every 2 days, 3 days, 4 days, 5 days, 6 days, 7 days, 8 days, 9 days, 10 days, or 14 days. In some embodiments, anti-TREM2 antibody “Ab-1” is administered to a human patient once weekly.

In some embodiments, the present invention provides a liquid formulation, comprising anti-TREM2 antibody “Ab-1” at a concentration of up to about 300 mg/mL. In some embodiments, the present invention provides a liquid formulation, comprising anti-TREM2 antibody “Ab-1” at a concentration of up to about 250 mg/mL. In some embodiments, the present invention provides a liquid formulation, comprising anti-TREM2 antibody “Ab-1” at a concentration of up to about 200 mg/mL. In some embodiments, the present invention provides a liquid formulation, comprising anti-TREM2 antibody “Ab-1” at a concentration of up to about 150 mg/mL. In some embodiments, the present invention provides a liquid formulation, comprising anti-TREM2 antibody “Ab-1” at a concentration of about 300 mg/mL, about 250 mg/mL, about 200 mg/mL, about 180 mg/mL, about 170 mg/mL, about 160 mg/mL, about 150 mg/mL, about 140 mg/mL, about 130 mg/mL, about 120 mg/mL, about 110 mg/mL, or about 100 mg/mL. In some embodiments, the present invention provides a liquid formulation, comprising anti-TREM2 antibody “Ab-1” at a concentration of about 140 mg/mL. In some embodiments, a method of the invention comprises administering to a human patient a liquid formulation as described herein. In some embodiments, a method of the invention comprises administering to a human patient a liquid formulation, comprising anti-TREM2 antibody “Ab-1” at a concentration of about 140 mg/mL.

In some embodiments, a patient is between 18 to 55 years of age, inclusive. In some embodiments, a patient is between 18 to 42 years of age. In some embodiments, a patient is between 42 and 55 years of age. In some embodiments, a patient is 42 years of age or younger. In some embodiments, a patient is 42 years of age or older.

In some embodiments, a patient is not a WOCBP (women of child-bearing potential). In some embodiments, a patient is a WOCBP who is using an effective contraceptive method during the course of the treatment with the anti-TREM2 antibody and for at least 10 weeks after the treatment.

In some embodiments, a patient is a nonsmoker (or other nicotine/tobacco use including vapor) as determined by history (no nicotine use over the past year). In some embodiments, a patient has a negative urine cotinine test immediately before, or at the time of, being administered anti-TREM2 antibody “Ab-1.”

In some embodiments, a patient has a Body mass index (BMI) between 18.5 and 30.0 kg/m2, inclusive. In some embodiments, a patient is assessed for vital signs (systolic and diastolic blood pressure and pulse rate) immediately before, or at the time of, being administered anti-TREM2 antibody “Ab-1.”

In some embodiments, a patient is a first-generation Japanese ethnic origin. In some embodiments, a patient is born in Japan. In some embodiments, a patient has Parents and grandparents who are ethnically Japanese and born in Japan. In some embodiments, a patient has no significant change in lifestyle since leaving Japan. In some embodiments, a patient is less than 10 Years outside of Japan.

In some embodiments, a patient does not have clinically significant history or evidence of cardiovascular, respiratory, hepatic, renal, gastrointestinal, endocrine, neurological, immunological, or psychiatric disorder(s). In some embodiments, a patient has not been administered a monoclonal antibody therapy within 120 days before being administered anti-TREM2 antibody “Ab-1.” In some embodiments, a patient does not have a history of alcohol and/or illicit drug abuse within 2 years before being administered anti-TREM2 antibody “Ab-1.”

In some embodiments, a patient does not have positive test for Hepatitis B surface antigen (HBsAg), Hepatitis C antibody, or human immunodeficiency virus (HIV) antibody.

In some embodiments, a patient does not have positive urine test for ethanol immediately before, or at the time of, being administered anti-TREM2 antibody “Ab-1.”

In some embodiments, a patient does not have positive urine drug (e.g., cocaine, amphetamines, barbiturates, opiates, benzodiazepines, and cannabinoids) or cotinine tests immediately before, or at the time of, being administered anti-TREM2 antibody “Ab-1.”

In some embodiments, a patient is not a female patient who is breastfeeding immediately before, or at the time of, being administered anti-TREM2 antibody “Ab-1.”

In some embodiments, a patient is not a female patient with a positive serum pregnancy test immediately before, or at the time of, being administered anti-TREM2 antibody “Ab-1.”

In some embodiments, a patient has not donated blood (>500 mL) or blood products within 2 months (56 days) prior to being administered anti-TREM2 antibody “Ab-1.”

In some embodiments, a patient has not been administered any investigational drug within 30 days or 5 half-lives, whichever is longer, prior to being administered anti-TREM2 antibody “Ab-1.”

In some embodiments, a patient does not have a history of hypersensitivity to any therapeutic monoclonal antibodies, or any of the excipients or to medicinal products with similar chemical structures.

In some embodiments, a patient does not have a positive reverse transcription polymerase chain reaction (RT-PCR) test for severe acute respiratory syndrome coronavirus 2 (SARS-CoV-2) immediately before, or at the time of, being administered anti-TREM2 antibody “Ab-1.”

In some embodiments, a patient does not have any clinical signs and symptoms consistent with SARS-CoV-2 infection, e.g., fever, dry cough, dyspnea, sore throat, fatigue, or laboratory confirmed acute infection with SARS-CoV-2 immediately before, or at the time of, being administered anti-TREM2 antibody “Ab-1.”

In some embodiments, a patient does not have a severe course of corona virus disease 2019 (COVID-19; extracorporeal membrane oxygenation, mechanically ventilated, Intensive Care Unit stay).

In some embodiments, a patient does not have any recent (within 14 days prior to being administered anti-TREM2 antibody “Ab-1”) exposure to someone who has COVID-19 symptoms or tested positive for SARS-CoV-2.

In some embodiments, a patient has not received any COVID-19 treatment immediately before, or at the time of, being administered anti-TREM2 antibody “Ab-1.”

In some embodiments, a patient has not received final dose of COVID-19 vaccine within 14 days prior to being administered anti-TREM2 antibody “Ab-1” (i.e., they must have completed their vaccination at least 14 days prior to admission).

In some embodiments, a patient undergoes genetic testing prior to administration of anti-TREM2 antibody “Ab-1.” In some embodiments, a patient does not have an alanyl-tRNA synthetase 2 (AARS2) gene mutation, e.g., as confirmed by genetic testing. In some embodiments, a patient has a colony stimulating factor 2 (CSF1R) gene mutation, e.g., as confirmed by genetic testing. In some embodiments, a patient has a mutation in an exon in the CSF 1R gene, e.g., in exons 1-21. In some embodiments, a patient has a mutation in an exon in the CSF1R gene, e.g., in exons 2-17 or 18-21. In some embodiments, a patient has a sign or symptom of ALSP. Exemplary signs or symptoms of ALSP include cognitive impairment, frontal lobe dysfunction, a psychiatric symptom, an extrapyramidal symptom, a pyramidal symptom, involuntary movements, gait disorder, seizures, pathological reflexes, speech disturbances, swallowing disturbances, apraxia, sensory disturbances, autonomic symptoms, headaches, stroke, dementia, encephalitis, memory loss, depression, executive functioning loss, bradykinesia, rigidity, and cerebellar symptoms.

In some embodiments, a patient exhibits a reduction of a symptom of ALSP upon administration of anti-TREM2 antibody “Ab-1.” In some embodiments, a patient exhibits a reduction in the severity of a symptom of ALSP, e.g., by about 5%, 10%, 15%, 20%, 25%, 30%, 35%, 40%, 45%, 50%, 55%, 60%, 65%, 70%, 75%, 80%, 85%, 90%, 95%, 99%, upon administration of anti-TREM2 antibody “Ab-1,” e.g., compared to the severity of the symptom in the absence of administration of anti-TREM2 antibody “Ab-1.”

In some embodiments, a patient undergoes CSF collection via lumbar punctures immediately before, at the time of, or after being administered anti-TREM2 antibody “Ab-1.” In some embodiments, a patient undergoes CSF collection via lumbar punctures immediately before, at the time of, or after receiving the first dose of anti-TREM2 antibody “Ab-1.” In some embodiments, a patient undergoes CSF collection via lumbar punctures immediately before, at the time of, or after receiving the second dose of anti-TREM2 antibody “Ab-1.” In some embodiments, a patient undergoes CSF collection via lumbar punctures immediately before, at the time of, or after receiving the third dose of anti-TREM2 antibody “Ab-1.” In some embodiments, a patient does not undergo CSF collection via lumbar punctures, wherein the patient is hypersensitive to anesthetic or derivatives used during CSF collection or any medication used to prepare the area of the lumbar puncture. In some embodiments, a patient does not undergo CSF collection via lumbar punctures, wherein the patient had previous CSF collection within 30 days prior to being administered anti-TREM2 antibody “Ab-1.”

In some embodiments, a patient does not undergo CSF collection via lumbar punctures, wherein the patient has a history of vertebral deformities, major lumbar back surgery, clinically significant back pain, clinically significant abnormal X-ray, and/or injury.

In some embodiments, a patient does not undergo CSF collection via lumbar punctures, wherein the patient has an on-going skin infection at the lumbar puncture injection site.

In some embodiments, a patient does not undergo CSF collection via lumbar punctures, wherein the patient has clinically significant coagulation tests values outside the normal reference range (prothrombin time/international normalized ratio, partial thromboplastin time) immediately before, or at the time of, being administered anti-TREM2 antibody “Ab-1.”

In some embodiments, the present invention provides a method for treating adult-onset leukoencephalopathy with axonal spheroids and pigmented glia (ALSP) in a human patient, comprising administering to the patient anti-TREM2 antibody “Ab-1” via an IV infusion at a dose of about 60 mg/kg, about 50 mg/kg, about 40 mg/kg, about 30 mg/kg, about 20 mg/kg, about 10 mg/kg, about 5 mg/kg, about 3 mg/kg, about 2 mg/kg, or about 1 mg/kg, wherein the IV infusion is about 60 minutes in length.

In some embodiments, the present invention provides a method for treating adult-onset leukoencephalopathy with axonal spheroids and pigmented glia (ALSP) in a human patient, comprising administering to the patient anti-TREM2 antibody “Ab-1” via an IV infusion at a dose of about 60 mg/kg, about 50 mg/kg, about 40 mg/kg, about 30 mg/kg, about 20 mg/kg, about 10 mg/kg, about 5 mg/kg, about 3 mg/kg, about 2 mg/kg, or about 1 mg/kg, wherein the IV infusion is about 50 minutes in length.

In some embodiments, the present invention provides a method for treating adult-onset leukoencephalopathy with axonal spheroids and pigmented glia (ALSP) in a human patient, comprising administering to the patient anti-TREM2 antibody “Ab-1” via an IV infusion at a dose of about 60 mg/kg, about 50 mg/kg, about 40 mg/kg, about 30 mg/kg, about 20 mg/kg, about 10 mg/kg, about 5 mg/kg, about 3 mg/kg, about 2 mg/kg, or about 1 mg/kg, wherein the IV infusion is about 45 minutes in length.

In some embodiments, the present invention provides a method for treating adult-onset leukoencephalopathy with axonal spheroids and pigmented glia (ALSP) in a human patient, comprising administering to the patient anti-TREM2 antibody “Ab-1” via an IV infusion at a dose of about 60 mg/kg, about 50 mg/kg, about 40 mg/kg, about 30 mg/kg, about 20 mg/kg, about 10 mg/kg, about 5 mg/kg, about 3 mg/kg, about 2 mg/kg, or about 1 mg/kg, wherein the IV infusion is about 40 minutes in length.

In some embodiments, the present invention provides a method for treating adult-onset leukoencephalopathy with axonal spheroids and pigmented glia (ALSP) in a human patient, comprising administering to the patient anti-TREM2 antibody “Ab-1” via an IV infusion at a dose of about 60 mg/kg, about 50 mg/kg, about 40 mg/kg, about 30 mg/kg, about 20 mg/kg, about 10 mg/kg, about 5 mg/kg, about 3 mg/kg, about 2 mg/kg, or about 1 mg/kg, wherein the IV infusion is about 35 minutes in length.

In some embodiments, the present invention provides a method for treating adult-onset leukoencephalopathy with axonal spheroids and pigmented glia (ALSP) in a human patient, comprising administering to the patient anti-TREM2 antibody “Ab-1” via an IV infusion at a dose of about 60 mg/kg, about 50 mg/kg, about 40 mg/kg, about 30 mg/kg, about 20 mg/kg, about 10 mg/kg, about 5 mg/kg, about 3 mg/kg, about 2 mg/kg, or about 1 mg/kg, wherein the IV infusion is about 30 minutes in length.

In some embodiments, the present invention provides a method for treating adult-onset leukoencephalopathy with axonal spheroids and pigmented glia (ALSP) in a human patient, comprising administering to the patient anti-TREM2 antibody “Ab-1” via an IV infusion at a dose of about 60 mg/kg, about 50 mg/kg, about 40 mg/kg, about 30 mg/kg, about 20 mg/kg, about 10 mg/kg, about 5 mg/kg, about 3 mg/kg, about 2 mg/kg, or about 1 mg/kg, wherein the IV infusion is about 25 minutes in length.

In some embodiments, the present invention provides a method for treating adult-onset leukoencephalopathy with axonal spheroids and pigmented glia (ALSP) in a human patient, comprising administering to the patient anti-TREM2 antibody “Ab-1” via an IV infusion at a dose of about 60 mg/kg, about 50 mg/kg, about 40 mg/kg, about 30 mg/kg, about 20 mg/kg, about 10 mg/kg, about 5 mg/kg, about 3 mg/kg, about 2 mg/kg, or about 1 mg/kg, wherein the IV infusion is about 20 minutes in length.

In some embodiments, the present invention provides a method for treating adult-onset leukoencephalopathy with axonal spheroids and pigmented glia (ALSP) in a human patient, comprising administering to the patient anti-TREM2 antibody “Ab-1” via an IV infusion at a dose of about 60 mg/kg, about 50 mg/kg, about 40 mg/kg, about 30 mg/kg, about 20 mg/kg, about 10 mg/kg, about 5 mg/kg, about 3 mg/kg, about 2 mg/kg, or about 1 mg/kg, wherein the IV infusion is about 15 minutes in length.

In some embodiments, the present invention provides a method for treating adult-onset leukoencephalopathy with axonal spheroids and pigmented glia (ALSP) in a human patient, comprising administering to the patient anti-TREM2 antibody “Ab-1” via an IV infusion at a dose of about 60 mg/kg, about 50 mg/kg, about 40 mg/kg, about 30 mg/kg, about 20 mg/kg, about 10 mg/kg, about 5 mg/kg, about 3 mg/kg, about 2 mg/kg, or about 1 mg/kg, wherein the IV infusion is about 10 minutes in length.

In some embodiments, the present invention provides a method for treating adult-onset leukoencephalopathy with axonal spheroids and pigmented glia (ALSP) in a human patient, comprising administering to the patient anti-TREM2 antibody “Ab-1” via an IV infusion at a dose of about 60 mg/kg, about 50 mg/kg, about 40 mg/kg, about 30 mg/kg, about 20 mg/kg, about 10 mg/kg, about 5 mg/kg, about 3 mg/kg, about 2 mg/kg, or about 1 mg/kg, wherein the IV infusion is about 5 minutes in length.

In some embodiments, the present invention provides a method for treating adult-onset leukoencephalopathy with axonal spheroids and pigmented glia (ALSP) in a human patient, comprising administering to the patient anti-TREM2 antibody “Ab-1” via an IV infusion at a dose of about 4.2 gram, about 3.5 gram, about 2.8 gram, about 2.1 gram, about 1.4 gram, about 700 mg, about 350 mg, about 210 mg, about 140 mg, or about 70 mg, wherein the IV infusion is about 60 minutes in length.

In some embodiments, the present invention provides a method for treating adult-onset leukoencephalopathy with axonal spheroids and pigmented glia (ALSP) in a human patient, comprising administering to the patient anti-TREM2 antibody “Ab-1” via an IV infusion at a dose of about 4.2 gram, about 3.5 gram, about 2.8 gram, about 2.1 gram, about 1.4 gram, about 700 mg, about 350 mg, about 210 mg, about 140 mg, or about 70 mg, wherein the IV infusion is about 50 minutes in length.

In some embodiments, the present invention provides a method for treating adult-onset leukoencephalopathy with axonal spheroids and pigmented glia (ALSP) in a human patient, comprising administering to the patient anti-TREM2 antibody “Ab-1” via an IV infusion at a dose of about 4.2 gram, about 3.5 gram, about 2.8 gram, about 2.1 gram, about 1.4 gram, about 700 mg, about 350 mg, about 210 mg, about 140 mg, or about 70 mg, wherein the IV infusion is about 45 minutes in length.

In some embodiments, the present invention provides a method for treating adult-onset leukoencephalopathy with axonal spheroids and pigmented glia (ALSP) in a human patient, comprising administering to the patient anti-TREM2 antibody “Ab-1” via an IV infusion at a dose of about 4.2 gram, about 3.5 gram, about 2.8 gram, about 2.1 gram, about 1.4 gram, about 700 mg, about 350 mg, about 210 mg, about 140 mg, or about 70 mg, wherein the IV infusion is about 40 minutes in length.

In some embodiments, the present invention provides a method for treating adult-onset leukoencephalopathy with axonal spheroids and pigmented glia (ALSP) in a human patient, comprising administering to the patient anti-TREM2 antibody “Ab-1” via an IV infusion at a dose of about 4.2 gram, about 3.5 gram, about 2.8 gram, about 2.1 gram, about 1.4 gram, about 700 mg, about 350 mg, about 210 mg, about 140 mg, or about 70 mg, wherein the IV infusion is about 35 minutes in length.

In some embodiments, the present invention provides a method for treating adult-onset leukoencephalopathy with axonal spheroids and pigmented glia (ALSP) in a human patient, comprising administering to the patient anti-TREM2 antibody “Ab-1” via an IV infusion at a dose of about 4.2 gram, about 3.5 gram, about 2.8 gram, about 2.1 gram, about 1.4 gram, about 700 mg, about 350 mg, about 210 mg, about 140 mg, or about 70 mg, wherein the IV infusion is about 30 minutes in length.

In some embodiments, the present invention provides a method for treating adult-onset leukoencephalopathy with axonal spheroids and pigmented glia (ALSP) in a human patient, comprising administering to the patient anti-TREM2 antibody “Ab-1” via an IV infusion at a dose of about 4.2 gram, about 3.5 gram, about 2.8 gram, about 2.1 gram, about 1.4 gram, about 700 mg, about 350 mg, about 210 mg, about 140 mg, or about 70 mg, wherein the IV infusion is about 25 minutes in length.

In some embodiments, the present invention provides a method for treating adult-onset leukoencephalopathy with axonal spheroids and pigmented glia (ALSP) in a human patient, comprising administering to the patient anti-TREM2 antibody “Ab-1” via an IV infusion at a dose of about 4.2 gram, about 3.5 gram, about 2.8 gram, about 2.1 gram, about 1.4 gram, about 700 mg, about 350 mg, about 210 mg, about 140 mg, or about 70 mg, wherein the IV infusion is about 20 minutes in length.

In some embodiments, the present invention provides a method for treating adult-onset leukoencephalopathy with axonal spheroids and pigmented glia (ALSP) in a human patient, comprising administering to the patient anti-TREM2 antibody “Ab-1” via an IV infusion at a dose of about 4.2 gram, about 3.5 gram, about 2.8 gram, about 2.1 gram, about 1.4 gram, about 700 mg, about 350 mg, about 210 mg, about 140 mg, or about 70 mg, wherein the IV infusion is about 15 minutes in length.

In some embodiments, the present invention provides a method for treating adult-onset leukoencephalopathy with axonal spheroids and pigmented glia (ALSP) in a human patient, comprising administering to the patient anti-TREM2 antibody “Ab-1” via an IV infusion at a dose of about 4.2 gram, about 3.5 gram, about 2.8 gram, about 2.1 gram, about 1.4 gram, about 700 mg, about 350 mg, about 210 mg, about 140 mg, or about 70 mg, wherein the IV infusion is about 10 minutes in length.

In some embodiments, the present invention provides a method for treating adult-onset leukoencephalopathy with axonal spheroids and pigmented glia (ALSP) in a human patient, comprising administering to the patient anti-TREM2 antibody “Ab-1” via an IV infusion at a dose of about 4.2 gram, about 3.5 gram, about 2.8 gram, about 2.1 gram, about 1.4 gram, about 700 mg, about 350 mg, about 210 mg, about 140 mg, or about 70 mg, wherein the IV infusion is about 5 minutes in length.

In some embodiments, the present invention provides a method of reducing a level of sTREM2 in a subject, comprising administering anti-TREM2 antibody VGL101 to the subject at a dose of about 1-100 mg/kg. In some embodiments, the level of sTREM2 is evaluated prior to administering VGL101. In some embodiments, the level of sTREM2 is the level in cerebrospinal fluid (CSF). In some embodiments, the level of sTREM2 is reduced by about 10%, about 20%, about 30%, about 40%, about 50%, about 60%, about 70%, about 80, about 90%, or about 100%.

In some embodiments, the method comprises administering to the subject a dose of about 1-75 mg/kg of the anti-TREM2 antibody. In some embodiments, the method comprises administering to the human patient at a dose of about 1-60 mg/kg of the anti-TREM2 antibody. In some embodiments, the method comprises administering to the human patient at a dose of about 1-50 mg/kg of the anti-TREM2 antibody. In some embodiments, the method comprises administering to the human patient at a dose of about 1-40 mg/kg of the anti-TREM2 antibody. In some embodiments, the method comprises administering to the human patient at a dose of about 1 mg/kg, about 3 mg/kg, about 10 mg/kg, about 20 mg/kg, or about 40 mg/kg of the anti-TREM2 antibody. In some embodiments, the method comprises administering the anti-TREM2 antibody to the human patient once weekly.

In some embodiments, the present invention provides a method of reducing a level of sCSF1R in a subject, comprising administering anti-TREM2 antibody VGL101 to the subject at a dose of about 1-100 mg/kg. In some embodiments, the level of sCSF1R is evaluated prior to administering VGL101. In some embodiments, the level of sCSF1R is the level in cerebrospinal fluid (CSF). In some embodiments, the level of sCSF1R is reduced by about 10%, about 20%, about 30%, about 40%, about 50%, about 60%, about 70%, about 80, about 90%, or about 100%.

In some embodiments, the method comprises administering to the subject a dose of about 1-75 mg/kg of the anti-TREM2 antibody. In some embodiments, the method comprises administering to the human patient at a dose of about 1-60 mg/kg of the anti-TREM2 antibody. In some embodiments, the method comprises administering to the human patient at a dose of about 1-50 mg/kg of the anti-TREM2 antibody. In some embodiments, the method comprises administering to the human patient at a dose of about 1-40 mg/kg of the anti-TREM2 antibody. In some embodiments, the method comprises administering to the human patient at a dose of about 1 mg/kg, about 3 mg/kg, about 10 mg/kg, about 20 mg/kg, or about 40 mg/kg of the anti-TREM2 antibody. In some embodiments, the method comprises administering the anti-TREM2 antibody to the human patient once weekly.

In some embodiments, the present invention provides a method of engaging or agonizing TREM2 in a subject, comprising administering anti-TREM2 antibody VGL101 to the subject at a dose of about 1-100 mg/kg. In some embodiments, the method comprises administering to the subject a dose of about 1-75 mg/kg of the anti-TREM2 antibody. In some embodiments, the method comprises administering to the human patient at a dose of about 1-60 mg/kg of the anti-TREM2 antibody. In some embodiments, the method comprises administering to the human patient at a dose of about 1-50 mg/kg of the anti-TREM2 antibody. In some embodiments, the method comprises administering to the human patient at a dose of about 1-40 mg/kg of the anti-TREM2 antibody. In some embodiments, the method comprises administering to the human patient at a dose of about 1 mg/kg, about 3 mg/kg, about 10 mg/kg, about 20 mg/kg, or about 40 mg/kg of the anti-TREM2 antibody. In some embodiments, the method comprises administering the anti-TREM2 antibody to the human patient once weekly.

### Liquid Formulations

In some embodiments, the invention provides a liquid formulation comprising anti-TREM2 antibody “Ab-1,” and a pharmaceutically acceptable excipient (e.g., a buffer) and/or carrier (e.g., water). The amount of anti-TREM2 antibody “Ab-1” in liquid formulations of this invention is such that it is effective to measurably inhibit TREM2, or a mutant thereof, in a patient. In certain embodiments, a liquid formulation of this invention is formulated for administration to a patient in need of such composition. In some embodiments, a composition of this invention is formulated for parenteral (e.g., intravenous) administration to a patient.

In some embodiments, a liquid formulation of the invention comprises anti-TREM2 antibody “Ab-1” at a concentration of from about 50 mg/mL to about 250 mg/mL. In some embodiments, a liquid formulation of the invention comprises anti-TREM2 antibody “Ab-1” at a concentration of from about 70 mg/mL to about 230 mg/mL, from about 90 mg/mL to about 210 mg/mL, from about 100 mg/mL to about 200 mg/mL, from about 120 mg/mL to about 180 mg/mL, from about 120 mg/mL to about 160 mg/mL, or from about 130 mg/mL to about 150 mg/mL. In some embodiments, a liquid formulation of the invention comprises anti-TREM2 antibody “Ab-1” at a concentration of about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, or about 200 mg/mL. In some embodiments, a liquid formulation of the invention comprises anti-TREM2 antibody “Ab-1” at a concentration of about 135 mg/mL, about 136 mg/mL, about 137 mg/mL, about 138 mg/mL, about 139 mg/mL, about 140 mg/mL, about 141 mg/mL, about 142 mg/mL, about 143 mg/mL, about 144 mg/mL, or about 145 mg/mL.

In some embodiments, a liquid formulation of the invention comprises sodium acetate. In some embodiments, a liquid formulation of the invention comprises sodium acetate at a concentration of from about 5 mM to about 25 mM. In some embodiments, a liquid formulation of the invention comprises sodium acetate at a concentration of from about 6 mM to about 24 mM, from about 7 mM to about 23 mM, from about 8 mM to about 22 mM, from about 9 mM to about 21 mM, from about 10 mM to about 20 mM, from about 11 mM to about 19 mM, from about 12 mM to about 18 mM, from about 13 mM to about 17 mM, or from about 14 mM to about 16 mM. In some embodiments, a liquid formulation of the invention comprises sodium acetate at a concentration of about 10 mM, about 11 mM, about 12 mM, about 13 mM, about 14 mM, about 15 mM, about 16 mM, about 17 mM, about 18 mM, about 19 mM, or about 20 mM. In some embodiments, a liquid formulation of the invention comprises sodium acetate at a concentration of about 14.5 mM, about 14.6 mM, about 14.7 mM, about 14.8 mM, about 14.9 mM, about 15 mM, about 15.1 mM, about 15.2 mM, about 15.3 mM, about 15.4 mM, or about 15.5 mM.

In some embodiments, a liquid formulation of the invention comprises sucrose. In some embodiments, a liquid formulation of the invention comprises sucrose at a concentration of from about 4% to about 14% (w/v) of total liquid formulation volume. In some embodiments, a liquid formulation of the invention comprises sucrose at a concentration of from about 5% to about 13%, from about 6% to about 12%, from about 7% to about 11%, or from about 8% to about 10% (w/v) of total liquid formulation volume. In some embodiments, a liquid formulation of the invention comprises sucrose at a concentration of about 5%, about 6%, about 7%, about 8%, about 9%, about 10%, about 11%, or about 12% (w/v) of total liquid formulation volume. In some embodiments, a liquid formulation of the invention comprises sucrose at a concentration of about 8.5%, about 8.6%, about 8.7%, about 8.8%, about 8.9%, about 9%, about 9.1%, about 9.2%, about 9.3%, about 9.4%, or about 9.5% (w/v) of total liquid formulation volume.

In some embodiments, a liquid formulation of the invention comprises Polysorbate 80. In some embodiments, a liquid formulation of the invention comprises Polysorbate 80 at a concentration of from about 0.005% to about 0.015% (w/v) of total liquid formulation volume. In some embodiments, a liquid formulation of the invention comprises Polysorbate 80 at a concentration of from about 0.006% to about 0.014%, from about 0.007% to about 0.013%, from about 0.008% to about 0.012%, or from about 0.009% to about 0.011% (w/v) of total liquid formulation volume. In some embodiments, a liquid formulation of the invention comprises Polysorbate 80 at a concentration of about 0.005%, about 0.006%, about 0.007%, about 0.008%, about 0.009%, about 0.01%, about 0.011%, about 0.012%, about 0.013%, about 0.014%, or about 0.015% (w/v) of total liquid formulation volume.

In some embodiments, a liquid formulation of the invention is at a pH of from about 4.4 to about 6.0. In some embodiments, a liquid formulation of the invention is at a pH of from about 4.4 to about 6.0, from about 4.5 to about 5.9, from about 4.6 to about 5.8, from about 4.7 to about 5.7, from about 4.8 to about 5.6, from about 4.9 to about 5.5, from about 5.0 to about 5.4, or from about 5.1 to about 5.3. In some embodiments, a liquid formulation of the invention is at about pH 4.8, about pH 4.9, about pH 5.0, about pH 5.1, about pH 5.2, about pH 5.3, about pH 5.4, about pH 5.5, or about pH 5.6. In some embodiments, a liquid formulation of the invention is at about pH 5.16, about pH 5.17, about pH 5.18, about pH 5.19, about pH 5.20, about pH 5.21, about pH 5.22, about pH 5.23, or about pH 5.24.

In certain embodiments, a liquid formulation of the invention comprises anti-TREM2 antibody “Ab-1,” at a concentration of about 140 mg/mL. In some embodiments, a liquid formulation of the invention comprises sodium acetate at a concentration of about 15 mM. In some embodiments, a liquid formulation of the invention comprises sucrose at a concentration of about 9% (w/v) of total liquid formulation volume. In some embodiments, a liquid formulation of the invention comprises Polysorbate 80 at a concentration of about 0.01% (w/v) of total liquid formulation volume. In some embodiments, a liquid formulation of the invention is at about pH 5.2.

In certain embodiments, the present invention provides a liquid formulation, comprising: anti-TREM2 antibody “Ab-1” at a concentration of about 140 mg/mL;
sodium acetate at a concentration of about 15 mM;
sucrose at a concentration of about 9% (w/v) of total liquid formulation volume;
Polysorbate 80 at a concentration of about 0.01% (w/v) of total liquid formulation volume; and
at about pH 5.2.

The liquid formulation of the present invention may be administered parenterally by injection, infusion, or implantation (intravenous, intramuscular, subcutaneous, or the like) as the liquid formulation or via suitable delivery devices or implants containing conventional, non-toxic pharmaceutically acceptable carriers and adjuvants.

Where necessary, the liquid formulation may also include a solubilizing agent. The components of the formulation can be either separately or mixed together in unit dosage form, for example, as a dry lyophilized powder (which can be reconstituted before use with a carrier such as saline) or concentrated solution in a hermetically sealed container such as an ampoule or sachet indicating the amount of active agent. If the composition is to be administered by infusion, it can be dispensed with an infusion bottle or bag containing sterile pharmaceutical grade water or saline. Where the formulation is administered by injection, an ampoule of sterile water or saline can be provided so that the ingredients may be mixed prior to injection.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride. In preferred aspects, water is added to a liquid formulation of the present invention.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to buffers, surfactants, dispersants, emulsifiers, viscosity modifying agents, and combination thereof.

Suitable surfactants may be anionic, cationic, amphoteric, or nonionic surface-active agents. Suitable anionic surfactants include, but are not limited to, those containing carboxylate, sulfonate, and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene, and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta-alanine, sodium N-lauryloiminodipropionate, myristoamphoacetate, lauryl betaine, and lauryl sulfobetaine. The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

Water-soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

In some embodiments, the liquid formulation of the present invention is mixed with an IV infusion vehicle. In some embodiments, the liquid formulation is mixed with an injectable medium such as normal saline (0.9% sodium chloride), 5% dextrose (D5W), and lactated ringer's injection. In some embodiments, the invention provides a liquid pharmaceutical composition prepared by mixing a liquid formulation of the invention with water, followed by dilution with saline or 5% dextrose. In some embodiments, a liquid pharmaceutical composition is diluted into a saline or 5% dextrose IV bag for IV administration. In some embodiments, a liquid pharmaceutical composition in a saline or 5% dextrose IV bag is stored under room temperature (about 20-25° C.) for up to about 4 hours before IV administration. In some embodiments, a liquid pharmaceutical composition in a saline or 5% dextrose IV bag is stored under refrigerated (about 2-8° C.) conditions for up to about 20 hours before IV administration. In some embodiments, a liquid pharmaceutical composition in a saline or 5% dextrose IV bag is stored under refrigerated (about 2-8° C.) conditions for up to about 20 hours, followed by storage under room temperature (about 20-25° C.) for up to about 4 hours, before IV administration.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated.

### ENUMERATED EMBODIMENTS

The disclosure herein is further presented as a non-limiting list of numbered embodiments.
1. A method for treating adult-onset leukoencephalopathy with axonal spheroids and pigmented glia (ALSP) in a human patient, comprising administering to the patient in need thereof an anti-TREM2 antibody at a dose of about 1-100 mg/kg.
2. The method of embodiment 1, wherein the anti-TREM2 antibody comprises a light chain variable region comprising a CDRL1 having an amino acid sequence according to SEQ ID NO: 2; a CDRL2 having an amino acid sequence according to SEQ ID NO: 3; and a CDRL3 having an amino acid sequence according to SEQ ID NO: 4, and a heavy chain variable region comprising a CDRH1 having an amino acid sequence according to SEQ ID NO: 6; a CDRH2 having an amino acid sequence according to SEQ ID NO: 7; and a CDRH3 having an amino acid sequence according to SEQ ID NO: 8.
3. The method of any one of the preceding embodiments, wherein the anti-TREM2 antibody comprises a light chain variable region having an amino acid sequence according to SEQ ID NO: 1, and a heavy chain variable region having an amino acid sequence according to SEQ ID NO: 5.
4. The method of any one of the preceding embodiments, wherein the anti-TREM2 antibody is an IgG, optionally an IgG₁.
5. The method of any one of the preceding embodiments, wherein the anti-TREM2 antibody comprises a kappa light constant region.
6. The method of any one of the preceding embodiments, wherein the anti-TREM2 antibody is an IgG₁ comprising a variant constant region having one or more mutations selected from R292C, N297G, V302C, D356E, or L358M, according to EU numbering.
7. The method of any one of the preceding embodiments, wherein the anti-TREM2 antibody is anti-TREM2 antibody VGL101, comprising a light chain having an amino acid sequence of SEQ ID NO: 9, and a heavy chain having amino acid sequence of SEQ ID NO: 10.
8. The method of any one of embodiments 1-7, wherein the method comprises administering to the human patient at a dose of about 1-75 mg/kg of the anti-TREM2 antibody.
9. The method of any one of embodiments 1-7, wherein the method comprises administering to the human patient at a dose of about 1-60 mg/kg of the anti-TREM2 antibody.
10. The method of any one of embodiments 1-7, wherein the method comprises administering to the human patient at a dose of about 1-50 mg/kg of the anti-TREM2 antibody.
11. The method of any one of embodiments 1-7, wherein the method comprises administering to the human patient at a dose of about 1-40 mg/kg of the anti-TREM2 antibody.
12. The method of any one of embodiments 1-7, wherein the method comprises administering to the human patient at a dose of about 10-60 mg/kg of the anti-TREM2 antibody.
13. The method of any one of embodiments 1-7, wherein the method comprises administering to the human patient at a dose of about 20-40 mg/kg of the anti-TREM2 antibody.
14. The method of any one of embodiments 1-7, wherein the method comprises administering to the human patient at a dose of about 1 mg/kg, about 3 mg/kg, about 10 mg/kg, about 20 mg/kg, or about 40 mg/kg of the anti-TREM2 antibody.
15. The method of any one of embodiments 1-14, wherein the method comprises administering the anti-TREM2 antibody to the human patient once weekly.
16. The method of any one of embodiments 1-14, wherein the method comprises administering the anti-TREM2 antibody to the human patient once every two weeks.
17. The method of any one of embodiments 1-14, wherein the method comprises administering the anti-TREM2 antibody to the human patient once monthly.
18. The method of any one of embodiments 1-17, wherein the method comprises administering the anti-TREM2 antibody to the human patient via an IV infusion.
19. The method of embodiment 18, wherein the IV infusion of the anti-TREM2 antibody is between 1-4 hours, e.g., about 4 hours, 3 hours, 2 hours, about 60 minutes, about 45 minutes, about 30 minutes.
20. A liquid formulation comprising anti-TREM2 antibody VGL101 at a concentration of about 140 mg/mL, and a pharmaceutically acceptable excipient and/or carrier.
21. The liquid formulation of embodiment 20, further comprising sodium acetate at a concentration of about 15 mM.
22. The liquid formulation of embodiment 20 or 21, further comprising sucrose at a concentration of about 9% (w/v) of total liquid formulation volume.
23. The liquid formulation of any one of embodiments 20-22, further comprising Polysorbate 80 at a concentration of about 0.01% (w/v) of total liquid formulation volume.
24. The liquid formulation of any one of embodiments 20-23, wherein the liquid formulation is at about pH 5.2.
25. A liquid formulation at about pH 5.2, comprising:
anti-TREM2 antibody VGL101 at a concentration of about 140 mg/mL;
sodium acetate at a concentration of about 15 mM;
sucrose at a concentration of about 9% (w/v) of total liquid formulation volume; and Polysorbate 80 at a concentration of about 0.01% (w/v) of total liquid formulation volume.
26. A method for treating adult-onset leukoencephalopathy with axonal spheroids and pigmented glia (ALSP) in a human patient, comprising administering to the patient in need thereof a therapeutically effective amount of the liquid formulation of any one of embodiments 20-25.
27. The method of embodiment 26, wherein the method comprises administering to the human patient at a dose of about 1-60 mg/kg of anti-TREM2 antibody VGL101.
28. The method of embodiment 26, wherein the method comprises administering to the human patient at a dose of about 20-40 mg/kg of anti-TREM2 antibody VGL101.
29. The method of embodiment 26, wherein the method comprises administering to the human patient at a dose of about 1 mg/kg, about 3 mg/kg, about 10 mg/kg, about 20 mg/kg, or about 40 mg/kg of anti-TREM2 antibody VGL101.
30. The method of any one of embodiments 26-29, wherein the method comprises administering anti-TREM2 antibody VGL101 to the human patient once weekly.
31. The method of any one of embodiments 26-29, wherein the method comprises administering the anti-TREM2 antibody to the human patient once every two weeks.
32. The method of any one of embodiments 26-29, wherein the method comprises administering the anti-TREM2 antibody to the human patient once monthly.
33. The method of any one of embodiments 26-32, wherein the method comprises administering anti-TREM2 antibody VGL101 to the human patient via an IV infusion.
34. The method of embodiment 33, wherein the IV infusion of anti-TREM2 antibody VGL101 is about 60 minutes.
35. A method of reducing a level of sTREM2 in a subject, comprising administering anti-TREM2 antibody VGL101 to the subject at a dose of about 1-100 mg/kg.
36. The method of embodiment 35, wherein the level of sTREM2 is evaluated prior to administering VGL101.
37. The method of embodiment 35 or 36, wherein the level of sTREM2 is the level in cerebrospinal fluid (CSF).
38. The method of embodiment 35 or 36, wherein the level of sTREM2 is the level in serum.
39. The method of any one of embodiments 35-38, wherein the level of sTREM2 is reduced by about 10%, about 20%, about 30%, about 40%, about 50%, about 60%, about 70%, about 80, about 90%, or about 100%.
40. The method of any one of embodiments 35-39, wherein the method comprises administering to the subject a dose of about 1-75 mg/kg of the anti-TREM2 antibody.
41. The method of any one of embodiments 35-39, wherein the method comprises administering to the human patient at a dose of about 1-60 mg/kg of the anti-TREM2 antibody.
42. The method of any one of embodiments 35-39, wherein the method comprises administering to the human patient at a dose of about 1-50 mg/kg of the anti-TREM2 antibody.
43. The method of any one of embodiments 35-39, wherein the method comprises administering to the human patient at a dose of about 1-40 mg/kg of the anti-TREM2 antibody.
44. The method of any one of embodiments 35-39, wherein the method comprises administering to the human patient at a dose of about 10-60 mg/kg of the anti-TREM2 antibody.
45. The method of any one of embodiments 35-39, wherein the method comprises administering to the human patient at a dose of about 20-40 mg/kg of the anti-TREM2 antibody.
46. The method of any one of embodiments 35-39, wherein the method comprises administering to the human patient at a dose of about 1 mg/kg, about 3 mg/kg, about 10 mg/kg, about 20 mg/kg, or about 40 mg/kg of the anti-TREM2 antibody.
47. The method of any one of embodiments 35-46, wherein the method comprises administering the anti-TREM2 antibody to the human patient once weekly.
48. The method of any one of embodiments 35-46, wherein the method comprises administering the anti-TREM2 antibody to the human patient once every two weeks.
49. The method of any one of embodiments 35-46, wherein the method comprises administering the anti-TREM2 antibody to the human patient once monthly.
50. The method of any one of embodiments 35-49, wherein the method comprises administering anti-TREM2 antibody VGL101 to the human patient via an IV infusion.
51. The method of embodiment 50, wherein the IV infusion of anti-TREM2 antibody VGL101 is about 60 minutes.
52. A method of reducing a level of sCSF1R in a subject, comprising administering anti-TREM2 antibody VGL101 to the subject at a dose of about 1-100 mg/kg.
53. The method of embodiment 52, wherein the level of sCSF1R is evaluated prior to administering VGL101.
54. The method of embodiment 52 or 53, wherein the level of sCSF1R is the level in cerebrospinal fluid (CSF).
55. The method of embodiment 52 or 53, wherein the level of sCSF1R is the level in serum.
56. The method of any one of embodiments 52-55, wherein the level of sCSF1R is reduced by about 10%, about 20%, about 30%, about 40%, about 50%, about 60%, about 70%, about 80, about 90%, or about 100%.
57. The method of any one of embodiments 52-56, wherein the method comprises administering to the subject a dose of about 1-75 mg/kg of the anti-TREM2 antibody.
58. The method of any one of embodiments 52-56, wherein the method comprises administering to the human patient at a dose of about 1-60 mg/kg of the anti-TREM2 antibody.
59. The method of any one of embodiments 52-56, wherein the method comprises administering to the human patient at a dose of about 1-50 mg/kg of the anti-TREM2 antibody.
60. The method of any one of embodiments 52-56, wherein the method comprises administering to the human patient at a dose of about 1-40 mg/kg of the anti-TREM2 antibody.
61. The method of any one of embodiments 52-56, wherein the method comprises administering to the human patient at a dose of about 10-60 mg/kg of the anti-TREM2 antibody.
62. The method of any one of embodiments 52-56, wherein the method comprises administering to the human patient at a dose of about 20-40 mg/kg of the anti-TREM2 antibody.
63. The method of any one of embodiments 52-56, wherein the method comprises administering to the human patient at a dose of about 1 mg/kg, about 3 mg/kg, about 10 mg/kg, about 20 mg/kg, or about 40 mg/kg of the anti-TREM2 antibody.
64. The method of any one of embodiments 52-63, wherein the method comprises administering the anti-TREM2 antibody to the human patient once weekly.
65. The method of any one of embodiments 52-63, wherein the method comprises administering the anti-TREM2 antibody to the human patient once every two weeks.
66. The method of any one of embodiments 52-63, wherein the method comprises administering the anti-TREM2 antibody to the human patient once monthly.
67. The method of any one of embodiments 52-66, wherein the method comprises administering anti-TREM2 antibody VGL101 to the human patient via an IV infusion.
68. The method of embodiment 67, wherein the IV infusion of anti-TREM2 antibody VGL101 is about 60 minutes.
69. A method of engaging or agonizing TREM2 in a subject, comprising administering anti-TREM2 antibody VGL101 to the subject at a dose of about 1-100 mg/kg.
70. The method of embodiment 69, wherein the method comprises administering to the subject a dose of about 1-75 mg/kg of the anti-TREM2 antibody.
71. The method of embodiment 69, wherein the method comprises administering to the human patient at a dose of about 1-60 mg/kg of the anti-TREM2 antibody.
72. The method of embodiment 69, wherein the method comprises administering to the human patient at a dose of about 1-50 mg/kg of the anti-TREM2 antibody.
73. The method of embodiment 69, wherein the method comprises administering to the human patient at a dose of about 1-40 mg/kg of the anti-TREM2 antibody.
74. The method of embodiment 69, wherein the method comprises administering to the human patient at a dose of about 10-60 mg/kg of the anti-TREM2 antibody.
75. The method of embodiment 69, wherein the method comprises administering to the human patient at a dose of about 20-40 mg/kg of the anti-TREM2 antibody.
76. The method of embodiment 69, wherein the method comprises administering to the human patient at a dose of about 1 mg/kg, about 3 mg/kg, about 10 mg/kg, about 20 mg/kg, or about 40 mg/kg of the anti-TREM2 antibody.
77. The method of any one of embodiments 69-76, wherein the method comprises administering the anti-TREM2 antibody to the human patient once weekly.
78. The method of any one of embodiments 69-76, wherein the method comprises administering the anti-TREM2 antibody to the human patient once every two weeks.
79. The method of any one of embodiments 69-76, wherein the method comprises administering the anti-TREM2 antibody to the human patient once monthly.
80. The method of any one of embodiments 69-79, wherein the method comprises administering anti-TREM2 antibody VGL101 to the human patient via an IV infusion.
81. The method of embodiment 80, wherein the IV infusion of anti-TREM2 antibody VGL101 is about 60 minutes.
82. A method for treating adult-onset leukoencephalopathy with axonal spheroids and pigmented glia (ALSP) in a human patient, comprising administering to the patient in need thereof an anti-TREM2 antibody at a dose of about 20 mg/kg, wherein the anti-TREM2 antibody is anti-TREM2 antibody VGL101, comprising a light chain having an amino acid sequence of SEQ ID NO: 9, and a heavy chain having amino acid sequence of SEQ ID NO: 10, and wherein the method comprises administering the anti-TREM2 antibody to the human patient once every 28 days via an IV infusion.
83. A method for treating adult-onset leukoencephalopathy with axonal spheroids and pigmented glia (ALSP) in a human patient, comprising administering to the patient in need thereof an anti-TREM2 antibody at a dose of about 30 mg/kg, wherein the anti-TREM2 antibody is anti-TREM2 antibody VGL101, comprising a light chain having an amino acid sequence of SEQ ID NO: 9, and a heavy chain having amino acid sequence of SEQ ID NO: 10, and wherein the method comprises administering the anti-TREM2 antibody to the human patient once every 28 days via an IV infusion.
84. A method for treating adult-onset leukoencephalopathy with axonal spheroids and pigmented glia (ALSP) in a human patient, comprising administering to the patient in need thereof an anti-TREM2 antibody at a dose of about 40 mg/kg, wherein the anti-TREM2 antibody is anti-TREM2 antibody VGL101, comprising a light chain having an amino acid sequence of SEQ ID NO: 9, and a heavy chain having amino acid sequence of SEQ ID NO: 10, and wherein the method comprises administering the anti-TREM2 antibody to the human patient once every 28 days via an IV infusion.
85. A method for treating adult-onset leukoencephalopathy with axonal spheroids and pigmented glia (ALSP) in a human patient, comprising administering to the patient in need thereof an anti-TREM2 antibody at a dose of about 50 mg/kg, wherein the anti-TREM2 antibody is anti-TREM2 antibody VGL101, comprising a light chain having an amino acid sequence of SEQ ID NO: 9, and a heavy chain having amino acid sequence of SEQ ID NO: 10, and wherein the method comprises administering the anti-TREM2 antibody to the human patient once every 28 days via an IV infusion.
86. A method for treating adult-onset leukoencephalopathy with axonal spheroids and pigmented glia (ALSP) in a human patient, comprising administering to the patient in need thereof an anti-TREM2 antibody at a dose of about 60 mg/kg, wherein the anti-TREM2 antibody is anti-TREM2 antibody VGL101, comprising a light chain having an amino acid sequence of SEQ ID NO: 9, and a heavy chain having amino acid sequence of SEQ ID NO: 10, and wherein the method comprises administering the anti-TREM2 antibody to the human patient once every 28 days via an IV infusion.
87. A method of reducing a level of sCSF1R in a subject, comprising administering to the patient in need thereof an anti-TREM2 antibody at a dose of about 20 mg/kg, wherein the anti-TREM2 antibody is anti-TREM2 antibody VGL101, comprising a light chain having an amino acid sequence of SEQ ID NO: 9, and a heavy chain having amino acid sequence of SEQ ID NO: 10, and wherein the method comprises administering the anti-TREM2 antibody to the human patient once every 28 days via an IV infusion.
88. A method of reducing a level of sCSF1R in a subject, comprising administering to the patient in need thereof an anti-TREM2 antibody at a dose of about 30 mg/kg, wherein the anti-TREM2 antibody is anti-TREM2 antibody VGL101, comprising a light chain having an amino acid sequence of SEQ ID NO: 9, and a heavy chain having amino acid sequence of SEQ ID NO: 10, and wherein the method comprises administering the anti-TREM2 antibody to the human patient once every 28 days via an IV infusion.
89. A method of reducing a level of sCSF1R in a subject, comprising administering to the patient in need thereof an anti-TREM2 antibody at a dose of about 40 mg/kg, wherein the anti-TREM2 antibody is anti-TREM2 antibody VGL101, comprising a light chain having an amino acid sequence of SEQ ID NO: 9, and a heavy chain having amino acid sequence of SEQ ID NO: 10, and wherein the method comprises administering the anti-TREM2 antibody to the human patient once every 28 days via an IV infusion.
90. A method of reducing a level of sCSF1R in a subject, comprising administering to the patient in need thereof an anti-TREM2 antibody at a dose of about 50 mg/kg, wherein the anti-TREM2 antibody is anti-TREM2 antibody VGL101, comprising a light chain having an amino acid sequence of SEQ ID NO: 9, and a heavy chain having amino acid sequence of SEQ ID NO: 10, and wherein the method comprises administering the anti-TREM2 antibody to the human patient once every 28 days via an IV infusion.
91. A method of reducing a level of sCSF1R in a subject, wherein the anti-TREM2 antibody is anti-TREM2 antibody VGL101, comprising a light chain having an amino acid sequence of SEQ ID NO: 9, and a heavy chain having amino acid sequence of SEQ ID NO: 10, and wherein the method comprises administering the anti-TREM2 antibody to the human patient once every 28 days via an IV infusion.

### EXEMPLIFICATION

Anti-TREM2 antibodies, such as “Ab-1”, can be prepared by methods known to one of ordinary skill in the art, for example, as described in WO2018/195506A1, the contents of which are incorporated herein by reference in their entireties.

### List of Abbreviations

λz: The terminal elimination rate constant
AE: Adverse event
AL: P Alkaline phosphatase
ALSP: Axonal spheroids and pigmented glia
ALT: Alanine aminotransferase
APOE: Apolipoprotein E
AST: Aspartate aminotransferase
AUC0-inf: Area under the serum or CSF concentration-time curve from predose (time 0) extrapolated to
infinite time (AUClast+Clast/5z)
AUC0-last: Area under the serum or CSF concentration-time curve from predose (time 0) to the time of
the last quantifiable concentration (tlast)
AUCtau: Area under the serum or CSF concentration-time curve over a dosing interval
AUMC: Area under the first moment curve
BMI: Body mass index
CCL2: Chemokine ligand 2
CFR: Code of Federal Regulations
CL: Total body clearance
Cmax: Maximum serum/cerebrospinal fluid concentration
CNS: Central nervous system
COVID-19: Corona virus disease-2019
CSF: Cerebrospinal fluid
CSF1R: Colony stimulating factor-1 receptor
CTR: Clinical trial report
C-SSRS: Columbia-Suicide Severity Rating Scale
Ctrough: Concentration prior to dose administration
CV %: Coefficient of variation %
ECG: Electrocardiogram
eCRF: Electronic case report form
ER: Emergency room
EOS: End of study
FDA: Food and Drug Administration
FIH: First-in-human
FSH: Follicle stimulating hormone
GCP: Good Clinical Practice
GLP: Good Laboratory Practice
HBsAg: Hepatitis B surface antigen
HIV: Human immunodeficiency virus
HSCT: Hematopoietic stem cell transplantation
IB: Investigator's Brochure
ICF: Informed consent form
ICH: International Council for Harmonisation
IL: Interleukin
IMP: Investigational medicinal product
IND: Investigational New Drug
IP-10: Interferon-gamma inducible protein of 10 kDa
IRB: Institutional Review Board
IV: Intravenously
MAD: Multiple-ascending dose
MCP-1: Monocyte chemoattractant protein-1
MDM: Monocyte-derived macrophage
MedDRA: Medical Dictionary for Regulatory Activities
MRT: Mean residence time
N: Number of observations
NfL: Neurofilament light chain
NHP: Non-human primates
NOAEL: No-observed-adverse-effect level
PCR: Polymerase chain reaction
PD: Pharmacodynamics
PI: Principal investigator
PK: Pharmacokinetics
PT: Preferred term
q7d: once every 7 days
RR: Respiratory rate
RoAUC0-tau: Accumulation ratio, AUC0-tau (last dose) divided by AUC0-tau (first dose)
RoCmax: Accumulation ratio, Cmax at last dose divided by Cmax at Day 1.
RT-PCR: Reverse transcription polymerase chain reaction
SAD: Single-ascending dose
SAE: Serious adverse event
SAP: Statistical analysis plan
SARS-CoV-2: Severe acute respiratory syndrome coronavirus 2
SD: Standard deviation
SID: Subject identification
SM: Safety margin
SOP: Standard operating procedure
SRC: Safety Review Committee
t½: Terminal elimination half-life
Tmax: Time of maximum serum/cerebrospinal fluid concentration
TREM2: Triggering receptor expressed on myeloid cells 2
Vss: Volume of distribution at steady-state
Vz: Volume of distribution
WHO: World Health Organization
WOCBP: Women of child-bearing potential

### Example 1. A Phase 1, First-In-Human, Randomized, Double-Blind, Placebo-Controlled, Single and Multiple Ascending Intravenous Dose Study to Assess the Safety, Tolerability, Pharmacokinetics of Anti-TREM2 Antibody “Ab-1” in Healthy Adults

### 1. Objectives:

### Primary:

To determine the safety and tolerability of single- and multiple-ascending intravenous (IV) doses of Anti-TREM2 antibody “Ab-1” in healthy adult participants

### Secondary:

To characterize the single-dose and the multiple dose serum pharmacokinetics (PK) of Anti-TREM2 antibody “Ab-1” IV in healthy adult participants
To assess immunogenicity of single- and multiple-ascending IV doses of Anti-TREM2 antibody “Ab-1” in healthy adult participants
To evaluate PK of Anti-TREM2 antibody “Ab-1” in cerebral spinal fluid (CSF) after single doses and multiple doses.

### Exploratory:

To explore the pharmacodynamics (PD) biomarkers in serum and CSF that may inform the Anti-TREM2 antibody “Ab-1” mechanism of action for future development
To explore whether Apolipoprotein E4 genotype confers differential response to Anti-TREM2 antibody “Ab-1”
To determine the safety and tolerability of single-ascending IV dose of Anti-TREM2 antibody “Ab-1” in healthy Japanese adult participants
To characterize the single-dose PK of Anti-TREM2 antibody “Ab-1” IV in healthy Japanese adult participants.

### 2. Design:

This is a 2-part, first-in-human (FIH), Phase 1, randomized, single-site, double-blind (Sponsor-open), placebo-controlled, dose-escalation study to evaluate the safety, tolerability, immunogenicity, PK, and PD of Anti-TREM2 antibody “Ab-1” administered IV to healthy adult participants.

Part A is a single-ascending dose (SAD) study in approximately 40 healthy adult participants. The study includes 5 cohorts (Cohorts 1A through 5A). Part A evaluates single doses of Anti-TREM2 antibody “Ab-1” administered as an IV infusion over 60 minutes at 5 different dose levels. The doses for Cohorts 1A through 5A are 1, 3, 10, 20, and 40 mg/kg; doses may be adjusted based on ongoing assessments. There is an option to include two additional optional cohorts (Cohorts 6A and 7A), where Cohort 6A is dosed below the maximum allowed exposure 60 mg/kg and Cohort 7A is a Japanese cohort. Doses during this part of the study are determined by the Sponsor as the study progresses. Safety, PK, and PD data are assessed.
(a) Sentinel dosing is planned for all SAD cohorts. The first 2 participants in each cohort are randomized and administered study treatment such that 1 participant receives Anti-TREM2 antibody “Ab-1” and 1 participant receives placebo. The remaining 6 healthy adult participants are randomized in a 5:1 ratio (Anti-TREM2 antibody “Ab-1”:placebo). The 6 remaining participants in the cohort are not administered study treatment until the investigator has reviewed safety data up to 48-hours postdose for the first 2 participants in each cohort. The cohorts are tested sequentially in a dose-escalating manner, with the available PK/PD data and at least 7 days of safety data reviewed prior to escalation to the next cohort. There should be at least 6 participants completing at least 7 days post last dose per cohort to trigger the safety review for dose-escalation; participants who discontinue or drop out are replaced at the discretion of the Sponsor.
(b) (Optional) For 36-hour continuous CSF sample collection, participants from Cohorts 2A to 5A and optional Cohort 6A are prepared with an indwelling catheter implanted in the lumbar subarachnoid space. Time points for CSF collection correspond to PK time points.
(c) Total study duration for each participant is approximately 113 days including a time period for screening, admission (Day −2) and baseline assessments up to 28 days, a treatment period of 1 day, and a follow-up period of 84 days after the Day 1 visit up to the end of study (EOS) visit.

Part B is a multiple-ascending dose (MAD) study in approximately 16 healthy adult participants. Two cohorts are planned for Part B of the study (Cohorts 1B and 2B). Cohorts 1B and 2B randomize 8 healthy adult participants to receive three IV administrations of either Anti-TREM2 antibody “Ab-1” or placebo in a 6:2 ratio during the study. Dose administration of Anti-TREM2 antibody “Ab-1” or placebo in Part B occurs every 28 days, assuming 3 infusions total are needed to reach the steady state. An optional Cohort 3B may enroll in parallel with Cohort 2B, comprising 6 participants, all of whom receive IV administrations of Anti-TREM2 antibody “Ab-1” (at or below the dose selected for Cohort 2B). Participants in Cohort 3B undergo extended CSF collection via lumbar punctures on Day 1 (predose), Day 58/59, Day 64 (admission on Day 63), Day 85 (admission on Day 84), and Day 103 (admission on Day 102). There is an option to include an additional cohort (Cohort 4B), in which 8 participants receive IV administrations of either Anti-TREM2 antibody “Ab-1” or placebo in a 6:2 ratio (60 mg/kg). Doses during this part of the study are determined as the study progresses. Safety, PK, and PD data are assessed. (a) Sentinel dosing will not be used for Part B of the study. The level of exposures intended in the MAD would have been previously shown to be safe and well-tolerated in healthy adult participants as a single dose in Part A. The cohorts are tested sequentially in a dose-escalating manner, with the available PK/PD data and at least 7 days of safety data following the last dose of study drug reviewed prior to escalation to the next cohort. There should be at least 6 participants completing at least 7 days post last dose per cohort to trigger the safety review for dose-escalation; participants who discontinue or drop out are replaced at the discretion of the Sponsor.
(b) For CSF sample collection, participants from all cohorts undergo lumbar punctures on Day 1 (predose CSF collection) and Day 58/59 (between 24-hour to 48 hours post 3rd dose). Participants in Cohort 3B undergo extended CSF collection via lumbar punctures as described above.
(c) Total study duration for each participant is approximately 169 days including a time period for screening, admission (Day −2) and baseline assessments up to 28 days, a treatment period of 57 days, and a follow-up period of 84 days after the last dosing visit (Day 57) up to the EOS visit.

### Number of Participants

Approximately 54 to 96 healthy adult participants are enrolled.

Part A: 40 to 56 healthy adult participants Approximately 40 healthy adult participants across 5 dose cohorts. An additional 16 healthy adult participants may be enrolled in two additional optional cohorts. This includes 8 Japanese participants in cohort 7A.

Part B: 16 to 30 healthy adult participants Approximately 16 healthy adult participants in 2 dose cohorts. An additional 14 healthy adult participants may be enrolled in two additional optional cohorts.

### Inclusion Criteria:

Participants who meet the following criteria are considered eligible to participate in the clinical study:
1. Participant voluntarily agrees to participate in this study and signs an Institutional Review Board (IRB) approved informed consent prior to performing any of the Screening Visit procedures.
2. Males and females between 18 to 55 years of age, inclusive, at the Screening Visit.
3. Female participants are deemed eligible if they conform to the Reproductive Inclusion criteria.
4. Nonsmokers (or other nicotine/tobacco use including vapor) as determined by history (no nicotine use over the past year) and negative urine cotinine test at the Screening Visit and/or admission (Day −2).
5. Body mass index (BMI) between 18.5 and 30.0 kg/m2, inclusive, at the Screening Visit.
6. Deemed healthy by the principal investigator (PI), determined by pre-study medical evaluation (medical history, physical examination, vital signs, 12-lead electrocardiogram [ECG], and clinical laboratory evaluations).
7. At Screening Visit, and admission (Day −2), vital signs (systolic and diastolic blood pressure and pulse rate) are assessed. If vital signs are out-of-range (at Screening Visit and admission [Day −2]), the investigator may obtain two additional vital sign sets. If vital signs are out-of-range at each of these 3 consecutive assessments and are considered to be clinically relevant per investigator judgment, the participant is not eligible for study participation.
8. The following applies to Japanese participants in the optional Cohort 7A: To be qualified as Japanese participants, the participant must meet the following criteria:
   First-generation Japanese ethnic origin:
   Born in Japan
   Parents and grandparents are ethnically Japanese and born in Japan
   No significant change in lifestyle since leaving Japan
   <10 Years outside of Japan

### Exclusion Criteria

Participants who meet one or more of the following criteria are not considered eligible to participate in the clinical study:
1. Participant has clinically significant history or evidence of cardiovascular, respiratory, hepatic, renal, gastrointestinal, endocrine, neurological, immunological, or psychiatric disorder(s) as determined by the PI or designee.
2. Participant has been administered a monoclonal antibody therapy within 120 days prior to admission (Day −2).
3. Participant has any concurrent disease or condition that, in the opinion of the PI, would make the participant unsuitable for participation in the clinical study.
4. Participant has history of alcohol and/or illicit drug abuse within 2 years of entry.
5. Participant has positive test for Hepatitis B surface antigen (HBsAg), Hepatitis C antibody, or human immunodeficiency virus (HIV) antibody.
6. Participant has positive urine test for ethanol at Screening Visit or admission (Day −2).
7. Participant has positive urine drug (e.g., cocaine, amphetamines, barbiturates, opiates, benzodiazepines, and cannabinoids) and cotinine tests at Screening Visit or admission (Day −2).
8. Female participants are breastfeeding or female participants with a positive serum pregnancy test at Screening Visit or admission (Day −2).
9. Participant has donated blood (>500 mL) or blood products within 2 months (56 days) prior to admission (Day −2).
10. Participant has been administered an investigational drug within 30 days or 5 half-lives whichever is longer prior to admission (Day −2).
11. Participant has a history of hypersensitivity to the study drug, other therapeutic monoclonal antibodies, or any of the excipients or to medicinal products with similar chemical structures.
12. Participant is unable to understand the protocol requirements, instructions and study related restrictions, the nature, scope, and possible consequences of the clinical study.
13. Participant is unlikely to comply with the protocol requirements, instructions and study related restrictions, e.g., uncooperative attitude, inability to return for follow-up visits and improbability of completing the clinical study.
14. Participant has previously been enrolled in this clinical study.
15. Vulnerable participants defined as individuals whose willingness to volunteer in a clinical study may be unduly influenced by the expectation, whether justified or not, of benefits associated with participation, or of a retaliatory response from senior members of a hierarchy in case of refusal to participate (e.g., persons in detention, minors, and those incapable of giving consent).
16. Participant has a positive reverse transcription polymerase chain reaction (RT-PCR) test for severe acute respiratory syndrome coronavirus 2 (SARS-CoV-2) prior to admission (Day −2).
17. Participant has clinical signs and symptoms consistent with SARS-CoV-2 infection, e.g., fever, dry cough, dyspnea, sore throat, fatigue, or laboratory confirmed acute infection with SARS-CoV-2.
18. Participant who had a severe course of corona virus disease 2019 (COVID-19; extracorporeal membrane oxygenation, mechanically ventilated, Intensive Care Unit stay).
19. Participant has had recent (within 14 days prior to admission [Day −2] to the Clinical Unit) exposure to someone who has COVID-19 symptoms or tested positive for SARS-CoV-2.
20. Participant has had recent (within 14 days prior to admission [Day −2] to the Clinical Unit) visit to a healthcare facility where COVID-19 patients are being treated.
21. Participant has received final dose of COVID-19 vaccine within 14 days prior to admission to the Clinical Unit (i.e., they must have completed their vaccination at least 14 days prior to admission).

### Exclusion Criteria for CSF Participants:

Participants who meet one or more of the following criteria are not considered eligible to be considered for CSF assessment:
22. Hypersensitivity to anesthetic or derivatives used during CSF collection or any medication used to prepare the area of the lumbar puncture.
23. Previous CSF collection within 30 days prior to admission (Day −2) to the Clinical Unit.
24. History of vertebral deformities, major lumbar back surgery, clinically significant back pain, clinically significant abnormal X-ray, and/or injury, in the opinion of the investigator that would preclude the participant participation or CSF collection during study.
25. An on-going skin infection at the lumbar puncture injection site.
26. Clinically significant coagulation tests values outside the normal reference range (prothrombin time/international normalized ratio, partial thromboplastin time) at Screening. Evaluation Criteria:

### Primary Endpoint:

Safety and tolerability of Anti-TREM2 antibody “Ab-1”, as assessed by adverse events (AEs), clinical laboratory tests, and vital sign measurements.

### Secondary Endpoints:

Single-dose and multiple-dose Anti-TREM2 antibody “Ab-1” serum PK parameters: e.g., area under the serum or CSF concentration-time curve from predose (time 0) to the time of the last quantifiable concentration (tlast) (AUC0-last), area under the serum or CSF concentration-time curve from predose (time 0) extrapolated to infinite time (AUClast+Clast/5z) (AUC0-inf), maximum serum/cerebrospinal fluid concentration (Cmax), time of maximum serum/cerebrospinal fluid concentration (Tmax), terminal elimination half-life (t½), total body clearance, volume of distribution at steady-state, mean residence time, area under the serum or CSF concentration-time curve over a dosing interval, concentration prior to dose administration, accumulation ratio (Cmax at last dose divided by Cmax at Day 1 and AUC0-tau [last dose] divided by AUC0-tau [first dose])
Single-dose Anti-TREM2 antibody “Ab-1” CSF PK parameters: e.g., Cmax, Tmax, AUC0-inf, AUC0-last, λz, t½ and Anti-TREM2 antibody “Ab-1” CSF concentrations on or after Day 1 and Day 58/59 after multiple dose administration
Characterization of anti-drug antibody levels after single- and multiple-dose administration of Anti-TREM2 antibody “Ab-1”

### Exploratory Endpoints:

Change from baseline in exploratory PD biomarkers (e.g., soluble Triggering Receptor Expressed on Myeloid Cells 2 [sTREM2] biomarkers, interferon-gamma inducible protein of I0 kDa [IP-10], monocyte chemoattractant protein-1 [MCP-1], colony stimulating factor-1 receptor [sCSF1R]) and neurofilament light chain [NfL]) in CSF and serum
The number and proportion of Japanese participants reporting AEs/serious adverse events (SAEs) within the dose cohorts receiving single doses
Anti-TREM2 antibody “Ab-1” single-dose serum PK parameters in Japanese participants Statistical Methods:

### Sample Size Considerations

This sample size is selected on the basis of clinical judgment and not based on statistical power calculation. Formal sample size calculations were not performed. The number of participants were chosen based on feasibility and are considered sufficient to meet the study objectives.

### Data Presentation/Descriptive Statistics

All demographic, safety and PK/PD data are listed and summarized in tabular format using descriptive statistics by cohort, ethnicity, and visit/time as appropriate. PK data (serum and CSF) are also displayed graphically as appropriate. The number and percentage of AEs are tabulated by system organ class and preferred term with a breakdown by cohort and ethnicity.

### Interim Results:

### Overview

The trial has enrolled to date 82 healthy volunteers who received either Anti-TREM2 antibody “Ab-1” (n=68) at doses of 1, 3, 10, 20, 30 or 40 mg/kg or placebo (n=14). Anti-TREM2 antibody “Ab-1” was found to be safe and well-tolerated across both the SAD and MAD cohorts dosed.
All adverse events (AEs) were mild in severity with the exception of one moderate AE, and all AEs resolved without intervention. No serious adverse events have been reported to date.
Anti-TREM2 antibody “Ab-1” showed dose proportional PK with a favorable half-life and brain penetration.
Anti-TREM2 antibody “Ab-1” achieved dose dependent, durable decreases in levels of sTREM2 in the cerebrospinal fluid (CSF) demonstrating proof of target engagement. 20 mg/kg repeat dosing was associated with robust reduction in sTREM2 levels and decreases were still observed 28 days after the third and final dose. Anti-TREM2 antibody “Ab-1” is the first antibody reported to demonstrate durability of TREM2 engagement in a clinical setting.
Anti-TREM2 antibody “Ab-1” shows durable increases in sCSF1R levels in the CSF after repeat dosing.
Dose escalation continues in the Phase 1 trial in healthy volunteers and a 60 mg/kg cohort is cleared to initiate in Australia.

In each double-blind cohort, 8 subjects were randomized to receive a single IV dose of 1, 3, 10, 20, 30 or 40 mg/kg of Anti-TREM2 antibody “Ab-1” or placebo in the SAD portion and 20 mg/kg of Anti-TREM2 antibody “Ab-1” or placebo every 28 days for a total of three administrations in the MAD cohort. Additionally, an open-label portion of the trial is being conducted for the collection of CSF. Preliminary safety and tolerability data are available for one CSF SAD cohort receiving single IV doses of 3, 10, or 20 mg/kg of VGL101 and one CSF MAD cohort receiving 20 mg/kg of VGL101 every 28 days for a total of three drug administrations.

### Safety & Tolerability

Across cohorts, all AEs were mild with the exception of one moderate AE and all AEs resolved without intervention. There were no reports of serious AEs (SAEs) or clinically meaningful abnormalities in Vital signs, ECGs, or Laboratory parameters.

### Single Ascending Dose Pharmacokinetics (PK)

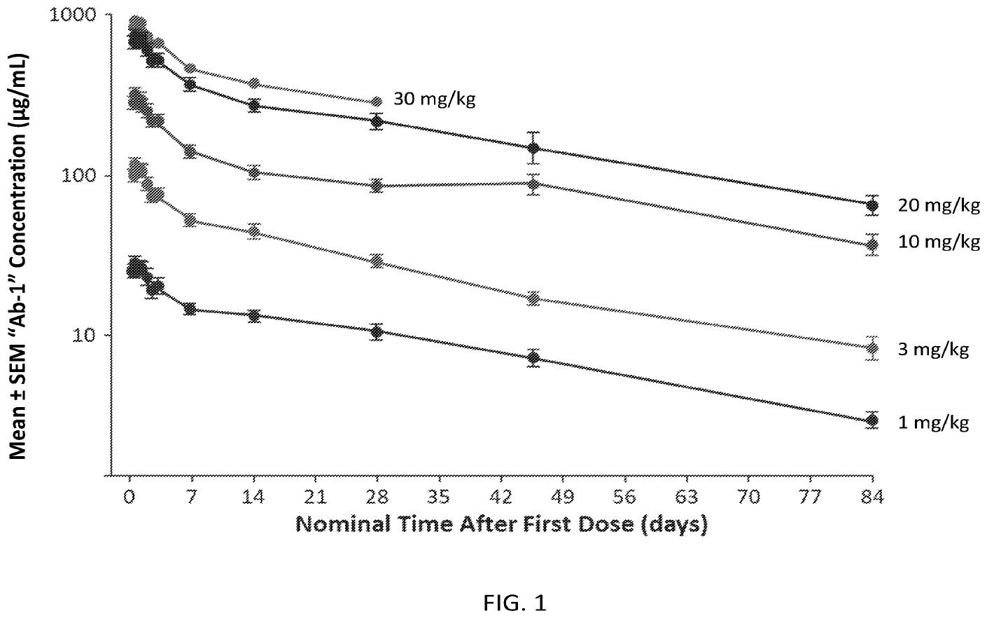

FIG. 1 shows serum PK data from the SAD cohorts. Doses up to 30 mg/kg through 28 days and doses of 1, 3, 10 and 20 mg/kg through 84 days have been evaluated. All available doses up to 30 mg/kg exhibit linear and dose-proportional PK with low variability.

### Multiple Ascending Dose Pharmacokinetics (PK)

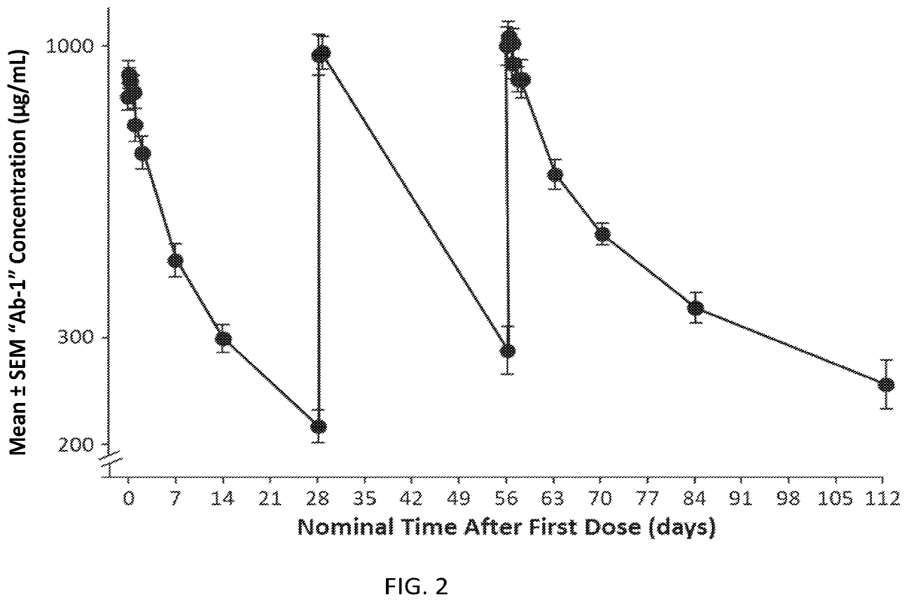

FIG. 2 shows serum PK data from the first MAD cohort in which 20 mg/kg of Anti-TREM2 antibody “Ab-1” was administered every 28 days for a total of three infusions. The data indicate a half-life of approximately 27 days, with a steady state achieved between the 3rd and 4th dose, supporting a monthly dosing regimen.

The brain penetration of Anti-TREM2 antibody “Ab-1” is between 0.1-0.2%.

### Pharmacodynamics

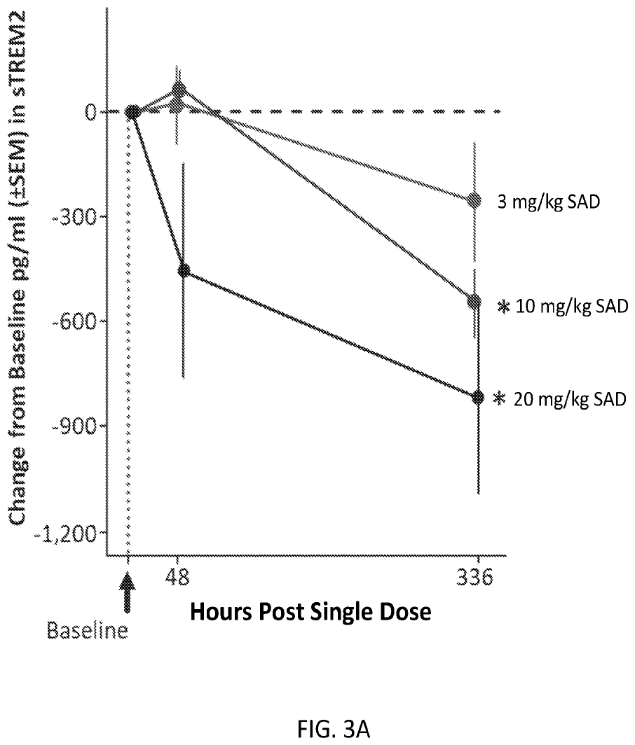

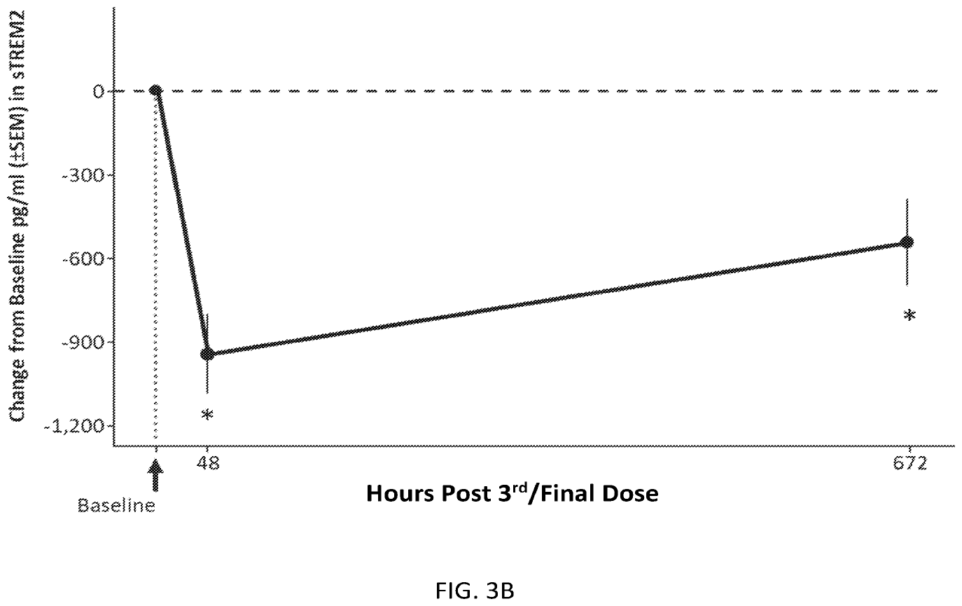

Based on preclinical work, a reduction in levels of soluble TREM2 (sTREM2), a proximal biomarker for engagement of the TREM2 receptor, was predicted. FIG. 3A shows the effects of Anti-TREM2 antibody “Ab-1” on CSF sTREM2 in 18 of healthy volunteers administered single IV doses of Anti-TREM2 antibody “Ab-1” at 3, 10 and 20 mg/kg. There were dose-dependent reductions of sTREM2 over baseline, with sTREM2 reduction being statistically significant at 2 weeks following a single dose of Anti-TREM2 antibody “Ab-1” at 10 and 20 mg/kg.

FIG. 3B shows Anti-TREM2 antibody “Ab-1” 's effect on sTREM2 following 3 IV doses at 20 mg/kg given once every 28 days. CSF levels of sTREM2 were evaluated at baseline (prior to dosing), 48 hours, and 28 days following the 3rd and final dose. These data show statistically significant reduction of sTREM2 over the pre-dosing baseline following repeat dosing of Anti-TREM2 antibody “Ab-1” at 20 mg/kg and, notably, at a greater magnitude and with less variability than the single dose at the 48-hour timepoint. Similarly, sTREM2 reduction is sustained through 28 days after the 3rd and final dose with less variability than the single dose. These data serve as evidence of durability of target engagement by Anti-TREM2 antibody “Ab-1” in humans following repeat dosing through 28 days.

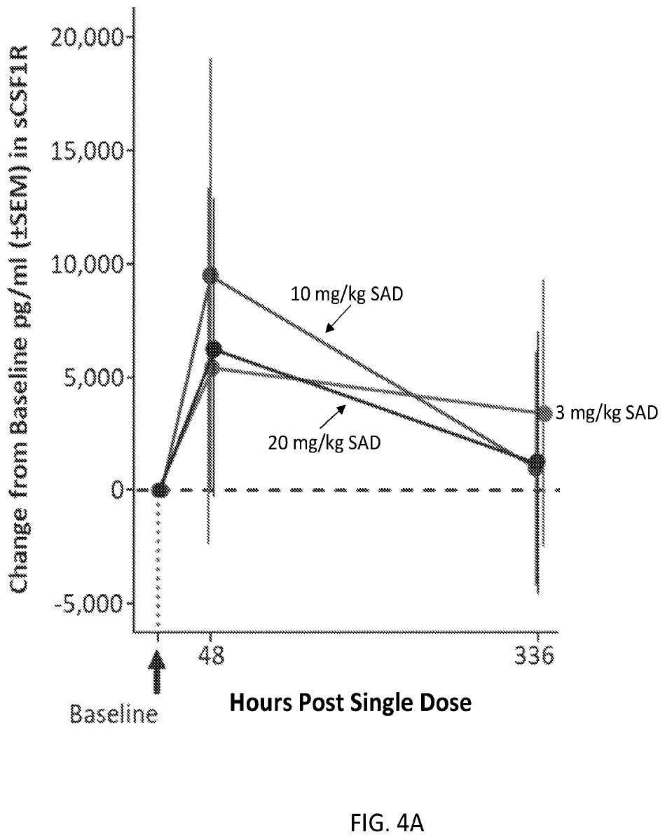

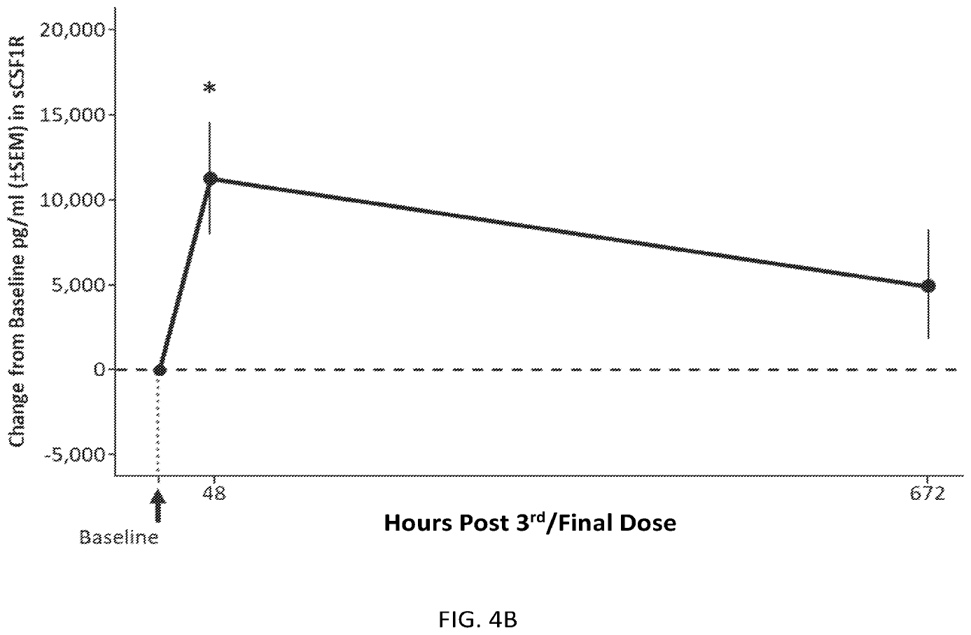

Soluble CSF1R (sCSF1R) is another biomarker for target engagement that lies downstream of sTREM2 and microglia activation. Based on preclinical data, an increase in sCSF1R levels in the CSF was expected. FIGS. 4A-4B demonstrate the increase in sCSF1R and target engagement by Anti-TREM2 antibody “Ab-1” following single and repeat dosing, respectively, as was observed for sTREM2. Decreasing variability was again observed with repeat dosing.

While we have described a number of embodiments of this invention, it is apparent that our basic examples may be altered to provide other embodiments that utilize the compounds and methods of this invention. Therefore, it will be appreciated that the scope of this invention is to be defined by the application and claims rather than by the specific embodiments that have been represented by way of example.

1. A method for treating adult-onset leukoencephalopathy with axonal spheroids and pigmented glia (ALSP) in a human patient, comprising administering to the patient in need thereof an anti-TREM2 antibody at a dose of about 1-100 mg/kg, wherein the anti-TREM2 antibody comprises:
(a) a light chain variable region comprising a CDRL1 having an amino acid sequence according to SEQ ID NO: 2; a CDRL2 having an amino acid sequence according to SEQ ID NO: 3; and a CDRL3 having an amino acid sequence according to SEQ ID NO: 4, and a heavy chain variable region comprising a CDRH1 having an amino acid sequence according to SEQ ID NO: 6; a CDRH2 having an amino acid sequence according to SEQ ID NO: 7; and a CDRH3 having an amino acid sequence according to SEQ ID NO: 8; or
(b) a light chain variable region having an amino acid sequence according to SEQ ID NO: 1, and a heavy chain variable region having an amino acid sequence according to SEQ ID NO: 5.

2. The method of claim 1, wherein the anti-TREM2 antibody:
(i) is an IgG, optionally an IgG₁;
(ii) is an IgG₁ comprising a variant constant region having one or more mutations selected from R292C, N297G, V302C, D356E, or L358M, according to EU numbering; and/or
(iii) comprises a kappa light constant region.

3. The method of claim 1, wherein the anti-TREM2 antibody is anti-TREM2 antibody VGL101, comprising a light chain having an amino acid sequence of SEQ ID NO: 9, and a heavy chain having amino acid sequence of SEQ ID NO: 10.

4. The method of claim 1, wherein the method comprises administering to the human patient at a dose of about 1-60 mg/kg of the anti-TREM2 antibody.

5. The method of claim 1, wherein the method comprises administering to the human patient at a dose of about 1 mg/kg, about 3 mg/kg, about 10 mg/kg, about 20 mg/kg, or about 40 mg/kg of the anti-TREM2 antibody.

6. The method of claim 1, wherein the method comprises administering the anti-TREM2 antibody to the human patient once weekly via an IV infusion, optionally wherein the IV infusion is about 60 minutes.

7. A liquid formulation comprising anti-TREM2 antibody VGL101 at a concentration of about 140 mg/mL, and a pharmaceutically acceptable excipient and/or carrier.

8. The liquid formulation of claim 7, further comprising one or more of:
(a) sodium acetate at a concentration of about 15 mM;
(b) sucrose at a concentration of about 9% (w/v) of total liquid formulation volume;
(c) Polysorbate 80 at a concentration of about 0.01% (w/v) of total liquid formulation volume;
and/or
(d) a pH at about 5.2.

9. A liquid formulation at about pH 5.2, comprising:
anti-TREM2 antibody VGL101 at a concentration of about 140 mg/mL;
sodium acetate at a concentration of about 15 mM;
sucrose at a concentration of about 9% (w/v) of total liquid formulation volume; and
Polysorbate 80 at a concentration of about 0.01% (w/v) of total liquid formulation volume.

10. A method for treating adult-onset leukoencephalopathy with axonal spheroids and pigmented glia (ALSP) in a human patient, comprising administering to the patient in need thereof a therapeutically effective amount of the liquid formulation of claim 9.

11. The method of claim 10, wherein the method comprises administering to the human patient at a dose of about 1-60 mg/kg of anti-TREM2 antibody VGL101, optionally at a dose of about 20-40 mg/kg of the anti-TREM2 antibody.

12. The method of claim 10, wherein the method comprises administering to the human patient at a dose of about 1 mg/kg, about 3 mg/kg, about 10 mg/kg, about 20 mg/kg, or about 40 mg/kg of anti-TREM2 antibody VGL101.

13. The method of claim 10, wherein the method comprises administering anti-TREM2 antibody VGL101 to the human patient once weekly via an IV infusion, optionally wherein the IV infusion is about 60 minutes.

14. A method of reducing a level of sTREM2 or sCSF1R in a subject, comprising administering anti-TREM2 antibody VGL101 to the subject at a dose of about 1-100 mg/kg.

15. The method of claim 14, wherein the level of sTREM2 or sCSF1R is evaluated prior to administering VGL101.

16. The method of claim 14, wherein the level of sTREM2 or sCSF1R is the level in cerebrospinal fluid (CSF).

17. The method of any one of claim 14, wherein the level of sTREM2 or sCSF1R is reduced by about 10%, about 20%, about 30%, about 40%, about 50%, about 60%, about 70%, about 80, about 90%, or about 100%.

18. The method of claim 14, wherein the method comprises administering to the human patient at a dose of about 1-60 mg/kg of anti-TREM2 antibody VGL101, optionally at a dose of about 20-40 mg/kg of the anti-TREM2 antibody.

19. The method of claim 14, wherein the method comprises administering to the human patient at a dose of about 1 mg/kg, about 3 mg/kg, about 10 mg/kg, about 20 mg/kg, or about 40 mg/kg of the anti-TREM2 antibody VGL101.

20. The method of claim 14, wherein the method comprises administering the anti-TREM2 antibody to the human patient once weekly via an IV infusion, optionally wherein the IV infusion is about 60 minutes.

21. A method of engaging or agonizing TREM2 in a subject, comprising administering anti-TREM2 antibody VGL101 to the subject at a dose of about 1-100 mg/kg.

22. The method of claim 21, wherein the method comprises administering to the human patient at a dose of about 1-60 mg/kg of the anti-TREM2 antibody, optionally at a dose of about 20-40 mg/kg of the anti-TREM2 antibody.

23. The method of claim 21, wherein the method comprises administering to the human patient at a dose of about 1 mg/kg, about 3 mg/kg, about 10 mg/kg, about 20 mg/kg, or about 40 mg/kg of the anti-TREM2 antibody.

24. The method of claim 21, wherein the method comprises administering the anti-TREM2 antibody to the human patient once weekly via an IV infusion, optionally wherein the IV infusion is about 60 minutes.
